(19) United States
(12) Patent Application Publication
(10) Pub. No.: US 2026/0144872 A1
(43) Pub. Date: May 28, 2026

(54) GENETIC EDITING OF TARGET GENES TO ENHANCE NATURAL KILLER CELL FUNCTION

(71) Applicant: NKARTA, INC., South San Francisco, CA (US)

(72) Inventors: James Barnaby Trager, South San Francisco, CA (US); Ivan Chan, South San Francisco, CA (US); Chao Guo, South San Francisco, CA (US)

(21) Appl. No.: 18/997,909

(22) PCT Filed: Aug. 2, 2023
(86) PCT No.: PCT/US2023/071537

§ 371 Date: Jan. 23, 2025

Related U.S. Application Data
(60) Provisional application No. 63/370,357, filed on Aug. 3, 2022.
(60) Provisional application No. 63/489,965, filed on Mar. 13, 2023.
(60) Provisional application No. 63/498,166, filed on Apr. 25, 2023.

(51) Int. Cl. A61K 40/15; A61K 35/17; A61K 40/32; C07K 14/47; C07K 14/54; C07K 14/725; C12N 5/0783; C12N 5/10; C12N 9/22; C12N 15/113

(52) U.S. Cl. CPC A61K 40/15; A61K 35/17; A61K 40/32; C07K 14/4702; C07K 14/5443; C07K 14/7051; C12N 5/0646; C12N 5/10; C12N 9/222; C12N 15/113; C07K 2319/02; C07K 2319/03; C12N 2510/00

(57) ABSTRACT

Several embodiments of the methods and compositions disclosed herein relate to immune cells that are genetically edited, for example, using Crispr/Cas, to modulate, reduce or otherwise eliminate expression of one or more endogenous genes. In several embodiments, the edited cells are engineered to express a chimeric antigen receptor targeting a tumor antigen, for example CD19, ligands of the NKG2D receptor, CD70, and/or BCMA, among others. In several embodiments, the editing enhances one or more aspects of the efficacy of the immune cells in cellular immunotherapy including cytotoxicity (e.g., ADCC) and/or persistence.

### CROSS-REFERENCE TO RELATED APPLICATIONS

This application claims priority to U.S. Provisional Patent Application No. 63/370,357, filed Aug. 3, 2022, U.S. Provisional Patent Application No. 63/489,965, filed Mar. 13, 2023, and U.S. Provisional Patent Application No. 63/498,166, filed Apr. 25, 2023, the entire contents of each of which is incorporated by reference herein.

### FIELD

Several embodiments disclosed herein relate to methods and compositions comprising genetically engineered and edited immune cells for cancer immunotherapy. In several embodiments, the present disclosure relates to cells engineered to express chimeric antigen receptors (CAR). In several embodiments, the cells expressing the CAR are also genetically edited in order to enhance their expansion, cytotoxicity against target cells, persistence (e.g., lifespan) after administration, and/or to reduce potential side effects when the cells are used in cancer immunotherapy.

### BACKGROUND

As further knowledge is gained about various cancers and what characteristics a cancerous cell has that can be used to specifically distinguish that cell from a healthy cell, therapeutics are under development that leverage the distinct features of a cancerous cell. Immunotherapies that employ engineered and/or edited immune cells are one approach to treating cancers.

### INCORPORATION BY REFERENCE OF MATERIAL IN SEQUENCE LISTING FILE

This application incorporates by reference the material contained in the Sequence Listing XML file being submitted concurrently herewith: File name: NKT089WO_ST26.xml; created Aug. 2, 2023, 1,364,361 bytes in size.

### SUMMARY

Immunotherapy presents a new technological advancement in the treatment of disease, wherein immune cells are engineered to express certain targeting and/or effector molecules that specifically identify and react to diseased or damaged cells. This represents a promising advance due, at least in part, to the potential for specifically targeting diseased or damaged cells, as opposed to more traditional approaches, such as chemotherapy, where all cells are impacted, and the desired outcome is that sufficient healthy cells survive to allow the patient to live. One immunotherapy approach is the recombinant expression of cytotoxic receptors (e.g., chimeric receptors) in immune cells to achieve the targeted recognition and destruction of aberrant cells of interest.

In several embodiments, there is provided herein population of genetically engineered and gene edited immune cells, comprising genetically engineered immune cells that express a cytotoxic receptor comprising an extracellular ligand binding domain, a transmembrane domain, and a cytotoxic signaling complex, wherein the extracellular ligand binding domain targets an antigen expressed by cells of a target tumor or cancer the immune cells are genetically edited within a target sequence in a MED12 gene and within a target sequence in a CISH gene; and the edits yield reduced expression and/or function of each of the MED12 protein encoded by the MED12 gene and the CIS protein encoded by the CISH gene, as compared to an immune cell not edited within the target sequence in the MED12 gene and the target sequence within the CISH gene.

In several embodiments, the target sequence within the MED12 gene comprises any one of SEQ ID NOS: 997, 938-944, 996, or 998. In several embodiments, the target sequence within the MED12 gene comprises a plurality of target sites selected from SEQ ID NOS: 997, 938-944, 996, and 998.

In several embodiments, the target sequence in the CISH gene comprises any one of SEQ ID NOS: 1013, 153-157 or 463-466 or 1012. In several embodiments, the target sequence in the CISH gene comprises a plurality of target sites selected from SEQ ID NOS: 1013, 153-157 or 463-466 and 1012.

In several embodiments, the extracellular ligand binding domains targets an antigen selected from BCMA, a NKG2D ligand, CD19, and CD70. In several embodiments, the extracellular ligand binding domains targets a BCMA antigen. In several embodiments, the extracellular ligand binding domains targets an NKG2D ligand. In several embodiments, the extracellular ligand binding domains targets a CD19 antigen. In several embodiments, the extracellular ligand binding domains targets a CD70 antigen.

In several embodiments, the transmembrane domain comprises CD8, CD28, or a portion thereof, optionally wherein the transmembrane domain comprises CD8a or a portion thereof. In several embodiments, the transmembrane domain comprises CD8 or a portion thereof. In several embodiments, the transmembrane domain comprises CD28, or a portion thereof. Depending on the embodiment the transmembrane domain optionally comprises CD8a or a portion thereof, in combination with CD8 or CD28.

In several embodiments, the cytotoxic signaling complex comprises a CD3zeta domain and/or the cytotoxic signaling complex comprises an intracellular signaling domain of an OX40, 4-1BB, CD28, or a signaling portion thereof, optionally an intracellular signaling domain of OX40 or a signaling portion thereof.

In several embodiments, at least a portion of the genetically engineered immune cells are engineered to express membrane bound IL-15 (mbIL15). In some such embodiments, the cytotoxic receptor and the mbIL15 are optionally encoded by the same nucleic acid molecule, optionally wherein the nucleic acid sequences encoding the cytotoxic receptor and the mbIL15 are separated by a nucleic acid sequence encoding a 2A peptide. In some such embodiments, wherein the cytotoxic receptor and the mbIL15 are encoded by the same nucleic acid molecule, optionally wherein the nucleic acid sequences encoding the cytotoxic receptor and the mbIL15 are separated by a nucleic acid sequence encoding a 2A peptide. In some such embodiments, wherein the cytotoxic receptor and the mbIL15 are encoded by the same nucleic acid molecule, wherein the nucleic acid sequences encoding the cytotoxic receptor and the mbIL15 are separated by a nucleic acid sequence encoding a 2A peptide.

In several embodiments, the cells are further genetically edited within a target sequence in the CBLB gene, wherein the target sequence in the CBLB gene comprises any one of SEQ ID NOS: 164, 165-166 or 453-456 or 1005-1008.

In several embodiments, the cells are further genetically edited within a target sequence in a disintegrin and metalloproteinase domain-containing protein 17 (ADAM17) gene comprising any one of SEQ ID NOS: 682-687.

In several embodiments, the cells are further genetically edited within a target sequence in a hypoxia-inducible factor 1-alpha (HIF1-a) gene comprising any one of SEQ ID NOS: 750-760.

In several embodiments, the cells are further genetically edited within a target sequence in a DGKz gene, and the target sequence comprises any one of SEQ ID NOS: 688-723.

In several embodiments, the cells are further genetically edited within a target sequence in a GSK-3B gene, and the target sequence comprises any one of SEQ ID NOS: 724-749.

In several embodiments, the cells are further genetically edited within a target sequence in a LAG3 gene, and the target sequence comprises any one of SEQ ID NOS: 761-789.

In several embodiments, the cells are further genetically edited within a target sequence in a TIM3 gene, and the target sequence comprises any one of SEQ ID NOS: 790-825.

In several embodiments, the cells are further genetically edited within a target sequence in a TRIM29 gene, and the target sequence comprises any one of SEQ ID NOS: 826-835 or 1009-1011.

In several embodiments, the cells are further genetically edited within a target sequence in a IL-1R8 gene, and the target sequence comprises any one of SEQ ID NOS: 836-865.

In several embodiments, the cells are further genetically edited within a target sequence in a CD38 gene, and the target sequence comprises any one of SEQ ID NOS: 866-874.

In several embodiments, the cells are further genetically edited within a target sequence in a FBP-1 gene, and the target sequence comprises any one of SEQ ID NOS: 875-889.

In several embodiments, the cells are further genetically edited within a target sequence in a INSIG1 gene, and the target sequence comprises any one of SEQ ID NOS: 890-934.

In several embodiments, the cells are further genetically edited within a target sequence in a CDK8 gene, and the target sequence comprises any one of SEQ ID NOS: 949-955.

In several embodiments, the cells are further genetically edited within a target sequence in a CCNC gene, and the target sequence comprises any one of SEQ ID NOS: 956-961 or 999-1001.

In several embodiments, the cells are further genetically edited within a target sequence in a ID3 gene, and the target sequence comprises any one of SEQ ID NOS: 963-969.

In several embodiments, the cells are further genetically edited within a target sequence in a SOX4 gene, and the target sequence comprises any one of SEQ ID NOS: 970-976.

In several embodiments, the edit to the target sequence or target sequences is made using an RNA-guided endonuclease. In several embodiments, the edit to the target sequence or target sequences is made using a Crispr/Cas9 system.

In several embodiments, the immune cells comprise Natural Killer (NK) cells, T cells, induced pluripotent stem cells (iPSCs), iPSC-derived NK cells, iPSC-derived T cells, NK-92 cells, or any combination thereof.

In several embodiments, there is provided a population of gene edited immune cells, wherein the immune cells are genetically edited within a target sequence in a MED12 gene and within a target sequence in a CISH gene and the edits yield reduced expression and/or function of each of the MED12 protein encoded by the MED12 gene and the CIS protein encoded by the CISH gene, as compared to an immune cell not edited within the target sequence in the MED12 gene and the target sequence within the CISH gene. In several embodiments, the gene edited immune cells are genetically engineered immune cells that express a cytotoxic receptor comprising an extracellular ligand binding domain, a transmembrane domain, and a cytotoxic signaling complex and the extracellular ligand binding domain targets an antigen expressed by cells of a target tumor or cancer.

In several embodiments, there is provided for a population of genetically engineered and gene edited immune cells that express a cytotoxic receptor comprising an extracellular ligand binding domain, a transmembrane domain, and a cytotoxic signaling complex, wherein the extracellular ligand binding domain targets an antigen expressed by cells of a target tumor or cancer, the immune cells are genetically edited within a target sequence in a MED12 gene, wherein the target sequence within the MED12 gene comprises any one of SEQ ID NOS: 997, 938-944, 996, or 998, and the edits yield reduced expression and/or function of the MED12 protein encoded by the MED12 gene, as compared to an immune cell not edited within the target sequence in the MED12 gene.

In several embodiments, there is a provided a composition comprising a population of genetically engineered and/or gene edited immune cells as disclosed herein.

In several embodiments, there is provided a method for the treatment of a subject having a disease or condition comprising administering to the subject a population of genetically engineered and gene edited immune cells as disclosed herein.

In several embodiments, there is provided a use of a population of genetically engineered and edited immune cells as disclosed herein for the treatment of a subject having a disease or condition. In several embodiments, the wherein the disease or condition is an infectious disease, an autoimmune disease, a cancer, or a tumor.

In several embodiments, the immune cells are NK cells.

Also provided herein is a population of gene edited and genetically engineered immune cells comprising immune cells that are (i) genetically engineered to express a cytotoxic receptor and (ii) genetically edited within a target sequence in a gene selected from among the group consisting of ADAM17, HIF-1a, DGKz, GSK-3B, LAG3. TIM3, TRIM29, IL-1R8, CD38, FBP-1, INSIG1, MED12, MED 13, CCNC, CDK8. ID3, and SOX4.

In several embodiments, the immune cells are genetically edited using an RNA-guided endonuclease.

In several embodiments, there is provided a population of gene edited immune cells comprising immune cells that are genetically edited within a target sequence in the MED12 gene. In some embodiments, the immune cells are genetically engineered to express a cytotoxic receptor. In several embodiments, there is provided a population of gene edited and genetically engineered immune cells comprising immune cells that are (i) genetically engineered to express a cytotoxic receptor and (ii) genetically edited within a target sequence in the MED12 gene.

In some embodiments, the cytotoxic receptor comprises an extracellular ligand binding domain, a transmembrane domain, and a cytotoxic signaling complex. In some embodiments, the extracellular ligand binding domain binds to an antigen expressed by cells of a target tumor or cancer. In some embodiments, the genetic edit is within a target sequence in the MED12 gene comprising any one of SEQ ID NOs: 938-994 or 996-998. In some embodiments, the genetic edit to MED12 reduces expression and/or function of the MED12 protein encoded by the MED12 gene, as compared to an immune cell not edited within the target sequence. In some embodiments, the genetic edit to MED12 reduces expression of the MED12 protein encoded by the MED12 gene, as compared to an immune cell not edited within the target sequence. In some embodiments, the genetic edit to MED12 reduces expression function of the MED12 protein encoded by the MED12 gene, as compared to an immune cell not edited within the target sequence. In some embodiments, the genetic edit to MED12 reduces expression and function of the MED12 protein encoded by the MED12 gene, as compared to an immune cell not edited within the target sequence. In several embodiments, the edit to the MED12 gene is made using an RNA-guided endonuclease. In some embodiments, the immune cells are natural killer cells.

In several embodiments, there is provided a population of gene edited immune cells comprising immune cells that are genetically edited within a target sequence in the ADAM17 gene. In some embodiments, the immune cells are genetically engineered to express a cytotoxic receptor. In some embodiments, the cytotoxic receptor comprises an extracellular ligand binding domain, a transmembrane domain, and a cytotoxic signaling complex. In some embodiments, the extracellular ligand binding domain binds to an antigen expressed by cells of a target tumor or cancer. In some embodiments, the genetic edit within a target sequence in the ADAM17 gene reduces expression and/or function of the ADAM17 protein encoded by the ADAM17 gene, as compared to an immune cell not edited within the target sequence. In some embodiments, the genetic edit within a target sequence in the ADAM17 gene reduces expression of the ADAM17 protein encoded by the ADAM17 gene, as compared to an immune cell not edited within the target sequence. In some embodiments, the genetic edit within a target sequence in the ADAM17 gene reduces function of the ADAM17 protein encoded by the ADAM17 gene, as compared to an immune cell not edited within the target sequence. In some embodiments, the genetic edit within a target sequence in the ADAM17 gene reduces expression and function of the ADAM17 protein encoded by the ADAM17 gene, as compared to an immune cell not edited within the target sequence. In several embodiments, there is provided a population of genetically engineered and gene edited immune cells, comprising genetically engineered immune cells that express a cytotoxic receptor comprising an extracellular ligand binding domain, a transmembrane domain, and a cytotoxic signaling complex, wherein the extracellular ligand binding domain targets an antigen expressed by cells of a target tumor or cancer, the immune cells are genetically edited within a target sequence in a disintegrin and metalloproteinase domain-containing protein 17 (ADAM17) gene, the edit yields reduced expression and/or function of an ADAM17 protein encoded by the ADAM17 gene, as compared to an immune cell not edited within the target sequence in the ADAM17 gene, and the edit to the ADAM17 gene is made using an RNA-guided endonuclease.

In several embodiments, there is provided a population of genetically edited immune cells comprising immune cells that are genetically edited within a target sequence in the MED12 gene. In several embodiments, the immune cells are also genetically edited within a target sequence in the CISH gene. In several embodiments, there is provided a population of gene edited immune cells comprising immune cells that are (i) genetically edited within a target sequence in the MED12 gene; and (ii) genetically edited within a target sequence in the CISH gene. In several embodiments, there is provided a population of genetically edited immune cells comprising immune cells that are genetically edited within a target sequence in the MED12 gene and within a target sequence in the CISH gene. In some embodiments, the immune cells are genetically engineered to express a cytotoxic receptor. In some embodiments, the cytotoxic receptor comprises an extracellular ligand binding domain, a transmembrane domain, and a cytotoxic signaling complex. In some embodiments, the extracellular ligand binding domain binds to an antigen expressed by cells of a target tumor or cancer. In some embodiments, the genetic edit within a target sequence in the MED12 gene reduces expression and/or function of the MED12 protein encoded by the MED12 gene, as compared to an immune cell not edited within the target sequence. In some embodiments, the genetic edit within a target sequence in the CISH gene reduces expression and/or function of the CIS protein encoded by the CIS gene, as compared to an immune cell not edited within the target sequence. In some embodiments, the genetic edit within a target sequence in the CISH gene reduces expression of the CIS protein encoded by the CIS gene, as compared to an immune cell not edited within the target sequence. In some embodiments, the genetic edit within a target sequence in the CISH gene reduces or function of the CIS protein encoded by the CIS gene, as compared to an immune cell not edited within the target sequence. In some embodiments, the genetic edit within a target sequence in the CISH gene reduces expression and function of the CIS protein encoded by the CIS gene, as compared to an immune cell not edited within the target sequence. In several embodiments, there is provided a population of genetically engineered and gene edited immune cells, comprising genetically engineered immune cells that express a cytotoxic receptor comprising an extracellular ligand binding domain, a transmembrane domain, and a cytotoxic signaling complex, wherein the extracellular ligand binding domain targets an antigen expressed by cells of a target tumor or cancer, the immune cells are genetically edited within a target sequence in the MED12 gene, the edit yields reduced expression and/or function of the mediator complex subunit 12 (MED12) protein encoded by the MED12 gene, as compared to an immune cell not edited within the target sequence in the MED12 gene, and the edit to the MED12 gene is made using an RNA-guided endonuclease. In several embodiments, there is provided a population of genetically engineered and gene edited immune cells, comprising genetically engineered immune cells that express a cytotoxic receptor comprising an extracellular ligand binding domain, a transmembrane domain, and a cytotoxic signaling complex, wherein the extracellular ligand binding domain targets an antigen expressed by cells of a target tumor or cancer, the immune cells are genetically edited within a target sequence in the MED12 gene and within a target sequence in the CISH gene, the edits yield reduced expression and/or function of the mediator complex subunit 12 (MED12) protein and the CIS protein, as compared to an immune cell not edited within the target sequence in the MED12 and CISH genes, and the edits are made using an RNA-guided endonuclease. In some embodiments, the immune cells are natural killer cells.

Also provide herein is a population of genetically engineered and gene edited immune cells, comprising genetically engineered immune cells that express a cytotoxic receptor comprising an extracellular ligand binding domain, a transmembrane domain, and a cytotoxic signaling complex, wherein the extracellular ligand binding domain targets an antigen expressed by cells of a target tumor or cancer, the immune cells are genetically edited within a target sequence in a hypoxia-inducible factor 1-alpha (HIF1-a) gene, the edit yields reduced expression and/or function of the HIF1-a protein encoded by the HIF1-a gene, as compared to an immune cell not edited within the target sequence in the HIF1-a gene, and the edit to the HIF1-a gene is made using an RNA-guided endonuclease.

In some such embodiments, the immune cells are optionally edited within an additional target sequence in a target gene to yield reduced levels of expression of a protein encoded by the target gene, as compared to an immune cell not edited within the additional target sequence.

In additional embodiments, there is provided a population of genetically engineered and gene edited immune cells, comprising genetically engineered immune cells that express a cytotoxic receptor comprising an extracellular ligand binding domain, a transmembrane domain, and a cytotoxic signaling complex, wherein the extracellular ligand binding domain targets an antigen expressed by cells of a target tumor or cancer, the immune cells are genetically edited within a target sequence in a target gene selected from ADAM17, HIF-1a, DGKz, GSK-3B, LAG3, TIM3, TRIM29, IL-1R8, CD38, FBP-1, INSIG1, MED12, MED 13. CCNC, CDK8, ID3, SOX4, and any combination thereof, the edit yields reduced expression and/or function of the protein encoded by the target gene, as compared to an immune cell not edited within the target sequence in the target gene, the immune cells are edited at an additional target sequence within a target gene to yield reduced levels of expression of the protein encoded by the target gene, as compared to an immune cell not edited at the additional target sequence, and the edits to the target gene are made using an RNA-guided endonuclease.

In several embodiments, the edit to the target gene is made using a Crispr/Cas9 system.

In several embodiments, the extracellular ligand binding domains targets an antigen selected from a ligand of NKG2D, CD19, CD70, and BCMA. In several embodiments, the extracellular ligand binding domains targets a ligand of NKG2D. In several embodiments, the extracellular ligand binding domains targets CD19. In several embodiments, the extracellular ligand binding domains targets CD70. In several embodiments, the extracellular ligand binding domains targets BCMA.

In several embodiments, there is provided a population of genetically engineered and gene edited immune cells, comprising genetically engineered immune cells that express a cytotoxic receptor comprising an extracellular ligand binding domain, a transmembrane domain, and a cytotoxic signaling complex, wherein the extracellular ligand binding domain targets an antigen expressed by cells of a target tumor or cancer selected from a ligand of the NKG2D receptor, CD19, CD70, and BCMA, the immune cells are genetically edited within a target sequence in a target gene selected from ADAM17, HIF-1a, DGKz, GSK-3B, LAG3, TIM3, TRIM29, IL-1R8, CD38, FBP-1, INSIG1, MED12, MED13, CCNC, CDK8, ID3, SOX4, and any combination thereof, the edit yields reduced expression and/or function of the protein encoded by the target gene, as compared to an immune cell not edited within the target sequence in the target gene, the immune cells are edited within an additional target sequence in a target gene to yield reduced levels of expression of the protein encoded by the target gene, as compared to an immune cell not edited within the additional target sequence, and the edit(s) to the target gene(s) are made using a Crispr/Cas system.

In several embodiments, the genetically engineered and gene edited immune cells provided for herein exhibit enhanced expansion capability, enhanced cytotoxicity against target tumor cells, enhanced persistence, or any combination thereof, as compared to immune cells that do not comprise the edit(s).

In several embodiments, there is provided a method of manufacturing a population of genetically edited immune cells comprising contacting the population of immune cells with a targeted endonuclease that edits within a target sequence in a target gene selected from ADAM17, HIF-1a, DGKz, GSK-3B, LAG3, TIM3, TRIM29, IL-1R8, CD38, FBP-1, INSIG1, MED12, MED13, CCNC, CDK8, ID3, SOX4, CISH, CBLB, and any combination thereof, wherein the genetically edited immune cells exhibit: enhanced expansion capability, enhanced cytotoxicity against target tumor cells, enhanced persistence, or any combination thereof, as compared to immune cells that have not been edited within the target sequence in the target gene.

In several embodiments, there is provided a method of manufacturing a population of genetically edited immune cells comprising contacting the population of immune cells with a RNA guided endonuclease that edits within a target sequence in a target gene selected from ADAM17, HIF-la, DGKz, GSK-3B, LAG3, TIM3, TRIM29, IL-1R8, CD38, FBP-1, INSIG1, MED12, MED13, CCNC, CDK8, ID3, SOX4, and any combination thereof, wherein the genetically edited immune cells exhibit: enhanced expansion capability, enhanced cytotoxicity against target tumor cells, enhanced persistence, or any combination thereof, as compared to immune cells that have not been edited within the target sequence in the target gene.

In several embodiments, there is provided a method of manufacturing a population of genetically edited immune cells comprising contacting the population of immune cells with a Cas-gRNA ribonucleoprotein complex (RNP), wherein: the RNP edits within a target sequence in a target gene selected from ADAM17, HIF-1a, DGKz, GSK-3B, LAG3, TIM3, TRIM29, IL-1R8, CD38, FBP-1, INSIG1, MED12, MED13, CCNC, CDK8, ID3, SOX4, and any combination thereof, the Cas of the RNP comprises Cas9, CasX, CasY. or a combination thereof, and the genetically edited immune cells exhibit: enhanced expansion capability, enhanced cytotoxicity against target tumor cells, enhanced persistence, or any combination thereof, as compared to immune cells that have not been edited within the target sequence in the target gene.

In some embodiments, the gene is ADAM17. In some embodiments, the gene is HIF-la. In some embodiments, the gene is DGKz. In some embodiments, the gene is GSK-3B. In some embodiments, the gene is LAG3. In some embodiments, the gene is TIM3. In some embodiments, the gene is TRIM29. In some embodiments, the gene is IL-1R8. In some embodiments, the gene is CD38. In some embodiments, the gene is FBP-1. In some embodiments, the gene is INSIG1. In some embodiments, the gene is MED12. In some embodiments, the gene is MED13. In some embodiments, the gene is CCNC. In some embodiments, the gene is CDK8. In some embodiments, the gene is ID3. In some embodiments, the gene is SOX4.

In several embodiments, there is provided a method of manufacturing a population of genetically edited immune cells comprising: (a) contacting the population of immune cells with a first RNA guided endonuclease, wherein the RNA guided endonuclease edits within a target sequence in a target gene selected from ADAM17, HIF-1a, DGKz, GSK-3B, LAG3, TIM3, TRIM29, IL-1R8, CD38, FBP-1, INSIG1, MED12, MED13, CCNC, CDK8, ID3, SOX4, and any combination thereof; and(b) contacting the population of immune cells with a second RNA guided endonuclease, wherein the second RNA guided endonuclease edits within a target sequence in the CISH gene to yield reduced levels of expression of the CIS protein encoded by the CISH gene, as compared to an immune cell not edited within the target sequence in the CISH gene, wherein the genetically edited immune cells exhibit: enhanced expansion capability, enhanced cytotoxicity against target tumor cells, enhanced persistence, or any combination thereof, as compared to immune cells that have not been edited within the target sequences in the target gene and the CISH gene.

In several embodiments, there is provided a method of manufacturing a population of genetically edited immune cells comprising (a) contacting the population of immune cells with a first Cas-gRNA ribonucleoprotein (RNP) complex, wherein the RNP complex edits within a target sequence in a target gene selected from ADAM17, HIF-1a, DGKz, GSK-3B, LAG3, TIM3, TRIM29, IL-1R8, CD38, FBP-1, INSIG1, MED12, MED13, CCNC, CDK8, ID3, SOX4, or any combination thereof, and wherein the Cas of the first RNP complex comprises Cas9, CasX, CasY, or combinations thereof; and (b) contacting the population of immune cells with a second Cas-gRNA RNP complex, wherein the second RNP complex edits within a target sequence in the CISH gene to yield reduced levels of expression of the CIS protein encoded by the CISH gene, as compared to an immune cell not edited within the target sequence in the CISH gene, and wherein the Cas of the second RNP complex comprises Cas9, CasX, CasY, or combinations thereof, wherein the genetically edited immune cells exhibit: enhanced expansion capability, enhanced cytotoxicity against target tumor cells, enhanced persistence, or any combination thereof, as compared to immune cells that have not been edited within the target sequence in the target gene and the CISH gene.

In several embodiments, there is provided a method of manufacturing a population of genetically edited immune cells, comprising (a) contacting the population of immune cells with a first Cas-gRNA ribonucleoprotein (RNPP) complex, wherein the RNP edits within a target sequence in a target gene selected from ADAM17, HIF-1a, DGKz, GSK-3B, LAG3, TIM3, TRIM29, IL-1R8, CD38, FBP-1, INSIG1, MED12, MED13, CCNC, CDK8, ID3, SOX4, or any combination thereof, and wherein the Cas of the first RNP complex comprises Cas9, CasX, CasY, or combinations thereof; and (b) contacting the population of immune cells with a second Cas-gRNA RNP complex, wherein the second RNP complex edits within a target sequence in the CBLB gene to yield reduced levels of expression of the CBLB protein encoded by the CBLB gene, as compared to an immune cell not edited within the location in the CBLB gene, and wherein the Cas of the second RNP complex comprises Cas9, CasX, CasY, or combinations thereof, wherein the genetically edited immune cells exhibit: enhanced expansion capability, enhanced cytotoxicity against target tumor cells, enhanced persistence, or any combination thereof, as compared to immune cells that have not been edited within the target sequences in the target gene and the CBLB gene.

In several embodiments, there is provided a method of manufacturing a population of genetically edited immune cells, comprising (a) contacting the population of immune cells with a first RNA-guided endonuclease, wherein the first endonuclease edits within a target sequence in a target gene selected from ADAM17, HIF-1a, DGKz, GSK-3B, LAG3, TIM3, TRIM29, IL-1R8, CD38, FBP-1, INSIG1, MED12, MED13, CCNC, CDK8, ID3, SOX4, or any combination thereof; (b) contacting the population of immune cells with a second RNA-guided endonuclease, wherein the second RNA-guided endonuclease edits within a target sequence in the CISH gene to yield reduced levels of expression of the CIS protein encoded by the CISH gene, as compared to an immune cell not edited within the target sequence in the CISH gene; and (c) contacting the population of immune cells with a second RNA-guided endonuclease, wherein the third RNA-guided endonuclease edits within a target sequence in the CBLB gene to yield reduced levels of expression of the CBLB protein encoded by the CBLB gene, as compared to an immune cell not edited within the target sequence in the CBLB gene, wherein the genetically edited immune cells exhibit: enhanced expansion capability, enhanced cytotoxicity against target tumor cells, enhanced persistence, or any combination thereof, as compared to immune cells that have not been edited within the target sequences in the target, CISH, and CBLB genes.

In several embodiments, there is provided a method of manufacturing a population of genetically edited immune cells, comprising (a) contacting the population of immune cells with a first Cas-gRNA ribonucleoprotein (RNP) complex, wherein the first RNP complex edits within a target sequence in a target gene selected from ADAM17, HIF-1a, DGKz, GSK-3B, LAG3, TIM3, TRIM29, IL-1R8, CD38, FBP-1, INSIG1, MED12, MED13, CCNC, CDK8, ID3, SOX4, or any combination thereof; (b) contacting the population of immune cells with a second RNP complex, wherein the second RNP complex edits within a target sequence in the CISH gene to yield reduced levels of expression of the CIS protein encoded by the CISH gene, as compared to an immune cell not edited within the target sequence in the CISH gene; and (c) contacting the population of immune cells with a third RNP complex, wherein the third RNP complex edits within a target sequence in the CBLB gene to yield reduced levels of expression of the CBLB protein encoded by the CBLB gene, as compared to an immune cell not edited within the target sequence in the CBLB gene, wherein the Cas of each of the first, second, and third RNP complexes comprises Cas9, CasX, CasY, or combinations thereof, and wherein the genetically edited immune cells exhibit: enhanced expansion capability, enhanced cytotoxicity against target tumor cells, enhanced persistence, or any combination thereof, as compared to immune cells that have not been edited within the target sequences in the target, CISH, and CBLB genes.

In several embodiments, there is provided a method of manufacturing a population of genetically edited immune cells comprising (a) contacting the population of immune cells with a first Cas-gRNA ribonucleoprotein (RNP) complex, wherein the RNP complex edits within a target sequence in the MED12 gene; and (b) contacting the population of immune cells with a second Cas-gRNA RNP complex, wherein the second RNP complex edits within a target sequence in the CISH gene.

In several embodiments, there is provided a method of manufacturing a population of genetically edited immune cells, comprising contacting the population of immune cells with a plurality of Cas-gRNA ribonucleoprotein (RNP) complexes, wherein the plurality of RNP edits within a target sequence in the CISH gene to yield reduced levels of expression of the CIS protein encoded by the CISH gene, as compared to an immune cell not edited within the target sequence in the CISH gene, the plurality of RNP complexes edits within a target sequence in the CBLB gene to yield reduced levels of expression of CBLB protein encoded by the CBLB gene, as compared to an immune cell not edited within the target sequence in the CBLB gene, the plurality of RNP complexes induce edits within a target sequence in a target gene selected from ADAM17, HIF-1a, DGKz, GSK-3B, LAG3, TIM3, TRIM29, IL-1R8, CD38, FBP-1, INSIG1, MED12, MED13, CCNC, CDK8, ID3, SOX4, or any combination thereof, wherein the Cas of each of the plurality of RNP complexes comprises Cas9, CasX, CasY, or combinations thereof, and wherein the genetically edited immune cells exhibit: enhanced expansion capability, enhanced cytotoxicity against target tumor cells, enhanced persistence, or any combination thereof, as compared to immune cells that have not been edited within the target sequences in the CISH, CBLB, and target genes.

In some embodiments, the gene is ADAM17. In some embodiments, the gene is HIF-la. In some embodiments, the gene is DGKz. In some embodiments, the gene is GSK-3B. In some embodiments, the gene is LAG3. In some embodiments, the gene is TIM3. In some embodiments, the gene is TRIM29. In some embodiments, the gene is IL-1R8. In some embodiments, the gene is CD38. In some embodiments, the gene is FBP-1. In some embodiments, the gene is INSIG1. In some embodiments, the gene is MED12. In some embodiments, the gene is MED13. In some embodiments, the gene is CCNC. In some embodiments, the gene is CDK8. In some embodiments, the gene is ID3. In some embodiments, the gene is SOX4.

In several embodiments, the manufacturing methods further comprise contacting the population of immune cells with a vector comprising a polynucleotide encoding a cytotoxic receptor comprising an extracellular ligand binding domain, a transmembrane domain, and a cytotoxic signaling complex.

In several embodiments, the immune cells are genetically edited within a target sequence in the ADAM17 gene, and the target sequence comprises any of SEQ TD NO: 682-687. In several embodiments, the immune cells are genetically edited within a target sequence in the HIF-1a gene, and the target sequence comprises any of SEQ ID NO: 750-760. In several embodiments, the immune cells are genetically edited within a target sequence in the DGKz gene, and the target sequence comprises any of SEQ ID NO: 688-723. In several embodiments, the immune cells are genetically edited within a target sequence in the GSK-3B gene, and the target sequence comprises any of SEQ ID NO: 724-749. In several embodiments, the immune cells are genetically edited within a target sequence in the LAG3 gene, and the target sequence comprises any of SEQ ID NO: 761-789. In several embodiments, the immune cells are genetically edited within a target sequence in the TIM3 gene, and the target sequence comprises any of SEQ ID NO: 790-825. In several embodiments, the immune cells are genetically edited within a target sequence in the TRIM29 gene, and the target sequence comprises any of SEQ ID NO: 826-835. In several embodiments, the immune cells are genetically edited within a target sequence in the IL-1R8 gene, and the target sequence comprises any of SEQ ID NO: 836-865. In several embodiments, the immune cells are genetically edited within a target sequence in the CD38 gene, and the target sequence comprises any of SEQ ID NO: 866-874. In several embodiments, the immune cells are genetically edited within a target sequence in the FBP-1 gene, and wherein the target sequence comprises any of SEQ ID NO: 875-889. In several embodiments, the immune cells are genetically edited within a target sequence in the INSIG1 gene, and the target sequence comprises any of SEQ ID NO: 890-934. In several embodiments, the immune cells are genetically edited within a target sequence in the MED12 gene, wherein the target sequence comprises any of SEQ ID NO: 938-944. In several embodiments, the immune cells are genetically edited within a target sequence in the MED12 gene, and a target sequence comprising any of SEQ ID NO: 938-944 is used to target the MED12 gene, and optionally wherein the immune cells are genetically edited within a target sequence in the MED13 gene, and the target sequence comprises any of SEQ ID NO: 945-948. In several embodiments, the immune cells are genetically edited within a target sequence in the MED13 gene, wherein the target sequence comprises any of SEQ ID NO: 945-948. In several embodiments, the immune cells are genetically edited within a target sequence in the CDK8 gene, and the target sequence comprises any of SEQ ID NO: 949-955. In several embodiments, the immune cells are genetically edited within a target sequence in the CCNC gene, and the target sequence comprises any of SEQ ID NO: 956-962. In several embodiments, the immune cells are genetically edited within a target sequence in the ID3 gene, and the target sequence comprises any of SEQ ID NO: 963-969. In several embodiments, the immune cells are genetically edited within a target sequence in the SOX4 gene, and the target sequence comprises any of SEQ ID NO: 970-976. In several embodiments, the immune cells are genetically edited within a target sequence in the CISH gene, and the target sequence comprises any of SEQ ID NO: 153-157 or 463-466. In several embodiments, the immune cells are genetically edited within a target sequence in the CBLB gene, and the target sequence comprises any of SEQ ID NO: 164 to 166 or 453-456. In several embodiments, the cells are genetically edited within a target sequence in the MED12 gene. In several embodiments, the target sequence in the MED12 gene comprises any of SEQ ID NOS:996-998. In several embodiments, the cells are genetically edited within a target sequence in the CCNC gene. In several embodiments, the target sequence in the CCNC gene comprises any of SEQ ID NOS:999-1001. In several embodiments, the cells are genetically edited within a target sequence in the SOCS2 gene. In several embodiments, the target sequence in the SOCS2 gene comprises any of SEQ ID NOS:1002-1004. In several embodiments, the cells are genetically edited within a target sequence in the CISH gene. In several embodiments, the target sequence in the CISH gene comprises any of SEQ ID NOS:1012-1013. In several embodiments, the cells are genetically edited within a target sequence in the CBLB gene. In several embodiments, the target sequence in the CBLB gene comprises any of SEQ ID NOS: 1005-1008. In several embodiments, the cells are genetically edited within a target sequence in the TRIM29 gene. In several embodiments, the target sequence in the TRIM29 gene comprises any of SEQ ID NOS:1009-1011. In several embodiments, the cells are genetically edited within a target sequence in the CD70 gene. In several embodiments, the cells are genetically edited within a target sequence in the TGFBR2 gene, the TIGIT gene, the adenosine A2a receptor (ADORA2A) gene, the SMAD3 gene, the MAPKAPK3 gene, the CEACAM1 gene, the DDIT4 gene, then NKG2A gene, the SOCS2 gene, the B2M gene, the PDCD1gene, and/or the TRAC gene.

In several embodiments, at least a portion of the genetically engineered immune cells are engineered to express interleukin-15 (IL15). In some embodiments, IL15 is a membrane-bound IL15 (mbIL15). In several embodiments, at least a portion of the genetically engineered immune cells are engineered to express membrane bound IL-15 (mbIL15). the cytotoxic receptor and the mbIL15 are encoded by the same nucleic acid molecule. In several embodiments, the nucleic acid sequences encoding the cytotoxic receptor and the mbIL15 are separated by a nucleic acid sequence encoding a 2A peptide.

In several embodiments, the cells are edited within target sequences in the CISH, CBLB, and ADAM17 genes.

In several embodiments, the cells are edited within target sequences in the CISH, CBLB, and HIF1a genes.

In several embodiments, the cells are edited within target sequences in the CISH, CBLB, and FBP-1 genes.

In several embodiments, the cells are edited within target sequences in the CISH, CBLB, and/or MED12 genes. In several embodiments, the cells are edited within target sequences in the CISH and MED12 genes. In several embodiments, the cells are edited within target sequences in the CBLB and MED12 genes. In several embodiments, the cells are edited within target sequences in the CISH, CBLB, and MED12 genes.

In several embodiments, the cells are also edited within a target sequence in the CD70 gene, and wherein the method further comprises contacting the population of immune cells with a vector comprising a polynucleotide encoding a cytotoxic receptor comprising an extracellular ligand binding domain that targets CD70, a transmembrane domain, and a cytotoxic signaling complex.

In several embodiments, the methods do not comprise editing the CD70 gene. In several embodiments, the methods do not comprise editing the CD70 gene and the immune cells express their normal endogenous amount of CD70. In several embodiments, the cells are not edited within a target sequence in the CD70 gene.

In several embodiments, the cytotoxic receptor binds to BCMA, CD19, CD70, a NKG2D ligand, CD38, GPRC5D, CD138 DLL3, EGFR, PSMA, FLT3, KREMEN2, or a combination thereof. In some embodiments, the cytotoxic receptor binds to BCMA. In some embodiments, the cytotoxic receptor binds to CD19. In some embodiments, the cytotoxic receptor binds to CD70. In some embodiments, the cytotoxic receptor binds to NKG2D ligand. In some embodiments, the cytotoxic receptor binds to CD38. In some embodiments, the cytotoxic receptor binds to GPRC5D. In some embodiments, the cytotoxic receptor binds to CD138. In some embodiments, the cytotoxic receptor binds to GPRC5D. In some embodiments, the cytotoxic receptor binds to DLL3. In some embodiments, the cytotoxic receptor binds to EGFR. In some embodiments, the cytotoxic receptor binds to PSMA. In some embodiments, the cytotoxic receptor binds to FLT3. In some embodiments, the cytotoxic receptor binds to KREMEN2.

In several embodiments, the cytotoxic receptor does not target CD19.

In several embodiments, the cytotoxic receptor does not target NKG2D ligands.

In several embodiments, the immune cells comprise Natural Killer (NK) cells, T cells, induced pluripotent stem cells (iPSCs), iPSC-derived NK cells, iPSC-derived T cells, NK-92 cells, or combinations thereof. In several embodiments, the immune cells comprise Natural Killer (NK) cells. In several embodiments, the immune cells comprise T cells. In several embodiments, the immune cells comprise Natural Killer (NK) cells and T cells.

In several embodiments, the immune cells comprise a mixture of NK cells and T cells or a mixture of iPSC-derived NK cells and T cells. In several embodiments, the immune cells comprise a mixture of iPSC-derived NK cells and/or iPSC-derived T cells.

Also provided for herein is a method for treating cancer in a subject comprising, administering to the subject a population of genetically engineered immune cells as provided for herein. In some embodiments, cells of the cancer express the antigen bound by the cytotoxic receptor.

In several embodiments, the immune cells are allogeneic with respect the subject. In some embodiments, the immune cells are obtained from a donor that does not have cancer.

In several embodiments, the treatment methods or uses provided for herein further comprise administering IL2.

In several embodiments, the transmembrane domain of the expressed cytotoxic receptor comprises CD8, CD28, or a portion thereof, optionally wherein the transmembrane domain comprises CD8 alpha or a portion thereof. In several embodiments, the transmembrane domain of the expressed cytotoxic receptor comprises CD8. In several embodiments, the transmembrane domain of the expressed cytotoxic receptor comprises CD8 alpha. In several embodiments, the cytotoxic signaling complex of the expressed cytotoxic receptor comprises a CD3zeta domain and an intracellular signaling domain. In several embodiments, the cytotoxic signaling complex of the expressed cytotoxic receptor comprises a CD3zeta domain and an intracellular signaling domain of an OX40, 4-1BB, CD28, or a signaling portion thereof, optionally an intracellular signaling domain of OX40 or a signaling portion thereof. In several embodiments, the cytotoxic signaling complex of the expressed cytotoxic receptor comprises a CD3zeta domain and an intracellular signaling domain of an OX40. In several embodiments, at least a portion of the genetically engineered immune cells are engineered to express interleukin-15 (IL15). In some embodiments, IL15 is a membrane-bound IL15 (mbIL15). In several embodiments, at least a portion of the genetically engineered immune cells are engineered to express membrane bound IL-15 (mbIL15).

In several embodiments, provided for herein is a composition comprising the population of genetically engineered and gene edited immune cells as provided for herein. In several embodiments, provided for herein is a composition comprising the population of gene edited immune cells as provided for herein. In several embodiments, provided for herein is a composition comprising the population of genetically engineered and gene edited immune cells as provided for herein and a pharmaceutically acceptable excipient. In several embodiments, provided for herein is a composition comprising the population of gene edited immune cells as provided for herein and a pharmaceutically acceptable excipient.

Also provided for herein are methods for the treatment of a subject having a disease or condition comprising administering to the subject the population of genetically engineered and gene edited immune cells or compositions as disclosed herein. Also provided for is the use a population of genetically engineered and gene edited immune cells or a composition as disclosed herein for the treatment of a subject having a disease or condition. Also provided for is the use a population of genetically engineered and gene edited immune cells or a composition as disclosed herein for the preparation of a medicament for the treatment of a subject having a disease or condition. Also provided for herein are methods for the treatment of a subject having a disease or condition comprising administering to the subject the population of gene edited immune cells or compositions as disclosed herein. Also provided for is the use a population of gene edited immune cells or a composition as disclosed herein for the treatment of a subject having a disease or condition. Also provided for is the use a population of gene edited immune cells or a composition as disclosed herein for the preparation of a medicament for the treatment of a subject having a disease or condition.

In several embodiments, the disease or condition is an infectious disease, an autoimmune disease, a cancer, or a tumor. In several embodiments, the disease or condition is a cancer. In several embodiments, the disease or condition is a NKG2D ligand-expressing cancer In several embodiments, the disease or condition is a CD19-expressing cancer. In several embodiments, the disease or condition is a CD70-expressing cancer. In several embodiments, the disease or condition is a BCMA-expressing cancer. In several embodiments, the immune cells comprise natural killer (NK) cells. In several embodiments, the immune cells are allogeneic to the subject.

Provided herein is a population of gene edited immune cells that are genetically edited within a target sequence in the gene encoding a disintegrin and metalloproteinase domain-containing protein 17 (ADAM17) protein, wherein the edit yields reduced expression and/or function of the ADAM17 protein as compared to an immune cell not edited within the target sequence in the ADAM17 gene. In some embodiments, the gene edited immune cells are genetically engineered to express a cytotoxic receptor comprising an extracellular ligand binding domain, a transmembrane domain, and a cytotoxic signaling complex. In some embodiments, the extracellular ligand binding domain binds an antigen expressed by cells of a cancer or a tumor.

Also provided herein is a population of genetically engineered and gene edited immune cells, comprising genetically engineered immune cells that express a cytotoxic receptor comprising an extracellular ligand binding domain, a transmembrane domain, and a cytotoxic signaling complex, wherein the extracellular ligand binding domain targets a tumor marker expressed by a target tumor cell, wherein the immune cells are genetically edited within a target sequence in the ADAM17 gene, and wherein the edits yield reduced expression and/or function of the ADAM17 protein as compared to an immune cell not edited within the target sequence in the ADAM17 gene. In several embodiments, the immune cells are edited within an additional target sequence in the genome of the immune cell to yield reduced levels of expression of a protein that is encoded by a gene comprising the edit within the additional target sequence, compared to a non-edited immune cell. In several embodiments, the protein that is encoded by a gene comprising the edit within the additional target sequence is ADAM17. In several embodiments, the protein that is encoded by a gene comprising the edit within the additional target sequence is not ADAM17. In some embodiments, the edit to the ADAM17 gene is made using a RNA-guided endonuclease. In some embodiments, the edit to the additional location is made using a RNA-guided endonuclease. In several embodiments, the genetically engineered and edited immune cells exhibit one or more of enhanced expansion capability, enhanced cytotoxicity against target tumor cells, and enhanced persistence, as compared to immune cells that do not comprise a genetically edited location.

In several embodiments, there is provided a population of genetically engineered and gene edited immune cells, comprising genetically engineered immune cells that express a cytotoxic receptor comprising an extracellular ligand binding domain, a transmembrane domain, and a cytotoxic signaling complex, wherein the extracellular ligand binding domain targets a tumor marker expressed by a target tumor cell, wherein the immune cells are genetically edited at one or more locations in an ADAM17 target gene that encodes a corresponding protein, wherein the edits yield reduced expression and/or function of a corresponding ADAM17 protein as compared to an immune cell not edited at the location or locations in the ADAM17 gene, wherein the immune cells are optionally edited at one or more additional target sites in the genome of the immune cell to yield reduced levels of expression of the protein which is encoded by a gene which comprises an edited target site as compared to a non-edited immune cell, wherein the edits to the target gene or target genes are made using an RNA-guided endonuclease, and wherein the genetically engineered and edited immune cells exhibit one or more of enhanced expansion capability, enhanced cytotoxicity against target tumor cells, and enhanced persistence, as compared to immune cells that do not comprise said genetically edited target site or sites.

Provided herein is a population of gene edited immune cells that are genetically edited within a target sequence in the gene encoding a mediator of RNA polymerase II transcription subunit 12 (MED12) protein, wherein the edit yield reduced expression and/or function of the MED12 protein as compared to an immune cell not edited within the target sequence in the MED12 gene. In some embodiments, the gene edited immune cells are genetically engineered to express a cytotoxic receptor comprising an extracellular ligand binding domain, a transmembrane domain, and a cytotoxic signaling complex. In some embodiments, the extracellular ligand binding domain binds an antigen expressed by cells of a cancer or a tumor.

Also provided herein is a population of genetically engineered and gene edited immune cells, comprising genetically engineered immune cells that express a cytotoxic receptor comprising an extracellular ligand binding domain, a transmembrane domain, and a cytotoxic signaling complex, wherein the extracellular ligand binding domain targets a tumor marker expressed by a target tumor cell, wherein the immune cells are genetically edited within a target sequence in the MED12 gene. Also provided herein is a population of genetically engineered and gene edited immune cells, comprising genetically engineered immune cells that express a cytotoxic receptor comprising an extracellular ligand binding domain, a transmembrane domain, and a cytotoxic signaling complex, wherein the extracellular ligand binding domain targets a tumor marker expressed by a target tumor cell, wherein the immune cells are genetically edited within a target sequence in the MED12 gene, and wherein the edits yield reduced expression and/or function of the MED12 protein as compared to an immune cell not edited within the target sequence in the MED12 gene. In several embodiments, the immune cells are edited within an additional target sequence in the genome of the immune cell to yield reduced levels of expression of a protein that is encoded by a gene comprising the edit within the additional target sequence, compared to a non-edited immune cell. In several embodiments, the protein that is encoded by a gene comprising the edit within the additional target sequence is MED12. In several embodiments, the protein that is encoded by a gene comprising the edit within the additional target sequence is not MED12. In some embodiments, the edit to the MED12 gene is made using a RNA-guided endonuclease. In some embodiments, the edit to the additional location is made using a RNA-guided endonuclease. In several embodiments, the genetically engineered and edited immune cells exhibit one or more of enhanced expansion capability, enhanced cytotoxicity against target tumor cells, and enhanced persistence, as compared to immune cells that do not comprise a genetically edited location.

In several embodiments, there is provided a population of genetically engineered and gene edited immune cells, comprising genetically engineered immune cells that express a cytotoxic receptor comprising an extracellular ligand binding domain, a transmembrane domain, and a cytotoxic signaling complex, wherein the extracellular ligand binding domain targets a tumor marker expressed by a target tumor cell, wherein the immune cells are genetically edited at one or more locations in a MED12 target gene that encodes a corresponding protein, wherein the edits yield reduced expression and/or function of a corresponding MED12 protein as compared to an immune cell not edited at the location or locations in the MED12 gene, wherein the immune cells are optionally edited at one or more additional target sites in the genome of the immune cell to yield reduced levels of expression of the protein which is encoded by a gene which comprises an edited target site as compared to a non-edited immune cell, wherein the edits to the target gene or target genes are made using an RNA-guided endonuclease, and wherein the genetically engineered and edited immune cells exhibit one or more of enhanced expansion capability, enhanced cytotoxicity against target tumor cells, and enhanced persistence, as compared to immune cells that do not comprise said genetically edited target site or sites.

Provided herein is a population of gene edited immune cells that are genetically edited within a target sequence in the gene encoding a hypoxia-inducible factor 1-alpha (HIF1-a) protein, wherein the edit yield reduced expression and/or function of the HIF1-a protein as compared to an immune cell not edited within the target sequence in the HIF1A gene. In some embodiments, the gene edited immune cells are genetically engineered to express a cytotoxic receptor comprising an extracellular ligand binding domain, a transmembrane domain, and a cytotoxic signaling complex. In some embodiments, the extracellular ligand binding domain binds an antigen expressed by cells of a cancer or a tumor.

In several embodiments, there is provided a population of genetically engineered and gene edited immune cells, comprising, genetically engineered immune cells that express a cytotoxic receptor comprising an extracellular ligand binding domain, a transmembrane domain, and a cytotoxic signaling complex, wherein the extracellular ligand binding domain targets a tumor marker expressed by a target tumor cell, wherein the immune cells are genetically edited at one or more locations in an HIF1-a target gene that encodes a corresponding HIF1-a protein, wherein the edits yield reduced expression and/or function of the corresponding HIF1-a protein as compared to an immune cell not edited at the location or locations in the HIF1-a gene, wherein the immune cells are optionally edited at one or more additional target sites in the genome of the immune cell to yield reduced levels of expression of the protein which is encoded by a gene which comprises an edited target site as compared to a non-edited immune cell, wherein the edits to the target gene or target genes are made using an RNA-guided endonuclease, and wherein the genetically engineered and edited immune cells exhibit one or more of enhanced expansion capability, enhanced cytotoxicity against target tumor cells, and enhanced persistence, as compared to immune cells that do not comprise said genetically edited target site or sites.

In several embodiments, the immune cells are genetically edited within a target sequence within a target gene selected from ADAM17, HIF-1a, DGKz, GSK-3B, LAG3, TIM3, TRIM29, IL-1R8, CD38, FBP-1, INSIG1, MED12, MED 13, CCNC, CDK8, ID3, SOX4, or any combination thereof. In several embodiments, the edit yields reduced expression and/or function of the corresponding protein as compared to an immune cell not edited within target sequence in the target gene. In several embodiments, the edit is made using an RNA-guided endonuclease. In some embodiments, the edit is made using a CRISPR/Cas system. In some embodiments, the Cas is Cas9. In some embodiments, the edit is made using a CRISPR/Cas9 system. In several embodiments, the genetically engineered and edited immune cells exhibit enhanced expansion capability, enhanced cytotoxicity against target tumor cells, enhanced persistence, or any combination thereof, as compared to immune cells that have not been edited within the target sequence(s).

In several embodiments, there is provided population of genetically engineered and gene edited immune cells, comprising genetically engineered immune cells that express a cytotoxic receptor comprising an extracellular ligand binding domain, a transmembrane domain, and a cytotoxic signaling complex, wherein the extracellular ligand binding domain targets a tumor marker expressed by a target tumor cell, wherein the immune cells are genetically edited at one or more locations in a target gene selected from ADAM17, HIF-1a, DGKz, GSK-3B, LAG3, TIM3, TRIM29, IL-1R8, CD38, FBP-1, INSIG1, MED12, MED 13, CCNC. CDK8, ID3, SOX4, or any combination thereof, wherein each of said genes encodes a corresponding protein, wherein the edits yield reduced expression and/or function of the corresponding protein as compared to an immune cell not edited at the location or locations in the respective gene, wherein the immune cells are edited at one or more additional target sites in the genome of the immune cell to yield reduced levels of expression of the protein which is encoded by a gene which comprises an edited target site as compared to a non-edited immune cell, wherein the edits to the target gene or target genes are made using an RNA-guided endonuclease, and wherein the genetically engineered and edited immune cells exhibit one or more of enhanced expansion capability, enhanced cytotoxicity against target tumor cells, and enhanced persistence, as compared to immune cells that do not comprise said genetically edited target site or sites.

In several embodiments, there is provided a population of genetically engineered and gene edited immune cells, comprising genetically engineered immune cells that express a cytotoxic receptor comprising an extracellular ligand binding domain, a transmembrane domain, and a cytotoxic signaling complex, wherein the extracellular ligand binding domain targets a tumor marker expressed by a target tumor cell, wherein the immune cells are genetically edited at one or more locations in a target gene selected from ADAM17, HIF-1a, DGKz, GSK-3B, LAG3, TIM3, TRIM29, IL-1R8, CD38, FBP-1, INSIG1, MED12, MED13, CCNC, CDK8, ID3, SOX4, or any combination thereof, wherein each of said genes encodes a corresponding protein, wherein the edits yield reduced expression and/or function of the corresponding protein as compared to an immune cell not edited at the location or locations in the respective gene, wherein the immune cells are edited at one or more additional target sites in the genome of the immune cell to yield reduced levels of expression of the protein which is encoded by a gene which comprises an edited target site as compared to a non-edited immune cell, wherein the edits to the target gene or target genes are made using a Crispr/Cas9 system, and wherein the genetically engineered and edited immune cells exhibit one or more of enhanced expansion capability, enhanced cytotoxicity target tumor cells, and enhanced persistence, as compared to immune cells that do not comprise said genetically edited target site or sites.

In several embodiments, there is also provided a population of genetically engineered and gene edited immune cells, comprising genetically engineered immune cells that express a cytotoxic receptor comprising an extracellular ligand binding domain, a transmembrane domain, and a cytotoxic signaling complex, wherein the extracellular ligand binding domain targets a tumor marker expressed by a target tumor cell, wherein the tumor marker expressed by the target tumor cell is selected from a ligand of the NKG2D receptor, CD19, or CD70, wherein the immune cells are genetically edited at one or more locations in a target gene selected from ADAM17, HIF-1a, DGKz, GSK-3B, LAG3, TIM3, TRIM29, IL-1R8, CD38, FBP-1, INSIG1, MED12, MED13, CCNC, CDK8, ID3, SOX4, or any combination thereof, wherein each of said genes encodes a corresponding protein, wherein the edits yield reduced expression and/or function of the corresponding protein as compared to an immune cell not edited at the location or locations in the respective gene, wherein the immune cells are edited at one or more additional target sites in the genome of the immune cell to yield reduced levels of expression of the protein which is encoded by a gene which comprises an edited target site as compared to a non-edited immune cell, wherein the edits to the target gene or target genes are made using a Crispr/Cas system or other guided endonuclease, and wherein the genetically engineered and edited immune cells exhibit one or more of enhanced expansion capability, enhanced cytotoxicity against target tumor cells, and enhanced persistence, as compared to immune cells that do not comprise said genetically edited target site or sites.

In several embodiments, there is provided a method of manufacturing a population of genetically edited immune cells for cancer immunotherapy, comprising, contacting the population of immune cells with a targeted endonuclease, wherein the targeted endonuclease cuts nucleic acid at two or more target sites in a target gene selected from ADAM17, HIF-1a, DGKz, GSK-3B, LAG3, TIM3, TRIM29, IL-1R8, CD38, FBP-1, INSIG1, MED12, MED13, CCNC, CDK8, ID3, SOX4, CISH, CBLB, or any combination thereof, and wherein the genetically edited immune cells exhibit one or more of enhanced expansion capability, enhanced cytotoxicity against target tumor cells, and enhanced persistence, as compared to immune cells that do not comprise said genetically edited target site or sites.

In several embodiments, there is provided a method of manufacturing a population of genetically edited immune cells for cancer immunotherapy, comprising contacting the population of immune cells with a RNA guided endonuclease, wherein the RNA guided endonuclease edits at one or more target sites in a target gene selected from ADAM17, HIF-1a, DGKz, GSK-3B, LAG3, TIM3, TRIM29, IL-1R8, CD38, FBP-1, INSIG1, MED12, MED13, CCNC, CDK8, TD3, SOX4 or any combination thereof, and wherein the genetically edited immune cells exhibit one or more of enhanced expansion capability, enhanced cytotoxicity against target tumor cells, and enhanced persistence, as compared to immune cells that do not comprise said genetically edited target site or sites.

In several embodiments, there is provided a method of manufacturing a population of genetically edited immune cells for cancer immunotherapy, comprising contacting the population of immune cells with a Cas-gRNA ribonucleoprotein complex (RNP), wherein the RNP edits at one or more target sites in a target gene selected from ADAM17, HIF-1a, DGKz, GSK-3B, LAG3, TIM3, TRIM29, IL-1R8, CD38, FBP-1, INSIG1, MED12, MED13, CCNC, CDK8, ID3, SOX4 or any combination thereof, wherein the Cas of the RNP comprises Cas9, CasX, CasY, or combinations thereof, and wherein the genetically edited immune cells exhibit one or more of enhanced expansion capability, enhanced cytotoxicity against target tumor cells, and enhanced persistence, as compared to immune cells that do not comprise said genetically edited target site or sites.

In some embodiments, the gene is ADAM17. In some embodiments, the gene is HIF-la. In some embodiments, the gene is DGKz. In some embodiments, the gene is GSK-3B. In some embodiments, the gene is LAG3. In some embodiments, the gene is TIM3. In some embodiments, the gene is TRIM29. In some embodiments, the gene is IL-1R8. In some embodiments, the gene is CD38. In some embodiments, the gene is FBP-1. In some embodiments, the gene is INSIG1. In some embodiments, the gene is MED12. In some embodiments, the gene is MED13. In some embodiments, the gene is CCNC. In some embodiments, the gene is CDK8. In some embodiments, the gene is ID3. In some embodiments, the gene is SOX4.

In several embodiments, there is provided a method of manufacturing a population of genetically edited immune cells for cancer immunotherapy, comprising contacting the population of immune cells with a first RNA guided endonuclease, wherein the endonuclease edits at one or more target sites in a target gene selected from ADAM17, HIF-1a, DGKz, GSK-3B, LAG3, TIM3, TRIM29, IL-1R8, CD38, FBP-1, INSIG1, MED12, MED13, CCNC, CDK8, ID3, SOX4, or any combination thereof; and contacting the population of immune cells with a second RNA guided endonuclease, wherein the second endonuclease edits at one or more target sites in a in a CISH gene of the immune cell to yield reduced levels of expression of CIS protein encoded by the CISH gene as compared to an immune cell not edited at the CISH gene, and wherein the genetically edited immune cells exhibit one or more of enhanced expansion capability, enhanced cytotoxicity against target tumor cells, and enhanced persistence, as compared to immune cells that do not comprise said genetically edited target site or sites.

In several embodiments, there is provided a method of manufacturing a population of genetically edited immune cells for cancer immunotherapy, comprising contacting the population of immune cells with a first Cas-gRNA ribonucleoprotein complex (RNP), wherein the RNP edits at one or more target sites wherein the RNP edits at one or more target sites in a target gene selected from ADAM17, HIF-1 a, DGKz, GSK-3B, LAG3, TIM3, TRIM29, IL-1 R8, CD38, FBP-1, INSIG1, MEDI 2, MED13, CCNC, CDK8, ID3, SOX4, or any combination thereof, wherein the Cas of the RNP comprises Cas9, CasX, CasY, or combinations thereof, and contacting the population of immune cells with a second RNP complex, wherein the second RNP edits at one or more target sites in a in a CISH gene of the immune cell to yield reduced levels of expression of CIS protein encoded by the CISH gene as compared to an immune cell not edited at the CISH gene, wherein the Cas of the RNP comprises Cas9, CasX, CasY, or combinations thereof, and wherein the genetically edited immune cells exhibit one or more of enhanced expansion capability, enhanced cytotoxicity against target tumor cells, and enhanced persistence, as compared to immune cells that do not comprise said genetically edited target site or sites.

In several embodiments, there is provided a method of manufacturing a population of genetically edited immune cells for cancer immunotherapy, comprising contacting the population of immune cells with a first Cas-gRNA ribonucleoprotein complex (RNP), wherein the RNP edits at one or more target sites wherein the RNP edits at one or more target sites in a target gene selected from ADAM17, HIF-1a, DGKz, GSK-3B, LAG3, TIM3, TRIM29, IL-1R8, CD38, FBP-1, INSIG1, MED12, MED13, CCNC, CDK8, ID3, SOX4, or any combination thereof, wherein the Cas of the RNP comprises Cas9, CasX, CasY, or combinations thereof, and contacting the population of immune cells with a second RNP complex, wherein the second RNP edits at one or more target sites in a in a CBLB gene of the immune cell to yield reduced levels of expression of CBLB protein encoded by the CBLB gene as compared to an immune cell not edited at the CBLB gene, wherein the Cas of the RNP comprises Cas9, CasX, CasY, or combinations thereof, and wherein the genetically edited immune cells exhibit one or more of enhanced expansion capability, enhanced cytotoxicity against target tumor cells, and enhanced persistence, as compared to immune cells that do not comprise said genetically edited target site or sites.

In several embodiments, there is provided a method of manufacturing a population of genetically edited immune cells for cancer immunotherapy, comprising contacting the population of immune cells with a first RNA-guided endonuclease, wherein the first endonuclease edits at one or more target sites wherein the RNP edits at one or more target sites in a target gene selected from ADAM17, HIF-1a, DGKz, GSK-3B, LAG3, TIM3, TRIM29, IL-1R8, CD38, FBP-1, INSIG1, MED12, MED13, CCNC, CDK8, ID3, SOX4, or any combination thereof, and contacting the population of immune cells with a second and a third RNA-guided endonuclease, wherein the second RNA-guided endonuclease edits at one or more target sites in a in a CISH gene of the immune cell to yield reduced levels of expression of CIS protein encoded by the CISH gene as compared to an immune cell not edited at the CISH gene, wherein the third RNA-guided endonuclease edits at one or more target sites in CBLB gene of the immune cell to yield reduced levels of expression of CBLB protein encoded by the CBLB gene as compared to an immune cell not edited at the CBLB gene, and wherein the genetically edited immune cells exhibit one or more of enhanced expansion capability, enhanced cytotoxicity against target tumor cells, and enhanced persistence, as compared to immune cells that do not comprise said genetically edited target site or sites.

In several embodiments, there is provided a method of manufacturing a population of genetically edited immune cells for cancer immunotherapy, comprising contacting the population of immune cells with a first Cas-gRNA ribonucleoprotein complex (RNP), wherein the RNP edits at one or more target sites wherein the RNP edits at one or more target sites in a target gene selected from ADAM17, HIF-1a, DGKz, GSK-3B, LAG3, TIM3, TRIM29, IL-1R8, CD38, FBP-1, INSIG1, MED12, MED13, CCNC, CDK8, ID3, SOX4, or any combination thereof, and contacting the population of immune cells with a second and a third RNP complex, wherein the second RNP edits at one or more target sites in a in a CISH gene of the immune cell to yield reduced levels of expression of CIS protein encoded by the CISH gene as compared to an immune cell not edited at the CISH gene, wherein the third RNP edits at one or more target sites in CBLB gene of the immune cell to yield reduced levels of expression of CBLB protein encoded by the CBLB gene as compared to an immune cell not edited at the CBLB gene, wherein the Cas of each of the RNP comprises Cas9, CasX, CasY, or combinations thereof and wherein the genetically edited immune cells exhibit one or more of enhanced expansion capability, enhanced cytotoxicity against target tumor cells, and enhanced persistence, as compared to immune cells that do not comprise said genetically edited target site or sites.

In several embodiments, there is provided a method of manufacturing a population of genetically edited immune cells for cancer immunotherapy, comprising, contacting the population of immune cells with a plurality of Cas-gRNA ribonucleoprotein complex (RNP), wherein the plurality of RNP induces edits at one or more target sites in a CISH gene of the immune cell to yield reduced levels of expression of CIS protein encoded by the CISH gene as compared to an immune cell not edited at the CISH gene, wherein the plurality of RNP induces edits at one or more target sites in CBLB gene of the immune cell to yield reduced levels of expression of CBLB protein encoded by the CBLB gene as compared to an immune cell not edited at the CBLB gene, wherein the plurality of RNP induces edits at one or more target sites in a target gene selected from ADAM17, HIF-1a, DGKz, GSK-3B, LAG3, TIM3, TRIM29, IL-1R8, CD38, FBP-1, INSIG1, MED12, MED13, CCNC, CDK8, ID3, SOX4, or any combination thereof, wherein the Cas of each of the plurality of RNP comprises Cas9, CasX, CasY, or combinations thereof, and wherein the genetically edited immune cells exhibit one or more of enhanced expansion capability, enhanced cytotoxicity against target tumor cells, and enhanced persistence, as compared to immune cells that do not comprise said genetically edited target site or sites.

In some embodiments, the gene is ADAM17. In some embodiments, the gene is HIF-la. In some embodiments, the gene is DGKz. In some embodiments, the gene is GSK-3B. In some embodiments, the gene is LAG3. In some embodiments, the gene is TIM3. In some embodiments, the gene is TRIM29. In some embodiments, the gene is IL-1R8. In some embodiments, the gene is CD38. In some embodiments, the gene is FBP-1. In some embodiments, the gene is INSIG1. In some embodiments, the gene is MED12. In some embodiments, the gene is MED13. In some embodiments, the gene is CCNC. In some embodiments, the gene is CDK8. In some embodiments, the gene is TD3. In some embodiments, the gene is SOX4.

In several embodiments, the methods of production further comprise contacting the population of immune cells with a vector comprising a polynucleotide encoding a cytotoxic receptor comprising an extracellular ligand binding domain, a transmembrane domain, and a cytotoxic signaling complex.

Also provided herein is a method for treating a subject having a disease or condition comprising, administering to the subject population of gene edited natural killer (NK) cells that are genetically edited within a target sequence in the MED12 gene. In some embodiments, the immune cells are genetically engineered to express a cytotoxic receptor. In some embodiments, the cytotoxic receptor comprises an extracellular ligand binding domain, a transmembrane domain, and a cytotoxic signaling complex. In some embodiments, the extracellular ligand binding domain binds to an antigen expressed by cells of a target tumor or cancer. In some embodiments, the genetic edit within a target sequence in the MED12 gene comprising any one of SEQ ID NOs: 938-994 or 996-998. In some embodiments, the genetic edit to MED12 reduces expression and/or function of the MED12 protein encoded by the MED12 gene, as compared to an immune cell not edited within the target sequence. In several embodiments, the edit to the MED12 gene is made using an RNA-guided endonuclease.

In some embodiments, provided herein is a method for treating a subject having a disease or condition comprising, administering to the subject population of natural killer (NK) cells that are genetically edited within a target sequence in the MED12 gene. In some embodiments, provided herein is a method for treating a subject having a disease or condition comprising, administering to the subject population of natural killer (NK) cells that are genetically edited within a target sequence in the MED12 gene and a target sequence in the CISH gene. In several embodiments, there is provided a population of genetically edited immune cells comprising immune cells that are genetically edited within a target sequence in the MED12 gene and within a target sequence in the CISH gene. In some embodiments, the immune cells are genetically engineered to express a cytotoxic receptor. In some embodiments, the cytotoxic receptor comprises an extracellular ligand binding domain, a transmembrane domain, and a cytotoxic signaling complex. In some embodiments, the extracellular ligand binding domain binds to an antigen expressed by cells of a target tumor or cancer. In some embodiments, the genetic edit within a target sequence in the MED12 gene reduces expression and/or function of the MED12 protein encoded by the MED12 gene, as compared to an immune cell not edited within the target sequence. In some embodiments, the genetic edit within a target sequence in the CISH gene reduces expression and/or function of the CIS protein encoded by the CIS gene, as compared to an immune cell not edited within the target sequence. In several embodiments, there is provided a population of genetically engineered and gene edited immune cells, comprising genetically engineered immune cells that express a cytotoxic receptor comprising an extracellular ligand binding domain, a transmembrane domain, and a cytotoxic signaling complex, wherein the extracellular ligand binding domain targets an antigen expressed by cells of a target tumor or cancer, the immune cells are genetically edited within a target sequence in the MED12 gene, the edit yields reduced expression and/or function of the mediator complex subunit 12 (MED12) protein encoded by the MED12 gene, as compared to an immune cell not edited within the target sequence in the MED12 gene, and the edit to the MED12 gene is made using an RNA-guided endonuclease. In several embodiments, there is provided a population of genetically engineered and gene edited immune cells, comprising genetically engineered immune cells that express a cytotoxic receptor comprising an extracellular ligand binding domain, a transmembrane domain, and a cytotoxic signaling complex, wherein the extracellular ligand binding domain targets an antigen expressed by cells of a target tumor or cancer, the immune cells are genetically edited within a target sequence in the MED12 gene and within a target sequence in the CISH gene, the edits yield reduced expression and/or function of the mediator complex subunit 12 (MED12) protein and the CIS protein, as compared to an immune cell not edited within the target sequence in the MED12 and CISH genes, and the edits are made using an RNA-guided endonuclease. In several embodiments, the disease or condition is cancer. In several embodiments, the immune cells are allogenic to the subject.

Also provided herein is a method for treating a disease or condition in a subject comprising, administering to the subject a population of genetically engineered immune cells, wherein the immune cells express a cytotoxic receptor comprising an extracellular ligand binding domain, a transmembrane domain, and a cytotoxic signaling complex, wherein the immune cells are genetically edited within a target sequence in the CISH gene, and wherein the immune cells are edited within a target sequence in a target gene selected from ADAM17, HIF-1a, DGKz, GSK-3B, LAG3, TIM3, TRIM29, IL-1R8, CD38, FBP-1, INSIG1, MED12, MED13, CCNC, CDK8, ID3, SOX4, or any combination thereof. In some embodiments, the disease or condition is an autoimmune disease, an infectious disease, or a cancer. In several embodiments, the disease or condition is cancer. In some embodiments, the disease or condition is an autoimmune disease. In some embodiments, the disease or condition is an infectious disease. In several embodiments, there is provided for herein a method for treating cancer in a subject comprising, administering to the subject a population of genetically engineered immune cells, wherein the immune cells express a cytotoxic receptor comprising an extracellular ligand binding domain, a transmembrane domain, and a cytotoxic signaling complex, wherein the immune cells are genetically edited at one or more target locations in a CISH gene that encodes a CIS protein, wherein the edits yield reduced expression and/or function of CIS as compared to an immune cell not edited at the location or locations in the CISH gene, wherein the immune cells are edited at one or more target locations in one more target genes selected from ADAM17, HIF-1a, DGKz, GSK-3B, LAG3, TIM3, TRIM29, IL-1R8, CD38, FBP-1, INSIG1, MED12, MED13, CCNC, CDK8, ID3, SOX4, or any combination thereof, wherein the edits are made using an RNA-guided endonuclease, and wherein the genetically engineered and edited immune cells exhibit one or more of enhanced expansion capability, enhanced cytotoxicity against target tumor cells, and enhanced persistence, particularly in a hypoxic tumor microenvironment, as compared to immune cells that do not comprise said genetically edited target site or sites.

Also provided herein a method for treating a disease or condition in a subject comprising, administering to the subject a population of genetically engineered immune cells, wherein the immune cells express a cytotoxic receptor comprising an extracellular ligand binding domain, a transmembrane domain, and a cytotoxic signaling complex, wherein the immune cells are genetically edited within a target sequence in the CISH gene, wherein the immune cells are genetically edited within a target sequence in the CBLB gene, and wherein the immune cells are edited within a target sequence in a target gene selected from ADAM17, HIF-1a, DGKz, GSK-3B, LAG3, TIM3, TRIM29, IL-1R8, CD38, FBP-1, INSIG1, MED12, MED13, CCNC, CDK8, ID3, SOX4, or any combination thereof. In some embodiments, the disease or condition is an autoimmune disease, an infectious disease, or a cancer. In several embodiments, the disease or condition is cancer. In some embodiments, the disease or condition is an autoimmune disease. In some embodiments, the disease or condition is an infectious disease. Also provided for herein is a method for treating cancer in a subject comprising, administering to the subject a population of genetically engineered immune cells, wherein the immune cells express a cytotoxic receptor comprising an extracellular ligand binding domain, a transmembrane domain, and a cytotoxic signaling complex, wherein the immune cells are genetically edited at one or more target locations in a CISH gene that encodes a CIS protein, wherein the edits yield reduced expression and/or function of CIS as compared to an immune cell not edited at the location or locations in the CISH gene, wherein the immune cells are genetically edited at one or more target locations in a CBLB gene that encodes a CBLB protein, wherein the edits yield reduced expression and/or function of CIS as compared to an immune cell not edited at the location or locations in the CBLB gene, wherein the immune cells are edited at one or more target locations in one more target genes selected from ADAM17, HIF-1a, DGKz, GSK-3B, LAG3, TIM3, TRIM29, IL-1R8, CD38, FBP-1, INSIG1, MED12, MED13, CCNC, CDK8, ID3, SOX4, or any combination thereof, wherein the edits are made using an RNA-guided endonuclease, and wherein the genetically engineered and edited immune cells exhibit one or more of enhanced expansion capability, enhanced cytotoxicity against target tumor cells, and enhanced persistence, particularly in a hypoxic tumor microenvironment, as compared to immune cells that do not comprise said genetically edited target site or sites. In several embodiments, the administered immune cells are allogeneic with respect the subject. In several embodiments, the treatment methods further comprise administering IL2.

In several embodiments, the immune cells are genetically edited within a target sequence in the ADAM17 gene. In several embodiments, the immune cells are genetically edited within a target sequence in the HIF1A gene. In several embodiments, the immune cells are genetically edited within a target sequence in the DGKz gene. In several embodiments, the immune cells are genetically edited within a target sequence in the GSK3B gene. In several embodiments, the immune cells are genetically edited within a target sequence in the LAG3 gene. In several embodiments, the immune cells are genetically edited within a target sequence in the TIM3 gene. In several embodiments, the immune cells are genetically edited within a target sequence in the TRIM29 gene. In several embodiments, the immune cells are genetically edited within a target sequence in the IL1R8 gene. In several embodiments, the immune cells are genetically edited within a target sequence in the CD38 gene. In several embodiments, the immune cells are genetically edited within a target sequence in the FBP1 gene. In several embodiments, the immune cells are genetically edited within a target sequence in the INSIG1 gene. In several embodiments, the immune cells are genetically edited within a target sequence in the MED12 gene. In several embodiments, the immune cells are genetically edited within a target sequence in the MED13 gene. In several embodiments, the immune cells are genetically edited within a target sequence in the CCNC gene. In several embodiments, the immune cells are genetically edited within a target sequence in the CDK8 gene. In several embodiments, the immune cells are genetically edited within a target sequence in the ID3 gene. In several embodiments, the immune cells are genetically edited within a target sequence in the SOX4 gene.

In several embodiments, ADAM17 is edited and wherein a guide sequence of any of SEQ ID NO: 682-687 is used to target the ADAM17 gene. In some embodiments, ADAM17 is edited at a target sequence comprising SEQ ID NO:682. In some embodiments, ADAM17 is edited at a target sequence comprising SEQ ID NO:683. In some embodiments, ADAM17 is edited at a target sequence comprising SEQ ID NO:684. In some embodiments, ADAM17 is edited at a target sequence comprising SEQ ID NO:685. In some embodiments, ADAM17 is edited at a target sequence comprising SEQ ID NO:686. In some embodiments, ADAM17 is edited at a target sequence comprising SEQ ID NO:687.

In several embodiments, ADAM17 is edited and the target sequence comprises any of SEQ ID NO: 682-687. In some embodiments, ADAM17 is edited and the target sequence comprises SEQ ID NO:682. In some embodiments, ADAM17 is edited and the target sequence comprises SEQ ID NO:683. In some embodiments, ADAM17 is edited and the target sequence comprises SEQ ID NO:684. In some embodiments, ADAM17 is edited and the target sequence comprises SEQ ID NO:685. In some embodiments, ADAM17 is edited and the target sequence comprises SEQ ID NO:686. In some embodiments, ADAM17 is edited and the target sequence comprises SEQ ID NO:687.

In several embodiments, MED12 is edited and wherein a guide sequence of any of SEQ ID NO:938-944 is used to target the MED12 gene. In some embodiments, MED12 is edited at a target sequence comprising SEQ ID NO:938. In some embodiments, MED12 is edited at a target sequence comprising SEQ ID NO:939. In some embodiments. MED12 is edited at a target sequence comprising SEQ ID NO:940. In some embodiments, MED12 is edited at a target sequence comprising SEQ ID NO:941. In some embodiments, MED12 is edited at a target sequence comprising SEQ ID NO:942. In some embodiments, MED12 is edited at a target sequence comprising SEQ ID NO:943. In some embodiments, MED12 is edited at a target sequence comprising SEQ ID NO:944. In some embodiments, MED12 is edited at a target sequence comprising SEQ ID NO:996. In some embodiments, MED12 is edited at a target sequence comprising SEQ ID NO:997. In some embodiments, MED12 is edited at a target sequence comprising SEQ ID NO:998.

In several embodiments, MED12 is edited and the target sequence comprises any of SEQ ID NO:938-944. In some embodiments, MED12 is edited and the target sequence comprises SEQ ID NO: 938. In some embodiments, MED12 is edited and the target sequence comprises SEQ ID NO:939. In some embodiments, MED12 is edited and the target sequence comprises SEQ ID NO:940. In some embodiments, MED12 is edited and the target sequence comprises SEQ ID NO:941. In some embodiments, MED12 is edited and the target sequence comprises SEQ ID NO:942. In some embodiments, MED12 is edited and the target sequence comprises SEQ ID NO:943. In some embodiments, MED12 is edited and the target sequence comprises SEQ ID NO:944. In some embodiments, MED12 is edited and the target sequence comprises SEQ ID NO: 996. In some embodiments, MED12 is edited and the target sequence comprises SEQ ID NO: 997. In some embodiments, MED12 is edited and the target sequence comprises SEQ ID NO: 998.

In several embodiments, CISH is edited and the target sequence comprises any of SEQ ID NO: 153-157, 463-466 or 1012-1013. In some embodiments, the target sequence comprises SEQ ID NO:153. In some embodiments, the target sequence comprises SEQ ID NO: 154. In some embodiments, the target sequence comprises SEQ ID NO:155. In some embodiments, the target sequence comprises SEQ ID NO:156. In some embodiments, the target sequence comprises SEQ ID NO:157. In some embodiments, the target sequence comprises SEQ ID NO:463. In some embodiments, the target sequence comprises SEQ ID NO:464. In some embodiments, the target sequence comprises SEQ ID NO:465. In some embodiments, the target sequence comprises SEQ ID NO:466. In some embodiments, the target sequence comprises SEQ ID NO: 1012. In some embodiments, the target sequence comprises SEQ ID NO:1013.

In several embodiments, HIF-1a is edited and wherein a guide sequence of any of SEQ ID NO: 750-760 is used to target the HIF-1a gene. In several embodiments, HIF-1a is edited and the target sequence comprises any of SEQ ID NO: 750-760. In several embodiments, DGKz is edited and wherein a guide sequence of any of SEQ ID NO: 688-723 is used to target the DGKz gene. In several embodiments, DGKz is edited and the target sequence comprises any of SEQ ID NO: 688-723. In several embodiments, GSK-3B is edited and wherein a guide sequence of any of SEQ ID NO: 724-749 is used to target the GSK-3B gene. In several embodiments, GSK-3B is edited and the target sequence comprises any of SEQ ID NO: 724-749. In several embodiments, LAG3 is edited and wherein a guide sequence of any of SEQ ID NO: 761-789 is used to target the LAG3 gene. In several embodiments, LAG3 is edited and the target sequence comprises any of SEQ ID NO: 761-789. In several embodiments, TIM3 is edited and wherein a guide sequence of any of SEQ ID NO: 790-825 is used to target the TIM3 gene. In several embodiments, TIM3 is edited and the target sequence comprises any of SEQ ID NO: 790-825. In several embodiments, TRIM29 is edited and wherein a guide sequence of any of SEQ ID NO: 826-835 is used to target the TRIM29 gene. In several embodiments, TRIM29 is edited and the target sequence comprises any of SEQ ID NO: 826-835. Tn several embodiments, TRIM29 is edited and wherein a guide sequence of any of SEQ ID NO: 167-169, 826-835, or 1009-1011 is used to target the TRIM29 gene. In several embodiments, TRIM29 is edited and the target sequence comprises any of SEQ ID NO: 167-169, 826-835 or 1009-1011. In several embodiments, IL-1R8 is edited and wherein a guide sequence of any of SEQ ID NO: 836-865 is used to target the IL-1R8 gene. In several embodiments. IL-1R8 is edited and the target sequence comprises any of SEQ ID NO: 836-865. In several embodiments, CD38 is edited and wherein a guide sequence of any of SEQ ID NO: 866-874 is used to target the CD38 gene. In several embodiments, CD38 is edited and the target sequence comprises any of SEQ ID NO: 866-874. In several embodiments, FBP-1 is edited and wherein a guide sequence of any of SEQ ID NO: 875-889 is used to target the FBP-1 gene. In several embodiments, FBP-1 is edited and the target sequence comprises any of SEQ ID NO: 875-889. In several embodiments, INSIG1 is edited and wherein a guide sequence of any of SEQ ID NO: 890-934 is used to target the INSIG1 gene. In several embodiments, INSIG1 is edited and the target sequence comprises any of SEQ ID NO: 890-934. In several embodiments, MED12 is edited and wherein a guide sequence of any of SEQ ID NO: 938-944 is used to target the MED12 gene. In several embodiments, MED12 is edited and wherein a guide sequence of any of SEQ ID NO: 938-944 or 996-998 is used to target the MED12 gene. In several embodiments, MED12 is edited and the target sequence comprises any of SEQ ID NO: 938-944. In several embodiments, MED12 is edited and the target sequence comprises any of SEQ ID NO: 938-944 or 996-998. In several embodiments, MED13 is edited and wherein a guide sequence of any of SEQ ID NO: 945-948 is used to target the MED13 gene. In several embodiments, MED13 is edited and the target sequence comprises any of SEQ ID NO: 945-948. In several embodiments, CDK8 is edited and wherein a guide sequence of any of SEQ ID NO: 949-955 is used to target the CDK8 gene. In several embodiments, CDK8 is edited and the target sequence comprises any of SEQ ID NO: 949-955. In several embodiments, CCNC is edited and wherein a guide sequence of any of SEQ ID NO: 956-961 is used to target the CCNC gene. In several embodiments, CCNC is edited and wherein a guide sequence of any of SEQ ID NO: 956-961 or 999-1001 is used to target the CCNC gene. In several embodiments, CCNC is edited and the target sequence comprises any of SEQ ID NO: 956-961. In several embodiments, CCNC is edited and the target sequence comprises any of SEQ ID NO: 956-961 or 999-1001. In several embodiments, ID3 is edited and wherein a guide sequence of any of SEQ ID NO: 963-969 is used to target the ID3 gene. In several embodiments, ID3 is edited and the target sequence comprises any of SEQ ID NO: 963-969. In several embodiments, SOX4 is edited and wherein a guide sequence of any of SEQ ID NO: 970-976 is used to target the SOX4 gene. In several embodiments, SOX4 is edited and the target sequence comprises any of SEQ ID NO: 970-976. In several embodiments, the immune cells are further edited at a CISH gene that encodes a CIS protein. In several embodiments, a guide sequence of any of SEQ ID NO: 153-157 or 463-466 is used to target the CISH gene. In several embodiments, a guide sequence of any of SEQ ID NO: 153-157, 463-466 or 1012-1013 is used to target the CISH gene. In several embodiments, CISH is edited and the target sequence comprises any of SEQ ID NO: 153-157 or 463-466. In several embodiments, CISH is edited and the target sequence comprises any of SEQ ID NO: 153-157, 463-466 or 1012-1013. In several embodiments, the cells are edited at an additional target site in a CBLB gene. In several embodiments, a guide sequence of any of SEQ ID NO: 164 to 166 or 453-456 is used to target the CBLB gene. In several embodiments, a guide sequence of any of SEQ ID NO: 164 to 166, 453-456 or 1005-1008 is used to target the CBLB gene. In several embodiments, CBLB is edited and the target sequence comprises any of SEQ ID NO: 164 to 166 or 453-456. In several embodiments, CBLB is edited and the target sequence comprises any of SEQ ID NO: 164 to 166, 453-456 or 1005-1008.

According to several embodiments, the cells are optionally further edited at a gene encoding CD70. In several embodiments, the cells are optionally edited at a TGFBR2 gene, a TIGIT gene, an adenosine A2 receptor gene, a SMAD3 gene, a MAPKAPK3 gene, a CEACAM1 gene, a DDIT4 gene, an NKG2A gene, a SOCS2 gene, a B2M gene, a PD-1gene, and/or a TCR alpha gene.

In several embodiments, at least a portion of the genetically engineered immune cells are engineered to express membrane bound IL-15. In several embodiments, the genetically engineered immune cells are engineered to express membrane bound IL-15. In some embodiments, the IL15 is expressed from a separate cassette on the construct comprising any one of the CARs disclosed herein. In some embodiments, the IL15 is expressed from a separate cassette on the construct comprising any one of the cytotoxic receptors disclosed herein. In some embodiments, the IL15 is expressed from the same cassette as any one of the CARs disclosed herein. In some embodiments, the IL15 is expressed from the same cassette as any one of the cytotoxic receptors disclosed herein. In some embodiments, the IL15 and cytotoxic receptor are expressed bicistronically. In some embodiments, the chimeric receptor and IL15 are separated by a nucleic acid sequence encoding a cleavage site, for example, a proteolytic cleavage site or a T2A, P2A, E2A, or F2A self-cleaving peptide cleavage site. In some embodiments, the chimeric receptor and IL15 are separated by a T2A sequence. In some embodiments, the IL15 is a membrane-bound IL15 (mbIL15).

In several embodiments, the immune cells comprise Natural Killer (NK) cells, T cells, induced pluripotent stem cells (iPSCs), iPSC-derived NK cells, NK-92 cells, or combinations thereof. In several embodiments, the immune cells comprise Natural Killer (NK) cells. In several embodiments, the genetically engineered and edited immune cells are suitable for use in allogeneic cancer cell therapy and wherein the cells maintain enhanced cytotoxicity and/or persistence in a hypoxic tumor microenvironment. In several embodiments, the genetically engineered and edited immune cells exhibit increased persistence in vivo as compared to genetically engineered cells not edited at a target sequence.

In several embodiments, the genetically engineered and edited immune cells provided for herein are used for the treatment of a disease or condition. In some embodiments, the disease or condition is an autoimmune disease. In several embodiments, the disease or condition is cancer. In several embodiments, the genetically engineered and edited immune cells provided for herein are used for the treatment of cancer. In several embodiments, the genetically engineered and edited immune cells provided for herein are used for the preparation of a medicament for the treatment of a disease or condition. In several embodiments, the disease or condition is an autoimmune disease. In several embodiments, the disease or condition is cancer. In several embodiments, the genetically engineered and edited immune cells provided for herein are used for the preparation of a medicament for the treatment of cancer.

### BRIEF DESCRIPTION OF THE DRAWINGS

FIG. 1A-1D depict non-limiting examples of tumor-directed chimeric antigen receptors.

FIG. 2 depicts a schematic workflow for assessing gene edits as disclosed herein.

FIG. 3A-3B show data related to expression of a non-limiting example of a CD19-directed CAR when cells are edited to disrupt expression of the indicated target genes. FIG. 3A shows data after editing of DGKz, GSK-3B, HIF-1a, TRIM29, IL-1R8, CD38, FBP-1 or electroporation (EP) control. FIG. 3B shows data after editing of ADAM17, LAG3, TIM3, INSIG1, CISH-15, or EP untransduced control. Similar results were achieved in cells from other donors (data not shown).

FIG. 4A-4C show data related to the knockout efficiency of ADAM17, LAG3, and TIM3 in a non-limiting example of NK cells expressing a CD19-directed CAR when cells are edited to disrupt expression of the indicated target genes. FIG. 4A depicts ADAM17KO efficiency in cells at day 11, with APC isotype controls and EP untransduced controls. FIG. 4B depicts TIM3 and CD38KO efficiency in cells at day 11. FIG. 4C depicts LAG3KO efficiency in cells at day 11. Similar results were achieved in cells from other donors (data not shown).

FIG. 5A-C depict summary expression data of the indicated genes post-editing in a non-limiting example of a CD19-directed CAR. FIG. 5A summarizes data from a first donor (512), FIG. 5C summarizes data from a second donor (558), and FIG. 5B summarizes data from a third donor (548).

FIG. 6 depicts the results of on-target INDEL analysis via CRISPR for the indicated genes in donors 558, 548, and 512 in a non-limiting example of a CD19-directed CAR.

FIG. 7A-B depict a summary of the fold expansion results for the cells expressing CD19-directed CAR when cells were edited to disrupt expression of the indicated target genes. Fold expansion was determined at days 0-7, 7-14, and 0-14 for cells from donors 512 (FIG. 7A) and 558 (FIG. 7B).

FIG. 8A-8B depict in vitro cytotoxicity data against tumor cells. FIG. 8 shows data related to NK cells expressing a non-limiting example of a CD19-directed CAR and tested as indicated (single edits) beginning at day 14 (FIG. 8A) and day 21 (FIG. 8B). Similar results were seen in cells from other donors (data not shown).

FIG. 9A-9B relate to glycolysis stress test and hypoxia data. FIG. 9A shows corresponding extracellular acidification rate (ECAR) data for NK cells expressing a CD19-directed CAR and edited as indicated. FIG. 9B shows additional data from the evaluation of the oxygen consumption rate (OCR) for cells and determination of mitochondrial vs. non-mitochondrial respiration in NK cells expressing a CD19-CAR and edited as indicated.

FIG. 10 depicts the results of a cytokine production assessment following coculture of NK cells from donor 512 expressing a CD19-CAR and edited as indicated incubated with Raji cells for 3 days, where the results for CD19-CAR expressing, gene edited, groups are compared to cytokine levels in EP control, Raji cells, and CD19-CAR expressing cells without additional gene editing.

FIG. 11 depicts a schematic workflow for assessing gene edits as disclosed herein.

FIG. 12 depicts summary expression data for the cells expressing a non-limiting example of a CD70-directed CAR and edited to disrupt expression of the indicated target genes. % viability, % CD70 positive cells, and % CAR positive cells were indicated for each group in comparison to an unedited, untransduced control.

FIG. 13 depicts a summary of the fold expansion results for the cells expressing a non-limiting example of a CD70-directed CAR when cells were edited to disrupt expression of the indicated target genes. Fold expansion was determined at days 0-6, 6-7, 7-14, and 0-14.

FIG. 14 depicts in vitro cytotoxicity data against tumor cells. FIG. 14 shows data related to NK cells expressing a non-limiting example of a CD70-directed CAR and edited as indicated from donor 512 at day 14 against HL60 and Molm13 cells. Each assay was performed at a 1:2 ratio of effector cells to target cells (E:T) and controls were target (tumor) cells alone and incubation of target (tumor) cells with unedited, untransduced NK cells (EP).

FIG. 15 depicts the results of a cell mitochondrial stress test where NK cells expressing a non-limiting example of a CD70-directed CAR and edited as indicated were treated with lactate for three days before assessment of OCR.

FIG. 16 depicts a schematic workflow for assessing gene edits as disclosed herein.

FIG. 17 depicts summary expression data for cells expressing a non-limiting example of a CD70-directed CAR and edited to disrupt expression of the indicated target genes. % ADAM17 positive, % CD70 positive cells, and % CAR positive cells were indicated for each group in comparison to an unedited, untransduced control (EP).

FIG. 18 depicts a summary of the fold expansion results for the cells expressing a non-limiting example of a CD70-directed CAR when cells were edited to disrupt expression of the indicated target genes. Fold expansion was determined at days 0-7, 7-15, and 1-15.

FIG. 19A-19D depict in vitro cytotoxicity data against tumor cells. FIG. 19A-19B show data related to NK cells expressing a non-limiting example of a CD70-directed CAR and tested as indicated beginning at day 14 against 786-0 cells with an effector to target ratio (E:T) of 1:2 for FIG. 19A and 1:4 for FIG. 19B. FIG. 19C-19D show cytotoxicity data related to NK cells expressing a non-limiting example of a CD70-directed CAR and tested as indicated beginning at day 14 against HL60 cells with an E:T of 1:1 for FIG. 19C and 1:2 for FIG. 19D.

FIG. 20 depicts the results of a cell mitochondrial stress test under hypoxic conditions for NK cells expressing a non-limiting example of a CD70-directed CAR and edited as indicated.

FIG. 21 depicts a schematic workflow for assessing gene edits as disclosed herein.

FIG. 22A-22B show data related to expression of a non-limiting example of a CD19-directed CAR when cells are edited to disrupt expression of ADAM17. FIG. 22A depicts the expression of the CD19 CAR on NK cells day 4 post-transduction. FIG. 22B depicts verification of knockout of ADAM17 from NK cells of donor 512 and 558.

FIG. 23A-23B depict assessment of CD16 and CD62L expression on cells expressing a non-limiting example of CD19-directed CAR and edited at ADAM17 (ADAM17 KO) and treated with DMSO (control), or with phorbol myristate acetate (PMA) stimulation at 1 μg/mL for 1 hour. Similar results were seen in cells from other donors (data not shown). FIG. 23B summarizes the data of FIG. 23A and reports CD16 and CD62L % positive and MFI for control and PMA treated conditions for EP control and ADAM17 edited cells with and without NKX19 for cells from two donors.

FIG. 23C depicts assessment of the expression of various ADAM17 substrates on cells expressing a non-limiting example of a CD70-directed CAR and edited at ADAM17 (ADAM17 KO) and treated with DMSO (control) or PMA stimulation at 1 μg/mL for 1 hour.

FIG. 24A-24G depict in vitro cytotoxicity data of NK cells edited as indicated against tumor cells. FIG. 24A depicts a study where Raji and Nalm6 cells were evaluated for CD20 expression.

FIG. 24B shows the cytotoxicity assay results for ADAM17 edited cells from donor 512 and 558 tested against Raji cells at E:T 2:1. In FIG. 24C-24G, Raji cells were precoated with Cetuximab (anti-EGFR) or Rituximab (anti-CD20) for 30 minutes in order to determine if antibody dependent cytotoxicity (ADCC) was enhanced when antibody coated Raji cells were incubated with CAR NKs with ADAM17 editing. FIG. 24C shows results for NK cells expressing a CD70-directed CAR and edited as indicated with or without the presence of Cetuximab at an E:T of 1:2 (FIG. 24C) or 1:4 (FIG. 24D). Additional ADCC assays were performed following incubation of target cells with Rituximab, the assays were performed at 1:2 (FIG. 24E) and 1:4 (FIG. 24F) with expression of a CD19-directed CAR, and at 1:1 without expression of a CD19-directed CAR (FIG. 24G).

FIG. 25 depicts a schematic workflow for assessing gene edits as disclosed herein.

FIG. 26 summarizes % viability and fold expansion data from day 1 and day 3 for NK cells expressing a non-limiting example of a CD70-directed CAR and edited as indicated.

FIG. 27A-27F depict flow cytometry results for NK cells expressing a non-limiting example of a CD70-directed CAR and edited as indicated. FIG. 27A-27B depict CD56 and CD70 staining of NK cells expressing a CD70-directed CAR and edited as indicated. FIG. 27C-27E depicts % CD70 positive cells for edited for CD38 (FIG. 27C), LAG3 (FIG. 27D), ADAM17 (FIG. 27E). FIG. 27F summarizes the data from FIG. 27C-27E.

FIG. 28 depicts the results of a cell mitochondrial stress test under hypoxic conditions for NK cells expressing a non-limiting example of a CD70-directed CAR and edited as indicated.

FIG. 29A-29B depict in vitro cytotoxicity data against tumor cells for NK cells expressing a non-limiting example of a CD70-directed CAR and edited as indicated. FIG. 29A shows cytotoxicity data for CD70 directed CARs edited to be triple knockouts. FIG. 29B shows cytotoxicity data for CD70 directed CARs edited to be quadruple knockouts.

FIG. 30A depicts the results of an in vivo anti-tumor activity assay where mice were injected with 786-0 cells at day −7, followed by injection of NK cells expressing a non-limiting example of a CD70-directed CAR and edited as indicated to be double (CISH/CBLB) or triple (CISH/CBLB/HIF1a, CISH/CBLB/ADAM17, or CISH/CBLB/FBP1) knockouts at day 0 and assessment of tumor volume (TV) over a 25 day period.

FIG. 30B depicts the results of an in vivo anti-tumor activity assay where mice were injected with HL60 cells at day −2, followed by injection of NK cells expressing a non-limiting example of a CD70-directed CAR and edited as indicated to be double (CISH/CBLB) or triple (CISH/CBLB/ADAM17) knockouts at day 0 and assessment of tumor volume (TV) over a 30 day period.

FIG. 31 depicts a schematic workflow for assessing gene edits as disclosed herein.

FIG. 32A-32B depicts in vitro cytotoxicity data against tumor cells for NK cells expressing a non-limiting example of a CD70-directed CAR and edited as indicated for the following genes: MED12, CCNC, CDK8. ID3, SOX4.

FIG. 33 depicts expression of a CD70 targeting CAR by NK cells from three different donors edited as indicated for the following genes: CD70, MED12, CDK8, CCNC, CISH, ID3, SOX4.

FIG. 34A-34B relate to a glycolysis stress test and glycolytic capacity, respectively. FIG. 34A and FIG. 34B show corresponding extracellular acidification rate (ECAR) and the oxygen consumption rate (OCR) data for NK cells expressing a CD70-directed CAR and edited as indicated.

FIG. 35 depicts expression of a CD19 targeting CAR by NK cells from a donor edited at the indicated target genes.

FIG. 36A-36B depict in vitro cytotoxicity data against Nalm6 tumor cells. FIG. 36A-36B show data related to NK cells expressing a non-limiting example of a CD19-directed CAR and edited as indicated at day 6 against Nalm6 cells with an effector to target ratio (E:T) of 1:1 and in the absence of TGF-β for FIG. 36A and in the presence of TGF-β for FIG. 36B.

FIG. 37 relates to the extracellular acidification rate (ECAR) data for NK cells expressing a CD19-directed CAR and edited as indicated.

FIG. 38A-38B relate to the proliferative ability of NK cells expressing a CD19-directed CAR and edited as indicated. FIG. 38A depicts the proliferative ability of the edited CD19-CAR NK cells from three different healthy donors and FIG. 38B depicts the quantification of the data presented in FIG. 38A.

FIG. 39 depicts a schematic workflow for assessing gene edits as disclosed herein.

FIG. 40A-40B depict in vitro cytotoxicity data against Nalm6 tumor cells. FIG. 40A-40B show data related to NK cells expressing a non-limiting example of a CD19-directed CAR and tested as indicated beginning at day 14 against Nalm6 cells in the absence (FIG. 40A) or presence (FIG. 40B) of TGF-β with Nalm6 target cells at a 1:2 or 1:1 E:T ratio, respectively.

FIG. 40C depicts assessment of cytokine production of the CD19-CAR NK cells from a donor edited as indicated at day 6 in the absence or presence of TGF-β with Nalm6 cells at a 1:1 E:T ratio by Luminex® multiplex assay.

FIG. 41A depicts a schematic of in vivo treatment with CD19 CAR NK cells edited as indicated.

FIG. 41B-41C depict tumor burden (FIG. 41B) and persistence of CD19 CAR NK cells (FIG. 41C), respectively, in a murine model of acute lymphoblastic leukemia (ALL).

FIG. 42A-42B depict the cytotoxicity of BCMA1 CAR-expressing NK cells edited at the indicated targets against BCMA-expressing Daudi cells at effector-to-target ratios (E:T) of 1:2 and 1:4, respectively.

FIG. 43A-43B depict the cytotoxicity of BCMA2 CAR-expressing NK cells edited at the indicated targets against BCMA-expressing MM.1S cells at effector-to-target ratios (E:T) of 1:1 and 1:2, respectively.

### DETAILED DESCRIPTION

Some embodiments of the methods and compositions provided herein relate to engineered immune cells and combinations of the same for use in immunotherapy. In several embodiments, the engineered cells are engineered in multiple ways, for example, to express a cytotoxicity-inducing receptor complex. As used herein, the term “cytotoxic receptor complexes” shall be given its ordinary meaning and shall also refer to (unless otherwise indicated), Chimeric Antigen Receptors (CAR) and chimeric receptors (also called activating chimeric receptors in the case of NKG2D chimeric receptors). In several embodiments, the cells are further engineered to achieve a modification of the reactivity of the cells against non-tumor tissue. Several embodiments relate to the modification of T cells, through various genetic engineering methodologies, such that the resultant T cells have reduced and/or eliminated alloreactivity. Such non-alloreactive T cells can also be engineered to express a chimeric antigen receptor (CAR) that enables the non-alloreactive T cells to impart cytotoxic effects against tumor cells. In several embodiments, natural killer (NK) cells are also engineered to express a cytotoxicity-inducing receptor complex (e.g., a chimeric antigen receptor or chimeric receptor). In several embodiments, combinations of these engineered immune cell types are used in immunotherapy, which results in both a rapid (NK-cell based) and persistent (T-cell based) anti-tumor effect, all while advantageously having little to no graft versus host disease (GvHD). Some embodiments include methods of use of the compositions or cells in immunotherapy.

While autologous CAR T cell therapies have been developed and shown to exhibit substantial in vivo persistence and efficacy, the majority of patients treated with autologous CAR T cell therapy will experience cytokine release syndrome (CRS) and/or a neurotoxicity. Further, autologous CAR T cell therapies face numerous challenges, including the need to leukapherese and then manufacture a conforming CAR T cell product from patients who are often extremely sick, heavily pre-treated, or both. Manufacturing sufficient numbers of CAR T cells from such patients can be difficult, or in some cases, impossible. In addition, a potential patient may not survive the length of time it takes to manufacture the final CAR T cell product from the T cells obtained from the patient.

By contrast, NK cell therapies, including allogeneic NK cell therapies manufactured from healthy donors, can obviate many of these challenges. For example, manufacturing success rates for allogeneic CAR NK cells may be higher due to better quality of incoming donor cells. Allogeneic CAR NK cell therapies can also be provided when a patient is in need, without having to wait for the patient's own cells to be manufactured. Thus, allogeneic NK cell therapies are being investigated for use as off-the-shelf products. Despite the potential advantages offered by NK cells, they have not been shown to persist in vivo to the same extent as T cells. Solutions are therefore needed to overcome this challenge. Described herein are genetic edits that can increase the persistence, efficacy (e.g., cytotoxicity), or both, of NK cells. Embodiments of such genetically edited NK cells include compositions and methods of using the same to treat a disease or condition (e.g., cancer) in a subject. For example, experiments described herein found that disruption of particular genes, including e.g., ADAM17, MED12, CISH, CBLB, or a combination thereof, imparted surprisingly beneficial effects to NK cells, including enhanced cytotoxicity, both in vitro and in vivo. These results were observed in NK cells expressing CARs targeting different antigens (e.g., BCMA, CD19 or CD70). Without wishing to be bound by theory, these findings are consistent with an observation that such gene edits can impart advantages to CAR-expressing NK cells, regardless of the particular antigen targeted by the CAR.

The term “anticancer effect” refers to a biological effect which can be manifested by various means, including but not limited to, a decrease in tumor volume, a decrease in the number of cancer cells, a decrease in the number of netastases, an increase in life expectancy, decrease in cancer cell proliferation, decrease in cancer cell survival, and/or amelioration of various physiological symptoms associated with the cancerous condition.

### Cell Types

Some embodiments of the methods and compositions provided herein relate to a cell such as an immune cell. In some embodiments, an immune cell is engineered to express a chimeric receptor that binds to an antigen (e.g., an antigen expressed by a cancer cell). For example, an immune cell, such as a T cell, may be engineered to include a chimeric receptor such as a CD19-directed chimeric receptor, or engineered to include a nucleic acid encoding said chimeric receptor as described herein. In some embodiments, a NK cell is engineered to express a chimeric receptor that binds to an antigen (e.g., an antigen expressed by a cancer cell). Additional embodiments relate to engineering a second set of cells to express another cytotoxic receptor complex, such as an NKG2D chimeric receptor complex as disclosed herein. Thus, in some embodiments, combinations or compositions comprising two different types of immune cells, (e.g., T cells and NK cells) are contemplated. In some embodiments, the engineered T cells and the engineered NK cells express the same chimeric receptor. In some embodiments, the engineered T cells and the engineered NK cells express different chimeric receptors. In some embodiments, the engineered T cells and the engineered NK cells express chimeric receptors that bind to the same antigen (e.g., different epitopes of the same antigen). In some embodiments, the engineered T cells and the engineered NK cells express chimeric receptors that binds different antigens.

Additional embodiments relate to the further genetic manipulation of NK cells (e.g., donor NK cells) to increase persistence and/or potency of engineered NK cells. Still additional embodiments relate to the further genetic manipulation of T cells (e.g., donor T cells) to reduce, disrupt, minimize and/or eliminate the ability of the donor T cell to be alloreactive against recipient cells (graft versus host disease). For example, in some embodiments, T cells are engineered to reduce alloreactivity against recipient cells.

Traditional anti-cancer therapies relied on a surgical approach, radiation therapy, chemotherapy, or combinations of these methods. As research led to a greater understanding of some of the mechanisms of certain cancers, this knowledge was leveraged to develop targeted cancer therapies. Targeted therapy is a cancer treatment that employs certain drugs that target specific genes or proteins found in cancer cells or cells supporting cancer growth, (like blood vessel cells) to reduce or arrest cancer cell growth. More recently, genetic engineering has enabled approaches to be developed that harness certain aspects of the immune system to fight cancers. In some cases, a patient's own immune cells are modified to specifically eradicate that patient's type of cancer. Various types of immune cells can be used, such as T cells, Natural Killer (NK cells), or combinations thereof, as described in more detail below.

To facilitate cancer immunotherapies, there are provided for herein polynucleotides (e.g., encoding chimeric receptors), polypeptides (e.g., chimeric receptors), and vectors that encode chimeric antigen receptors (CAR) that comprise a target binding moiety (e.g., an extracellular binder of a ligand, or a tumor marker-directed chimeric receptor, expressed by a cancer cell) and a cytotoxic signaling complex. For example, some embodiments include a polynucleotide, polypeptide, or vector that encodes, for example a chimeric antigen receptor directed against a tumor marker, for example, CD19, CD123, CD70, Her2, mesothelin, Claudin 6, BCMA, EGFR, among others, to facilitate targeting of an immune cell to a cancer and exerting cytotoxic effects on the cancer cell. Also provided are engineered immune cells (e.g., T cells or NK cells) expressing such CARs. In some embodiments, a chimeric antigen receptor binds to ligands of NKG2D. In some embodiments, a chimeric antigen receptor binds to CD19. In some embodiments, a chimeric antigen receptor binds to CD70. In some embodiments, a chimeric antigen receptor binds to BCMA. There are also provided herein, in several embodiments, polynucleotides, polypeptides, and vectors that encode a construct comprising an extracellular domain comprising two or more subdomains, e.g., first CD19-targeting subdomain comprising a CD19 binding moiety as disclosed herein and a second subdomain comprising a C-type lectin-like receptor and a cytotoxic signaling complex. Also provided are engineered immune cells (e.g., T cells or NK cells) expressing such bi-specific constructs. Methods of treating cancer and other uses of such cells for cancer immunotherapy are also provided for herein.

Also provided are chimeric receptors that comprise an antigen-binding domain and a cytotoxic signaling complex. For example, some embodiments include a chimeric receptor directed against a tumor antigen (e.g., CD19, BCMA, or CD70). Also provided are immune cells (e.g., NK cells) genetically engineered to express such CARs. In some embodiments, the immune cells are genetically edited (e.g., at MED12 and/or CISH).

To facilitate cancer immunotherapies, there are also provided for herein polynucleotides (e.g., encoding chimeric receptors), polypeptides (e.g., chimeric receptors), and vectors that encode chimeric receptors that comprise a target binding moiety (e.g., an extracellular binder of a ligand expressed by a cancer cell) and a cytotoxic signaling complex. For example, some embodiments include a polynucleotide, polypeptide, or vector that encodes, for example an activating chimeric receptor comprising an NKG2D extracellular domain that is directed against a tumor marker, for example, MICA, MICB, ULBP1, ULBP2, ULBP3, ULBP4, ULBP5, and ULBP6, among others, to facilitate targeting of an immune cell to a cancer and exerting cytotoxic effects on the cancer cell. In some embodiments, the chimeric receptor comprises an extracellular domain of NKG2D.

Also provided are engineered immune cells (e.g., T cells or NK cells) expressing such chimeric receptors. There are also provided herein, in several embodiments, polynucleotides, polypeptides, and vectors that encode a construct comprising an extracellular domain comprising two or more subdomains, e.g., first and second ligand binding receptor and a cytotoxic signaling complex. Also provided are engineered immune cells (e.g., T cells or NK cells) expressing such bi-specific constructs (in some embodiments the first and second ligand binding domain target the same ligand). Methods of treating cancer and other uses of such cells for cancer immunotherapy are also provided for herein.

### Engineered Cells for Immunotherapy

In several embodiments, cells of the immune system are engineered to have enhanced cytotoxic effects against target cells, such as tumor cells. For example, a cell of the immune system may be engineered to include a tumor-directed chimeric receptor and/or a tumor-directed CAR as described herein. In several embodiments, white blood cells or leukocytes, are used, since their native function is to defend the body against growth of abnormal cells and infectious disease. There are a variety of types of white bloods cells that serve specific roles in the human immune system, and are therefore a preferred starting point for the engineering of cells disclosed herein. White blood cells include granulocytes and agranulocytes (presence or absence of granules in the cytoplasm, respectively). Granulocytes include basophils, eosinophils, neutrophils, and mast cells. Agranulocytes include lymphocytes and monocytes. Cells such as those that follow or are otherwise described herein may be engineered to include a chimeric receptor, such as an NKG2D chimeric receptor, and/or a CAR, such as a CD19-directed CAR, or a nucleic acid encoding the chimeric receptor or the CAR. In several embodiments, the cells are optionally engineered to co-express a membrane-bound interleukin 15 (mbIL15) domain. In some embodiments, the immune cells engineered to express a chimeric receptor are engineered to bicistronically express a mbIL15 domain. As discussed in more detail below, in several embodiments, the cells, particularly T cells, are further genetically modified to reduce and/or eliminate the alloreactivity of the cells.

### Monocytes for Immunotherapy

In some embodiments, the immune cells comprise monocytes. Monocytes are a subtype of leukocyte. Monocytes can differentiate into macrophages and myeloid lineage dendritic cells. Monocytes are associated with the adaptive immune system and serve the main functions of phagocytosis, antigen presentation, and cytokine production. Phagocytosis is the process of uptake of cellular material, or entire cells, followed by digestion and destruction of the engulfed cellular material.

In some embodiments, a monocyte is positive for cell surface expression of a marker selected from among the group consisting of CCR2, CCR5, CD11c, CD14, CD16, CD62L, CD68+, CX3CR1, HLA-DR, or any combination thereof. In some embodiments, a monocyte is positive for cell surface expression of CD14. In some embodiments, a monocyte is positive for cell surface expression of CCR2. In some embodiments, a monocyte is positive for cell surface expression of CCR5. In some embodiments, a monocyte is positive for cell surface expression of CD62L.

In several embodiments, monocytes are used in connection with one or more additional engineered cells as disclosed herein. Some embodiments of the methods and compositions described herein relate to a monocyte that includes a tumor-directed CAR, or a nucleic acid encoding the tumor-directed CAR. In some embodiments, the monocytes express a CAR that binds to a tumor antigen, for example, CD19, CD123, CD70, Her2, mesothelin, Claudin 6, BCMA, or EGFR.

In some embodiments, the monocytes are engineered to express a membrane-bound interleukin 15 (mbIL15) domain. In some embodiments, the monocytes engineered to express a chimeric receptor are also engineered to also express (e.g., bicistronically express) a membrane-bound interleukin 15 (mbIL15) domain. Thus, in some embodiments, the monocytes are engineered to bicistronically express the chimeric receptor and mbIL15. Several embodiments of the methods and compositions disclosed herein relate to monocytes engineered to express a CAR that targets a tumor marker, for example, CD19, CD123, CD70, Her2, mesothelin, Claudin 6, BCMA, EGFR. among any of the others disclosed herein, and a membrane-bound interleukin 15 (mbIL15) domain. Several embodiments of the methods and compositions disclosed herein relate to monocytes engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-bound interleukin 15 (mbIL15) domain.

In some embodiments, the monocytes are allogeneic cells. In some embodiments, the monocytes are obtained from a donor who does not have cancer.

### Lymphocytes for Immunotherapy

In some embodiments, the immune cells comprise lymphocytes. Lymphocytes, the other primary sub-type of leukocyte include T cells (cell-mediated, cytotoxic adaptive immunity), natural killer cells (cell-mediated, cytotoxic innate immunity), and B cells (humoral, antibody-driven adaptive immunity). While B cells are engineered according to several embodiments, disclosed herein, several embodiments also relate to engineered T cells or engineered NK cells (mixtures of T cells and NK cells are used in some embodiments, either from the same donor, or different donors). Thus, in some embodiments, the immune cells comprise T cells. In some embodiments, the immune cells comprise NK cells. In some embodiments, the immune cells comprise T cells and NK cells. In some embodiments, the immune cells comprise B cells.

In several embodiments, lymphocytes are used in connection with one or more additional engineered cells as disclosed herein. Some embodiments of the methods and compositions described herein relate to a lymphocyte that includes a tumor-directed CAR, or a nucleic acid encoding the tumor-directed CAR. In some embodiments, the lymphocytes express a CAR that binds to a tumor antigen, for example, CD19, CD123, CD70. Her2, mesothelin, Claudin 6, BCMA, or EGFR.

In some embodiments, the lymphocytes are engineered to express a membrane-bound interleukin 15 (mbIL15) domain. In some embodiments, the lymphocytes engineered to express a chimeric receptor are also engineered to also express (e.g., bicistronically express) a membrane-bound interleukin 15 (mbIL15) domain. Thus, in some embodiments, lymphocytes are engineered to bicistronically express the chimeric receptor and mbIL15. Several embodiments of the methods and compositions disclosed herein relate to lymphocytes engineered to express a CAR that targets a tumor marker, for example, CD19, CD123, CD70, Her2, mesothelin, Claudin 6, BCMA, EGFR, among any of the others disclosed herein, and a membrane-bound interleukin 15 (mbIL15) domain. Several embodiments of the methods and compositions disclosed herein relate to lymphocytes engineered to express an activating chimeric receptor that targets a ligand on a tumor cell, for example, MICA, MICB, ULBP1. ULBP2, ULBP3, ULBP4, ULBP5, and ULBP6 (among others) and optionally a membrane-bound interleukin 15 (mbIL15) domain.

In some embodiments, the lymphocytes are allogeneic cells. In some embodiments, the lymphocytes are obtained from a donor who does not have cancer.

### T Cells for Immunotherapy

In some embodiments, the immune cells comprise T cells. T cells are distinguishable from other lymphocytes sub-types (e.g., B cells or NK cells) based on the presence of a T-cell receptor on the cell surface.

T cells can be divided into various different subtypes, including effector T cells, helper T cells, cytotoxic T cells, memory T cells, regulatory T cells, natural killer T cell, mucosal associated invariant T cells and gamma delta T cells. In some embodiments, a specific subtype of T cell is engineered. In some embodiments, a T cell is positive for cell surface expression of a marker selected from among the group consisting of CD3, CD4, and/or CD8. In some embodiments, a T cell is positive for cell surface expression of CD3. In some embodiments, a T cell is positive or cell surface expression of CD4. In some embodiments, a T cell is positive or cell surface expression of CD8.

In some embodiments, CD3+ T cells are engineered. In some embodiments, CD4+ T cells are engineered. In some embodiments, CD8+ T cells are engineered. In some embodiments, regulatory T cells are engineered. In some embodiments, gamma delta T cells are engineered. In some embodiments, a mixed pool of T cell subtypes is engineered. For example, in some embodiments, CD4+ and CD8+ T cells are engineered. In some embodiments, there is no specific selection of a type of T cells to be engineered to express the cytotoxic receptor complexes disclosed herein. In several embodiments, specific techniques, such as use of cytokine stimulation are used to enhance expansion/collection of T cells with a specific marker profile. For example, in several embodiments, activation of certain human T cells, e.g. CD4+ T cells, CD8+ T cells is achieved through use of CD3 and/or CD28 as stimulatory molecules.

In several embodiments, there is provided a method of treating or preventing cancer or an infectious disease, comprising administering a therapeutically effective amount of T cells expressing the cytotoxic receptor complex and/or a homing moiety as described herein. In several embodiments, there is provided a method of treating or preventing cancer or an infectious disease, comprising administering T cells expressing a cytotoxic receptor complex as described herein. In several embodiments, the engineered T cells are autologous cells, while in some embodiments, the T cells are allogeneic cells. In some embodiments, the T cells are allogeneic cells. In some embodiments, the T cells are obtained from a donor who does not have cancer.

Several embodiments of the methods and compositions disclosed herein relate to T cells engineered to express a CAR that targets a tumor marker, for example, CD19, CD123, CD70, Her2, mesothelin, Claudin 6, BCMA, EGFR, among any of the others as disclosed herein, and a membrane-bound interleukin 15 (mbIL15) domain. In some embodiments, T cells express a CAR that binds to CD19. In some embodiments. T cells express a CAR that binds to CD70. In some embodiments, T cells express a CAR that binds to BCMA. Several embodiments of the methods and compositions disclosed herein relate to T cells engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-hound interleukin 15 (mbIL15) domain. In some embodiments, T cells express a chimeric receptor that binds to a NKG2D ligand. In some embodiments, T cells express a chimeric receptor comprising an extracellular domain of NKG2D.

In some embodiments, the T cells are engineered to express a membrane-bound interleukin 15 (mbIL15) domain. In some embodiments, the T cells engineered to express a chimeric receptor are also engineered to also express (e.g., bicistronically express) a membrane-bound interleukin 15 (mbIL15) domain. Thus, in some embodiments, the T cells are engineered to bicistronically express the chimeric receptor and mbIL15.

In some embodiments, the immune cells comprise T cells and NK cells (either from the same donor or from different donors).

### NK Cells for Immunotherapy

In some embodiments, the immune cells comprise natural killer (NK) cells. In several embodiments, there is provided a method of treating or preventing cancer or an infectious disease, comprising administering a therapeutically effective amount of natural killer (NK) cells expressing the cytotoxic receptor complex and/or a homing moiety as described herein. In several embodiments, there is provided a method of treating or preventing cancer comprising administering a natural killer (NK) cells expressing a cytotoxic receptor complex as described herein. In several embodiments, there is provided a method of treating or preventing an infectious disease comprising administering a natural killer (NK) cells expressing a cytotoxic receptor complex as described herein. In several embodiments, the engineered NK cells are autologous cells, while in some embodiments, the NK cells are allogeneic cells.

In several embodiments, NK cells are preferred because the natural cytotoxic potential of NK cells is relatively high. In several embodiments, it is unexpectedly beneficial that the engineered cells disclosed herein can further upregulate the cytotoxic activity of NK cells, leading to an even more effective activity against target cells (e.g., tumor or other diseased cells).

In some embodiments, a NK cell is positive for cell surface expression of a marker selected from among the group consisting of CCR7, CD16, CD56, CD57, CD 11, CX3CR1, a Killer Ig-like receptor (KIR), NKp30, NKp44, NKp46, or any combination thereof. In some embodiments, a NK cell is positive for cell surface expression of CD16. In some embodiments, a NK cell is positive for cell surface expression of CD56. In some embodiments, a NK cell is positive for cell surface expression of a Killer Ig-like receptor.

Some embodiments of the methods and compositions described herein relate to NK cells engineered to express a CAR that targets a tumor marker, for example, CD19, CD123, CD70, Her2, mesothelin, Claudin 6, BCMA, EGFR, among any of the others disclosed herein, and optionally a membrane-bound interleukin 15 (mbIL15) domain. In some embodiments, NK cells express a CAR that hinds to CD19. In some embodiments, NK cells express a CAR that binds to CD70. Tn some embodiments, NK cells express a CAR that binds to BCMA. Several embodiments of the methods and compositions disclosed herein relate to NK cells engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-bound interleukin 15 (mbIL15)domain. In some embodiments, NK cells express a chimeric receptor that binds to a NKG2D ligand. In some embodiments, NK cells express a chimeric receptor comprising an extracellular domain of NKG2D.

In some embodiments, the NK cells are engineered to express a membrane-bound interleukin 15 (mbIL15) domain. In some embodiments, the NK cells engineered to express a chimeric receptor are also engineered to also express (e.g., bicistronically express) a membrane-bound interleukin 15 (mbIL15) domain. Thus, in some embodiments, the NK cells are engineered to bicistronically express the chimeric receptor and mbIL15.

In some embodiments, the NK cells are derived from cell line NK-92. NK-92 cells are derived from NK cells, but lack major inhibitory receptors displayed by normal NK cells, while retaining the majority of activating receptors. Some embodiments of NK-92 cells described herein related to NK-92 cell engineered to silence certain additional inhibitory receptors, for example, SMAD3, allowing for upregulation of interferon-γ (IFNγ), granzyme B, and/or perforin production. Additional information relating to the NK-92 cell line is disclosed in WO 1998/49268 and U.S. Patent Application Publication No. 2002-0068044 and incorporated in their entireties herein by reference.

In some embodiments, the NK cells are used in combination with T cells. Thus, in some embodiments, the immune cells comprise T cells and NK cells (either from the same donor or from different donors). NK-92 cells are used, in several embodiments, in combination with one or more of the other cell types disclosed herein. For example, in one embodiment, NK-92 cells are used in combination with NK cells as disclosed herein. In an additional embodiment, NK-92 cells are used in combination with T cells as disclosed herein.

### Hematopoietic Stem Cells for Cancer Immunotherapy

In some embodiments, hematopoietic stem cells (HSCs) are used in the methods of immunotherapy disclosed herein. In several embodiments, the cells are engineered to express a homing moiety and/or a cytotoxic receptor complex. In several embodiments, the cells are engineered to express a cytotoxic receptor complex. HSCs are used, in several embodiments, to leverage their ability to engraft for long-term blood cell production, which could result in a sustained source of targeted anti-cancer effector cells, for example to combat cancer remissions. In several embodiments, this ongoing production helps to offset anergy or exhaustion of other cell types, for example due to the tumor microenvironment.

In some embodiments, a HSC is positive for cell surface expression of a marker selected from among the group consisting of CD34, CD59, and CD90. In some embodiments, a HSC is positive for cell surface expression of CD34. In some embodiments, a HSC is positive for cell surface expression of CD59. In some embodiments, a HSC is positive for cell surface expression of CD90.

In several embodiments allogeneic HSCs are used, while in some embodiments, autologous HSCs are used. In several embodiments, HSCs are used in combination with one or more additional engineered cell type disclosed herein. Some embodiments of the methods and compositions described herein relate to a stem cell, such as a hematopoietic stem cell engineered to express a CAR that targets a tumor marker, for example, CD19, CD123, CD70, Her2, mesothelin, Claudin 6, BCMA, EGFR, among any of the others disclosed herein, and optionally a membrane-bound interleukin 15 (mbIL15) domain. Several embodiments of the methods and compositions disclosed herein relate to hematopoietic stem cells engineered to express an activating chimeric receptor that targets a ligand on a tumor cell, for example, MICA, MICB, ULBP1, ULBP2, ULBP3, ULBP4. ULBP5, and ULBP6 (among others) and optionally a membrane-bound interleukin 15 (mbIL15) domain.

### Induced Pluripotent Stem Cells

In some embodiments, immune cells are derived (differentiated) from pluripotent stem cells (PSCs). In some embodiments, immune cells (e.g., NK and/or T cells) derived from induced pluripotent stem cells (iPSCs) are used in the method of immunotherapy disclosed herein. For example, in some embodiments, NK cells are derived from iPSCs. In some embodiments, induced pluripotent stem cells (iPSCs) are used in the method of immunotherapy disclosed herein. iPSCs are used, in several embodiments, to leverage their ability to differentiate and derive into non-pluripotent cells, including, but not limited to, CD34 cells, hemogenic endothelium cells, HSCs (hematopoietic stem and progenitor cells), hematopoietic multipotent progenitor cells, T cell progenitors, NK cell progenitors, T cells, NKT cells, NK cells, and B cells comprising one or several genetic modifications at selected sites through differentiating iPSCs or less differentiated cells comprising the same genetic modifications at the same selected sites. In several embodiments, the iPSCs are used to generate iPSC-derived NK or T cells. In several embodiments, the iPSCs are used to generate iPSC-derived NK cells. In several embodiments, the iPSCs are used to generate iPSC-derived T cells.

In several embodiments, the cells are engineered to express a homing moiety and/or a cytotoxic receptor complex In several embodiments, the cells are engineered to express a cytotoxic receptor complex. In several embodiments, iPSCs are used in combination with one or more additional engineered cell type disclosed herein.

Several embodiments of the methods and compositions disclosed herein relate to induced pluripotent stem cells engineered to express an activating chimeric receptor that targets a ligand on a tumor cell, for example, CD19, CD123, CD70, Her2, mesothelin, Claudin 6, BCMA, or EGFR. In some embodiments, the iPSCs engineered to express a chimeric receptor are engineered to also express (e.g., bicistronically express) a membrane-hound interleukin 15 (mbIL15) domain. Some embodiments of the methods and compositions described herein relate to a stem cell, such as an induced pluripotent stem cell engineered to express a CAR that targets a tumor marker, for example, CD19, CD123, CD70, Her2, mesothelin, Claudin 6, BCMA, EGFR, among any of the others disclosed herein, and optionally a membrane-bound interleukin 15 (mbIL15) domain.

Several embodiments of the methods and compositions disclosed herein relate to induced pluripotent stem cells engineered to express an activating chimeric receptor that targets a ligand on a tumor cell, for example, MICA, MICB, ULBP1, ULBP2, ULBP3, ULBP4, ULBP5, and ULBP6 (among others). In some embodiments, the iPSCs engineered to express a chimeric receptor are engineered to also express (e.g., bicistronically express) a membrane-bound interleukin 15 (mbIL15) domain. Several embodiments of the methods and compositions disclosed herein relate to induced pluripotent stem cells engineered to express an activating chimeric receptor that targets a ligand on a tumor cell, for example, MICA, MICB. ULBP1, ULBP2, ULBP3, ULBP4, ULBP5, and ULBP6 (among others) and optionally a membrane-bound interleukin 15 (mbIL15) domain.

In several embodiments, the engineered iPSCs are differentiated into NK, T, or other immune cells, such as for use in a composition or method provided herein. In several embodiments, the engineered iPSCs are differentiated into NK cells. In several embodiments, the engineered iPSCs are differentiated into T cells. In several embodiments, the engineered iPSCs are differentiated into NK and T cells.

### Genetic Editing of Immune Cells

As discussed above, a variety of cell types can be utilized in cellular immunotherapy. Further, as elaborated on in more detail below, and shown in the Examples, genetic modifications can be made to these cells in order to enhance one or more aspects of their efficacy (e.g., cytotoxicity) and/or persistence (e.g., active life span). As discussed herein, in several embodiments NK cells are used for immunotherapy. In several embodiments provided for herein, gene editing of the NK cell can advantageously impart to the edited NK cell the ability to resist and/or overcome various inhibitory signals that are generated in the tumor microenvironment. It is known that tumors generate a variety of signaling molecules that are intended to reduce the anti-tumor effects of immune cells. As discussed in more detail below, in several embodiments, gene editing of the NK cell limits this tumor microenvironment suppressive effect on the NK cells, T cells, combinations of NK and T cells, or any edited/engineered immune cell provided for herein.

As discussed below, in several embodiments, gene editing is employed to reduce or knockout expression of target proteins, for example by disrupting the underlying gene encoding the protein.

In several embodiments, gene editing can reduce transcription of a target gene by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of a target gene by at least about 30%. In several embodiments, gene editing reduces transcription of a target gene by at least about 40%. In several embodiments, gene editing reduces transcription of a target gene by at least about 50%. In several embodiments, gene editing reduces transcription of a target gene by at least about 60%. In several embodiments, gene editing reduces transcription of a target gene by at least about 70%. In several embodiments, gene editing reduces transcription of a target gene by at least about 80%. In several embodiments, gene editing reduces transcription of a target gene by at least about 90%. In several embodiments, the gene is completely knocked out, such that transcription of the target gene is undetectable.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of a target protein by at least about 30%. In several embodiments, gene editing reduces expression of a target protein by at least about 40%. In several embodiments, gene editing reduces expression of a target protein by at least about 50%. In several embodiments, gene editing reduces expression of a target protein by at least about 60%. In several embodiments, gene editing reduces expression of a target protein by at least about 70%. In several embodiments, gene editing reduces expression of a target protein by at least about 80%. In several embodiments, gene editing reduces expression of a target protein by at least about 90%. In several embodiments, the gene is completely knocked out, such that expression of the target protein is undetectable.

In several embodiments, gene editing is used to “knock in” or otherwise increase transcription of a target gene. In several embodiments, transcription of a target gene is increased by about 30%, about 40%, about 50%, about 60%, about 70%, about 75%, about 80%, about 85%, about 90%, about 95%, about 97%, about 98%, about 99%, or more (including any amount between those listed). In several embodiments, transcription of a target gene is increased by at least about 30%. In several embodiments, transcription of a target gene is increased by at least about 40%. In several embodiments, transcription of a target gene is increased by at least about 50%. In several embodiments, transcription of a target gene is increased by at least about 60%. In several embodiments, transcription of a target gene is increased by at least about 70%. In several embodiments, transcription of a target gene is increased by at least about 80%. In several embodiments, transcription of a target gene is increased by at least about 90%. In several embodiments, transcription of a target gene is increased by at least about 100%.

In several embodiments, gene editing is used to “knock in” or otherwise enhance expression of a target protein. In several embodiments, expression of a target protein can be enhanced by about 30%, about 40%, about 50%, about 60%, about 70%, about 75%, about 80%, about 85%, about 90%, about 95%, about 97%, about 98%, about 99%, or more (including any amount between those listed). In several embodiments, expression of a target protein is increased by at least about 30%. In several embodiments, expression of a target protein is increased by at least about 40%. In several embodiments, expression of a target protein is increased by at least about 50%. In several embodiments, expression of a target protein is increased by at least about 60%. In several embodiments, expression of a target protein is increased by at least about 70%. In several embodiments, expression of a target protein is increased by at least about 80%. In several embodiments, expression of a target protein is increased by at least about 90%. In several embodiments, expression of a target protein is increased by at least about 100%.

Unless indicated otherwise to the contrary, the sequences provided for guide RNAs (gRNAs) that are recited using deoxyribonucleotides refer to the target DNA sequence (which is complementary to the corresponding non-target DNA sequence to which the gRNA binds) and shall be considered as also referencing those guides used in practice (e.g., employing ribonucleotides, where the ribonucleotide uracil is used in lieu of deoxyribonucleotide thymine or vice-versa where thymine is used in lieu of uracil, wherein both are complementary base pairs to adenine when reciting either an RNA or DNA sequence). In other words, the sequences provided for particular gRNAs provided herein are identical to the gRNA sequences used in practice, except that the gRNA sequences include uracil in lieu of thymine. For example, a gRNA with the sequence ATGCTCAATGCGTC (SEQ ID NO:977) shall also refer to the following sequence AUGCUCAAUGCGUC (SEQ ID NO:978) or a gRNA with sequence AUGCUCAAUGCGUC (SEQ ID NO:978) shall also refer to the following sequence ATGCTCAATGCGTC (SEQ ID NO:977). Further, the non-target DNA sequence to which a particular gRNA sequence binds is complementary to the sequence of the particular gRNA. For example, a gRNA with the provided sequence of ATGCTCAATGCGTC (SEQ ID NO: 977) binds to a non-target DNA sequence of TACGAGTTACGCAG (SEQ ID NO: 979). In this situation, the corresponding target DNA sequence, which is complementary to the non-target DNA sequence, is ATGCTCAATGCGTC (SEQ ID NO: 977).

In several embodiments, gene editing of the immune cells can also provide unexpected enhancement in the expansion, persistence and/or cytotoxicity of the edited immune cell. As disclosed herein, engineered cells (e.g., those expressing a CAR) may also be edited, the combination of which provides for a robust cell for immunotherapy. In several embodiments, the edits allow for unexpectedly improved NK cell expansion, persistence and/or cytotoxicity. In several embodiments, knockout of gene expression in NK cells removes a potent negative regulator or other suppressor of signaling and/or activity of NK cells, thereby disinhibiting the NK cells and allowing for one or more of enhanced NK cell homing, NK cell migration, activation of NK cells, expansion, cytotoxicity and/or persistence. Additionally, in several embodiments, the editing can enhance NK and/or T cell function in the otherwise suppressive tumor microenvironment.

In several embodiments, genetic editing (whether knock out or knock in) of any of the target genes disclosed herein, is accomplished through targeted introduction of DNA breakage, and a subsequent DNA repair mechanism. In several embodiments, double strand breaks of DNA are repaired by non-homologous end joining (NHEJ), wherein enzymes are used to directly join the DNA ends to one another to repair the break. NHEJ is an error-prone process. In general, in the absence of a repair template, the NHEJ process re-ligates the ends of the cleaved DNA strands, which frequently results in nucleotide deletions and insertions at the cleavage site. In several embodiments, however, double strand breaks (DSB) are repaired by homology directed repair (HDR), which is advantageously more accurate, thereby allowing sequence specific breaks and repair. HDR uses a homologous sequence as a template for regeneration of missing DNA sequences at the break point, such as a vector with the desired genetic elements (e.g., an insertion element to disrupt the coding sequence of a gene) within a sequence that is homologous to the flanking sequences of a double strand break. This will result in the desired change (e.g., insertion) being inserted at the site of the DSB. The HDR pathway can occur by way of the canonical HDR pathway or the alternative HDR pathway. Unless otherwise indicated, the term “HDR” or “homology-directed repair” as used herein encompasses both canonical HDR and alternative HDR.

Canonical HDR or “canonical homology-directed repair” or cHDR,” are used interchangeably, and refers to the process of repairing DNA damage using a homologous nucleic acid (e.g., an endogenous homologous sequence, such as a sister chromatid; or an exogenous nucleic acid, such as a donor template). Canonical HDR typically acts when there has been a significant resection at the DSB, forming at least one single-stranded portion of DNA. In a normal cell, canonical HDR typically involves a series of steps such as recognition of the break, stabilization of the break, resection, stabilization of single-stranded DNA, formation of a DNA crossover intermediate, resolution of the crossover intermediate, and ligation. The canonical HDR process requires RAD51 and BRCA2, and the homologous nucleic acid, e.g., repair template, is typically double-stranded. In canonical HDR, a double-stranded polynucleotide, e.g., a double-stranded repair template, is introduced, which comprises a sequence that is homologous to the targeting sequence, and which will either be directly integrated into the targeting sequence or will be used as a template to insert the sequence, or a portion the sequence, of the repair template into the target gene. After resection at the break, repair can progress by different pathways, e.g., by the double Holliday junction model (also referred to as the double strand break repair, or DSBR, pathway), or by the synthesis-dependent strand annealing (SDSA) pathway.

In the double Holliday junction model, strand invasion occurs by the two single stranded overhangs of the targeting sequence to the homologous sequences in the double-stranded polynucleotide, e.g., double stranded donor template, which results in the formation of an intermediate with two Holliday junctions. The junctions migrate as new DNA is synthesized from the ends of the invading strand to fill the gap resulting from the resection. The end of the newly synthesized DNA is ligated to the resected end, and the junctions are resolved, resulting in the insertion at the targeting sequence, or a portion of the targeting sequence that includes the gene variant. Crossover with the polynucleotide, e.g., repair template, may occur upon resolution of the junctions.

In the SDSA pathway, only one single stranded overhang invades the polynucleotide, e.g., donor template, and new DNA is synthesized from the end of the invading strand to fill the gap resulting from resection. The newly synthesized DNA then anneals to the remaining single stranded overhang, new DNA is synthesized to fill in the gap, and the strands are ligated to produce the modified DNA duplex.

Alternative HDR, or “alternative homology-directed repair,” or “alternative HDR,” are used interchangeably, and refers, in some embodiments, to the process of repairing DNA damage using a homologous nucleic acid (e.g., an endogenous homologous sequence, such as a sister chromatid; or an exogenous nucleic acid, such as a repair template). Alternative HDR is distinct from canonical HDR in that the process utilizes different pathways from canonical HDR, and can be inhibited by the canonical HDR mediators, RAD51 and BRCA2. Moreover, alternative HDR is also distinguished by the involvement of a single-stranded or nicked homologous nucleic acid template, e.g., repair template, whereas canonical HDR generally involves a double-stranded homologous template. In the alternative HDR pathway, a single strand template polynucleotide, e.g., repair template, is introduced. A nick, single strand break, or DSB at the cleavage site, for altering a desired target site, e.g., a gene variant in a target gene, is mediated by a nuclease molecule, and resection at the break occurs to reveal single stranded overhangs. Incorporation of the sequence of the template polynucleotide, e.g., repair template, to alter the target site of the DNA typically occurs by the SDSA pathway, as described herein. In some embodiments, HDR is carried out by introducing, into a cell, one or more agent(s) capable of inducing a DSB, and a repair template, e.g., a single-stranded oligonucleotide. The introducing can be carried out by any suitable delivery. The conditions under which HDR is allowed to occur can be any conditions suitable for carrying out HDR in a cell.

In several embodiments, gene editing is accomplished by one or more of a variety of engineered nucleases. In several embodiments, restriction enzymes are used, particularly when double strand breaks are desired at multiple regions. In several embodiments, a bioengineered nuclease is used. Depending on the embodiment, one or more of a Zinc Finger Nuclease (ZFN), transcription-activator like effector nuclease (TALEN), meganuclease and/or clustered regularly interspaced short palindromic repeats (CRISPR/Cas9) system are used to specifically edit the genes encoding one or more of the TCR subunits.

Meganucleases are characterized by their capacity to recognize and cut large DNA sequences (from 14 to 40 base pairs). In several embodiments, a meganuclease from the LAGLIDADG family is used, and is subjected to mutagenesis and screening to generate a meganuclease variant that recognizes a unique sequence(s), such as a specific site in a TCR subunit (e.g., TRAC), or CISH, or any other target gene disclosed herein. Target sites in a TCR subunit can readily be identified. Further information of target sites within a region of the TCR can be found in US Patent Publication No. 2018/0325955, and US Patent Publication No. 2015/0017136, each of which is incorporated by reference herein in its entirety. In several embodiments, two or more meganucleases, or functions fragments thereof, are fused to create a hybrid enzyme that recognize a desired target sequence within the target gene (e.g., CISH).

In contrast to meganucleases, ZFNs and TALEN function based on a non-specific DNA cutting catalytic domain which is linked to specific DNA sequence recognizing peptides such as zinc fingers or transcription activator-like effectors (TALEs). Advantageously, the ZFNs and TALENs thus allow sequence-independent cleavage of DNA, with a high degree of sequence-specificity in target recognition. Zinc finger motifs naturally function in transcription factors to recognize specific DNA sequences for transcription. The C-terminal part of each finger is responsible for the specific recognition of the DNA sequence. While the sequences recognized by ZFNs are relatively short, (e.g., ˜3 base pairs), in several embodiments, combinations of 2, 3, 4, 5, 6, 7, 8, 9, 10 or more zinc fingers whose recognition sites have been characterized are used, thereby allowing targeting of specific sequences, such as a portion of the TCR (or an immune checkpoint). The combined ZFNs are then fused with the catalytic domain(s) of an endonuclease, such as FokI (optionally a FokI heterodimer), in order to induce a targeted DNA break. Additional information on uses of ZFNs to edit a TCR subunit and/or immune checkpoints can be found in U.S. Pat. No. 9,597,357, which is incorporated by reference herein.

Transcription activator-like effector nucleases (TALENs) are specific DNA-binding proteins that feature an array of 33 or 34-amino acid repeats. Like ZFNs, TALENs are a fusion of a DNA cutting domain of a nuclease to TALE domains, which allow for sequence-independent introduction of double stranded DNA breaks with highly precise target site recognition. TALENs can create double strand breaks at the target site that can be repaired by error-prone non-homologous end-joining (NHEJ), resulting in gene disruptions through the introduction of small insertions or deletions. Advantageously, TALENs are used in several embodiments, at least in part due to their higher specificity in DNA binding, reduced off-target effects, and ease in construction of the DNA-binding domain.

CRISPRs (Clustered Regularly Interspaced Short Palindromic Repeats) are genetic elements that bacteria use as protection against viruses. The repeats are short sequences that originate from viral genomes and have been incorporated into the bacterial genome. Cas (CRISPR associated proteins) process these sequences and cut matching viral DNA sequences. By introducing plasmids containing Cas genes and specifically constructed CRISPRs into eukaryotic cells, the eukaryotic genome can be cut at any desired position. Additional information on CRISPR can be found in US Patent Publication No. 2014/0068797, which is incorporated by reference herein. In several embodiments, CRISPR is used to manipulate the gene(s) encoding a target gene to be knocked out or knocked in, for example CISH, TGFBR2, TCR, B2M, CIITA, CD47, HLA-E, etc. In several embodiments, CRISPR is used to edit one or more of the TCRs of a T cell and/or the genes encoding one or more immune checkpoints. In several embodiments, the immune checkpoint is selected from one or more of CTLA4 and PD1. In several embodiments, CRISPR is used to truncate one or more of TCRα, TCRβ, TCRγ, and TCRδ. In several embodiments, a TCR is truncated without impacting the function of the CD3z signaling domain of the TCR.

Depending on the embodiment and which target gene is to be edited, a Class 1 or Class 2 Cas is used. In several embodiments, a Class 1 Cas is used and the Cas type is selected from the following types: I, IA, IB, IC, ID, IE, IF, IU, III, IIIA, IIIB, IIIC, HID, IV IVA, IVB, and combinations thereof. In several embodiments, the Cas is selected from the group consisting of Cas3, Cas8a, Cas5, Cas8b, Cas8c, Cas10d, Cse1, Cse2, Csy1, Csy2, Csy3, GSU0054, Cas10, Csm2, Cmr5, Cas10, Csx11, Csx10, Csf1, and combinations thereof. In several embodiments, a Class 2 Cas is used and the Cas type is selected from the following types: II, IIA, IIB, IIC, V, VI, and combinations thereof. In several embodiments, the Cas is selected from the group consisting of Cas9, Csn2, Cas4, Cas12a (previously known as Cpf1), C2c1, C2c3, Cas13a (previously known as C2c2), Cas13b, Cas13c, CasX, CasY and combinations thereof. In some embodiments, the Cas is Cas9. In some embodiments, class 2 CasX is used, wherein CasX is capable of forming a complex with a guide nucleic acid and wherein the complex can bind to a target DNA, and wherein the target DNA comprises a non-target strand and a target strand. In some embodiments, class 2 CasY is used, wherein CasY is capable of binding and modifying a target nucleic acid and/or a polypeptide associated with target nucleic acid.

### Targets for Gene Editing

As discussed above, gene editing can be used to disrupt a target gene (or genes) in order to enhance the functionality (e.g., expandability, cytotoxicity) or persistence (lifespan or ability to resist hypoxia) of immune cells, such as NK cells. In some embodiments, immune cells are genetically edited at ADAM17, HIF-1a, DGKz, GSK-3B, LAG3, TIM3, TRIM29, IL-1R8, CD38, FBP-1, INSIG1, MED12, MED13, CCNC, CDK8, ID3, SOX4, or any combination thereof. In some embodiments, immune cells are genetically edited at ADAM17, HIF-1a, DGKz, GSK-3B, LAG3, TIM3, TRIM29, IL-1R8, CD38, FBP-1, INSIG1, MED12, MED13, CCNC, CDK8, CBLB, CISH, ID3, SOX4, or any combination thereof.

By way of non-limiting example, in several embodiments, a disintegrin and metalloprotease domain (ADAM) family member, in particular embodiments ADAM17, is a target of gene editing. ADAM17, located on chromosome 2, is implicated in antibody-dependent cell-mediated cytotoxicity (ADCC), which is a key mechanism of action in anti-tumor responses. CD16A is a membrane-bound protein expressed by NK cells and a receptor for the Fc portion of IgGs. While engagement of CD16A (e.g., by antibody-coated target cells) triggers NK cell-mediated ADCC, CD16A is rapidly downregulated after NK cell activation by cleavage from the NK cell surface (either in vivo or in vitro, for example by PMA). ADAM17 is believed to be the primary protease responsible for cleavage of CD16A from the NK cell surface; inhibition of ADAM17 (such as by disruption in ADAM17 expression) reduces, ameliorates, or otherwise inhibits the cleavage of CD16A, which allows ADCC to continue as an operative anti-tumor pathway (Wu et al., J Leukoc Biol (2019) 105(6):1297-1303). Moreover, CD62 ligand (CD62L) is a substrate of ADAM17, and the disruption of expression of ADAM17 functions, in several embodiments, to stabilize CD62L expression. CD62L, an L-selectin molecule mediates homing of leukocytes to lymphoid organs. CD56dimCD62L+ cells represent a unique subset of mature, polyfunctional NK cells that affect the magnitude of the local NK cell response, in particular, by the ability to produce IFN-γ after cytokine stimulation, proliferate in vivo during viral infection, and kill target cells upon engagement of activating receptors. Thus, stabilizing CD62L may, in several embodiments, further enhance NK cell function.

In several embodiments, gene editing reduces transcription of ADAM17 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of ADAM17 by at least about 30%, In several embodiments, gene editing reduces transcription of ADAM17 by at least about 40%, In several embodiments, gene editing reduces transcription of ADAM17 by at least about 50%, In several embodiments, gene editing reduces transcription of ADAM17 by at least about 60%, In several embodiments, gene editing reduces transcription of ADAM17 by at least about 70%, In several embodiments, gene editing reduces transcription of ADAM17 by at least about 80%, In several embodiments, gene editing reduces transcription of ADAM17 by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of ADAM17 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of ADAM17 by at least about 30%, In several embodiments, gene editing reduces expression of ADAM17 by at least about 40%, In several embodiments, gene editing reduces expression of ADAM17 by at least about 50%, In several embodiments, gene editing reduces expression of ADAM17 by at least about 60%, In several embodiments, gene editing reduces expression of ADAM17 by at least about 70%, In several embodiments, gene editing reduces expression of ADAM17 by at least about 80%, In several embodiments, gene editing reduces expression of ADAM17 by at least about 90%.

In several embodiments, a guide RNA (gRNA) comprising the sequence of any of SEQ ID NOS:682-687 is used to disrupt (e.g., reduce expression of) the ADAM17 gene.

In several embodiments, Hypoxia Inducible Factor 1 alpha (HIF1a) is a target of gene editing. HIF1a, which is located on chromosome 15 is a transcriptional activator of CD274 (also known as PDL1). When hypoxic conditions exist, HIF1a interacts with a hypoxia response element in the promoter of PDL1, which then drives increased PDL1 expression. This upregulation of PDL1 expression on various cells, including tumor cells, immune cells (including MDSCs, macrophages, DCs, and bone marrow-derived macrophages (BMDMs)), and other cells in the TME, serves to inhibit the ability of T cells and/or NK cells to kill the tumor through the binding of PD1 to PDL1. Studies using single-cell RNA sequencing of tumor-infiltrating NK cells revealed that inhibition of HIF1a promoted tumor-infiltrating NK cell activity (Ni et al., Immunity (2020) 52(6):1075-87). In some embodiments, loss of HIF1a in NK cells inhibits tumor growth through, for example, stimulation of non-productive angiogenesis (e.g., such that the tumor cell is starved of blood supply).

In several embodiments, gene editing reduces transcription of HIF1A by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of HIF1A by at least about 30%, In several embodiments, gene editing reduces transcription of HIF1A by at least about 40%, In several embodiments, gene editing reduces transcription of HIF1A by at least about 50%, In several embodiments, gene editing reduces transcription of HIF1A by at least about 60%, In several embodiments, gene editing reduces transcription of HIF1A by at least about 70%, In several embodiments, gene editing reduces transcription of HIF1A by at least about 80%, In several embodiments, gene editing reduces transcription of HIF1A by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of HIF1A by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of HIF1A by at least about 30%, In several embodiments, gene editing reduces expression of HIF1A by at least about 40%, In several embodiments, gene editing reduces expression of HIF1A by at least about 50%, In several embodiments, gene editing reduces expression of HIF1A by at least about 60%, In several embodiments, gene editing reduces expression of HIF1A by at least about 70%, In several embodiments, gene editing reduces expression of HIF1A by at least about 80%, In several embodiments, gene editing reduces expression of HIF1A by at least about 90%.

In several embodiments, a guide RNA (gRNA) comprising the sequence of any of SEQ ID NOS:750-760 is used to disrupt (e.g., reduce expression of) the HIF1A gene.

By way of non-limiting example, Diacylglycerol Kinase Zeta (DGKz) is targeted for genetic editing to reduce to knock out expression. DGKz is located on chromosome 11 and is a negative regulator of diacylglycerol kinase mediated signaling. Mice lacking DGKz have been shown in studies to show increase cytokine production and degranulation, in some instances in a ERK-dependent (also known as Ras-Ref-MEK-ERK pathway) manner. Additionally, CRISPR/Cas9-mediated knock out of DGK can improve the function (e.g., antitumor activities) of T cells. However, in NK cells DGKz disruption is believed to be particularly beneficial because, according to some embodiment, disruption of DGKz does not negatively impact inhibitory NK cell receptor expression or function, thereby maintaining in several respects the natural balance of NK activating and inhibitory signals that regulate NK cell activity (Singh and Kambayashi, Front Cell Dev Bio (2016) 4:96). Thus, enhancement of function of NK cells is achieved, in several embodiments, through enhancing NK cell activity and signaling by disinhibiting a negative regulating aspect of an activating pathway rather than by disrupting the “brake” of NK cell function, inhibitory NK cells receptor expression or function, which could lead to unchecked NK cell activity and potential off-target cytotoxicity.

In several embodiments, gene editing reduces transcription of DGKZ by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of DGKZ by at least about 30%, In several embodiments, gene editing reduces transcription of DGKZ by at least about 40%, In several embodiments, gene editing reduces transcription of DGKZ by at least about 50%, In several embodiments, gene editing reduces transcription of DGKZ by at least about 60%, In several embodiments, gene editing reduces transcription of DGKZ by at least about 70%, In several embodiments, gene editing reduces transcription of DGKZ by at least about 80%, In several embodiments, gene editing reduces transcription of DGKZ by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of DGKZ by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of DGKZ by at least about 30%, In several embodiments, gene editing reduces expression of DGKZ by at least about 40%, In several embodiments, gene editing reduces expression of DGKZ by at least about 50%, In several embodiments, gene editing reduces expression of DGKZ by at least about 60%, In several embodiments, gene editing reduces expression of DGKZ by at least about 70%, In several embodiments, gene editing reduces expression of DGKZ by at least about 80%, In several embodiments, gene editing reduces expression of DGKZ by at least about 90%.

In several embodiments, a guide RNA (gRNA) comprising the sequence of any of SEQ ID NOS:688-723 is used to disrupt (e.g., reduce expression of) the DGKZ gene.

In several embodiments, Glycogen Synthase Kinase-3 beta (GSK3B) is a target of gene editing. GSK3B, located on chromosome 11, is a ubiquitously expressed serine/threonine kinase which is involved in a variety of cellular functions, including differentiation, survival, glycogen metabolism, protein synthesis, immune responses, and cell death, among others. In AML patients, repression of GSK3B is believed to restore NK cell cytotoxicity. Moreover, GSK3B inhibition (e.g., by small molecule inhibitor) can drive NK cell maturation and in some embodiments, enhance anti-tumor activity (Cichocki et al., Cancer Res (2017) 77(20):5664-75). Normal levels of GSK3B are thought to negatively regulated several aspects of NK cell function, including those functions triggered by one or more (e.g., combinations) of activating NK cell receptors. Returning to small molecule effects, small molecule inhibitors of GSK3B can specifically inhibit transcription of inhibitory co-receptor LAG-3 (Rudd et al., Cell Rep (2020) 30(7):2075-82; discussed in more detail below).

In several embodiments, gene editing reduces transcription of GSK3B by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of GSK3B by at least about 30%, In several embodiments, gene editing reduces transcription of GSK3B by at least about 40%, In several embodiments, gene editing reduces transcription of GSK3B by at least about 50%, In several embodiments, gene editing reduces transcription of GSK3B by at least about 60%, In several embodiments, gene editing reduces transcription of GSK3B by at least about 70%, In several embodiments, gene editing reduces transcription of GSK3B by at least about 80%, In several embodiments, gene editing reduces transcription of GSK3B by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of GSK3B by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of GSK3B by at least about 30%, In several embodiments, gene editing reduces expression of GSK3B by at least about 40%, In several embodiments, gene editing reduces expression of GSK3B by at least about 50%, In several embodiments, gene editing reduces expression of GSK3B by at least about 60%, In several embodiments, gene editing reduces expression of GSK3B by at least about 70%, In several embodiments, gene editing reduces expression of GSK3B by at least about 80%, In several embodiments, gene editing reduces expression of GSK3B by at least about 90%.

In several embodiments, a guide RNA (gRNA) comprising the sequence of any of SEQ ID NOS:724-749 is used to disrupt (e.g., reduce expression of) the GSK3B gene.

In several embodiments, lymphocyte activation gene 3 (LAG3), is a target of gene editing. LAG3, which is located on chromosome 12, operates as an immune checkpoint and inhibits the activation of its host cell (such as NK and/or T cells) and generally promotes a more suppressive immune response. For example, on T cells, LAG3 reduces cytokine and granzyme production, and proliferation, all while encouraging differentiation into T regulatory cells rather than cytotoxic cells. LAG3 functions in NK cells as a checkpoint to reduce cytokine production by CD56+Dim cytotoxic cells.

In several embodiments, gene editing reduces transcription of LAG3 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of LAG3 by at least about 30%, In several embodiments, gene editing reduces transcription of LAG3 by at least about 40%, In several embodiments, gene editing reduces transcription of LAG3 by at least about 50%, In several embodiments, gene editing reduces transcription of LAG3 by at least about 60%, In several embodiments, gene editing reduces transcription of LAG3 by at least about 70%, In several embodiments, gene editing reduces transcription of LAG3 by at least about 80%, In several embodiments, gene editing reduces transcription of LAG3 by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of LAG3 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of LAG3 by at least about 30%, In several embodiments, gene editing reduces expression of LAG3 by at least about 40%, In several embodiments, gene editing reduces expression of LAG3 by at least about 50%, In several embodiments, gene editing reduces expression of LAG3 by at least about 60%, In several embodiments, gene editing reduces expression of LAG3 by at least about 70%, In several embodiments, gene editing reduces expression of LAG3 by at least about 80%, In several embodiments, gene editing reduces expression of LAG3 by at least about 90%.

In several embodiments, a guide RNA (gRNA) comprising the sequence of any of SEQ ID NOS:761-789 is used to disrupt (e.g., reduce expression of) the LAG3 gene.

In several embodiments, T-cell immunoglobulin and mucin domain 3 (TIM3) is a target of gene editing. TIM3 is a receptor expressed on NK cells and is implicated as a marker of dysfunctional NK cells. TIM3 is an immune checkpoint and a member of the TIM family of proteins. TIM3 has multiple types of ligands, including CEACAM1, including CEACAM1, high-mobility group box 1 (HMGB1), phosphatidylserine (PtdSer). and Galectin-9 (Gal-9), which upon interaction with TIM3 can reduce cell signaling. Like LAG3 above, TIM3 is induced by hypoxia, such as exists in some areas of the tumor microenvironment (among other genes, including CTLA4, PD1, PDL1, CD47, and other immune checkpoints). In several embodiments, genetic disruption of TIM3 reduces the negative impact of the hypoxic TME and allows for enhance NK cell anti-tumor activity.

In several embodiments, gene editing reduces transcription of TIM3 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of TIM3 by at least about 30%, In several embodiments, gene editing reduces transcription of TIM3 by at least about 40%, In several embodiments, gene editing reduces transcription of TIM3 by at least about 50%, In several embodiments, gene editing reduces transcription of TIM 3 by at least about 60%, In several embodiments, gene editing reduces transcription of TIM 3 by at least about 70%, In several embodiments, gene editing reduces transcription of TIM3 by at least about 80%, In several embodiments, gene editing reduces transcription of TIM3 by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of TIM3 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of TIM3 by at least about 30%, In several embodiments, gene editing reduces expression of TIM3 by at least about 40%, In several embodiments, gene editing reduces expression of TIM3 by at least about 50%, In several embodiments, gene editing reduces expression of TIM3 by at least about 60%, In several embodiments, gene editing reduces expression of TIM3 by at least about 70%, In several embodiments, gene editing reduces expression of TIM3 by at least about 80%, In several embodiments, gene editing reduces expression of TIM3 by at least about 90%.

In several embodiments, a guide RNA (gRNA) comprising the sequence of any of SEQ ID NOS:790-825 is used to disrupt (e.g., reduce expression of) the TIM3 gene.

In several embodiments, Tripartite Motif Containing 29 (TRIM29) is a target of gene editing. TRIM29, located on chromosome 11, is a member of a family of proteins involved in many biological processes, including cell development, differentiation, apoptosis, and tumorigenesis. TRIM29 is induced in NK cells by IL-12 and IL-18, and due to its E3 ubiquitin ligase function, promotes proteasome-mediated degradation of various target genes, such as TAB2 (TGF-Beta Activated Kinase Binding Protein 2), which leads to an inhibition of IFN-g production by activated NK cells, thereby limiting their cytotoxicity (Dou et al., J Immunol (2019) 203(4):873-80). Deficiency of TRIM29 in NK cells, such as by gene editing, may lead in several embodiments, to markedly enhanced NK cell functions, even after IL-12 and IL-18 stimulation.

In several embodiments, gene editing reduces transcription of TRIM29 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of TRIM29 by at least about 30%, In several embodiments, gene editing reduces transcription of TRIM29 by at least about 40%, In several embodiments, gene editing reduces transcription of TRIM29 by at least about 50%, In several embodiments, gene editing reduces transcription of TRIM29 by at least about 60%, In several embodiments, gene editing reduces transcription of TRIM29 by at least about 70%, In several embodiments, gene editing reduces transcription of TRIM29 by at least about 80%, In several embodiments, gene editing reduces transcription of TRIM29 by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of TRIM29 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of TRIM29 by at least about 30%, In several embodiments, gene editing reduces expression of TRIM29 by at least about 40%, In several embodiments, gene editing reduces expression of TRIM29 by at least about 50%, In several embodiments, gene editing reduces expression of TRIM29 by at least about 60%, In several embodiments, gene editing reduces expression of TRIM29 by at least about 70%, In several embodiments, gene editing reduces expression of TRIM29 by at least about 80%, In several embodiments, gene editing reduces expression of TRIM29 by at least about 90%.

In several embodiments, a guide RNA (gRNA) comprising the sequence of any of SEQ ID NOS:826-835 is used to disrupt (e.g., reduce expression of) the TRIM29 gene.

In several embodiments, Interleukin-1 receptor 8 (IL-1R8) is a target of gene editing. IL-1R8, located on chromosome 11, is a member of the IL-1 receptor (ILR) family that acts as a negative regulator of ILR and Toll-like receptor (TLR) downstream signaling pathways and inflammation. IL-1R8 is the co-receptor of IL-1R5/IL-18Ra for IL-37. IL-1R8 is a checkpoint in NK cells that negative regulates anti-tumor and anti-viral activity.

In several embodiments, gene editing reduces transcription of IL-1R8 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of IL-1R8 by at least about 30%, In several embodiments, gene editing reduces transcription of IL-1R8 by at least about 40%, In several embodiments, gene editing reduces transcription of IL-1R8 by at least about 50%, In several embodiments, gene editing reduces transcription of IL-1R8 by at least about 60%, In several embodiments, gene editing reduces transcription of IL-1R8 by at least about 70%, In several embodiments, gene editing reduces transcription of IL-1R8 by at least about 80%, In several embodiments, gene editing reduces transcription of IL-1R8 by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of IL-1R8 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of IL-1R8 by at least about 30%, In several embodiments, gene editing reduces expression of IL-1R8 by at least about 40%, In several embodiments, gene editing reduces expression of IL-1R8 by at least about 50%, In several embodiments, gene editing reduces expression of IL-1R8 by at least about 60%, In several embodiments, gene editing reduces expression of IL-1R8 by at least about 70%, In several embodiments, gene editing reduces expression of IL-1R8 by at least about 80%, In several embodiments, gene editing reduces expression of IL-1R8 by at least about 90%.

In several embodiments, a guide RNA (gRNA) comprising the sequence of any of SEQ ID NOS:836-865 is used to disrupt (e.g., reduce expression of) the IL-1R8 gene.

In several embodiments, CD38 is a target of gene editing. CD38, located on chromosome 4, is an ectoenzyme with nicotinamide-adenine-dinucleotide-positive (NAD+) glycohydrolase and ADP-ribosyl cyclase activity. CD38 is also expressed on several tumor cells, such as multiple myeloma and acute myeloid leukemia cells. The function of CD38 and these glycohydrolase and cyclase activities result, in some instances of generation of the immunosuppressive molecule adenosine, which i) inhibits tumor cell lysis by T and NK cells, ii) induces M2 macrophages and tolerogenic dendritic cells (DC) and/or iii) induces Treg expansion. Moreover, the endogenous expression of CD38 can be problematic for therapeutic cell persistence, due to, for example fratricide if CD38-targeting CARs are used.

In several embodiments, gene editing reduces transcription of CD38 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of CD38 by at least about 30%, In several embodiments, gene editing reduces transcription of CD38 by at least about 40%, In several embodiments, gene editing reduces transcription of CD38 by at least about 50%, In several embodiments, gene editing reduces transcription of CD38 by at least about 60%, In several embodiments, gene editing reduces transcription of CD38 by at least about 70%, In several embodiments, gene editing reduces transcription of CD38 by at least about 80%, In several embodiments, gene editing reduces transcription of CD38 by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of CD38 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of CD38 by at least about 30%, In several embodiments, gene editing reduces expression of CD38 by at least about 40%, In several embodiments, gene editing reduces expression of CD38 by at least about 50%, In several embodiments, gene editing reduces expression of CD38 by at least about 60%, In several embodiments, gene editing reduces expression of CD38 by at least about 70%, In several embodiments, gene editing reduces expression of CD38 by at least about 80%, In several embodiments, gene editing reduces expression of CD38 by at least about 90%.

In several embodiments, a guide RNA (gRNA) comprising the sequence of any of SEQ ID NOS:866-874 is used to disrupt (e.g., reduce expression of) the CD38 gene.

In several embodiments, fructose-1,6-bisphosphatase (FBP1) is a target of gene editing. FBP1, located on chromosome 9, is a rate-limiting enzyme involved in gluconeogenesis. It functions mainly facilitate gluconeogenesis while inhibiting glycolysis. The FBP1-related impairment of NK cell glycolysis elicits dysfunction of NK cells (Cong et al., Cell Metab (2018) 28(2):243-55). However, according to several embodiments, disruption of FBP1 expression restores the function of NK cells.

In several embodiments, gene editing reduces transcription of FBP1 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of FBP1 by at least about 30%, In several embodiments, gene editing reduces transcription of FBP1 by at least about 40%, In several embodiments, gene editing reduces transcription of FBP1 by at least about 50%, In several embodiments, gene editing reduces transcription of FBP1 by at least about 60%, In several embodiments, gene editing reduces transcription of FBP1 by at least about 70%, In several embodiments, gene editing reduces transcription of FBP1 by at least about 80%, In several embodiments, gene editing reduces transcription of FBP1 by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of FBP1 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of FBP1 by at least about 30%, In several embodiments, gene editing reduces expression of FBP1 by at least about 40%, In several embodiments, gene editing reduces expression of FBP1 by at least about 50%, In several embodiments, gene editing reduces expression of FBP1 by at least about 60%, In several embodiments, gene editing reduces expression of FBP1 by at least about 70%, In several embodiments, gene editing reduces expression of FBP1 by at least about 80%, In several embodiments, gene editing reduces expression of FBP1 by at least about 90%.

In several embodiments, a guide RNA (gRNA) comprising the sequence of any of SEQ ID NOS:875-889 is used to disrupt (e.g., reduce expression of) the FBP1 gene.

In several embodiments, Insulin induced gene 1 (INSIG1) is a target of gene editing. INSIG1, located on chromosome 7, is a negative regulator of Sterol regulatory element-binding protein (SRBP) transcription. SRBP, when transcribed and expressed, is a protein involved in an essential aspect of glucose metabolism by NK cells, in particular related to NK cell functional responses (e.g., cytotoxicity) (Assmann et al., Nat Immunol (2017) 18(11):1197-1206). According to several embodiments, disruption of INSIG1 expression disinhibits and thus restores the function of NK cells.

In several embodiments, gene editing reduces transcription of INSIG1 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of INSIG1 by at least about 30%, In several embodiments, gene editing reduces transcription of INSIG1 by at least about 40%, In several embodiments, gene editing reduces transcription of INSIG1 by at least about 50%, In several embodiments, gene editing reduces transcription of INSIG1 by at least about 60%, In several embodiments, gene editing reduces transcription of INSIG1 by at least about 70%, In several embodiments, gene editing reduces transcription of INSIG1 by at least about 80%, In several embodiments, gene editing reduces transcription of INSIG1 by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of INSIG1 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of INSIG1 by at least about 30%, In several embodiments, gene editing reduces expression of INSIG1 by at least about 40%, In several embodiments, gene editing reduces expression of INSIG1 by at least about 50%, In several embodiments, gene editing reduces expression of INSIG1 by at least about 60%, In several embodiments, gene editing reduces expression of INSIG1 by at least about 70%, In several embodiments, gene editing reduces expression of INSIG1 by at least about 80%, In several embodiments, gene editing reduces expression of INSIG1 by at least about 90%.

In several embodiments, a guide RNA (gRNA) comprising the sequence of any of SEQ ID NOS:890-934 is used to disrupt (e.g., reduce expression of) the INSIG1 gene.

Cells require many different types of molecular complexes to achieve the cellular processes of transcription and translation. These complexes, made of multiple, sometimes differing, subunits have the capacity to impart cell-specific functions, depending on their assembly and activity. One such molecular complex is the Mediator complex, which is expressed and required in cells where genes are actively being expressed, such as immune cells, like NK cells. It primarily functions as a “molecular bridge” that anchors two regions of otherwise unconnected DNA within the cell. For example, it can link a promotor and an enhancer, in order to physically localize the various elements and associated transcription factors required for expression of genes transcribed by RNA polymerases. Mediator complex subunit 12 (MED12) is one part of the four-part cyclin dependent kinase (CDK) module of Mediator along with MED13, cyclin-dependent kinase 8 (CDK8) and cyclin C (CCNC). Mutations in MED12 have been associated with lymphoproliferative disorders (Kampjarvi et al., Oncotarget (2015) 6(3):1884-88). More recently, targeted deletion of MED12, CCNC, or CDK8 in human CAR T cells was observed to increase proliferation, cytokine production, and antitumor activity. In particular, MED12 deficient T cells exhibited changes at genes regulating effector T cell differentiation. See Freitas et al., Cancer Res (2022) 82(12_suppl):2822; and Freitas et al., Science (2022) 378(6620):eabn5647. It is contemplated that reductions in MED12, MED13, CDK8 and/or CCNC could provide edited cells, such as NK cells with an enhanced persistence, allowing (when engineered in accordance with embodiments provided for herein) enhanced cytotoxicity against target tumor cells.

In several embodiments, gene editing reduces transcription of MED12 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of MED12 by at least about 30%, In several embodiments, gene editing reduces transcription of MED12 by at least about 40%, In several embodiments, gene editing reduces transcription of MED12 by at least about 50%, In several embodiments, gene editing reduces transcription of MED12 by at least about 60%, In several embodiments, gene editing reduces transcription of MED12 by at least about 70%, In several embodiments, gene editing reduces transcription of MED12 by at least about 80%, In several embodiments, gene editing reduces transcription of MED12 by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of MED12 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of MED12 by at least about 30%, In several embodiments, gene editing reduces expression of MED12 by at least about 40%, In several embodiments, gene editing reduces expression of MED12 by at least about 50%, In several embodiments, gene editing reduces expression of MED12 by at least about 60%, In several embodiments, gene editing reduces expression of MED12 by at least about 70%, In several embodiments, gene editing reduces expression of MED12 by at least about 80%, In several embodiments, gene editing reduces expression of MED12 by at least about 90%.

In several embodiments, MED12 expression is disrupted and/or knocked out using a Crispr-Cas mediated approach (e.g., Cas9), or other guided nuclease as disclosed elsewhere herein, with the use of one more of the following MED12-specific guide RNAs: SEQ ID NOS 938-948 (see e.g., Table E2). In several embodiments, a guide RNA (gRNA) comprising the sequence of any of SEQ ID NOS:938-948 is used to disrupt (e.g., reduce expression of) the MED12 gene. Non-limiting examples of guide RNAs to reduce and/or eliminate MED12 expression are provided below in Table 1.

TABLE 1
MED12 Guide RNAs

| SEQ ID | | | |
|---|---|---|---|
| NO: | Name | Sequence | Target |
| 938 | MED12 gRNA1 | TGCAATAATGCTGCTGAAGT | Exon 3 |
| 939 | MED12 gRNA2 | AGTTATCCTTCTGGTTCACT | Exon 3 |
| 940 | MED12 gRNA3 | GTCAGTGAACCAAGTGTTAA | Exon 3 |
| 996 | MED12 gRNA4 | AGGATTGAAGCTGACGTTCT | Exon 2 |
| 997 | MED12 gRNA5 | TAACTGCTCCCATAAGTACT | Exon 5 |
| 998 | MED12 gRNA6 | GTGGGATTACACCGAGAAGC | Exon 5 |

In several embodiments, gene editing reduces transcription of MED13 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of MED13 by at least about 30%, In several embodiments, gene editing reduces transcription of MED13 by at least about 40%, In several embodiments, gene editing reduces transcription of MED13 by at least about 50%, In several embodiments, gene editing reduces transcription of MED13 by at least about 60%, In several embodiments, gene editing reduces transcription of MED13 by at least about 70%, In several embodiments, gene editing reduces transcription of MED13 by at least about 80%, In several embodiments, gene editing reduces transcription of MED13 by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of MED13 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of MED13 by at least about 30%, In several embodiments, gene editing reduces expression of MED13 by at least about 40%, In several embodiments, gene editing reduces expression of MED13 by at least about 50%, In several embodiments, gene editing reduces expression of MED13 by at least about 60%, In several embodiments, gene editing reduces expression of MED13 by at least about 70%, In several embodiments, gene editing reduces expression of MED13 by at least about 80%, In several embodiments, gene editing reduces expression of MED13 by at least about 90%.

In several embodiments, gene editing reduces transcription of CDK8 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of CDK8 by at least about 30%, In several embodiments, gene editing reduces transcription of CDK8 by at least about 40%, In several embodiments, gene editing reduces transcription of CDK8 by at least about 50%, In several embodiments, gene editing reduces transcription of CDK8 by at least about 60%, In several embodiments, gene editing reduces transcription of CDK8 by at least about 70%, In several embodiments, gene editing reduces transcription of CDK8 by at least about 80%, In several embodiments, gene editing reduces transcription of CDK8 by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of CDK8 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of CDK8 by at least about 30%, In several embodiments, gene editing reduces expression of CDK8 by at least about 40%, In several embodiments, gene editing reduces expression of CDK8 by at least about 50%, In several embodiments, gene editing reduces expression of CDK8 by at least about 60%, In several embodiments, gene editing reduces expression of CDK8 by at least about 70%, In several embodiments, gene editing reduces expression of CDK8 by at least about 80%, In several embodiments, gene editing reduces expression of CDK8 by at least about 90%.

In several embodiments, CDK8 expression is disrupted and/or knocked out using a Crispr-Cas mediated approach (e.g., Cas9), or other guided nuclease as disclosed elsewhere herein, with the use of one more of the following CDK8-specific guide RNAs: SEQ ID NOS 949-955 (see e.g., Table E2). In several embodiments, a guide RNA (gRNA) comprising the sequence of any of SEQ ID NOS:949-955 is used to disrupt (e.g., reduce expression of) the CDK8 gene. Non-limiting examples of guide RNAs to reduce and/or eliminate CDK8 expression are provided below in Table 2.

TABLE 2
CDK8 Guide RNAs

| SEQ ID | | | |
|---|---|---|---|
| NO: | Name | Sequence | Target |
| 949 | CDK8 gRNA1 | AAGTGAAGCTGAGCAGCGAG | Exon 1 |
| 950 | CDK8 gRNA2 | AGACGTGACCATAAGTGCCT | Exon 1 |
| 951 | CDK8 gRNA3 | GCGCCGCCCAGCGCGGACAC | Intron 1 |

In several embodiments, gene editing reduces transcription of CCNC by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of CCNC by at least about 30%, In several embodiments, gene editing reduces transcription of CCNC by at least about 40%, In several embodiments, gene editing reduces transcription of CCNC by at least about 50%, In several embodiments, gene editing reduces transcription of CCNC by at least about 60%, In several embodiments, gene editing reduces transcription of CCNC by at least about 70%, In several embodiments, gene editing reduces transcription of CCNC by at least about 80%, In several embodiments, gene editing reduces transcription of CCNC by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of CCNC by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of CCNC by at least about 30%, In several embodiments, gene editing reduces expression of CCNC by at least about 40%, In several embodiments, gene editing reduces expression of CCNC by at least about 50%, In several embodiments, gene editing reduces expression of CCNC by at least about 60%, In several embodiments, gene editing reduces expression of CCNC by at least about 70%, In several embodiments, gene editing reduces expression of CCNC by at least about 80%, In several embodiments, gene editing reduces expression of CCNC by at least about 90%.

In several embodiments, CCNC expression is disrupted and/or knocked out using a Crispr-Cas mediated approach (e.g., Cas9), or other guided nuclease as disclosed elsewhere herein, with the use of one more of the following CCNC-specific guide RNAs: SEQ ID NOS 956-962 (see e.g., Table E2). In several embodiments, a guide RNA (gRNA) comprising the sequence of any of SEQ ID NOS:956-962 is used to disrupt (e.g., reduce expression of) the CCNC gene. Non-limiting examples of guide RNAs to reduce and/or eliminate CCNC expression are provided below in Table 3.

TABLE 3
CCNC Guide RNAs

| SEQ ID | | | |
|---|---|---|---|
| NO: | Name | Sequence | Target |
| 956 | CCNC gRNA1 | ATTGGTTCAAATTGTATAGT | Intron 2 |
| 957 | CCNC gRNA2 | AGAGAAACTTTAAATCCTTT | Exon 2 |
| 958 | CCNC gRNA3 | TTCTAGTTTGCAATGGATTT | Exon 2 |
| 999 | CCNC gRNA4 | TAGGCAAAGATCCGTTCTGT | Exon 9 |
| 961 | CCNC gRNA5 | TCTGTTGAAGGAGCGCCAAA | Exon 2 |
| 1000 | CCNC gRNA6 | ACCTTTGCTCCAGTATGTGC | Exon 8 |
| 1001 | CCNC gRNA7 | ATACCTAAAGCTATCATGAA | Exon 9 |

Two additional transcription factors, which are known as key regulators of T cell exhaustion present themselves as promising targets in NK cells, likewise, to disrupt or otherwise reduce NK cell exhaustion. Inhibitor of DNA Binding 3 (ID3), is also known to be expressed highly in progenitor NK cells, but decreased in mature cells (Boos et al., J Exp Med (2007) 204(5):1119-30). Deletion of TD3 therefor, in several embodiments, imparts a more mature phenotype and activity to NK cells and/or reduces exhaustion. Likewise, SOX4 editing to reduce SOX4 expression, in several embodiments, reduces NK cell exhaustion (Good et al., Cell 184(25):P6081-6100).

In several embodiments, gene editing reduces transcription of ID3 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of ID3 by at least about 30%, In several embodiments, gene editing reduces transcription of ID3 by at least about 40%, In several embodiments, gene editing reduces transcription of ID3 by at least about 50%, In several embodiments, gene editing reduces transcription of ID3 by at least about 60%, In several embodiments, gene editing reduces transcription of ID3 by at least about 70%, In several embodiments, gene editing reduces transcription of ID3 by at least about 80%, In several embodiments, gene editing reduces transcription of ID3 by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of ID3 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of ID3 by at least about 30%, In several embodiments, gene editing reduces expression of ID3 by at least about 40%, In several embodiments, gene editing reduces expression of ID3 by at least about 50%, In several embodiments, gene editing reduces expression of ID3 by at least about 60%, In several embodiments, gene editing reduces expression of ID3 by at least about 70%, In several embodiments, gene editing reduces expression of ID3 by at least about 80%, In several embodiments, gene editing reduces expression of ID3 by at least about 90%.

In several embodiments, ID3 expression is disrupted and/or knocked out using a Crispr-Cas mediated approach (e.g., Cas9), or other guided nuclease as disclosed elsewhere herein, with the use of one more of the following ID3-specific guide RNAs: SEQ ID NOS 963-969 (see e.g., Table E2). In several embodiments, a guide RNA (gRNA) comprising the sequence of any of SEQ ID NOS:963-969 is used to disrupt (e.g., reduce expression of) the ID3 gene. Non-limiting examples of guide RNAs to reduce and/or eliminate ID3 expression are provided below in Table 4.

TABLE 4
ID3 Guide RNAs

| SEQ ID | | | |
|---|---|---|---|
| NO: | Name | Sequence | Target |
| 963 | ID3 gRNA1 | CTCCGGGTACCAGTTCCCGC | Exon 1 |
| 964 | ID3 gRNA2 | CTCAGCGGCTCCTCAGCTGC | Exon 1 |
| 965 | ID3 gRNA3 | CAGCATGAAGGCGCTGAGCC | Exon 1 |
| 969 | ID3 gRNA7 | TGGCCAGACTGCGTTCCGAC | Exon 1 |

In several embodiments, gene editing reduces transcription of SOX4 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of SOX4 by at least about 30%, In several embodiments, gene editing reduces transcription of SOX4 by at least about 40%, In several embodiments, gene editing reduces transcription of SOX4 by at least about 50%, In several embodiments, gene editing reduces transcription of SOX4 by at least about 60%, In several embodiments, gene editing reduces transcription of SOX4 by at least about 70%, In several embodiments, gene editing reduces transcription of SOX4 by at least about 80%, In several embodiments, gene editing reduces transcription of SOX4 by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of SOX4 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of SOX4 by at least about 30%, In several embodiments, gene editing reduces expression of SOX4 by at least about 40%, In several embodiments, gene editing reduces expression of SOX4 by at least about 50%, In several embodiments, gene editing reduces expression of SOX4 by at least about 60%, In several embodiments, gene editing reduces expression of SOX4 by at least about 70%, In several embodiments, gene editing reduces expression of SOX4 by at least about 80%, In several embodiments, gene editing reduces expression of SOX4 by at least about 90%.

In several embodiments, SOX4 expression is disrupted and/or knocked out using a Crispr-Cas mediated approach (e.g., Cas9), or other guided nuclease as disclosed elsewhere herein, with the use of one more of the following SOX4-specific guide RNAs: SEQ ID NOS 970-976 (see e.g., Table E2). In several embodiments, a guide RNA (gRNA) comprising the sequence of any of SEQ ID NOS:970-976 is used to disrupt (e.g., reduce expression of) the SOX4 gene. Non-limiting examples of guide RNAs to reduce and/or eliminate ID3 expression are provided below in Table 5.

TABLE 5
SOX4 Guide RNAs

| SEQ ID | | | |
|---|---|---|---|
| NO: | Name | Sequence | Target |
| 970 | SOX4 gRNA1 | TTCCGTGTTCTCGGCATTGT | Exon 1 |
| 971 | SOX4 gRNA2 | GGCGATTCCCAGCTCGAGGC | Exon 1 |
| 972 | SOX4 gRNA4 | GCTGGTGCAAGACCCCGAGT | Exon 1 |

In accordance with additional embodiments, other modulators of one or more aspects of NK cell (or T cell) function are modulated through gene editing. A variety of cytokines impart either negative (as with TGF-beta above) or positive signals to immune cells. By way of non-limiting example, IL15 is a positive regulator of NK cells, which as disclosed herein, can enhance one or more of NK cell homing, NK cell migration, NK cell expansion/proliferation, NK cell cytotoxicity, and/or NK cell persistence. To keep NK cells in check under normal physiological circumstances, a cytokine-inducible SH2-containing protein (CIS, encoded by the CISH gene) acts as a critical negative regulator of IL-15 signaling in NK cells. As discussed herein, because IL15 biology impacts multiple aspects of NK cell functionality, including, but not limited to, proliferation/expansion, activation, cytotoxicity, persistence, homing, migration, among others. Thus, according to several embodiments, editing CISH enhances the functionality of NK cells across multiple functionalities, leading to a more effective and long-lasting NK cell therapeutic. In several embodiments, inhibitors of CIS are used in conjunction with engineered NK cell administration. In several embodiments, CIS expression is knocked down or knocked out through gene editing of the CISH gene, for example, by use of CRISPR-Cas editing. Small interfering RNA, antisense RNA, TALENs or zinc fingers are used in other embodiments. In some embodiments CIS expression in T cells is knocked down through gene editing.

In several embodiments, as discussed above, editing of CISH advantageously imparts to the edited cells, particularly edited NK cells, enhanced expansion, cytotoxicity and/or persistence. Additionally, in several embodiments, the modification of the TCR comprises a modification to TCRα, but without impacting the signaling through the CD3 complex, allowing for T cell proliferation. In one embodiment, the TCRα is inactivated by expression of pre-Ta in the cells, thus restoring a functional CD3 complex in the absence of a functional alpha/beta TCR. As disclosed herein, the non-alloreactive modified T cells are also engineered to express a CAR to redirect the non-alloreactive T cells specificity towards tumor marker, but independent of MHC. Combinations of editing are used in several embodiments, such as knockout of the TCR and CISH in combination, or knock out of CISH and knock in of CD47, by way of non-limiting examples. In some embodiments, a combination of CISH knockout and CDK8 knockout are used in combination. In some embodiments, a combination of CISH knockout and CCNC knockout are used in combination. In some embodiments, a combination of CISH knockout and MED12 knockout are used in combination. In some embodiments, a combination of CISH knockout and MED13 knockout are used in combination.

In several embodiments, CISH gene editing endows an NK cell with enhanced ability to home to a target site. In several embodiments, CISH gene editing endows an NK cell with enhanced ability to migrate, e.g., within a tissue in response to, for example chemoattractants or away from repellants. In several embodiments, CISH gene editing endows an NK cell with enhanced ability to be activated, and thus exert, for example, anti-tumor effects. In several embodiments, CISH gene editing endows an NK cell with enhanced proliferative ability, which in several embodiments, allows for generation of robust NK cell numbers from a donor blood sample. In addition, in such embodiments, NK cells edited for CISH and engineered to express a CAR are more readily, robustly, and consistently expanded in culture. In several embodiments, CISH gene editing endows an NK cell with enhanced cytotoxicity. In several embodiments, the editing of CISH synergistically enhances the cytotoxic effects of engineered NK cells and/or engineered T cells that express a CAR.

In several embodiments, CISH gene editing activates or inhibits a wide variety of pathways. The CIS protein is a negative regulator of IL15 signaling by way of, for example, inhibiting JAK-STAT signaling pathways. These pathways would typically lead to transcription of IL15-responsive genes (including CISH). In several embodiments, knockdown of CISH disinhibits JAK-STAT (e.g., JAK1-STAT5) signaling and there is enhanced transcription of IL15-responsive genes. In several embodiments, knockout of CISH yields enhanced signaling through mammalian target of rapamycin (mTOR), with corresponding increases in expression of genes related to cell metabolism and respiration. In several embodiments, knockout of CISH yields IL15 induced increased expression of IL-2Rα (CD25), but not IL-15Rα or IL-2/15Rβ, enhanced NK cell membrane binding of IL15 and/or IL2, increased phosphorylation of STAT-3 and/or STAT-5, and elevated expression of the antiapoptotic proteins, such as Bcl-2. In several embodiments, CISH knockout results in IL15-induced upregulation of selected genes related to mitochondrial functions (e.g., electron transport chain and cellular respiration) and cell cycle. Thus, in several embodiments, knockout of CISH by gene editing enhances the NK cell cytotoxicity and/or persistence, at least in part via metabolic reprogramming. In several embodiments, negative regulators of cellular metabolism, such as TXNIP, are downregulated in response to CISH knockout. In several embodiments, promotors for cell survival and proliferation including BIRC5 (Survivin), TOP2A, CKS2, and RACGAP1 are upregulated after CISH knockout, whereas antiproliferative or proapoptotic proteins such as TGFB1, ATM, and PTCH1 are downregulated. In several embodiments, CISH knockout alters the state (e.g., activates or inactivates) signaling via or through one or more of CXCL-10, IL2, TNF, IFNg, IL13, IL4, Jnk, PRF1, STAT5, PRKCQ, IL2 receptor Beta, SOCS2, MYD88, STAT3, STAT1, TBX21, LCK, JAK3, IL& receptor, ABL1, IL9, STAT5A, STAT5B, Tcf7, PRDM1, and/or EOMES.

By way of non-limiting example, TGF-beta is one such cytokine released by tumor cells that results in immune suppression within the tumor microenvironment. That immune suppression reduces the ability of immune cells, even engineered CAR-immune cells is some cases, to destroy the tumor cells, thus allowing for tumor progression. In several embodiments, as discussed in detail below, immune checkpoints are disrupted through gene editing. In several embodiments, blockers of immune suppressing cytokines in the tumor microenvironment are used, including blockers of their release or competitive inhibitors that reduce the ability of the signaling molecule to bind and inhibit an immune cell. Such signaling molecules include, but are not limited to TGF-beta, IL10, arginase, inducible NOS, reactive-NOS, Arg1, Indoleamine 2,3-dioxygenase (IDO), and PGE₂. However, in additional embodiments, there are provided immune cells, such as NK cells, wherein the ability of the NK cell (or other cell) to respond to a given immunosuppressive signaling molecule is disrupted and/or eliminated. For example, in several embodiments, in several embodiments, NK cells or T cells are genetically edited to become have reduced sensitivity to TGF-beta. TGF-beta is an inhibitor of NK cell function on at least the levels of proliferation and cytotoxicity. Thus, according to some embodiments, the expression of the TGF-beta receptor is knocked down or knocked out through gene editing, such that the edited NK is resistant to the immunosuppressive effects of TGF-beta in the tumor microenvironment. In several embodiments, the TGFB2 receptor is knocked down or knocked out through gene editing, for example, by use of CRISPR-Cas editing. Small interfering RNA, antisense RNA, TALENs or zinc fingers are used in other embodiments. Other isoforms of the TGF-beta receptor (e.g., TGF-beta 1 and/or TGF-beta 3) are edited in some embodiments. In some embodiments TGF-beta receptors in T cells are knocked down through gene editing

Additional cellular engineering strategies are provided for herein that serve to further enhance the persistence of allogeneic cellular therapy products, such as allogeneic CAR-T cells and/or allogeneic CAR-NK cells. There is provided for herein, in several embodiments, a population of genetically engineered immune cells for cancer immunotherapy where the genetically engineered immune cells are genetically modified (e.g., gene edited) at one, two, three or more gene loci to enhance the cytotoxic activity, persistence, or other feature of the cells, such as NK cells and/or T cells.

As discussed herein, there are various strategies that can be employed to reduce the tendency of an allogeneic cell therapy product to induce host cell-mediated graft rejection. For example, in several embodiments the expression of B2M is reduced and/or eliminated in order to reduce the host-mediated graft rejection. In several embodiments, B2M expression is disrupted and/or knocked out using a Crispr-Cas mediated approach (e.g., Cas9), or other guided nuclease as disclosed elsewhere herein, with the use of one more of the following B2M-specific guide RNAs: SEQ ID 199—CGCGAGCACAGCTAAGGCCA; SEQ ID 200—GAGTAGCGCGAGCACAGCTA; SEQ ID 201—GCTACTCTCTCTTTCTGGCC; SEQ ID 202—GGCCGAGATGTCTCGCTCCG; SEQ ID 203—GGCCACGGAGCGAGACATCT; SEQ ID 204—CACAGCCCAAGATAGTTAAG; SEQ ID 205—AGTCACATGGTTCACACGGC; SEQ ID 206—AAGTCAACTTCAATGTCGGA; SEQ ID 207—ACTTGTCTTTCAGCAAGGAC; and SEQ ID 208—TGGGCTGTGACAAAGTCACA.

In several embodiments, gene editing reduces transcription of B2M by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of B2M by at least about 30%, In several embodiments, gene editing reduces transcription of B2M by at least about 40%, In several embodiments, gene editing reduces transcription of B2M by at least about 50%, In several embodiments, gene editing reduces transcription of B2M by at least about 60%, In several embodiments, gene editing reduces transcription of B2M by at least about 70%, In several embodiments, gene editing reduces transcription of B2M by at least about 80%, In several embodiments, gene editing reduces transcription of B2M by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of B2M by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of B2M by at least about 30%, In several embodiments, gene editing reduces expression of B2M by at least about 40%, In several embodiments, gene editing reduces expression of B2M by at least about 50%, In several embodiments, gene editing reduces expression of B2M by at least about 60%, In several embodiments, gene editing reduces expression of B2M by at least about 70%, In several embodiments, gene editing reduces expression of B2M by at least about 80%, In several embodiments, gene editing reduces expression of B2M by at least about 90%.

In several embodiments, the expression of ADORA2A (Adenosine 2a Receptor) is reduced and/or eliminated in order to increase overall activation in resultant T cells and/or NK cells, or other cell type provided for herein. In several embodiments, ADORA2A is disrupted and/or knocked out using one or more of the gene editing methods disclosed herein. In several embodiments, ADORA2A is disrupted and/or knocked out using a Crispr-Cas mediated approach (e.g., Cas9), or other guided nuclease as disclosed elsewhere herein, with the nuclease guided by the use of one more of the following ADORA2A-specific guide RNAs: SEQ ID NO: 404-407. In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). Loss of expression of ADORA2A induces decreased sensitivity to adenosine, a well-established immunosuppressant for T cells and NK cells (Young et al., Cancer Res. (2018) 78(4):1003-16; Cekic and Linden, Cancer Res. (2014) 74(24):7239-49). In NK cells, loss of ADORA2A leads to loss of maturation, proliferation, effector function (as shown in constitutive knockout mice). In T cells, loss of ADORA2A leads to downstream loss of activation and function in CD8, increase in Treg and TH2, and loss of TH1. Thus, according to several embodiments, gene editing ADORA2A increases the cytotoxicity, persistence, immune avoidance or otherwise enhances the efficacy of engineered NK, T, or other cell as disclosed herein.

In several embodiments, gene editing reduces transcription of ADORA2A by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of ADORA2A by at least about 30%, In several embodiments, gene editing reduces transcription of ADORA2A by at least about 40%, In several embodiments, gene editing reduces transcription of ADORA2A by at least about 50%, In several embodiments, gene editing reduces transcription of ADORA2A by at least about 60%, In several embodiments, gene editing reduces transcription of ADORA2A by at least about 70%, In several embodiments, gene editing reduces transcription of ADORA2A by at least about 80%, In several embodiments, gene editing reduces transcription of ADORA2A by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of ADORA2A by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of ADORA2A by at least about 30%, In several embodiments, gene editing reduces expression of ADORA2A by at least about 40%, In several embodiments, gene editing reduces expression of ADORA2A by at least about 50%, In several embodiments, gene editing reduces expression of ADORA2A by at least about 60%, In several embodiments, gene editing reduces expression of ADORA2A by at least about 70%, In several embodiments, gene editing reduces expression of ADORA2A by at least about 80%, In several embodiments, gene editing reduces expression of ADORA2A by at least about 90%.

The tumor microenvironment (TME), as suggested with the nomenclature, is the environment around a tumor, which includes the surrounding blood vessels and capillaries, immune cells circulating through or retained in the area, fibroblasts, various signaling molecules related by the tumor cells, the immune cells or other cells in the area, as well as the surrounding extracellular matrix. Various mechanisms are employed by tumors to evade detection and/or destruction by host immune cells, including modification of the TME. Tumors may alter the TME by releasing extracellular signals, promoting tumor angiogenesis or even inducing immune tolerance, in part by limiting immune cell entry in the TME and/or limiting reproduction/expansion of immune cells in the TME. The tumor can also modify the extracellular matrix (ECM), which can allow pathways to develop for tumor extravasation to new sites. Transforming Growth-Factor beta (TGFb) has beneficial effects when reducing inflammation and preventing autoimmunity. However, it can also function to inhibit anti-tumor immune responses, and thus, upregulated expression of TGFb has been implicated in tumor progression and metastasis (Pickup et al., Nat. Rev. Cancer (2013) 13(11):788-99). TGFb signaling can inhibit the cytotoxic function of NK cells by interacting with the TGFb receptor expressed by NK cells, for example the TGFb receptor isoform II (TGFBR2). In accordance with several embodiments disclosed herein, the reduction or elimination of expression of TGFBR2 through gene editing (e.g., by CRISPr/Cas9 guided by a TGFBR2 guide RNA) interrupts the inhibitory effect of TGFb on NK cells.

In several embodiments, the expression of TGFBR2 is reduced and/or eliminated in order to increase overall activation in resultant T cells and/or NK cells, or other cell type provided for herein. In several embodiments, TGFBR2 is disrupted and/or knocked out using one or more of the gene editing methods disclosed herein. Non-limiting examples of guide RNAs to reduce and/or eliminate TGFBR2 expression are provided below in Table 6.

TABLE 6
TGFb Receptor Type 2 Isoform Guide RNAs

| SEQ ID | | | |
|---|---|---|---|
| NO: | Name | Sequence | Target |
| 147 | TGFBR2-1 | CCCCTACCATGACTTTATTC | Exon 4 |
| 148 | TGFBR2-2 | ATTGCACTCATCAGAGCTAC | Exon 4 |
| 149 | TGFBR2-3 | AGTCATGGTAGGGGAGCTTG | Exon 4 |
| 150 | TGFBR2-4 | TGCTGGCGATACGCGTCCAC | Exon 1 |
| 151 | TGFBR2-5 | GTGAGCAATCCCCCGGGCGA | Exon 4 |
| 152 | TGFBR2-6 | AACGTGCGGTGGGATCGTGC | Exon 1 |

In several embodiments, TGFBR2 is disrupted and/or knocked out using a Crispr-Cas mediated approach (e.g., Cas9), or other guided nuclease as disclosed elsewhere herein, with the nuclease guided by the use of one more of the following TGFBR2-specific guide RNAs: SEQ ID NO: 445-448.

In several embodiments, gene editing reduces transcription of TGFBR2 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of TGFBR2 by at least about 30%, In several embodiments, gene editing reduces transcription of TGFBR2 by at least about 40%, In several embodiments, gene editing reduces transcription of TGFBR2 by at least about 50%, In several embodiments, gene editing reduces transcription of TGFBR 2 by at least about 60%, In several embodiments, gene editing reduces transcription of TGFBR2 by at least about 70%, In several embodiments, gene editing reduces transcription of TGFBR2 by at least about 80%, In several embodiments, gene editing reduces transcription of TGFBR2 by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of TGFBR2 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of TGFBR2 by at least about 30%, In several embodiments, gene editing reduces expression of TGFBR2 by at least about 40%, In several embodiments, gene editing reduces expression of TGFBR2 by at least about 50%, In several embodiments, gene editing reduces expression of TGFBR2 by at least about 60%, In several embodiments, gene editing reduces expression of TGFBR2 by at least about 70%, In several embodiments, gene editing reduces expression of TGFBR2 by at least about 80%, In several embodiments, gene editing reduces expression of TGFBR2 by at least about 90%.

In NK cells, TGFBR2 is a potent checkpoint in NK cell-mediated tumor immunity, while for T cells, knockout of TGFBR2 rescues car T cell exhaustion induced by TGF-$1 (Tang et al., JCI Insight (2020) 5(4):e133977). Thus, according to several embodiments, gene editing TGFBR2 increases the cytotoxicity, persistence, and/or otherwise enhances the efficacy of engineered NK, T, or other cell as disclosed herein.

In accordance with additional embodiments, a disruption of, or elimination of, expression of a receptor, pathway or protein on an immune cell can result in the enhanced activity (e.g., cytotoxicity, persistence, etc.) of the immune cell against a target cancer cell. In several embodiments, this results from a disinhibition of the immune cell. Natural killer cells, express a variety of receptors, such particularly those within the Natural Killer Group 2 family of receptors. One such receptor, according to several embodiments disclosed herein, the NKG2D receptor, is used to generate cytotoxic signaling constructs that are expressed by NK cells and lead to enhanced anti-cancer activity of such NK cells. In addition, NK cells express the NKG2A receptor, which is an inhibitory receptor. One mechanism by which tumors develop resistance to immune cells is through the expression of peptide-loaded HLA Class I molecules (HLA-E), which suppresses the activity of NK cells through the ligation of the HLA-E with the NKG2A receptor. Thus, while one approach could be to block the interaction of the HLA-E with the expressed NKG2A receptors on NK cells, according to several embodiments disclosed herein, the expression of NKG2A is disrupted, which short circuits that inhibitory pathway and allows enhanced NK cell cytotoxicity.

Non-limiting examples of guide RNAs to reduce and/or eliminate NKG2A expression are provided below in Table 7.

TABLE 7
NKG2A Guide RNAs

| SEQ ID | | | |
|---|---|---|---|
| NO: | Name | Sequence | Target |
| 158 | NKG2A-1 | GGAGCTGATGGTAAATCTGC | Exon 4 |
| 159 | NKG2A-2 | TTGAAGGTTTAATTCCGCAT | Exon 3 |
| 160 | NKG2A-3 | AACAACTATCGTTACCACAG | Exon 4 |

In several embodiments, NKG2A is disrupted and/or knocked out using a Crispr-Cas mediated approach (e.g., Cas9), or other guided nuclease as disclosed elsewhere herein, with the nuclease guided by the use of one more of the following NKG2A-specific guide RNAs: SEQ ID NO: 450-452.

In several embodiments, gene editing reduces transcription of NKG2A by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of NKG2A by at least about 30%. In several embodiments, gene editing reduces transcription of NKG2A by at least about 40%. In several embodiments, gene editing reduces transcription of NKG2A by at least about 50%. In several embodiments, gene editing reduces transcription of NKG2A by at least about 60%. In several embodiments, gene editing reduces transcription of NKG2A by at least about 70%. In several embodiments, gene editing reduces transcription of NKG2A by at least about 80%. In several embodiments, gene editing reduces transcription of NKG2A by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of NKG2A by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of NKG2A by at least about 30%. In several embodiments, gene editing reduces expression of NKG2A by at least about 40%. In several embodiments, gene editing reduces expression of NKG2A by at least about 50%. In several embodiments, gene editing reduces expression of NKG2A by at least about 60%. In several embodiments, gene editing reduces expression of NKG2A by at least about 70%. In several embodiments, gene editing reduces expression of NKG2A by at least about 80%. In several embodiments, gene editing reduces expression of NKG2A by at least about 90%.

NKG2A binds to HLA-E and is recognized as an MHC-recognizing receptor. Since NKG2A is an inhibitor receptor, loss of expression of NKG2A induces increased activation of constituent cells. In NK and T cells. loss of NKG2A leads to increased activation and cytotoxicity against HLA-E expressing tumor cells (Kamiya et al., J. Clin. Invest. (2019) 129(5):2094-2106). Thus, according to several embodiments, gene editing NKG2A increases the cytotoxicity, persistence, and/or otherwise enhances the efficacy of engineered NK, T, or other cell as disclosed herein.

Interleukins, in particular interleukin-15, are important in NK cell function and survival. Suppressor of cytokine signaling (SOCS) proteins are negative regulators of cytokine release by NK cells. The protein tyrosine phosphatase CD45 is an important regulator of NK cell activity through Src-family kinase activity. CD45 expression is involved in ITAM-specific NK-cell functions and processes such as degranulation, cytokine production, and expansion (Hesslein et al., Blood (2011) 117(11):3087-95). Thus, knockout of CD45 expression should result in less effective NK cells. As discussed above, CRISPR/Cas9 was used to disrupt expression of CD45 (encoded by PTPRC) and SOCS2, though in additional embodiments, other gene editing approaches can be used. Non-limiting examples of CD45 and SOCS2-targeting guide RNAs are shown below in Table 8.

TABLE 8
CD45 and SOCS2 Guide RNAs

| SEQ ID | | | |
|---|---|---|---|
| NO: | Name | Sequence | Target |
| 170 | PTPRC-1 | AGTGCTGGTGTTGGGCGCAC | Exon 25 |
| 171 | SOCS2-1 | GTGAACAGTGCCGTTCCGGGGGG | Exon 3 |
| 1002 | SOCS2-4 | GTGAACAGTGCCGTTCCGGG | Exon 3 |
| 172 | SOCS2-2 | GGCACCGGTACATTTGTTAATGG | Exon 3 |
| 1003 | SOCS2-5 | GGCACCGGTACATTTGTTAA | Exon 3 |
| 173 | SOCS2-3 | TTCGCCAGACGCGCCGCCTGCGG | Exon 2 |
| 1004 | SOCS2-6 | TTCGCCAGACGCGCCGCCTG | Exon 2 |

In several embodiments, gene editing reduces transcription of PTPRC by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of PTPRC by at least about 30%. In several embodiments, gene editing reduces transcription of PTPRC by at least about 40%. In several embodiments, gene editing reduces transcription of PTPRC by at least about 50%. In several embodiments, gene editing reduces transcription of PTPRC by at least about 60%. In several embodiments, gene editing reduces transcription of PTPRC by at least about 70%. In several embodiments, gene editing reduces transcription of PTPRC by at least about 80%. In several embodiments, gene editing reduces transcription of PTPRC by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of CD45 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of CD45 by at least about 30%. In several embodiments, gene editing reduces expression of CD45 by at least about 40%. In several embodiments, gene editing reduces expression of CD45 by at least about 50%. In several embodiments, gene editing reduces expression of CD45 by at least about 60%. In several embodiments, gene editing reduces expression of CD45 by at least about 70%. In several embodiments, gene editing reduces expression of CD45 by at least about 80%. In several embodiments, gene editing reduces expression of CD45 by at least about 90%.

In several embodiments, SOCS2 is disrupted and/or knocked out using a Crispr-Cas mediated approach (e.g., Cas9), or other guided nuclease as disclosed elsewhere herein, with the nuclease guided by the use of one more of the following SOCS2-specific guide RNAs: SEQ ID NO: 457-462.

In several embodiments, gene editing reduces transcription of SOCS2 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of SOCS2 by at least about 30%. In several embodiments, gene editing reduces transcription of SOCS2 by at least about 40%. In several embodiments, gene editing reduces transcription of SOCS2 by at least about 50%. In several embodiments, gene editing reduces transcription of SOCS2 by at least about 60%. In several embodiments, gene editing reduces transcription of SOCS2 by at least about 70%. In several embodiments, gene editing reduces transcription of SOCS2 by at least about 80%. In several embodiments, gene editing reduces transcription of SOCS2 by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of SOCS2 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of SOCS2 by at least about 30%. In several embodiments, gene editing reduces expression of SOCS2 by at least about 40%. In several embodiments, gene editing reduces expression of SOCS2 by at least about 50%. In several embodiments, gene editing reduces expression of SOCS2 by at least about 60%. In several embodiments, gene editing reduces expression of SOCS2 by at least about 70%. In several embodiments, gene editing reduces expression of SOCS2 by at least about 80%. In several embodiments, gene editing reduces expression of SOCS2 by at least about 90%.

SOCS proteins are negative regulators of cytokine responses, and SOCS2 specifically negatively regulates the development of NK cells through inhibiting JAK2 activity. Loss of expression of SOCS2 in NK cells induces increased NK cell development and overall cytotoxicity (Kim et al., Scientific Reports (2017) 7:46153). Thus, according to several embodiments, gene editing SOCS2 increases the cytotoxicity, persistence, and/or otherwise enhances the efficacy of engineered NK, T, or other cell as disclosed herein.

In several embodiments, the expression of Casitas B-lineage lymphoma-b (Cbl-b) is reduced and/or eliminated in order to increase overall activation in resultant T cells and/or NK cells, or other cell type provided for herein. In several embodiments, Cbl-b is disrupted and/or knocked out using one or more of the gene editing methods disclosed herein. In several embodiments, Cbl-b is disrupted and/or knocked out using a Crispr-Cas mediated approach (e.g., Cas9), or other guided nuclease as disclosed elsewhere herein. Non-limiting examples of CBLB-targeting guide RNAs to reduce and/or eliminate expression of CBLB are shown below in Table 9.

TABLE 9
CBLB Guide RNAs

| SEQ ID | | | |
|---|---|---|---|
| NO: | Name | Sequence | Target |
| 164 | CBLB-1 | TAATCTGGTGGACCTCATGAAGG | Exon 5 |
| 1005 | CBLB-8 | TAATCTGGTGGACCTCATGA | Exon 5 |
| 165 | CBLB-2 | TCGGTTGGCAAACGTCCGAAAGG | Exon 10 |
| 1006 | CBLB-9 | TCGGTTGGCAAACGTCCGAA | Exon 10 |
| 166 | CBLB-3 | AGCAAGCTGCCGCAGATCGCAGG | Exon 2 |
| 1007 | CBLB-10 | AGCAAGCTGCCGCAGATCGC | Exon 2 |
| 935 | CBLB-4 | AAGACTCTTTAAAGAAGGCA | Exon 3 |
| 936 | CBLB-5 | AGTACTCATTCTCACTGAGT | Exon 3 |
| 937 | CBLB-6 | CGTAAATGCTGATATGTATC | Exon 3 |
| 1008 | CBLB-7 | TAATCTGGTGGACCTCATGA | Exon 5 |

In several embodiments, Cbl-b is disrupted and/or knocked out using a Crispr-Cas mediated approach (e.g., Cas9), or other guided nuclease as disclosed elsewhere herein, with the use of one more of the following CBLB-specific guide RNAs: SEQ ID NO: 453-456.

In several embodiments, gene editing reduces transcription of CBLB by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of CBLB by at least about 30%. In several embodiments, gene editing reduces transcription of CBLB by at least about 40%. In several embodiments, gene editing reduces transcription of CBLB by at least about 50%. In several embodiments, gene editing reduces transcription of CBLB by at least about 60%. In several embodiments, gene editing reduces transcription of CBLB by at least about 70%. In several embodiments, gene editing reduces transcription of CBLB by at least about 80%. In several embodiments, gene editing reduces transcription of CBLB by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of Cbl-b by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of Cbl-b by at least about 30%. In several embodiments, gene editing reduces expression of Cbl-b by at least about 40%. In several embodiments, gene editing reduces expression of Chl-b by at least about 50%. In several embodiments, gene editing reduces expression of Cbl-b by at least about 60%. In several embodiments, gene editing reduces expression of Cbl-b by at least about 70%. In several embodiments, gene editing reduces expression of Cbl-b by at least about 80%. In several embodiments, gene editing reduces expression of Cbl-b by at least about 90%.

Cbl-b is an E3 ubiquitin ligase that negatively regulates T cell activation Loss of expression of Cbl-b in NK cells and T cells demonstrate increased antitumor immunity. Moreover, Cbl-b deficient T cells and NK cells are resistant to PD-Li/PD-1 mediated suppression (Fujiwara et al., Front. Immunol. (2017) 8:42). Thus, according to several embodiments, gene editing Cbl-b increases the cytotoxicity, persistence, and/or otherwise enhances the efficacy of engineered NK, T, or other cell as disclosed herein.

Another E3 ubiquitin ligase, TRIpartite Motif-containing protein 29 (TRIM29), is a negative regulator of NK cell functions (Dou et al., J. Immunol. (2019) 203(4):873-80). TRIM29 is generally not expressed by resting NK cells, but is readily upregulated following activation (in particular by IL-12/IL-18 stimulation). Non-limiting examples of TRIM29-targeting guide RNAs to reduce and/or eliminate TRIM29 expression are shown below in Table 10.

TABLE 10
TRIM29 Guide RNAs

| SEQ ID | | | |
|---|---|---|---|
| NO: | Name | Sequence | Target |
| 167 | TRIM29-1 | GAACGGTAGGTCCCCTCTCGTGG | Exon 4 |
| 1009 | TRIM29-4 | GAACGGTAGGTCCCCTCTCG | Exon 4 |
| 168 | TRIM29-2 | AGCTGCCTTGGACGACGGGCAGG | Exon 7 |
| 1010 | TRIM29-5 | AGCTGCCTTGGACGACGGGC | Exon 7 |
| 169 | TRIM29-3 | TGAGCCGTAACTTCATTGAGAGG | Exon 4 |
| 1011 | TRIM29-6 | TGAGCCGTAACTTCATTGAG | Exon 4 |

In several embodiments, gene editing reduces transcription of TRIM29 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of TRIM29 by at least about 30%. In several embodiments, gene editing reduces transcription of TRIM29 by at least about 40%. In several embodiments, gene editing reduces transcription of TRIM29 by at least about 50%. In several embodiments, gene editing reduces transcription of TRIM29 by at least about 60%. In several embodiments, gene editing reduces transcription of TRIM29 by at least about 70%. In several embodiments, gene editing reduces transcription of TRIM29 by at least about 80%. In several embodiments, gene editing reduces transcription of TRIM29 by at least about 90%.

In several embodiments, gene editing can reduce expression of a target protein (e.g., TRIM29) by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of TRIM29 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of TRIM29 by at least about 30%. In several embodiments, gene editing reduces expression of TRIM29 by at least about 40%. In several embodiments, gene editing reduces expression of TRIM29 by at least about 50%. In several embodiments, gene editing reduces expression of TRIM29 by at least about 60%. In several embodiments, gene editing reduces expression of TRIM29 by at least about 70%. In several embodiments, gene editing reduces expression of TRIM29 by at least about 80%. In several embodiments, gene editing reduces expression of TRIM29 by at least about 90%.

In several embodiments, the expression of Beta-2 Microglobulin (B2-microglobulin) is reduced and/or eliminated in order to increase overall activation in resultant T cells and/or NK cells, or other cell type provided for herein. In several embodiments, B2-microglobulin is disrupted and/or knocked out using one or more of the gene editing methods disclosed herein. In several embodiments, B2-microglobulin is disrupted and/or knocked out using a Crispr-Cas mediated approach (e.g., Cas9), or other guided nuclease as disclosed elsewhere herein, with the nuclease guided by the use of one more of the following B2-microglobulin-specific guide RNAs: SEQ ID NO: 199-208.

In several embodiments, gene editing reduces transcription of B2M by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of B2M by at least about 30%. In several embodiments, gene editing reduces transcription of B2M by at least about 40%. In several embodiments, gene editing reduces transcription of B2M by at least about 50%. In several embodiments, gene editing reduces transcription of B2M by at least about 60%. In several embodiments, gene editing reduces transcription of B2M by at least about 70%. In several embodiments, gene editing reduces transcription of B2M by at least about 80%. In several embodiments, gene editing reduces transcription of B2M by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of B2M by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of B2M by at least about 30%. In several embodiments, gene editing reduces expression of B2M by at least about 40%. In several embodiments, gene editing reduces expression of B2M by at least about 50%. In several embodiments, gene editing reduces expression of B2M by at least about 60%. In several embodiments, gene editing reduces expression of B2M by at least about 70%. In several embodiments, gene editing reduces expression of B2M by at least about 80%. In several embodiments, gene editing reduces expression of B2M by at least about 90%.

Loss of expression of B2-microglobulin induces greatly reduced levels of MHC class I molecules, and in both NK cells and T cells, reduction of B2-microglobulin can modulate overall cell recognition of autologous and allogenic cells. Thus, according to several embodiments, gene editing B2-microglobulin increases the cytotoxicity, persistence, and/or otherwise enhances the efficacy of engineered NK, T, or other cell as disclosed herein.

In several embodiments, the expression of T cell immunoreceptor with Ig and ITIM domains (TIGIT) is reduced and/or eliminated in order to increase overall activation in resultant T cells and/or NK cells, or other cell type provided for herein. In several embodiments, TIGIT is disrupted and/or knocked out using one or more of the gene editing methods disclosed herein. In several embodiments, TIGIT is disrupted and/or knocked out using a Crispr-Cas mediated approach (e.g., Cas9), or other guided nuclease as disclosed elsewhere herein, with the nuclease guided by the use of one more of the following TIGIT-specific guide RNAs: SEQ ID NO: 408-411.

In several embodiments, gene editing reduces transcription of TIGIT by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of TIGIT by at least about 30%. In several embodiments, gene editing reduces transcription of TIGIT by at least about 40%. In several embodiments, gene editing reduces transcription of TIGIT by at least about 50%. In several embodiments, gene editing reduces transcription of TIGIT by at least about 60%. In several embodiments, gene editing reduces transcription of TIGIT by at least about 70%. In several embodiments, gene editing reduces transcription of TIGIT by at least about 80%. In several embodiments, gene editing reduces transcription of TIGIT by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of TIGIT by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of TIGIT by at least about 30%. In several embodiments, gene editing reduces expression of TIGIT by at least about 40%. In several embodiments, gene editing reduces expression of TIGIT by at least about 50%. In several embodiments, gene editing reduces expression of TIGIT by at least about 60%. In several embodiments, gene editing reduces expression of TIGIT by at least about 70%. In several embodiments, gene editing reduces expression of TIGIT by at least about 80%. In several embodiments, gene editing reduces expression of TIGIT by at least about 90%.

TIGIT is a checkpoint receptor associated with T cell and NK cell exhaustion. Loss of expression of TIGIT in NK cells prevents NK cell exhaustion and promotes NK cell-dependent tumor immunity (Zhang et al., Nat. Immunol. (2018) 19(7):723-32). Loss of expression of TIGIT in T cells can similarly lead to downstream activation of resultant T cells. Thus, according to several embodiments, gene editing TIGIT increases the cytotoxicity, persistence, and/or otherwise enhances the efficacy of engineered NK, T, or other cell as disclosed herein.

In several embodiments, the expression of Programmed cell death protein-1 (PD-1; encoded by PDCD1) is reduced and/or eliminated in order to increase overall activation in resultant T cells and/or NK cells, or other cell type provided for herein. In several embodiments, PD-1 is disrupted and/or knocked out using one or more of the gene editing methods disclosed herein. In several embodiments, PD-1 is disrupted and/or knocked out using a Crispr-Cas mediated approach (e.g., Cas9), or other guided nuclease as disclosed elsewhere herein, with the nuclease guided by the use of one more of the following PD-1-specific guide RNAs: SEQ ID NO: 412-415.

In several embodiments, gene editing reduces transcription of PDCD1 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of PDCD1 by at least about 30%. In several embodiments, gene editing reduces transcription of PDCD1 by at least about 40%. In several embodiments, gene editing reduces transcription of PDCD1 by at least about 50%. In several embodiments, gene editing reduces transcription of PDCD1 by at least about 60%. In several embodiments, gene editing reduces transcription of PDCD1 by at least about 70%. In several embodiments, gene editing reduces transcription of PDCD1 by at least about 80%. In several embodiments, gene editing reduces transcription of PDCD1 by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of PD-1 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of PD-1 by at least about 30%. In several embodiments, gene editing reduces expression of PD-1 by at least about 40%. In several embodiments, gene editing reduces expression of PD-1 by at least about 50%. In several embodiments, gene editing reduces expression of PD-1 by at least about 60%. In several embodiments, gene editing reduces expression of PD-1 by at least about 70%. In several embodiments, gene editing reduces expression of PD-1 by at least about 80%. In several embodiments, gene editing reduces expression of PD-1 by at least about 90%.

PD-1 plays an inhibitory role in immune regulation and down-regulates overall function by suppressing immune cell activity. Loss of expression of PD-1 in NK cells increases overall cytotoxicity due to increased secretion of interferon-gamma, granzyme B, and perforin (Niu et al., Int. J. Med. Sci. (2020) 17(13):1964-73). Similarly, T cells with loss of expression of PD-1 demonstrate increased cytotoxicity and overall caspase activation (Zhao et al., Ocotarget (2018) 9(4):5208-15). Thus, according to several embodiments, gene editing PD-1 increases the cytotoxicity, persistence, and/or otherwise enhances the efficacy of engineered NK, T, or other cell as disclosed herein.

In several embodiments, the expression of T-cell immunoglobulin and mucin-domain containing-3 (TIM-3; also known as HAVCR2) is reduced and/or eliminated in order to increase overall activation in resultant T cells and/or NK cells, or other cell type provided for herein. In several embodiments, TIM-3 is disrupted and/or knocked out using one or more of the gene editing methods disclosed herein. In several embodiments, TIM-3 is disrupted and/or knocked out using a Crispr-Cas mediated approach (e.g., Cas9), or other guided nuclease as disclosed elsewhere herein, with the nuclease guided by the use of one more of the following TIM-3-specific guide RNAs: SEQ ID NO:416-419.

In several embodiments, gene editing reduces transcription of TIM3 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of TIM3 by at least about 30%. In several embodiments, gene editing reduces transcription of TIM3 by at least about 40%. In several embodiments, gene editing reduces transcription of TIM3 by at least about 50%. In several embodiments, gene editing reduces transcription of TIM3 by at least about 60%. In several embodiments, gene editing reduces transcription of TIM3 by at least about 70%. In several embodiments, gene editing reduces transcription of TIM3 by at least about 80%. In several embodiments, gene editing reduces transcription of TIM3 by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of TIM-3 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of TIM-3 by at least about 30%. In several embodiments, gene editing reduces expression of TIM-3 by at least about 40%. In several embodiments, gene editing reduces expression of TIM-3 by at least about 50%. In several embodiments, gene editing reduces expression of TIM-3 by at least about 60%. In several embodiments, gene editing reduces expression of TIM-3 by at least about 70%. In several embodiments, gene editing reduces expression of TIM-3 by at least about 80%. In several embodiments, gene editing reduces expression of TIM-3 by at least about 90%.

TIM-3 is an inhibitory receptor involved in immune checkpoint function. Loss of expression of TIM-3 increases overall cytotoxicity in engineered NK and T cells as well as decreased exhaustion of NK cells and T cells, leading to increased effector function of constituent cells lacking TIM-3 expression (Pires de Silva et al., Cancer Imunol. Res. (2014) 2(5):410-22). Thus, according to several embodiments, gene editing TIM-3 increases the cytotoxicity, persistence, immune avoidance or otherwise enhances the efficacy of engineered NK, T, or other cell as disclosed herein.

In several embodiments, the expression of CD38 is reduced and/or eliminated in order to increase overall activation in resultant T cells and/or NK cells, or other cell type provided for herein. In several embodiments, CD38 is disrupted and/or knocked out using one or more of the gene editing methods disclosed herein. In several embodiments, CD38 is disrupted and/or knocked out using a Crispr-Cas mediated approach (e.g., Cas9), or other guided nuclease as disclosed elsewhere herein, with the nuclease guided by the use of one more of the following CD38-specific guide RNAs: SEQ ID NO:420-423.

In several embodiments, gene editing reduces transcription of CD38 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of CD38 by at least about 30%. In several embodiments, gene editing reduces transcription of CD38 by at least about 40%. In several embodiments, gene editing reduces transcription of CD38 by at least about 50%. In several embodiments, gene editing reduces transcription of CD38 by at least about 60%. In several embodiments, gene editing reduces transcription of CD38 by at least about 70%. In several embodiments, gene editing reduces transcription of CD38 by at least about 80%. In several embodiments, gene editing reduces transcription of CD38 by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of CD38 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of CD38 by at least about 30%. In several embodiments, gene editing reduces expression of CD38 by at least about 40%. In several embodiments, gene editing reduces expression of CD38 by at least about 50%. In several embodiments, gene editing reduces expression of CD38 by at least about 60%. In several embodiments, gene editing reduces expression of CD38 by at least about 70%. In several embodiments, gene editing reduces expression of CD38 by at least about 80%. In several embodiments, gene editing reduces expression of CD38 by at least about 90%.

CD38 plays a role in the maturation cycle of immune cells, and blood cancers can often present upregulated CD38. Loss of CD38 expression on constituent NK cells allows for greater cytotoxicity due to decreased fratricide (Nagai et al., Blood (2019) 134 (suppl. 1):870). Wild-type NK cells self-express CD38, leading to downstream self-targeting effects in wild-type NK cells. For T cells, loss of CD38 expression for constituent T cells leads to increased cytotoxicity. Thus, according to several embodiments, gene editing CD38 increases the cytotoxicity, persistence, and/or otherwise enhances the efficacy of engineered NK, T, or other cell as disclosed herein.

In several embodiments, the expression of T cell receptor alpha (TCR a) is reduced and/or eliminated in order to increase overall activation in resultant T cells and/or NK cells, or other cell type provided for herein. In several embodiments, TCR a is disrupted and/or knocked out using one or more of the gene editing methods disclosed herein. In several embodiments, TCR a is disrupted and/or knocked out using a Crispr-Cas mediated approach (e.g., Cas9), or other guided nuclease as disclosed elsewhere herein, with the nuclease guided by the use of one more of the following TCR a-specific guide RNAs: SEQ ID NO:467-470.

In several embodiments, gene editing reduces transcription of TRAC by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of TRAC by at least about 30%. In several embodiments, gene editing reduces transcription of TRAC by at least about 40%. In several embodiments, gene editing reduces transcription of TRAC by at least about 50%. In several embodiments, gene editing reduces transcription of TRAC by at least about 60%. In several embodiments, gene editing reduces transcription of TRAC by at least about 70%. In several embodiments, gene editing reduces transcription of TRAC by at least about 80%. In several embodiments, gene editing reduces transcription of TRAC by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of TRAC by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of TRAC by at least about 30%. In several embodiments, gene editing reduces expression of TRAC by at least about 40%. In several embodiments, gene editing reduces expression of TRAC by at least about 50%. In several embodiments, gene editing reduces expression of TRAC by at least about 60%. In several embodiments, gene editing reduces expression of TRAC by at least about 70%. In several embodiments, gene editing reduces expression of TRAC by at least about 80%. In several embodiments, gene editing reduces expression of TRAC by at least about 90%.

T cell receptors (TCR) are protein complexes found on T cells responsible for recognizing MHC molecules. Loss of certain TCRs and preferential expression of other TCRs can lead to increased cytotoxicity in engineered cells due to increased selective targeting and recognition by constituent cells. Thus, according to several embodiments, gene editing TCRs increases the cytotoxicity, persistence, and/or otherwise enhances the efficacy of engineered NK, T, or other cell as disclosed herein.

Cytokine-inducible SH2-containing protein (CIS) is a negative regulator of IL-15 signaling in NK cells, and is encoded by the CISH gene in humans. IL-15 signaling can have positive impacts on the NK cell expansion, survival, cytotoxicity and cytokine production. Thus, a disruption of CISH could render NK cells more sensitive to IL-15, thereby increasing their anti-tumor effects. In several embodiments, the expression of CISH is reduced and/or eliminated in order to increase overall activation in resultant T cells and/or NK cells, or other cell type provided for herein. It was observed in experiments described herein that disruption (e.g., knockout) of MED12—while increasing the cytotoxicity of NK cells—also tended to decrease the proliferation of such cells. However, it was surprisingly found that the effect of MED12 disruption on proliferation could be rescued by disruption (e.g., knockout) of CISH. Thus, in some aspects, it is contemplated that immune cells (e.g., NK cells) are knocked out for both MED12 and CISH. In some embodiments, the immune cells are genetically edited within a target sequence in a MED12 gene and are genetically edited within a target sequence in a CISH gene, wherein the edits yield reduced expression and/or function of the CIS and MED12 proteins encoded by the CISH and MED12 genes, respectively, as compared to an immune cell not edited within the target sequences.

In several embodiments, CISH is disrupted and/or knocked out using one or more of the gene editing methods disclosed herein. Non-limiting examples of CISH-targeting guide RNAs to reduce and/or eliminate expression of CIS (the protein encoded by CISH) are shown below in Table 11.

TABLE 11
CISH Guide RNAs

| SEQ ID | | | |
|---|---|---|---|
| NO: | Name | Sequence | Target |
| 153 | CISH-1 | CTCACCAGATTCCCGAAGGT | Exon 2 |
| 154 | CISH-2 | CCGCCTTGTCATCAACCGTC | Exon 3 |
| 155 | CISH-3 | TCTGCGTTCAGGGGTAAGCG | Exon 1 |
| 156 | CISH-4 | GCGCTTACCCCTGAACGCAG | Exon 1 |
| 157 | CISH-5 | CGCAGAGGACCATGTCCCCG | Exon 1 |
| 1012 | CISH-9 | GCAGGCACCAGGATGCCTGG | Exon 3 |
| 1013 | CISH-10 | GCATAGAGCTGGTCTCACTG | Exon 3 |

In several embodiments, CISH is disrupted and/or knocked out using a Crispr-Cas mediated approach (e.g., Cas9), or other guided nuclease as disclosed elsewhere herein, with the nuclease guided by the use of one more of the following CISH-specific guide RNAs: SEQ ID NO: 463-466, or other guide disclosed herein: SEQ ID NO 463: GCACCTACAGAAGATGCCGG; SEQ ID NO 464: GACAGCGTGAACAGGTAGCT; SEQ ID NO 465: GACAGCGTGAACAGGTAGCT; SE QID NO 466: ACTCAATGCGTACATTGGTG.

In several embodiments, gene editing reduces transcription of CISH by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of CISH by at least about 30%. In several embodiments, gene editing reduces transcription of CISH by at least about 40%. In several embodiments, gene editing reduces transcription of CISH by at least about 50%. In several embodiments, gene editing reduces transcription of CISH by at least about 60%. In several embodiments, gene editing reduces transcription of CISH by at least about 70%. In several embodiments, gene editing reduces transcription of CISH by at least about 80%. In several embodiments, gene editing reduces transcription of CISH by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of CISH by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of CISH by at least about 30%. In several embodiments, gene editing reduces expression of CISH by at least about 40%. In several embodiments, gene editing reduces expression of CISH by at least about 50%. In several embodiments, gene editing reduces expression of CISH by at least about 60%. In several embodiments, gene editing reduces expression of CISH by at least about 70%. In several embodiments, gene editing reduces expression of CISH by at least about 80%. In several embodiments, gene editing reduces expression of CISH by at least about 90%.

In CD8+ T cells, CISH actively silences TCR signaling to maintain tumor tolerance, and CISH has been shown to be a downstream negative regulator of IL-15 receptor signaling (Palmer et al., J. Exp. Med. (2015) 212(12):2095-2113). In NK and T cells, CISH plays a role in checkpoint maturation and proliferation (Delconte et al., Nature Immunol (2016) 17:816-24). Thus, according to several embodiments, gene editing CISH increases the cytotoxicity, persistence, and/or otherwise enhances the efficacy of engineered NK, T, or other cell as disclosed herein.

In several embodiments, the expression of CEACAM1 is reduced and/or eliminated in order to increase overall activation in resultant T cells and/or NK cells, or other cell type provided for herein. In several embodiments, CEACAM1 is disrupted and/or knocked out using one or more of the gene editing methods disclosed herein. In several embodiments, CEACAM1 is disrupted and/or knocked out using a Crispr-Cas mediated approach (e.g., Cas9), or other guided nuclease as disclosed elsewhere herein, with the nuclease guided by the use of one more of the following CEACAM1-specific guide RNAs: SEQ ID NO: 398-400.

In several embodiments, gene editing reduces transcription of CEACAM1 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of CEACAM1 by at least about 30%. In several embodiments, gene editing reduces transcription of CEACAM1 by at least about 40%. In several embodiments, gene editing reduces transcription of CEACAM1 by at least about 50%. In several embodiments, gene editing reduces transcription of CEACAM1 by at least about 60%. In several embodiments, gene editing reduces transcription of CEACAM1 by at least about 70%. In several embodiments, gene editing reduces transcription of CEACAM1 by at least about 80%. In several embodiments, gene editing reduces transcription of CEACAM1 by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of CEACAM1 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of CEACAM1 by at least about 30%. In several embodiments, gene editing reduces expression of CEACAM1 by at least about 40%. In several embodiments, gene editing reduces expression of CEACAM1 by at least about 50%. Tn several embodiments, gene editing reduces expression of CEACAM1 by at least about 60%. In several embodiments, gene editing reduces expression of CEACAM1 by at least about 70%. In several embodiments, gene editing reduces expression of CEACAM1 by at least about 80%. In several embodiments, gene editing reduces expression of CEACAM1 by at least about 90%.

CEACAM1 is an immune checkpoint for both NK and T cells and can inhibit lysis of CEACAM1-bearing tumor cell lines. Loss of expression of CEACAM1 can increase overall cytotoxicity for NK and T cells (Markel et al., J. Clin. Oncol. (2016) 34(suppl. 15):3044). Thus, according to several embodiments, gene editing CEACAM1 increases the cytotoxicity, persistence, and/or otherwise enhances the efficacy of engineered NK, T, or other cell as disclosed herein.

In several embodiments, the expression of DDIT4 is reduced and/or eliminated in order to increase overall activation in resultant T cells and/or NK cells, or other cell type provided for herein. In several embodiments, DDIT4 is disrupted and/or knocked out using one or more of the gene editing methods disclosed herein. In several embodiments, DDIT4 is disrupted and/or knocked out using a Crispr-Cas mediated approach (e.g., Cas9), or other guided nuclease as disclosed elsewhere herein, with the nuclease guided by the use of one more of the following DDIT4-specific guide RNAs: SEQ ID NO: 401-403.

In several embodiments, gene editing reduces transcription of DDIT4 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of DDIT4 by at least about 30%. In several embodiments, gene editing reduces transcription of DDIT4 by at least about 40%. In several embodiments, gene editing reduces transcription of DDIT4 by at least about 50%. In several embodiments, gene editing reduces transcription of DDIT4 by at least about 60%. In several embodiments, gene editing reduces transcription of DDIT4 by at least about 70%. In several embodiments, gene editing reduces transcription of DDIT4 by at least about 80%. In several embodiments, gene editing reduces transcription of DDIT4 by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of DDIT4 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of DDIT4 by at least about 30%. In several embodiments, gene editing reduces expression of DDIT4 by at least about 40%. In several embodiments, gene editing reduces expression of DDIT4 by at least about 50%. In several embodiments, gene editing reduces expression of DDIT4 by at least about 60%. In several embodiments, gene editing reduces expression of DDIT4 by at least about 70%. In several embodiments, gene editing reduces expression of DDTT4 by at least about 80%. In several embodiments, gene editing reduces expression of DDIT4 by at least about 90%.

In NK and T cells, DDIT4 is a negative regulator of mTORC1, which itself enhances IL-15 mediated survival and proliferation of NK cells. Moreover, DDIT4 is upregulated by oxidative stress conditions as is common in tumor microenvironments. Loss of DDIT4 function in engineered cells may increase overall glucose metabolism leading to enhanced proliferation, as well as increasing overall NK or T cell cytotoxicity. Thus, according to several embodiments, gene editing DDIT4 increases the cytotoxicity, persistence, and/or otherwise enhances the efficacy of engineered NK, T, or other cell as disclosed herein.

In several embodiments, the expression of MAPKAP Kinase 3 (MAPKAPK3) is reduced and/or eliminated in order to increase overall activation in resultant T cells and/or NK cells, or other cell type provided for herein. In several embodiments, MAPKAPK3 is disrupted and/or knocked out using one or more of the gene editing methods disclosed herein. In several embodiments, MAPKAPK3 is disrupted and/or knocked out using a Crispr-Cas mediated approach (e.g., Cas9), or other guided nuclease as disclosed elsewhere herein, with the nuclease guided by the use of one more of the following MAPKAPK3-specific guide RNAs: SEQ ID NO: 395-397.

In several embodiments, gene editing reduces transcription of MAPKAPK3 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of MAPKAPK3 by at least about 30%. In several embodiments, gene editing reduces transcription of MAPKAPK3 by at least about 40%. In several embodiments, gene editing reduces transcription of MAPKAPK3 by at least about 50%. In several embodiments, gene editing reduces transcription of MAPKAPK3 by at least about 60%. In several embodiments, gene editing reduces transcription of MAPKAPK3 by at least about 70%. In several embodiments, gene editing reduces transcription of MAPKAPK3 by at least about 80%. In several embodiments, gene editing reduces transcription of MAPKAPK3 by at least about 90%.

In several embodiments, gene editing reduces expression of MAPKAPK3 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of MAPKAPK3 by at least about 30%. In several embodiments, gene editing reduces expression of MAPKAPK3 by at least about 40%. In several embodiments, gene editing reduces expression of MAPKAPK3 by at least about 50%. In several embodiments, gene editing reduces expression of MAPKAPK3 by at least about 60%. In several embodiments, gene editing reduces expression of MAPKAPK3 by at least about 70%. In several embodiments, gene editing reduces expression of MAPKAPK3 by at least about 80%. In several embodiments, gene editing reduces expression of DDIT4 by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). MAPKAP Kinase 3 in expressed in both NK and T cells. Loss of MAPKAPK3 in engineered cells is expected to increase cytotoxicity, cytokine secretion, and overall NK signaling. Thus, according to several embodiments, gene editing MAPKAPK3 increases the cytotoxicity, persistence, and/or otherwise enhances the efficacy of engineered NK, T, or other cell as disclosed herein.

In several embodiments, the expression of SMAD3 is reduced and/or eliminated in order to increase overall activation in resultant T cells and/or NK cells, or other cell type provided for herein. In several embodiments, SMAD3 is disrupted and/or knocked out using one or more of the gene editing methods disclosed herein. In several embodiments, SMAD3 is disrupted and/or knocked out using a Crispr-Cas mediated approach (e.g., Cas9), or other guided nuclease as disclosed elsewhere herein, with the nuclease guided by the use of one more of the following SMAD3-specific guide RNAs: SEQ ID NO: 392-394.

In several embodiments, gene editing reduces transcription of SMAD3 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of SMAD3 by at least about 30%. In several embodiments, gene editing reduces transcription of SMAD3 by at least about 40%. In several embodiments, gene editing reduces transcription of SMAD3 by at least about 50%. In several embodiments, gene editing reduces transcription of SMAD3 by at least about 60%. In several embodiments, gene editing reduces transcription of SMAD3 by at least about 70%. In several embodiments, gene editing reduces transcription of SMAD3 by at least about 80%. In several embodiments, gene editing reduces transcription of SMAD3 by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of SMAD3 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of SMAD3 by at least about 30%. In several embodiments, gene editing reduces expression of SMAD3 by at least about 40%. In several embodiments, gene editing reduces expression of SMAD3 by at least about 50%. In several embodiments, gene editing reduces expression of SMAD3 by at least about 60%. In several embodiments, gene editing reduces expression of SMAD3 by at least about 70%. In several embodiments, gene editing reduces expression of SMAD3 by at least about 80%. In several embodiments, gene editing reduces expression of SMAD3 by at least about 90%.

SMAD3 is a downstream mediator of TGF-Beta and Activin A signaling. Inhibition of activin A provides an effective downstream TGFBR knockout. Smad3 silenced NK cells demonstrate increased proliferation and differentiation, as well as increased cytotoxicity in engineered T and NK cells (Tang et al., Nat. Commun. (2017) 8:14677). Thus, according to several embodiments, gene editing SMAD3 increases the cytotoxicity, persistence, and/or otherwise enhances the efficacy of engineered NK, T, or other cell as disclosed herein.

As discussed above, genetically edited cells can be edited at a plurality of locations. For example in several embodiments, cells (e.g., NK cells or T cells, or a mixture thereof) are edited at two locations. In several embodiments, one of the gene edits is made at a target site in the CISH gene. In several embodiments, one of the gene edits is made at a target site in the CBLB gene. In several embodiments, one of the gene edits is made at a target site in the TGFBR2 gene. In several embodiments, one of the gene edits is made at a target site in the TIGIT gene. Any combination of such edits is also within the provided embodiments, for example dual TGFBR2/CBLB, dual TIGIT/TGFBR2, CISH/CBLB, CISH/TGFBR2, CISH/TIGIT, etc. Moreover, any combination of edits of any of the target genes for editing (e.g., by Crispr or other nuclease) can be made according to some embodiments. Additionally, to the extent necessary to achieve a desired amount of reduction in gene expression, multiple edits may be made within a single target gene, or genes.

In several embodiments, gene edits are made at a target site in a CISH gene and a target site in a CBLB gene. In some embodiments, a double edit, e.g., CISH/CBLB is made in NK cells and/or T cells for use in therapy. In several embodiments, a combination CISH/CBLB gene edit is made in an NK cell that does not include an edit at a CD70 gene. In several embodiments, a combination CISH/CBLB gene edit is made in an NK cell that does not include an edit at any additional gene. In several embodiments, a combination CISH/CBLB gene edit is made in an NK cell that does not express any one or combination of any of an anti-CD70 CAR, an anti-CD19 CAR, or an anti-NKG2D chimeric receptor. In some embodiments, a triple edit, e.g., CD70/CISH/CBLB is made in NK cells and/or T cells for use in therapy. In some embodiments, a triple edit, e.g., CD70/CISH/CBLB is made in NK cells and/or T cells that are engineered to express a tumor-targeting CAR.

In several embodiments, gene edits are made at a target site in a CISH gene and a target site in a MED12 gene. In some embodiments, a double edit, e.g., CISH/MED12 is made in NK cells and/or T cells. In several embodiments, a combination CISH/MED12 gene edit is made in NK cells. In several embodiments, a combination CISH/MED12 gene edit is made in an NK cell that does not include an edit at a CD70 gene. In several embodiments, a combination CISH/MED12 gene edit is made in an NK cell that does not include an edit at any additional gene. In several embodiments, a combination CISH/MED12 gene edit is made in an NK cell that does not express any one or combination of any of an anti-CD70 CAR, an anti-CD19 CAR, or an anti-NKG2D chimeric receptor. In several embodiments, a combination CISH/MED12 gene edit is made in an NK cell that expresses a CD19-targeting CAR. In several embodiments, a combination CISH/MED12 gene edit is made in an NK cell that expresses a CD70-targeting CAR. In several embodiments, a combination CISH/MED12 gene edit is made in an NK cell that expresses a BCMA-targeting CAR.

In several embodiments, gene edits are made at a target site in a CISH gene, a target site in a CBLB gene, and a target site in a MED12 gene. In some embodiments, a triple edit, e.g., CBLB/CISH/MED12, is made in NK cells and/or T cells. In some embodiments, a triple edit, e.g., CBLB/CISH/MED12, is made in NK cells. In several embodiments, a combination CISH/MED12/CBLB gene edit is made in an NK cell that does not include an edit at a CD70 gene. In several embodiments, a combination CISH/MED12/CBLB gene edit is made in an NK cell that does not include an edit at any additional gene. In several embodiments, a combination CISH/MED12/CBLB gene edit is made in an NK cell that does not express any one or combination of any of an anti-CD70 CAR, an anti-CD19 CAR, or an anti-NKG2D chimeric receptor. In some embodiments, a triple edit, e.g., CBLB/CISH/MED12, is made in NK cells that express a CD19-targeting CAR. In some embodiments, a triple edit, e.g., CBLB/CISH/MED12, is made in NK cells that express a CD70-targeting CAR. In some embodiments, a triple edit, e.g., CBLB/CISH/MED12, is made in NK cells that express a BCMA-targeting CAR.

In several embodiments, gene edits are made at a target site in a CISH gene and a target site in a TGFBR2 gene. In some embodiments, a double edit, e.g., CISH/TGFBR2 is made in NK cells and/or T cells for use in therapy. In several embodiments, a combination CISH/TGFBR2 gene edit is made in an NK cell that does not include an edit at a CD70 gene. In several embodiments, a combination CISH/TGFBR2 gene edit is made in an NK cell that does not include an edit at any additional gene. In several embodiments, a combination CISH/TGFBR2 gene edit is made in an NK cell that does not express any one or combination of any of an anti-CD70 CAR, an anti-CD19 CAR, or an anti-NKG2D chimeric receptor. In some embodiments, a triple edit, e.g., CD70/CISH/TGFBR2 is made in NK cells and/or T cells for use in therapy. In some embodiments, a triple edit, e.g., CD70/CISH/TGFBR2 is made in NK cells and/or T cells that are engineered to express a tumor-targeting CAR.

In several embodiments, gene edits are made at a target site in a CISH gene and a target site in a TIGIT gene. In some embodiments, a double edit, e.g., CISH/TIGIT is made in NK cells and/or T cells for use in therapy. In several embodiments, a combination CISH/TIGIT gene edit is made in an NK cell that does not include an edit at a CD70 gene. In several embodiments, a combination CISH/TIGIT gene edit is made in an NK cell that does not include an edit at any additional gene. In several embodiments, a combination CISH/TIGIT gene edit is made in an NK cell that does not express any one or combination of any of an anti-CD70 CAR, an anti-CD19 CAR, or an anti-NKG2D chimeric receptor. In some embodiments, a triple edit, e.g., CD70/CISH/TIGIT is made in NK cells and/or T cells for use in therapy. In some embodiments, a triple edit, e.g., CD70/CISH/TIGIT is made in NK cells and/or T cells that are engineered to express a tumor-targeting CAR.

### Extracellular Domains (Tumor Binder)

Some embodiments of the compositions and methods described herein relate to a chimeric antigen receptor that includes an extracellular domain that comprises a tumor-binding domain (also referred to as an antigen-binding protein or antigen-binding domain) as described herein. The tumor binding domain, depending on the embodiment, targets, for example CD19, CD123, CD70, Her2, mesothelin, Claudin 6, BCMA, EGFR, among others. In some embodiments, the tumor binding domain binds CD19. In some embodiments, the tumor binding domain binds CD70. In some embodiments, the tumor binding domain binds BCMA. Several embodiments of the compositions and methods described herein relate to a chimeric receptor that includes an extracellular domain that comprises a ligand binding domain that binds a ligand expressed by a tumor cell (also referred to as an activating chimeric receptor) as described herein. In some embodiments, the ligand binding domain binds to a ligand of NKG2D. The ligand binding domain, depending on the embodiment, targets for example MICA, MICB, ULBP1, ULBP2, ULBP3, ULBP4, ULBP5, and ULBP6 (among others).

In some embodiments, the antigen-binding domain is derived from or comprises wild-type or non-wild-type sequence of an antibody, an antibody fragment, an scFv, a Fv, a Fab, a (Fab′)2, a single domain antibody (sdAB), a vH or vL domain, a camelid VHH domain, or a non-immunoglobulin scaffold such as a DARPIN, an affibody, an affilin, an adnectin, an affitin, a repebody, a fynomer, an alphabody, an avimer, an atrimer, a centyrin, a pronectin, an anticalin, a kunitz domain, an Armadillo repeat protein, an autoantigen, a receptor or a ligand. In some embodiments, the tumor-binding domain contains more than one antigen binding domain. In embodiments, the antigen-binding domain is operably linked directly or via an optional linker to the NH2-terminal end of a TCR domain (e.g. constant chains of TCR-alpha, TCR-betal, TCR-beta2, preTCR-alpha, pre-TCR-alpha-Del48, TCR-gamma, or TCR-delta).

### Antigen-Binding Proteins

There are provided, in several embodiments, antigen-binding proteins. Also provided, in several embodiments, are chimeric receptors (e.g., CARs) comprising antigen-binding proteins. As used herein, the term “antigen-binding protein” shall be given its ordinary meaning, and shall also refer to a protein comprising an antigen-binding fragment that binds to an antigen and, optionally, a scaffold or framework portion that allows the antigen-binding fragment to adopt a conformation that promotes binding of the antigen-binding protein to the antigen. In some embodiments, the antigen is a cancer antigen (e.g., CD19) or a fragment thereof. In some embodiments, the antigen-binding fragment comprises at least one CDR from an antibody that binds to the antigen. In some embodiments, the antigen-binding fragment comprises all three CDRs from the heavy chain of an antibody that binds to the antigen or from the light chain of an antibody that binds to the antigen. In some embodiments, the antigen-binding fragment is a heavy chain-only antibody, such as a camelid antibody (e.g., V_{H}H). In still some embodiments, the antigen-binding fragment comprises all six CDRs from an antibody that binds to the antigen (three from the heavy chain and three from the light chain). In some embodiments, the antigen-binding fragment comprises a heavy chain variable region (VH) and a light chain variable region (VL) (e.g., a Fv or a scFv). In several embodiments, the antigen-binding fragment comprises one, two, three, four, five, or six CDRs from an antibody that binds to the antigen, and in several embodiments, the CDRs can be any combination of heavy and/or light chain CDRs. The antigen-binding fragment in some embodiments is an antibody fragment.

Nonlimiting examples of antigen-binding proteins include antibodies, antibody fragments (e.g., an antigen-binding fragment of an antibody), antibody derivatives, and antibody analogs. Further specific examples include, but are not limited to, a single-chain variable fragment (scFv), a nanobody (e.g. VH domain of camelid heavy chain antibodies; VHH fragment,), a Fab fragment, a Fab′ fragment, a F(ab′)₂ fragment, a Fv fragment, a Fd fragment, and a complementarity determining region (CDR) fragment. In some embodiments, the antigen-binding fragment is a Fab. In some embodiments, the antigen-binding fragment is a Fab′ fragment. In some embodiments, the antigen-binding fragment is a F(ab′)₂ fragment. In some embodiments, the antigen-binding fragment is a Fd fragment. In some embodiments, the antigen-binding fragment is a Fv. In some embodiments, the antigen-binding fragment comprises a linker between the VH and VL In some embodiments, the antigen-binding fragment is a single-chain variable fragment (scFv). These molecules can be derived from any mammalian source, such as human, mouse, rat, rabbit, or pig, dog, or camelid. Antibody fragments may compete for binding of a target antigen with an intact (e.g., native) antibody and the fragments may be produced by the modification of intact antibodies (e.g. enzymatic or chemical cleavage) or synthesized de novo using recombinant DNA technologies or peptide synthesis. The antigen-binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen-binding protein as well as wholly synthetic scaffolds comprising, for example, a biocompatible polymer. In addition, peptide antibody mimetics (“PAMs”) can be used, as well as scaffolds based on antibody mimetics utilizing fibronectin components as a scaffold.

In some embodiments, the antigen-binding protein comprises one or more antibody fragments incorporated into a single polypeptide chain or into multiple polypeptide chains. For instance, antigen-binding proteins can include, but are not limited to, a diabody; an intrabody; a domain antibody (single VL or VH domain or two or more VH domains joined by a peptide linker;); a maxibody (2 scFvs fused to Fe region); a triabody; a tetrabody; a minibody (scFv fused to CH3 domain); a peptibody (one or more peptides attached to an Fe region); a linear antibody (a pair of tandem Fd segments (VH-CH1-VH-CH1) which, together with complementary light chain polypeptides, form a pair of antigen binding regions); a small modular immunopharmaceutical; and immunoglobulin fusion proteins (e.g. IgG-scFv, IgG-Fab, 2scFv-IgG, 4scFv-IgG, VH-IgG, IgG-VH, and Fab-scFv-Fc).

In some embodiments, the antigen-binding protein has the structure of an immunoglobulin. As used herein, the term “immunoglobulin” shall be given its ordinary meaning, and shall also refer to a tetrameric molecule, with each tetramer comprising two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

Within light and heavy chains, the variable (V) and constant regions (C) are joined by a “J” region of about 12 or more amino acids, with the heavy chain also including a “D” region of about 10 more amino acids. The variable regions of each light/heavy chain pair form the antibody binding site such that an intact immunoglobulin has two binding sites.

Immunoglobulin chains exhibit the same general structure of relatively conserved framework regions (FR) joined by three hypervariable regions, also called complementarity determining regions or CDRs. From N-terminus to C-terminus, both light and heavy chains comprise the domains FR1, CDR1, FR2, CDR2, FR3, CDR3 and FR4.

Human light chains are classified as kappa and lambda light chains. An antibody “light chain”, refers to the smaller of the two types of polypeptide chains present in antibody molecules in their naturally occurring conformations. Kappa (K) and lambda (λ) light chains refer to the two major antibody light chain isotypes. A light chain may include a polypeptide comprising, from amino terminus to carboxyl terminus, a single immunoglobulin light chain variable region (VL) and a single immunoglobulin light chain constant domain (CL).

Heavy chains are classified as mu (μ), delta (Δ), gamma (γ), alpha (α), and epsilon (ε), and define the antibody's isotype as IgM, IgD, IgG, IgA, and IgE, respectively. An antibody “heavy chain” refers to the larger of the two types of polypeptide chains present in antibody molecules in their naturally occurring conformations, and which normally determines the class to which the antibody belongs. A heavy chain may include a polypeptide comprising, from amino terminus to carboxyl terminus, a single immunoglobulin heavy chain variable region (VH), an immunoglobulin heavy chain constant domain 1 (CH1), an immunoglobulin hinge region, an immunoglobulin heavy chain constant domain 2 (CH2), an immunoglobulin heavy chain constant domain 3 (CH3), and optionally an immunoglobulin heavy chain constant domain 4 (CH4).

The IgG-class is further divided into subclasses, namely, IgG1, IgG2, IgG3, and IgG4. The IgA-class is further divided into subclasses, namely IgA1 and IgA2. The IgM has subclasses including, but not limited to, IgM1 and IgM2. The heavy chains in IgG, IgA, and IgD antibodies have three domains (CH1, CH2, and CH3), whereas the heavy chains in IgM and IgE antibodies have four domains (CHI, CH2, CH3, and CH4). The immunoglobulin heavy chain constant domains can be from any immunoglobulin isotype, including subtypes. The antibody chains are linked together via inter-polypeptide disulfide bonds between the CL domain and the CH1 domain (e.g., between the light and heavy chain) and between the hinge regions of the antibody heavy chains.

In some embodiments, the antigen-binding protein is an antibody. The term “antibody”, as used herein, refers to a protein, or polypeptide sequence derived from an immunoglobulin molecule which specifically binds with an antigen. Antibodies can be monoclonal, or polyclonal, multiple or single chain, or intact immunoglobulins, and may be derived from natural sources or from recombinant sources. Antibodies can be tetramers of immunoglobulin molecules. The antibody may be “humanized”, “chimeric” or non-human. An antibody may include an intact immunoglobulin of any isotype, and includes, for instance, chimeric, humanized, human, and bispecific antibodies. An intact antibody will generally comprise at least two full-length heavy chains and two full-length light chains. Antibody sequences can be derived solely from a single species, or can be “chimeric.” that is, different portions of the antibody can be derived from two different species as described further below. Unless otherwise indicated, the term “antibody” also includes antibodies comprising two substantially full-length heavy chains and two substantially full-length light chains provided the antibodies retain the same or similar binding and/or function as the antibody comprised of two full length light and heavy chains. For example, antibodies having 1, 2, 3, 4, or 5 amino acid residue substitutions, insertions or deletions at the N-terminus and/or C-terminus of the heavy and/or light chains are included in the definition provided that the antibodies retain the same or similar binding and/or function as the antibodies comprising two full length heavy chains and two full length light chains. Examples of antibodies include monoclonal antibodies, polyclonal antibodies, chimeric antibodies, humanized antibodies, human antibodies, bispecific antibodies, and synthetic antibodies. There is provided, in some embodiments, monoclonal and polyclonal antibodies. As used herein, the term “polyclonal antibody” shall be given its ordinary meaning, and shall also refer to a population of antibodies that are typically widely varied in composition and binding specificity. As used herein, the term “monoclonal antibody” (“mAb”) shall be given its ordinary meaning, and shall also refer to one or more of a population of antibodies having identical sequences. Monoclonal antibodies bind to the antigen at a particular epitope on the antigen.

In some embodiments, the antigen-binding protein is a fragment or antigen-binding fragment of an antibody. The term “antibody fragment” refers to at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)₂, Fv fragments, scFv antibody fragments, disulfide-linked Fvs (sdFv), a Fd fragment consisting of the VH and CHI domains, linear antibodies, single domain antibodies such as sdAb (either vL or vH), camelid V_{H}H domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23: 1126-1136, 2005). Antigen binding fragments can also be grafted into scaffolds based on polypeptides such as a fibronectin type III (Fn3)(see U.S. Pat. No. 6,703,199, which describes fibronectin polypeptide mini bodies). An antibody fragment may include a Fab, Fab′, F(ab′)2, and/or Fv fragment that contains at least one CDR of an immunoglobulin that is sufficient to confer specific antigen binding to a cancer antigen (e.g., CDI 9). Antibody fragments may be produced by recombinant DNA techniques or by enzymatic or chemical cleavage of intact antibodies.

In some embodiments, Fab fragments are provided. A Fab fragment is a monovalent fragment having the VL, VH, CL and CHI domains; a F(ab′)2 fragment is a bivalent fragment having two Fab fragments linked by a disulfide bridge at the hinge region; a Fd fragment has the VH and CH1 domains; an Fv fragment has the VL and VH domains of a single arm of an antibody; and a dAb fragment has a VH domain, a VL domain, or an antigen-binding fragment of a VH or VL domain. In some embodiments, these antibody fragments can be incorporated into single domain antibodies (e.g., V_{H}Hs), single-chain antibodies, maxibodies, minibodies, intrabodies, diabodies, triabodies, tetrabodies, v-NAR and bis-scFv. In some embodiments, the antibodies comprise at least one CDR as described herein.

There is also provided for herein, in several embodiments, single-chain variable fragments. As used herein, the term “single-chain variable fragment” (“scFv”) shall be given its ordinary meaning, and shall also refer to a fusion protein in which a VL and a VH region are joined via a linker (e.g., a synthetic sequence of amino acid residues) to form a continuous protein chain wherein the linker is long enough to allow the protein chain to fold back on itself and form a monovalent antigen binding site). For the sake of clarity, unless otherwise indicated as such, a “single-chain variable fragment” is not an antibody or an antibody fragment as defined herein. Diabodies are bivalent antibodies comprising two polypeptide chains, wherein each polypeptide chain comprises VH and VL domains joined by a linker that is configured to reduce or not allow for pairing between two domains on the same chain, thus allowing each domain to pair with a complementary domain on another polypeptide chain. According to several embodiments, if the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different.

In several embodiments, the antigen-binding protein comprises one or more CDRs. As used herein, the term “CDR” shall be given its ordinary meaning, and shall also refer to the complementarity determining region (also termed “minimal recognition units” or “hypervariable region”) within antibody variable sequences. The CDRs permit the antigen-binding protein to specifically bind to a particular antigen of interest. There are three heavy chain variable region CDRs (CDRH1, CDRH2 and CDRH3) and three light chain variable region CDRs (CDRL1, CDRL2 and CDRL3). The CDRs in each of the two chains typically are aligned by the framework regions to form a structure that binds specifically to a specific epitope or domain on the target protein. From N-terminus to C-terminus, naturally-occurring light and heavy chain variable regions both typically conform to the following order of these elements: FRI, CDR1, FR2, CDR2, FR3, CDR3 and FR4. A numbering system has been devised for assigning numbers to amino acids that occupy positions in each of these domains. This numbering system is defined in Kabat Sequences of Proteins of Immunological Interest (1987 and 1991, NIH, Bethesda, MD), or Chothia & Lesk, 1987, J. Mol. Biol. 196:901-917; Chothia et al., 1989, Nature 342:878-883. Complementarity determining regions (CDRs) and framework regions (FR) of a given antibody may be identified using this system. Other numbering systems for the amino acids in immunoglobulin chains include IMGT® (the international ImMunoGeneTics information system; Lefranc et al, Dev. Comp. Immunol. 29:185-203; 2005) and AHo (Honegger and Pluckthun, J. Mol. Biol. 309(3):657-670; 2001). One or more CDRs may be incorporated into a molecule either covalently or noncovalently to make it an antigen-binding protein.

In some embodiments, the antigen-binding protein comprises the CDR-H1, the CDR-H2, and the CDR-H3 of any VH sequence provided herein; and the CDR-L1, CDR-L2, and CDR-L3 of any VL sequence provided herein. In some embodiments, the VH comprises the CDR-H1, the CDR-H2, and the CDR-H3 of any VH sequence provided herein; and the VL comprises the CDR-L1, the CDR-L2, and the CDR-L3 of any VL sequence provided herein.

In some embodiments, the antigen-binding proteins provided herein comprise one or more CDR(s) as part of a larger polypeptide chain. In some embodiments, the antigen-binding proteins covalently link the one or more CDR(s) to another polypeptide chain. In some embodiments, the antigen-binding proteins incorporate the one or more CDR(s) noncovalently. In some embodiments, the antigen-binding proteins may comprise at least one of the CDRs described herein incorporated into a biocompatible framework structure. In some embodiments, the biocompatible framework structure comprises a polypeptide or portion thereof that is sufficient to form a conformationally stable structural support, or framework, or scaffold, which is able to display one or more sequences of amino acids that bind to an antigen (e.g., CDRs, a variable region, etc.) in a localized surface region. Such structures can be a naturally occurring polypeptide or polypeptide “fold” (a structural motif), or can have one or more modifications, such as additions, deletions and/or substitutions of amino acids, relative to a naturally occurring polypeptide or fold. Depending on the embodiment, the scaffolds can be derived from a polypeptide of a variety of different species (or of more than one species), such as a human, a non-human primate or other mammal, other vertebrate, invertebrate, plant, bacteria or virus.

Depending on the embodiment, the biocompatible framework structures are based on protein scaffolds or skeletons other than immunoglobulin domains. In some such embodiments, those framework structures are based on fibronectin, ankyrin, lipocalin, neocarzinostain, cytochrome b, CPI zinc finger, PST1, coiled coil, LACI-D1, Z domain and/or tendamistat domains.

There is also provided, in some embodiments, antigen-binding proteins with more than one binding site. In several embodiments, the binding sites are identical to one another while in some embodiments the binding sites are different from one another. For example, an antibody typically has two identical binding sites, while a “bispecific” or “bifunctional” antibody has two different binding sites. The two binding sites of a bispecific antigen-binding protein or antibody will bind to two different epitopes, which can reside on the same or different protein targets. In several embodiments, this is particularly advantageous, as a bispecific chimeric antigen receptor can impart to an engineered cell the ability to target multiple tumor markers. For example, CD19 and an additional tumor marker, such as CD123, CD70, Her2, mesothelin, Claudin 6, BCMA, EGFR, MICA, MICB, ULBP1, ULBP2, ULBP3, ULBP4, ULBP5, and ULBP6, among others, or any other marker disclosed herein or appreciated in the art as a tumor specific antigen or tumor associated antigen can be bound by a bispecific antibody.

As used herein, the term “chimeric antibody” shall be given its ordinary meaning, and shall also refer to an antibody that contains one or more regions from one antibody and one or more regions from one or more other antibodies. In some embodiments, one or more of the CDRs are derived from an anti-cancer antigen (e.g., CD19, CD123, CD70, Her2, mesothelin, PD-L1, Claudin 6, BCMA, CD138, EGFR, etc.) antibody. In several embodiments, all of the CDRs are derived from an anti-cancer antigen antibody (such as an anti-CD19 antibody). In some embodiments, the CDRs from more than one anti-cancer antigen antibodies are mixed and matched in a chimeric antibody. For instance, a chimeric antibody may comprise a CDR1 from the light chain of a first anti-cancer antigen antibody, a CDR2 and a CDR3 from the light chain of a second anti-cancer antigen antibody, and the CDRs from the heavy chain from a third anti-cancer antigen antibody. Further, the framework regions of antigen-binding proteins disclosed herein may be derived from one of the same anti-cancer antigen (e.g., CD19, CD123, CD70, Her2, mesothelin, Claudin 6, BCMA, EGFR, etc.) antibodies, from one or more different antibodies, such as a human antibody, or from a humanized antibody. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody or antibodies from another species or belonging to another antibody class or subclass. In some embodiments, an antigen-binding domain comprises a heavy chain variable region (VH) and a light chain variable region (VL), wherein the VH comprises the CDR-H1, the CDR-H2, and the CDR-H3 as comprised within any of the VH regions provided herein, and the VL comprises the CDR-L1, the CDR-L2, and the CDR-L3 comprised within any of the VL regions provided herein.

Also provided herein are fragments of such antibodies that exhibit the desired biological activity.

### CD38

In several embodiments, an antigen binding protein is directed against CD38 (also known as ADP-ribosyl cyclase 1, cADPr hydrolase 1, Cyclic ADP-ribose hydrolase 1, or T10).

According to one embodiment, the CD38 antigen binding protein is an antigen binding domain of a CAR which comprises a transmembrane domain, a signaling domain, and optionally a co-stimulatory domain as disclosed herein. In several embodiments, the antigen binding protein binds to an epitope of the human CD38, and in particular to an epitope of the extracellular domain of the human CD38.

In several embodiments, the CD38 binding protein comprises an scFv comprising a light chain variable region (vL domain) and heavy chain variable region (vH domain). In several embodiments, the vH domain comprises a complementarity-determining region 1 (CDR-H1), a CDR-H2, and a CDR-H3. In several embodiments, the vL domain comprises a complementarity-determining region 1 (CDR-L1), a CDR-L2, and a CDR-L3. In several embodiments, the anti-CD38 vL domain comprises the sequence of SEQ ID NO: 424, or an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 424. In several embodiments, the anti-CD38 vL domain comprises the sequence of SEQ ID NO: 424. In some embodiments, the vL comprises a CDR-L1, a CDR-L2, and a CDR-L3 as comprised in the sequence set forth in SEQ ID NO:424. In several embodiments, the anti-CD38 vH domain comprises the sequence of SEQ ID NO: 425, or an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 425. In several embodiments, the anti-CD38 vH domain comprises the sequence of SEQ ID NO: 425. In some embodiments, the vH comprises a CDR-H1, a CDR-H2, and a CDR-H3 as comprised in the sequence set forth in SEQ ID NO:425. In several embodiments, the anti-CD38 binding protein is an scFv that comprises the sequence of SEQ ID NO: 433, or an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 433. In several embodiments, the anti-CD38 binding protein is an scFv that comprises the sequence of SEQ ID NO: 433. In several embodiments, the anti-CD38 CAR comprises the sequence of SEQ ID NO: 426, or an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 426. In several embodiments, the anti-CD38 CAR comprises the sequence of SEQ ID NO: 426. In several embodiments, the anti-CD38 binding protein comprises at least one CDR from SEQ ID NO: 427-432 or a CDR having at least about 95%, at least about 96%, at least about 97%, at least about 98%, or at least about 99%, sequence identity, homology and/or functional equivalence to any of SEQ ID NO: 427-432. In several embodiments, the antigen binding protein is affinity matured to enhance binding to CD38. In several embodiments, the nucleotide sequence encoding the antigen binding protein is codon-optimized to enhance expression and/or stability of the protein.

### GPRC5D

In several embodiments, an antigen binding protein is directed against GPRC5D. According to one embodiment, the GPRC5D antigen binding protein is an antigen binding domain of a CAR which comprises a transmembrane domain, a signaling domain, and optionally a co-stimulatory domain as disclosed herein. In several embodiments, the antigen binding protein binds to an epitope of the human GPRC5D. In several embodiments, the GPRC5D antigen binding domain comprises a vL and/or vH. In several embodiments, the GPRC5D antigen binding domain comprises a vL and a vH. In several embodiments, the vH comprises a complementarity-determining region 1 (CDR-H1), a CDR-H2, and a CDR-H3. In several embodiments, the vL comprises a complementarity-determining region 1 (CDR-L1), a CDR-L2, and a CDR-L3. In several embodiments, the GPRC5D antigen binding domain is an scFv comprising the amino acid sequence of any one of SEQ ID NOs: 522-531, or an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to any of SEQ ID NOs: 522-531. In several embodiments, the GPRC5D antigen binding domain is an scFv comprising the amino acid sequence of any one of SEQ ID NOs: 522-531. In several embodiments, the antigen binding protein is affinity matured to enhance binding to GPRC5D. In several embodiments, the nucleotide sequence encoding the antigen binding protein is codon-optimized to enhance expression and/or stability of the protein.

### CD138

In several embodiments, an antigen binding protein is directed against CD138. In several embodiments, the anti-CD138 binding protein comprises a vL and/or vH chain. In several embodiments, the anti-CD138 binding protein comprises a vL and a vH. In several embodiments, the vH domain comprises a complementarity-determining region 1 (CDR-H1), a CDR-H2. and a CDR-H3. In several embodiments, the vL domain comprises a complementarity-determining region 1 (CDR-L1), a CDR-L2, and a CDR-L3. In several embodiments, the vL chain comprises the amino acid sequence of SEQ ID NO: 434, or an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 434. In several embodiments, the vL chain comprises the amino acid sequence of SEQ ID NO: 434. In some embodiments, the VL comprises a CDR-L1, a CDR-L2, and a CDR-L3 as comprised in SEQ ID NO:434. In several embodiments, the vH chain comprises the amino acid sequence of SEQ ID NO: 435, or an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 435. In several embodiments, the vH chain comprises the amino acid sequence of SEQ ID NO: 435. In some embodiments, the VH comprises a CDR-H1, a CDR-H2, and a CDR-H3 as comprised in SEQ ID NO:435. In several embodiments, the anti-CD138 binding protein comprises at least one CDR from SEQ ID NO: 437-442 or a CDR having at least about 95%, at least about 96%, at least about 97%, at least about 98%, or at least about 99%, sequence identity, homology and/or functional equivalence to any of SEQ ID NO: 437-442. In several embodiments, the anti-CD138 binding protein is an scFv comprising the amino acid sequence of SEQ ID NO: 443, or an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 443. In several embodiments, the anti-CD138 binding protein is an scFv comprising the amino acid sequence of SEQ ID NO: 443. In several embodiments, the anti-CD138 binding protein is integrated into a CAR which comprises a transmembrane domain, a signaling domain, and optionally a co-stimulatory domain as disclosed herein comprising the amino acid sequence of SEQ ID NO: 436 or 444, or an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 436 or 444. In several embodiments, the CAR comprises the sequence of SEQ ID NO:436. In several embodiments, the CAR comprises the sequence of SEQ ID NO:444. In several embodiments, the antigen binding protein is affinity matured to enhance binding to CD138. In several embodiments, the nucleotide sequence encoding the antigen binding protein is codon-optimized to enhance expression and/or stability of the protein.

### DLL3

In several embodiments, an antigen binding protein is directed against DLL3. In several embodiments, the anti-DLL3 binding protein is an antigen binding domain of a CAR which comprises a transmembrane domain, a signaling domain, and optionally a co-stimulatory domain as disclosed herein. In several embodiments, the anti-DLL3 binding protein comprises a vL and a vH. In several embodiments, the vH domain comprises a complementarity-determining region 1 (CDR-H1), a CDR-H2, and a CDR-H3. In several embodiments, the vL domain comprises a complementarity-determining region 1 (CDR-L1), a CDR-L2, and a CDR-L3. In several embodiments, the anti-DLL3 antigen binding protein comprises a vH chain comprising the amino acid sequence of any of SEQ ID NOs: 471-482, or a sequence having at least about 95%, at least about 96%, at least about 97%, at least about 98%, or at least about 99%. sequence identity, homology and/or functional equivalence to any of SEQ ID NO: 471-482. In several embodiments, the anti-DLL3 antigen binding protein comprises a vH chain comprising the amino acid sequence of any of SEQ ID NOs: 471-482. In some embodiments, the VH comprises a CDR-H1, a CDR-H2, and a CDR-H3 as comprised in any one of SEQ ID NO:471-482. In several embodiments, the anti-DLL3 antigen binding protein comprises a vL chain comprising the amino acid sequence of any of SEQ ID NOs: 483-494, or a sequence having at least about 95%, at least about 96%, at least about 97%, at least about 98%, or at least about 99%, sequence identity, homology and/or functional equivalence to any of SEQ ID NO: 483-494. In several embodiments, the anti-DLL3 antigen binding protein comprises a vL chain comprising the amino acid sequence of any of SEQ ID NOs: 483-494. In some embodiments, the VL comprises a CDR-L1, a CDR-L2, and a CDR-L3 as comprised in any one of SEQ ID NO:483-494. In several embodiments, the anti-DLL3 binding protein comprises a polypeptide that targets DLL3 and comprises the amino acid sequence of any of SEQ ID NO: 495-496, or a sequence having at least about 95%, at least about 96%, at least about 97%, at least about 98%, or at least about 99%, sequence identity, homology and/or functional equivalence to any of SEQ ID NO: 495-496. In several embodiments, the anti-DLL3 binding protein comprises an scFv comprising the sequence of any of SEQ ID NO: 497-500, or a sequence having at least about 95%, at least about 96%, at least about 97%, at least about 98%, or at least about 99%, sequence identity, homology and/or functional equivalence to any of SEQ ID NO: 497-500. In several embodiments, the anti-DLL3 binding protein comprises an scFv comprising the sequence of any of SEQ ID NO: 497-500In several embodiments, the antigen binding protein is affinity matured to enhance binding to DLL3. In several embodiments, the nucleotide sequence encoding the antigen binding protein is codon-optimized to enhance expression and/or stability of the protein.

### EGFR

In several embodiments, an antigen binding protein is directed against the epidermal growth factor receptor EGFR. In several embodiments, the anti-EGFR binding protein is an antigen binding domain of a CAR which comprises a transmembrane domain, a signaling domain, and optionally a co-stimulatory domain as disclosed herein. In several embodiments, the anti-EGFR binding protein comprises a vH chain comprising the amino acid sequence of any of SEQ ID NO: 507-508, or a sequence having at least about 95%, at least about 96%, at least about 97%, at least about 98%, or at least about 99%, sequence identity, homology and/or functional equivalence to any of SEQ ID NO: 507-508. In several embodiments, the anti-EGFR binding protein comprises a vH chain comprising the amino acid sequence of any of SEQ ID NO: 507-508. In some embodiments, the VH comprises a CDR-H1, a CDR-H2, and a CDR-H3 as comprised in SEQ ID NO:507 or 508. In several embodiments, the anti-EGFR binding protein comprises a vL chain comprising the amino acid sequence of any of SEQ ID NO: 509-510, or a sequence having at least about 95%, at least about 96%, at least about 97%, at least about 98%, or at least about 99%, sequence identity, homology and/or functional equivalence to any of SEQ ID NO: 509-510. In several embodiments, the anti-EGFR binding protein comprises a vL chain comprising the amino acid sequence of any of SEQ ID NO: 509-510. In some embodiments, the VL comprises a CDR-L1, a CDR-L2, and a CDR-L3 as comprised in SEQ ID NO:509 or 510. In several embodiments, the anti-EGFR binding protein is an scFv comprising the amino acid sequence of any of SEQ ID NOs: 511-521, or a sequence having at least about 95%, at least about 96%, at least about 97%, at least about 98%, or at least about 99%, sequence identity, homology and/or functional equivalence to any of SEQ ID NO: 511-521. In several embodiments, the anti-EGFR binding protein is an scFv comprising the amino acid sequence of any of SEQ ID NOs: 511-521. In several embodiments, the anti-EGFR binding protein is incorporated into a CAR having the sequence of any of SEQ ID NOs: 503-506, or a sequence having at least about 95%, at least about 96%, at least about 97%, at least about 98%, or at least about 99%, sequence identity, homology and/or functional equivalence to any of SEQ ID NO: 503-506. In some embodiments, the CAR comprises the sequence of any one of SEQ ID NOS:503-506. In several embodiments, the antigen binding protein is affinity matured to enhance binding to the EGFR. In several embodiments, the nucleotide sequence encoding the antigen binding protein is codon-optimized to enhance expression and/or stability of the protein.

### PSMA

In several embodiments, an antigen binding protein is directed against PSMA. In several embodiments, the anti-PSMA binding protein is an antigen binding domain of a CAR which comprises a transmembrane domain, a signaling domain, and optionally a co-stimulatory domain as disclosed herein. In several embodiments, the anti-PSMA binding protein comprises a vL chain comprising the amino acid sequence of SEQ ID NO: 534, or a sequence having at least about 95%, at least about 96%, at least about 97%, at least about 98%, or at least about 99%, sequence identity, homology and/or functional equivalence to SEQ ID NO: 534. In several embodiments, the anti-PSMA binding protein comprises a vL chain comprising the amino acid sequence of SEQ ID NO: 534. In some embodiments, the VL comprises a CDR-L1, a CDR-L2, and a CDR-L3 as comprised in SEQ ID NO:534. In several embodiments, the anti-PSMA binding protein comprises a vH chain comprising the amino acid sequence of SEQ ID NO: 535, or a sequence having at least about 95%, at least about 96%, at least about 97%, at least about 98%, or at least about 99%, sequence identity, homology and/or functional equivalence to SEQ ID NO: 535. In several embodiments, the anti-PSMA binding protein comprises a vH chain comprising the amino acid sequence of SEQ ID NO: 535. In some embodiments, the VH comprises a CDR-H1, a CDR-H2, and a CDR-H3 as comprised in SEQ ID NO:535. In several embodiments, the anti-PSMA binding protein comprises an scFv comprising the amino acid sequence of SEQ ID NO: 533, or a sequence having at least about 95%, at least about 96%, at least about 97%, at least about 98%, or at least about 99%, sequence identity, homology and/or functional equivalence to SEQ ID NO: 533. In several embodiments, the anti-PSMA binding protein comprises an scFv comprising the amino acid sequence of SEQ ID NO: 533. In several embodiments, the anti-PSMA binding protein comprises an antibody comprising the amino acid sequence of SEQ ID NO: 532, or a sequence having at least about 95%, at least about 96%, at least about 97%, at least about 98%, or at least about 99%, sequence identity, homology and/or functional equivalence to SEQ ID NO: 532. In several embodiments, the anti-PSMA binding protein comprises an antibody comprising the amino acid sequence of SEQ ID NO: 532. In several embodiments, the antigen binding protein is affinity matured to enhance binding to PSMA. In several embodiments, the nucleotide sequence encoding the antigen binding protein is codon-optimized to enhance expression and/or stability of the protein.

### FLT3

In several embodiments, an antigen binding protein is directed against FLT3. In several embodiments, the anti-FLT3 binding protein is an antigen binding domain of a CAR which comprises a transmembrane domain, a signaling domain, and optionally a co-stimulatory domain as disclosed herein. In several embodiments, the anti-FLT3 binding protein comprises one or more CDRs from the vL and/or vH chain selected from SEQ ID NOs: 537-545, or a CDR having at least about 95%, at least about 96%, at least about 97%, at least about 98%, or at least about 99%, sequence identity, homology and/or functional equivalence to any of SEQ ID NO: 537-545. In several embodiments, the anti-FLT3 binding protein comprises a vL chain comprising the amino acid sequence of SEQ ID NO: 546, or a sequence having at least about 95%, at least about 96%, at least about 97%, at least about 98%, or at least about 99%, sequence identity, homology and/or functional equivalence to SEQ ID NO: 546. In several embodiments, the anti-FLT3 binding protein comprises a vL chain comprising the amino acid sequence of SEQ ID NO: 546. In some embodiments, the VL comprises a CDR-L1, a CDR-L2, and a CDR-L3 as comprised in SEQ ID NO:546. In several embodiments, the anti-FLT3 binding protein comprises a vH chain comprising the amino acid sequence of SEQ ID NO: 547, or a sequence having at least about 95%, at least about 96%, at least about 97%, at least about 98%, or at least about 99%, sequence identity, homology and/or functional equivalence to SEQ ID NO: 547. In several embodiments, the anti-FLT3 binding protein comprises a vH chain comprising the amino acid sequence of SEQ ID NO: 547. In some embodiments, the VH comprises a CDR-H1, a CDR-1H2, and a CDR-H3 as comprised in SEQ ID NO:547. In several embodiments, the antigen binding protein is affinity matured to enhance binding to FLT3. In several embodiments, the nucleotide sequence encoding the antigen binding protein is codon-optimized to enhance expression and/or stability of the protein.

### KREMEN2

In several embodiments, an antigen binding protein is directed against KREMEN2. In several embodiments, the anti-KREMEN2 binding protein is an antigen binding domain of a CAR which comprises a transmembrane domain, a signaling domain, and optionally a co-stimulatory domain as disclosed herein. In several embodiments, the anti-KREMEN2 binding protein comprises a vL chain comprising the amino acid sequence of any of SEQ ID NOs: 548-552, or a sequence having at least about 95%, at least about 96%, at least about 97%, at least about 98%, or at least about 99%, sequence identity, homology and/or functional equivalence to any of SEQ ID NOs: 548-552. In several embodiments, the anti-KREMEN2 binding protein comprises a vL chain comprising the amino acid sequence of any of SEQ ID NOs: 548-552. In some embodiments, the VL comprises a CDR-L1, a CDR-L2, and a CDR-L3 as comprised in any one of SEQ ID NO:548-552. In several embodiments, the anti-KREMEN2 binding protein comprises a vH chain comprising the amino acid sequence of any of SEQ ID NOs: 553-556, or a sequence having at least about 95%, at least about 96%, at least about 97%, at least about 98%, or at least about 99%, sequence identity, homology and/or functional equivalence to any of SEQ ID NOs: 553-556. In several embodiments, the anti-KREMEN2 binding protein comprises a vH chain comprising the amino acid sequence of any of SEQ ID NOs: 553-556. In some embodiments, the VH comprises a CDR-H1, a CDR-H2, and a CDR-H3 as comprised in any one of SEQ ID NO:553-556. In several embodiments, the anti-KREMEN2 binding protein is an antibody comprising the amino acid sequence of SEQ ID NO: 557, or a sequence having at least about 95%, at least about 96%, at least about 97%, at least about 98%, or at least about 99%. sequence identity, homology and/or functional equivalence to SEQ ID NO: 557. In several embodiments, the anti-KREMEN2 binding protein is an antibody comprising the amino acid sequence of SEQ ID NO: 557. In several embodiments, the antigen binding protein is affinity matured to enhance binding to KREMEN2. In several embodiments, the nucleotide sequence encoding the antigen binding protein is codon-optimized to enhance expression and/or stability of the protein.

### CD70

In several embodiments, an antigen binding protein is directed against CD70 (also known as CD27 ligand, CD27L, or Tumor necrosis factor ligand superfamily member 7).

According to one embodiment, the CD70 antigen binding protein is an antigen binding domain of a CAR which comprises a transmembrane domain, a signaling domain, and optionally a co-stimulatory domain as disclosed herein. In several embodiments, the antigen binding protein binds to an epitope of the human CD70, and in particular to an epitope of the extracellular domain of the human CD70.

In several embodiments, the CD70 binding protein comprises an scFv comprising a light chain variable region (vL domain) and heavy chain variable region (vH domain). In several embodiments, the vH domain comprises a complementarity-determining region 1 (CDR-H1), a CDR-H2, and a CDR-H3. In several embodiments, the vL domain comprises a complementarity-determining region 1 (CDR-L1), a CDR-L2, and a CDR-L3. In several embodiments, the CDR-L1, CDR-L2, and CDR-L3 comprise the amino acid sequences set forth in SEQ ID NOS: 990, 991, and 992, respectively. In several embodiments, the anti-CD70 vL domain comprises the sequence of SEQ ID NO: 994, or an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 994. In several embodiments, the anti-CD70 vL domain comprises the sequence of SEQ ID NO: 994. In some embodiments, the vL comprises a CDR-L1, a CDR-L2, and a CDR-L3 as comprised in the sequence set forth in SEQ ID NO:994. In some embodiments, the CDR-H1, CDR-H2, and CDR-H3 comprise the amino acid sequences set forth in SEQ ID NOS: 987, 988, and 989, respectively. In several embodiments, the anti-CD70 vH domain comprises the sequence of SEQ ID NO: 993, or an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 993. In several embodiments, the anti-CD70 vH domain comprises the sequence of SEQ ID NO: 993. In some embodiments, the vH comprises a CDR-H1, a CDR-H2, and a CDR-H3 as comprised in the sequence set forth in SEQ ID NO:993. In some embodiments, the vL comprises a CDR-L1, a CDR-L2, and a CDR-L3 as comprised in the sequence set forth in SEQ ID NO:994, and the vH comprises a CDR-H1, a CDR-H2, and a CDR-H3 as comprised in the sequence set forth in SEQ ID NO:993. In several embodiments, the vL comprises the sequence of SEQ ID NO: 994, and the vH comprises the sequence of SEQ ID NO: 993. In several embodiments, the antigen binding protein is affinity matured to enhance binding to CD70. In several embodiments, the nucleotide sequence encoding the antigen binding protein is codon-optimized to enhance expression and/or stability of the protein. Additional anti-CD70 binding moieties are known in the art, such as those disclosed in, for example, PCT Application No. PCT/US2021/036879, the entirety of which is incorporated by reference herein.

### BCMA

In several embodiments, an antigen binding protein is directed against BCMA (also known as B cell maturation antigen, Tumor necrosis factor receptor superfamily member 17, TNFRSF17, and CD269). Thus, in some embodiments, the antigen-binding protein is a BCMA-binding protein.

According to one embodiment, the BCMA antigen binding protein is an antigen binding domain of a CAR which comprises a transmembrane domain, a signaling domain, and optionally a co-stimulatory domain as disclosed herein. In several embodiments, the antigen binding protein binds to an epitope of the human BCMA, and in particular to an epitope of the extracellular domain of the human BCMA.

In several embodiments, the antigen-binding protein comprises a heavy chain variable region (VH domain). In some embodiments, antigen-binding protein comprises a single domain antibody (e.g., a V_{H}H). In some embodiments, the antigen-binding protein comprises a VHH.

In several embodiments, the antigen-binding protein comprises a heavy chain variable region (vH domain) and a light chain variable region (vL domain). In some embodiments, the antigen-binding protein comprises a linker between the vH and vL domains. In several embodiments, the antigen-binding protein comprises an scFv. In several embodiments, the BCMA binding protein comprises an scFv comprising a light chain variable region (vL domain) and heavy chain variable region (vH domain).

In several embodiments, the vH domain comprises a complementarity-determining region 1 (CDR-H1), a CDR-H2, and a CDR-1H3. In several embodiments, the vL domain comprises a complementarity-determining region 1 (CDR-L1), a CDR-L2, and a CDR-L3.

In several embodiments, the vH domain comprises the CDR-H1, CDR-H2, and CDR-H3 set forth in SEQ ID NOS: 1016, 1017, and 1018, respectively. In several embodiments, the vL domain comprises the CDR-L1, CDR-L2, and CDR-L3 set forth in SEQ ID NOS: 1019, 1020, and 1021, respectively. In several embodiments, the vH domain comprises the CDR-H1, CDR-H2, and CDR-H3 set forth in SEQ ID NOS: 1016, 1017, and 1018, respectively; and the vL domain comprises the CDR-L1, CDR-L2, and CDR-L3 set forth in SEQ ID NOS: 1019, 1020, and 1021, respectively. In several embodiments, the vH domain comprises the amino acid sequence set forth in SEQ ID NO:1022. In several embodiments, the vL domain comprises the amino acid sequence set forth in SEQ ID NO:1023. In several embodiments, the vH domain comprises the amino acid sequence set forth in SEQ ID NO:1022, and the vL domain comprises the amino acid sequence set forth in SEQ ID NO:1023. In several embodiments, the scFv comprises the amino acid sequence set forth in SEQ ID NO:1024. In several embodiments, the scFv comprises the amino acid sequence set forth in SEQ ID NO:1025.

In several embodiments, the vH domain comprises the CDR-H1, CDR-H2, and CDR-H3 set forth in SEQ ID NOS: 1026, 1027, and 1028, respectively. In several embodiments, the vL domain comprises the CDR-L1, CDR-L2, and CDR-L3 set forth in SEQ ID NOS: 1029, 1030, and 1031, respectively. In several embodiments, the vH domain comprises the CDR-H1, CDR-H2, and CDR-H3 set forth in SEQ ID NOS: 1026, 1027, and 1028, respectively; and the vL domain comprises the CDR-L1, CDR-L2, and CDR-L3 set forth in SEQ ID NOS: 1029, 1030, and 1031, respectively. In several embodiments, the vH domain comprises the amino acid sequence set forth in SEQ ID NO:1032. In several embodiments, the vL domain comprises the amino acid sequence set forth in SEQ ID NO:1033. In several embodiments, the vH domain comprises the amino acid sequence set forth in SEQ ID NO:1032, and the vL domain comprises the amino acid sequence set forth in SEQ ID NO:1033. In several embodiments, the scFv comprises the amino acid sequence set forth in SEQ ID NO:1034. In several embodiments, the scFv comprises the amino acid sequence set forth in SEQ ID NO:1035.

In several embodiments, the vH domain comprises the CDR-H1, CDR-H2, and CDR-H3 set forth in SEQ ID NOS: 1026, 1027, and 1028, respectively. In several embodiments, the vL domain comprises the CDR-L1, CDR-L2, and CDR-L3 set forth in SEQ ID NOS: 1029, 1036, and 1031, respectively. In several embodiments, the vH domain comprises the CDR-H1, CDR-H2, and CDR-H3 set forth in SEQ ID NOS: 1026, 1027, and 1028, respectively; and the vL domain comprises the CDR-L1, CDR-L2, and CDR-L3 set forth in SEQ ID NOS: 1029, 1036, and 1031, respectively. In several embodiments, the vH domain comprises the amino acid sequence set forth in SEQ ID NO:1032. In several embodiments, the vL domain comprises the amino acid sequence set forth in SEQ ID NO:1037. In several embodiments, the vH domain comprises the amino acid sequence set forth in SEQ ID NO:1032, and the vL domain comprises the amino acid sequence set forth in SEQ ID NO:1037. In several embodiments, the scFv comprises the amino acid sequence set forth in SEQ ID NO:1034. In several embodiments, the scFv comprises the amino acid sequence set forth in SEQ ID NO:1038. In several embodiments, the scFv comprises the amino acid sequence set forth in SEQ ID NO:1039.

In several embodiments, the vH domain comprises the CDR-H1, CDR-H2, and CDR-H3 set forth in SEQ ID NOS: 1040, 1041, and 1042, respectively. In several embodiments, the vL domain comprises the CDR-L1, CDR-L2, and CDR-L3 set forth in SEQ ID NOS: 1043, 1044, and 1045, respectively. In several embodiments, the vH domain comprises the CDR-H1, CDR-H2, and CDR-H3 set forth in SEQ ID NOS: 1040, 1041, and 1042, respectively; and the vL domain comprises the CDR-L1, CDR-L2, and CDR-L3 set forth in SEQ ID NOS: 1043, 1044, and 1045, respectively. In several embodiments, the vH domain comprises the amino acid sequence set forth in SEQ ID NO:1046. In several embodiments, the vL domain comprises the amino acid sequence set forth in SEQ ID NO:1047. In several embodiments, the vH domain comprises the amino acid sequence set forth in SEQ ID NO:1046, and the vL domain comprises the amino acid sequence set forth in SEQ ID NO:1047. In several embodiments, the scFv comprises the amino acid sequence set forth in SEQ ID NO:1048. In several embodiments, the scFv comprises the amino acid sequence set forth in SEQ ID NO:1049.

In several embodiments, the vH domain comprises the CDR-H1, CDR-H2, and CDR-H3 set forth in SEQ ID NOS: 1050, 1051, and 1052, respectively. In several embodiments, the vL domain comprises the CDR-L1, CDR-L2, and CDR-L3 set forth in SEQ ID NOS: 1053, 1054, and 1055, respectively. In several embodiments, the vH domain comprises the CDR-H1, CDR-H2, and CDR-H3 set forth in SEQ ID NOS: 1050, 1051, and 1052, respectively; and the vL domain comprises the CDR-L1, CDR-L2, and CDR-L3 set forth in SEQ ID NOS: 1053, 1054, and 1055, respectively. In several embodiments, the vH domain comprises the amino acid sequence set forth in SEQ ID NO:1056. In several embodiments, the vL domain comprises the amino acid sequence set forth in SEQ ID NO:1057. In several embodiments, the vH domain comprises the amino acid sequence set forth in SEQ ID NO:1056, and the vL domain comprises the amino acid sequence set forth in SEQ ID NO:1057. In several embodiments, the scFv comprises the amino acid sequence set forth in SEQ ID NO:1058.

In several embodiments, the vH domain comprises the CDR-H1, CDR-H2, and CDR-H3 set forth in SEQ ID NOS: 1050, 1059, and 1052, respectively. In several embodiments, the vL domain comprises the CDR-L1, CDR-L2, and CDR-L3 set forth in SEQ ID NOS: 1053, 1054, and 1055, respectively. In several embodiments, the vH domain comprises the CDR-H1, CDR-H2, and CDR-H3 set forth in SEQ ID NOS: 1050, 1059, and 1052, respectively; and the vL domain comprises the CDR-L1, CDR-L2, and CDR-L3 set forth in SEQ ID NOS: 1053, 1054, and 1055, respectively. In several embodiments, the vH domain comprises the amino acid sequence set forth in SEQ ID NO:1060. In several embodiments, the vL domain comprises the amino acid sequence set forth in SEQ ID NO:1057. In several embodiments, the vH domain comprises the amino acid sequence set forth in SEQ ID NO:1060, and the vL domain comprises the amino acid sequence set forth in SEQ ID NO:1057. In several embodiments, the scFv comprises the amino acid sequence set forth in SEQ ID NO:1061.

In several embodiments, the vH domain comprises the CDR-H1, CDR-H2, and CDR-H3 set forth in SEQ ID NOS: 1062, 1063, and 1064, respectively. In several embodiments, the vL domain comprises the CDR-L1, CDR-L2, and CDR-L3 set forth in SEQ ID NOS: 1065, 1066, and 1067, respectively. In several embodiments, the vH domain comprises the CDR-H1, CDR-H2, and CDR-H3 set forth in SEQ ID NOS: 1062, 1063, and 1064, respectively; and the vL domain comprises the CDR-L1, CDR-L2, and CDR-L3 set forth in SEQ ID NOS: 1065, 1066, and 1067, respectively. In several embodiments, the vH domain comprises the amino acid sequence set forth in SEQ ID NO:1068. In several embodiments, the vL domain comprises the amino acid sequence set forth in SEQ ID NO:1059. In several embodiments, the vH domain comprises the amino acid sequence set forth in SEQ ID NO:1068, and the vL domain comprises the amino acid sequence set forth in SEQ ID NO:1069. In several embodiments, the scFv comprises the amino acid sequence set forth in SEQ ID NO:1070.

In several embodiments, the vH domain comprises the CDR-H1, CDR-H2, and CDR-H3 set forth in SEQ ID NOS: 1071, 1072, and 1073, respectively. In several embodiments, the vL domain comprises the CDR-L1, CDR-L2, and CDR-L3 set forth in SEQ ID NOS: 1074, 1020, and 1075, respectively. In several embodiments, the vH domain comprises the CDR-H1, CDR-H2, and CDR-H3 set forth in SEQ ID NOS: 1071, 1072, and 1073, respectively; and the vL domain comprises the CDR-L1, CDR-L2, and CDR-L3 set forth in SEQ ID NOS: 1074, 1020, and 1075, respectively. In several embodiments, the vH domain comprises the amino acid sequence set forth in SEQ ID NO:1076. In several embodiments, the vL domain comprises the amino acid sequence set forth in SEQ ID NO:1077. In several embodiments, the vH domain comprises the amino acid sequence set forth in SEQ ID NO:1076, and the vL domain comprises the amino acid sequence set forth in SEQ ID NO:1077. In several embodiments, the scFv comprises the amino acid sequence set forth in SEQ ID NO:1078.

In several embodiments, the vH domain comprises the CDR-H1, CDR-H2, and CDR-H3 set forth in SEQ ID NOS: 1079, 1080, and 1081, respectively. In several embodiments, the vL domain comprises the CDR-L1, CDR-L2, and CDR-L3 set forth in SEQ ID NOS: 1082, 1044, and 1083, respectively. In several embodiments, the vH domain comprises the CDR-H1, CDR-H2, and CDR-H3 set forth in SEQ ID NOS: 1079, 1080, and 1081, respectively; and the vL domain comprises the CDR-L1, CDR-L2, and CDR-L3 set forth in SEQ ID NOS: 1082, 1044, and 1083, respectively. In several embodiments, the vH domain comprises the amino acid sequence set forth in SEQ ID NO:1084. In several embodiments, the vL domain comprises the amino acid sequence set forth in SEQ ID NO:1085. In several embodiments, the vH domain comprises the amino acid sequence set forth in SEQ ID NO:1084, and the vL domain comprises the amino acid sequence set forth in SEQ ID NO:1085. In several embodiments, the scFv comprises the amino acid sequence set forth in SEQ ID NO:1086.

In several embodiments, the antigen binding protein is affinity matured to enhance binding to BCMA. In several embodiments, the nucleotide sequence encoding the antigen binding protein is codon-optimized to enhance expression and/or stability of the protein. Anti-BCMA binding moieties are known in the art, such as those disclosed in, for example, PCT Application No. PCT/US2022/073567, the entirety of which is incorporated by reference herein.

### CD19

In some embodiments, the antigen-binding protein binds to CD19. In some embodiments, an antigen-binding protein is provided comprising a heavy chain variable domain having at least 90% identity to the VH domain amino acid sequence set forth in SEQ ID NO: 33. In some embodiments, the antigen-binding protein comprises a heavy chain variable domain having at least 95% identity to the VH domain amino acid sequence set forth in SEQ ID NO: 33. In some embodiments, the antigen-binding protein comprises a heavy chain variable domain having at least 96, 97, 98, or 99% identity to the VH domain amino acid sequence set forth in SEQ ID NO: 33. In several embodiments, the heavy chain variable domain may have one or more additional mutations (e.g., for purposes of humanization) in the VH domain amino acid sequence set forth in SEQ ID NO: 33, but retains specific binding to a cancer antigen (e.g., CD19). In several embodiments, the heavy chain variable domain may have one or more additional mutations in the VH domain amino acid sequence set forth in SEQ ID NO: 33, but has improved specific binding to a cancer antigen (e.g., CD19). In some embodiments, the VH comprises the amino acid sequence set forth in SEQ ID NO:33. In some embodiments, the VH comprises a CDR-H1, a CDR-H2, and a CDR-H3 as comprised in SEQ ID NO:33. In some embodiments, the VH comprises a CDR-H1, a CDR-H2, and a CDR-H3 comprising the amino acid sequences set forth in SEQ ID NOS: 981, 982, and 983, respectively.

In some embodiments, the antigen-binding protein comprises a light chain variable domain having at least 90% identity to the VL domain amino acid sequence set forth in SEQ ID NO: 32. In some embodiments, the antigen-binding protein comprises a light chain variable domain having at least 95% identity to the VL domain amino acid sequence set forth in SEQ ID NO: 32. In some embodiments, the antigen-binding protein comprises a light chain variable domain having at least 96, 97, 98, or 99% identity to the VL domain amino acid sequence set forth in SEQ ID NO: 32. In several embodiments, the light chain variable domain may have one or more additional mutations (e.g., for purposes of humanization) in the VL domain amino acid sequence set forth in SEQ ID NO: 32, but retains specific binding to a cancer antigen (e.g., CD19). In several embodiments, the light chain variable domain may have one or more additional mutations in the VL domain amino acid sequence set forth in SEQ ID NO: 32, but has improved specific binding to a cancer antigen (e.g., CD19). In some embodiments, the VL comprises the amino acid sequence set forth in SEQ ID NO:32. In some embodiments, the VL comprises a CDR-L1, a CDR-L2, and a CDR-L3 as comprised in SEQ ID NO:32. In some embodiments, the VL comprises a CDR-L1, a CDR-L2, and a CDR-L3 comprising the amino acid sequences set forth in SEQ ID NOS: 984, 985, and 39, respectively.

In some embodiments, the antigen-binding protein comprises a heavy chain variable domain having at least 90% identity to the VH domain amino acid sequence set forth in SEQ ID NO: 33, and a light chain variable domain having at least 90% identity to the VL domain amino acid sequence set forth in SEQ ID NO: 32. In some embodiments, the antigen-binding protein comprises a heavy chain variable domain having at least 95% identity to the VH domain amino acid sequence set forth in SEQ ID NO: 33, and a light chain variable domain having at least 95% identity to the VL domain amino acid sequence set forth in SEQ ID NO: 32. In some embodiments, the antigen-binding protein comprises a heavy chain variable domain having at least 96, 97, 98, or 99% identity to the VH domain amino acid sequence set forth in SEQ ID NO: 33, and a light chain variable domain having at least 96, 97, 98, or 99% identity to the VL domain amino acid sequence set forth in SEQ ID NO: 32.

In some embodiments, the antigen-binding protein comprises a heavy chain variable domain having the VH domain amino acid sequence set forth in SEQ ID NO: 33, and a light chain variable domain having the VL domain amino acid sequence set forth in SEQ ID NO: 32. In some embodiments, the light-chain variable domain comprises a sequence of amino acids that is at least 70%, 75%, 80%, 85%, 90%, 91%, 92%, 93%, 94%, 95%, 96%, 97%, 98%, 99%, or more, identical to the sequence of a light chain variable domain of SEQ ID NO: 32. In some embodiments, the light-chain variable domain comprises a sequence of amino acids that is at least 70%, 75%, 80%, 85%, 90%, 91%, 92%, 93%, 94%, 95%, 96%, 97%, 98%, 99%, or more, identical to the sequence of a heavy chain variable domain in accordance with SEQ ID NO: 33.

In some embodiments, the light chain variable domain comprises a sequence of amino acids that is encoded by a nucleotide sequence that is at least 70%, 75%, 80%, 85%, 90%, 91%, 92%, 93%, 94%, 95%, 96%, 97%, 98%, 99%, or more, identical to the polynucleotide sequence SEQ ID NO: 32. In some embodiments, the light chain variable domain comprises a sequence of amino acids that is encoded by a polynucleotide that hybridizes under moderately stringent conditions to the complement of a polynucleotide that encodes a light chain variable domain in accordance with the sequence in SEQ ID NO: 32. In some embodiments, the light chain variable domain comprises a sequence of amino acids that is encoded by a polynucleotide that hybridizes under stringent conditions to the complement of a polynucleotide that encodes a light chain variable domain in accordance with the sequence in SEQ ID NO: 32.

In some embodiments, the heavy chain variable domain comprises a sequence of amino acids that is at least 70%, 75%, 80%, 85%, 90%, 91%, 92%, 93%, 94%, 95%, 96%, 97%, 98%, 99%, or more, identical to the sequence of a heavy chain variable domain in accordance with the sequence of SEQ ID NO: 33. In some embodiments, the heavy chain variable domain comprises a sequence of amino acids that is encoded by a polynucleotide that hybridizes under moderately stringent conditions to the complement of a polynucleotide that encodes a heavy chain variable domain in accordance with the sequence of SEQ ID NO: 33. In some embodiments, the heavy chain variable domain comprises a sequence of amino acids that is encoded by a polynucleotide that hybridizes under stringent conditions to the complement of a polynucleotide that encodes a heavy chain variable domain in accordance with the sequence of SEQ ID NO: 33.

In some embodiments, the anti-CD19 binding protein is an scFv comprising a VH and a VL. In several embodiments, additional anti-CD19 binding constructs are provided. For example, in several embodiments, there is provided an scFv that targets CD19 wherein the scFv comprises a heavy chain variable region comprising the sequence of SEQ ID NO. 35. In some embodiments, the antigen-binding protein comprises a heavy chain variable domain having at least 95% identity to the HCV domain amino acid sequence set forth in SEQ ID NO: 35. In some embodiments, the antigen-binding protein comprises a heavy chain variable domain having at least 96, 97, 98, or 99% identity to the HCV domain amino acid sequence set forth in SEQ ID NO: 35. In several embodiments, the heavy chain variable domain may have one or more additional mutations (e.g., for purposes of humanization) in the HCV domain amino acid sequence set forth in SEQ ID NO: 35, but retains specific binding to a cancer antigen (e.g., CD19). In several embodiments, the heavy chain variable domain may have one or more additional mutations in the HCV domain amino acid sequence set forth in SEQ ID NO: 35, but has improved specific binding to a cancer antigen (e.g., CD19). In some embodiments, the scFv comprises a VH comprising the amino acid sequence of SEQ ID NO:35 and a VL comprising the amino acid sequence of SEQ ID NO:36.

Additionally, in several embodiments, an scFv that targets CD19 comprises a light chain variable region comprising the sequence of SEQ ID NO. 36. In some embodiments, the antigen-binding protein comprises a light chain variable domain having at least 95% identity to the LCV domain amino acid sequence set forth in SEQ ID NO: 36. In some embodiments, the antigen-binding protein comprises a light chain variable domain having at least 96, 97, 98, or 99% identity to the LCV domain amino acid sequence set forth in SEQ ID NO: 36. In several embodiments, the light chain variable domain may have one or more additional mutations (e.g., for purposes of humanization) in the LCV domain amino acid sequence set forth in SEQ ID NO: 36, but retains specific binding to a cancer antigen (e.g., CD19). In several embodiments, the light chain variable domain may have one or more additional mutations in the LCV domain amino acid sequence set forth in SEQ ID NO: 36, but has improved specific binding to a cancer antigen (e.g., CD19). In some embodiments, the CD19-directed CAR comprises the amino acid sequence set forth in SEQ ID NO:986.

In several embodiments, there is also provided an anti-CD19 binding moiety that comprises a light chain CDR comprising a first, second and third complementarity determining region (LC CDR1, LC CDR2, and LC CDR3, respectively. In several embodiments, the anti-CD19 binding moiety further comprises a heavy chain CDR comprising a first, second and third complementarity determining region (HC CDR1, HC CDR2, and HC CDR3, respectively. In several embodiments, the LC CDR1 comprises the sequence of SEQ ID NO. 37. In several embodiments, the LC CDR1 comprises an amino acid sequence with at least about 85%, about 90%, about 95%, or about 98% sequence identity to the sequence of SEQ NO. 37. In several embodiments, the LC CDR2 comprises the sequence of SEQ ID NO. 38. In several embodiments, the LC CDR2 comprises an amino acid sequence with at least about 85%, about 90%, about 95%, or about 98% sequence identity to the sequence of SEQ NO. 38. In several embodiments, the LC CDR3 comprises the sequence of SEQ ID NO. 39. In several embodiments, the LC CDR3 comprises an amino acid sequence with at least about 85%, about 90%, about 95%, or about 98% sequence identity to the sequence of SEQ NO. 39. In several embodiments, the HC CDR1 comprises the sequence of SEQ ID NO. 40. In several embodiments, the HC CDR1 comprises an amino acid sequence with at least about 85%, about 90%, about 95%, or about 98% sequence identity to the sequence of SEQ NO. 40. In several embodiments, the HC CDR2 comprises the sequence of SEQ ID NO. 41, 42, or 43. In several embodiments, the HC CDR2 comprises an amino acid sequence with at least about 85%, about 90%, about 95%, or about 98% sequence identity to the sequence of SEQ NO. 41, 42, or 43. In several embodiments, the HC CDR3 comprises the sequence of SEQ ID NO. 44. In several embodiments, the HC CDR3 comprises an amino acid sequence with at least about 85%, about 90%, about 95%, or about 98% sequence identity to the sequence of SEQ NO. 44.

In several embodiments, there is also provided an anti-CD19 binding moiety that comprises a light chain variable region (VL) and a heavy chain variable region (HL), the VL region comprising a first, second and third complementarity determining region (VL CDR1, VL CDR2, and VL CDR3, respectively and the VH region comprising a first, second and third complementarity determining region (VH CDR1, VH CDR2, and VH CDR3, respectively. In several embodiments, the VL region comprises the sequence of SEQ ID NO. 45, 46, 47, or 48. In several embodiments, the VL region comprises an amino acid sequence with at least about 85%, about 90%, about 95%, or about 98% sequence identity to the sequence of SEQ NO. 45, 46, 47, or 48. In several embodiments, the VH region comprises the sequence of SEQ ID NO. 49, 50, 51 or 52. In several embodiments, the VH region comprises an amino acid sequence with at least about 85%, about 90%, about 95%, or about 98% sequence identity to the sequence of SEQ NO. 49, 50, 51 or 52.

In several embodiments, there is also provided an anti-CD19 binding moiety that comprises a light chain CDR comprising a first, second and third complementarity determining region (LC CDR1, LC CDR2, and LC CDR3, respectively. In several embodiments, the anti-CD19 binding moiety further comprises a heavy chain CDR comprising a first, second and third complementarity determining region (HC CDR1, HC CDR2, and HC CDR3, respectively. In several embodiments, the LC CDR1 comprises the sequence of SEQ ID NO. 53. In several embodiments, the LC CDR1 comprises an amino acid sequence with at least about 85%, about 90%, about 95%, or about 98% sequence identity to the sequence of SEQ NO. 53. In several embodiments, the LC CDR2 comprises the sequence of SEQ ID NO. 54. In several embodiments, the LC CDR2 comprises an amino acid sequence with at least about 85%, about 90%, about 95%, or about 98% sequence identity to the sequence of SEQ NO. 54. In several embodiments, the LC CDR3 comprises the sequence of SEQ ID NO. 55. In several embodiments, the LC CDR3 comprises an amino acid sequence with at least about 85%, about 90%, about 95%, or about 98% sequence identity to the sequence of SEQ NO. 55. In several embodiments, the HC CDR1 comprises the sequence of SEQ ID NO. 56. In several embodiments, the HC CDR1 comprises an amino acid sequence with at least about 85%, about 90%, about 95%, or about 98% sequence identity to the sequence of SEQ NO. 56. In several embodiments, the HC CDR2 comprises the sequence of SEQ ID NO. 57. In several embodiments, the HC CDR2 comprises an amino acid sequence with at least about 85%, about 90%, about 95%, or about 98% sequence identity to the sequence of SEQ NO. 57. In several embodiments, the HC CDR3 comprises the sequence of SEQ ID NO. 58. In several embodiments, the HC CDR3 comprises an amino acid sequence with at least about 85%, about 90%, about 95%, or about 98% sequence identity to the sequence of SEQ NO. 58.

In some embodiments, the antigen-binding protein comprises a heavy chain variable region comprising the amino acid sequence of SEQ ID NO: 104. In some embodiments, the antigen-binding protein comprises a heavy chain variable region having at least 90% identity to the VH domain amino acid sequence set forth in SEQ ID NO: 104. In some embodiments, the antigen-binding protein comprises a heavy chain variable domain having at least 95% sequence identity to the VH domain amino acid sequence set forth in SEQ ID NO: 104. In some embodiments, the antigen-binding protein comprises a heavy chain variable domain having at least 96, 97, 98, or 99% sequence identity to the VH domain amino acid sequence set forth in SEQ ID NO: 104. In several embodiments, the heavy chain variable domain may have one or more additional mutations (e.g., for purposes of humanization) in the VH domain amino acid sequence set forth in SEQ ID NO: 104, but retains specific binding to a cancer antigen (e.g., CD19). In several embodiments, the heavy chain variable domain may have one or more additional mutations in the VH domain amino acid sequence set forth in SEQ ID NO: 104, but has improved specific binding to a cancer antigen (e.g., CD19).

In some embodiments, the antigen-binding protein comprises a light chain variable region comprising the amino acid sequence of SEQ ID NO: 105. In some embodiments, the antigen-binding protein comprises a light chain variable region having at least 90% sequence identity to the VL domain amino acid sequence set forth in SEQ ID NO: 105. In some embodiments, the antigen-binding protein comprises a light chain variable domain having at least 95% sequence identity to the VL domain amino acid sequence set forth in SEQ ID NO: 105. In some embodiments, the antigen-binding protein comprises a light chain variable domain having at least 96, 97, 98, or 99% sequence identity to the VL domain amino acid sequence set forth in SEQ ID NO: 105. In several embodiments, the light chain variable domain may have one or more additional mutations (e.g., for purposes of humanization) in the VL domain amino acid sequence set forth in SEQ ID NO: 105, but retains specific binding to a cancer antigen (e.g., CD19). In several embodiments, the light chain variable domain may have one or more additional mutations in the VL domain amino acid sequence set forth in SEQ ID NO: 105, but has improved specific binding to a cancer antigen (e.g., CD19).

In some embodiments, the antigen-binding protein comprises a heavy chain variable domain having the VH domain amino acid sequence set forth in SEQ ID NO: 104, and a light chain variable domain having the VL domain amino acid sequence set forth in SEQ ID NO: 105. In some embodiments, the light-chain variable domain comprises a sequence of amino acids that is at least 70%, 75%, 80%, 85%, 90%, 91%, 92%, 93%, 94%, 95%, 96%, 97%, 98%, 99%, or more, identical to the sequence of a light chain variable domain of SEQ ID NO: 105. In some embodiments, the heavy-chain variable domain comprises a sequence of amino acids that is at least 70%, 75%, 80%, 85%, 90%, 91%, 92%, 93%, 94%, 95%, 96%, 97%, 98%, 99%, or more, identical to the sequence of a heavy chain variable domain in accordance with SEQ ID NO: 104.

In some embodiments, the antigen-binding protein comprises a heavy chain variable comprising the amino acid sequence of SEQ ID NO: 106. In some embodiments, the antigen-binding protein comprises a heavy chain variable having at least 90% sequence identity to the VH amino acid sequence set forth in SEQ ID NO: 106. In some embodiments, the antigen-binding protein comprises a heavy chain variable having at least 95% sequence identity to the VH amino acid sequence set forth in SEQ ID NO: 106. In some embodiments, the antigen-binding protein comprises a heavy chain variable having at least 96, 97, 98, or 99% identity to the VH amino acid sequence set forth in SEQ ID NO: 106. In several embodiments, the heavy chain variable may have one or more additional mutations (e.g., for purposes of humanization) in the VH amino acid sequence set forth in SEQ ID NO: 106, but retains specific binding to a cancer antigen (e.g., CD19). In several embodiments, the heavy chain variable may have one or more additional mutations in the VH amino acid sequence set forth in SEQ ID NO: 106, but has improved specific binding to a cancer antigen (e.g., CD19).

In some embodiments, the antigen-binding protein comprises a light chain variable comprising the amino acid sequence of SEQ ID NO: 107. In some embodiments, the antigen-binding protein comprises a light chain variable region having at least 90% sequence identity to the VL amino acid sequence set forth in SEQ ID NO: 107. In some embodiments, the antigen-binding protein comprises a light chain variable having at least 95% sequence identity to the VL amino acid sequence set forth in SEQ ID NO: 107. In some embodiments, the antigen-binding protein comprises a light chain variable having at least 96, 97, 98, or 99% identity to the VL amino acid sequence set forth in SEQ ID NO: 107. In several embodiments, the light chain variable may have one or more additional mutations (e.g., for purposes of humanization) in the VL amino acid sequence set forth in SEQ ID NO: 107, but retains specific binding to a cancer antigen (e.g., CD19). In several embodiments, the light chain variable may have one or more additional mutations in the VL amino acid sequence set forth in SEQ ID NO: 107, but has improved specific binding to a cancer antigen (e.g., CD19).

In several embodiments, there is also provided an anti-CD19 binding moiety that comprises a light chain CDR comprising a first, second and third complementarity determining region (LC CDR1, LC CDR2, and LC CDR3, respectively. In several embodiments, the anti-CD19 binding moiety further comprises a heavy chain CDR comprising a first, second and third complementarity determining region (HC CDR1, HC CDR2, and HC CDR3, respectively. In several embodiments, the LC CDR1 comprises the sequence of SEQ ID NO. 108. In several embodiments, the LC CDR1 comprises an amino acid sequence with at least about 85%, about 90%, about 95%, or about 98% sequence identity to the sequence of SEQ NO. 108. In several embodiments, the LC CDR2 comprises the sequence of SEQ ID NO. 109. In several embodiments, the LC CDR2 comprises an amino acid sequence with at least about 85%, about 90%, about 95%, or about 98% sequence identity to the sequence of SEQ NO. 109. In several embodiments, the LC CDR3 comprises the sequence of SEQ ID NO. 110. In several embodiments, the LC CDR3 comprises an amino acid sequence with at least about 85%, about 90%, about 95%, or about 98% sequence identity to the sequence of SEQ NO. 110. In several embodiments, the HC CDR1 comprises the sequence of SEQ ID NO. 111. In several embodiments, the HC CDR1 comprises an amino acid sequence with at least about 85%, about 90%, about 95%, or about 98% sequence identity to the sequence of SEQ NO. 111. In several embodiments, the HC CDR2 comprises the sequence of SEQ ID NO. 112, 113, or 114. In several embodiments, the HC CDR2 comprises an amino acid sequence with at least about 85%, about 90%, about 95%, or about 98% sequence identity to the sequence of SEQ NO. 112, 113, or 114. In several embodiments, the HC CDR3 comprises the sequence of SEQ ID NO. 115. In several embodiments, the HC CDR3 comprises an amino acid sequence with at least about 85%, about 90%, about 95%, or about 98% sequence identity to the sequence of SEQ NO. 115. In several embodiments, the anti-CD19 binding moiety comprises SEQ ID NO: 116, or is sequence with at least about 85%, about 90%, about 95%, or about 98% sequence identity to the sequence of SEQ NO. 116.

In some embodiments, the antigen-binding protein comprises a light chain variable comprising the amino acid sequence of SEQ ID NO: 117, 118, or 119. In some embodiments, the antigen-binding protein comprises a light chain variable region having at least 90% identity to the VL amino acid sequence set forth in SEQ ID NO: 117, 118, or 119. In some embodiments, the antigen-binding protein comprises a light chain variable having at least 95% identity to the VL amino acid sequence set forth in SEQ ID NO: 117, 118, or 119. In some embodiments, the antigen-binding protein comprises a light chain variable having at least 96, 97, 98, or 99% identity to the VL amino acid sequence set forth in SEQ ID NO: 117, 118, or 119. In several embodiments, the light chain variable may have one or more additional mutations (e.g., for purposes of humanization) in the VL amino acid sequence set forth in SEQ ID NO: 117, 118, or 119, but retains specific binding to a cancer antigen (e.g., CD19). In several embodiments, the light chain variable may have one or more additional mutations in the VL amino acid sequence set forth in SEQ ID NO: 117, 118, or 119, but has improved specific binding to a cancer antigen (e.g., CD19).

In some embodiments, the antigen-binding protein comprises a heavy chain variable comprising the amino acid sequence of SEQ ID NO: 120,121, 122, or 123. In some embodiments, the antigen-binding protein comprises a heavy chain variable having at least 90% identity to the VH amino acid sequence set forth in SEQ ID NO: 120,121, 122, or 123. In some embodiments, the antigen-binding protein comprises a heavy chain variable having at least 95% identity to the VH amino acid sequence set forth in SEQ ID NO: 120,121, 122, or 123. In some embodiments, the antigen-binding protein comprises a heavy chain variable having at least 96, 97, 98, or 99% identity to the VH amino acid sequence set forth in SEQ ID NO: 120,121, 122, or 123. In several embodiments, the heavy chain variable may have one or more additional mutations (e.g., for purposes of humanization) in the VH amino acid sequence set forth in SEQ ID NO: 120,121, 122, or 123, but retains specific binding to a cancer antigen (e.g., CD19). In several embodiments, the heavy chain variable may have one or more additional mutations in the VH amino acid sequence set forth in SEQ ID NO: 120,121, 122, or 123, but has improved specific binding to a cancer antigen (e.g., CD19).

In several embodiments, there is also provided an anti-CD19 binding moiety that comprises a light chain CDR comprising a first, second and third complementarity determining region (LC CDR1, LC CDR2, and LC CDR3, respectively. In several embodiments, the anti-CD19 binding moiety further comprises a heavy chain CDR comprising a first, second and third complementarity determining region (HC CDR1, HC CDR2, and HC CDR3, respectively. In several embodiments, the LC CDR1 comprises the sequence of SEQ ID NO. 124, 127, or 130. In several embodiments, the LC CDR1 comprises an amino acid sequence with at least about 85%, about 90%, about 95%, or about 98% sequence identity to the sequence of SEQ NO. 124, 127, or 130. In several embodiments, the LC CDR2 comprises the sequence of SEQ ID NO. 125, 128, or 131. In several embodiments, the LC CDR2 comprises an amino acid sequence with at least about 85%, about 90%, about 95%, or about 98% sequence identity to the sequence of SEQ NO. 125, 128, or 131. In several embodiments, the LC CDR3 comprises the sequence of SEQ ID NO. 126, 129, or 132. In several embodiments, the LC CDR3 comprises an amino acid sequence with at least about 85%, about 90%, about 95%, or about 98% sequence identity to the sequence of SEQ NO. 126, 129, or 132. In several embodiments, the HC CDR1 comprises the sequence of SEQ ID NO. 133, 136, 139, or 142. In several embodiments, the HC CDR1 comprises an amino acid sequence with at least about 85%, about 90%, about 95%, or about 98% sequence identity to the sequence of SEQ NO. 133, 136, 139, or 142. In several embodiments, the HC CDR2 comprises the sequence of SEQ ID NO. 134, 137, 140, or 143. In several embodiments, the HC CDR2 comprises an amino acid sequence with at least about 85%, about 90%, about 95%, or about 98% sequence identity to the sequence of SEQ NO. 134, 137, 140, or 143. In several embodiments, the HC CDR3 comprises the sequence of SEQ ID NO. 135, 138, 141, or 144. In several embodiments, the HC CDR3 comprises an amino acid sequence with at least about 85%, about 90%, about 95%, or about 98% sequence identity to the sequence of SEQ NO. 135, 138, 141, or 144.

Additional anti-CD19 binding moieties are known in the art, such as those disclosed in, for example, U.S. Pat. No. 8,399,645, US Patent Publication No. 2018/0153977, US Patent Publication No. 2014/0271635, US Patent Publication No. 2018/0251514, US Patent Publication No. 2018/0312588, and PCT Publication No. WO 2020/180882, the entirety of each of which is incorporated by reference herein.

### CLDN6

Several embodiments relate to CARs that are directed to Claudin 6, and show little or no binding to Claudin 3, 4, or 9 (or other Claudins). In some embodiments, the antigen-binding protein binds to Claudin 6 (CLDN6). In some embodiments, the antigen-binding protein comprises a heavy chain variable (VH) comprising the amino acid sequence of SEQ ID NO: 88. In some embodiments, the VH comprises a CDR-H1, a CDR-H2, and a CDR-H3 as comprised in SEQ ID NO:88. In some embodiments, the antigen-binding protein comprises a heavy chain variable having at least 90% identity to the VH amino acid sequence set forth in SEQ ID NO: 88. In some embodiments, the antigen-binding protein comprises a heavy chain variable having at least 95% identity to the VH amino acid sequence set forth in SEQ ID NO: 88. In some embodiments, the antigen-binding protein comprises a heavy chain variable having at least 96, 97, 98, or 99% identity to the VH amino acid sequence set forth in SEQ ID NO: 88. In several embodiments, the heavy chain variable may have one or more additional mutations (e.g., for purposes of humanization) in the VH amino acid sequence set forth in SEQ ID NO: 88, but retains specific binding to a cancer antigen (e.g., CLDN6). In several embodiments, the heavy chain variable may have one or more additional mutations in the VH amino acid sequence set forth in SEQ ID NO: 88, but has improved specific binding to a cancer antigen (e.g., CLDN6).

In some embodiments, the antigen-binding protein comprises a light chain variable (VL) comprising the amino acid sequence of SEQ ID NO: 89, 90 or 91. In some embodiments, the VL comprises a CDR-L1, a CDR-L2, and a CDR-L3 as comprised in any one of SEQ ID NO: 89-91. In some embodiments, the antigen-binding protein comprises a light chain variable region having at least 90% identity to the VL amino acid sequence set forth in SEQ ID NO: 89, 90 or 91. In some embodiments, the antigen-binding protein comprises a light chain variable having at least 95% identity to the VL amino acid sequence set forth in SEQ ID NO: 89, 90 or 91. In some embodiments, the antigen-binding protein comprises a light chain variable having at least 96, 97, 98, or 99% identity to the VL amino acid sequence set forth in SEQ ID NO: 89, 90 or 91. In several embodiments, the light chain variable may have one or more additional mutations (e.g., for purposes of humanization) in the VL amino acid sequence set forth in SEQ ID NO: 89, 90 or 91, but retains specific binding to a cancer antigen (e.g., CLDN6). In several embodiments, the light chain variable may have one or more additional mutations in the VL amino acid sequence set forth in SEQ ID NO: 89, 90 or 91, but has improved specific binding to a cancer antigen (e.g., CLDN6).

In several embodiments, there is also provided an anti-CLDN6 binding moiety that comprises a light chain CDR comprising a first, second and third complementarity determining region (LC CDR1, LC CDR2, and LC CDR3, respectively. In several embodiments, the anti-CD19 binding moiety further comprises a heavy chain CDR comprising a first, second and third complementarity determining region (HC CDR1, HC CDR2, and HC CDR3, respectively. In several embodiments, the LC CDR1 comprises the sequence of SEQ ID NO. 95, 98, or 101. In several embodiments, the LC CDR1 comprises an amino acid sequence with at least about 85%, about 90%, about 95%, or about 98% sequence identity to the sequence of SEQ NO. 95, 98, or 101. In several embodiments, the LC CDR2 comprises the sequence of SEQ ID NO. 96, 99, or 102. In several embodiments, the LC CDR2 comprises an amino acid sequence with at least about 85%, about 90%, about 95%, or about 98% sequence identity to the sequence of SEQ NO. 96, 99, or 102. In several embodiments, the LC CDR3 comprises the sequence of SEQ ID NO. 97, 100, or 103. In several embodiments, the LC CDR3 comprises an amino acid sequence with at least about 85%, about 90%, about 95%, or about 98% sequence identity to the sequence of SEQ NO. 97, 100, or 103. In several embodiments, the HC CDR1 comprises the sequence of SEQ ID NO. 92. In several embodiments, the HC CDR1 comprises an amino acid sequence with at least about 85%, about 90%, about 95%, or about 98% sequence identity to the sequence of SEQ NO. 92. In several embodiments, the HC CDR2 comprises the sequence of SEQ ID NO. 93. In several embodiments, the HC CDR2 comprises an amino acid sequence with at least about 85%, about 90%, about 95%, or about 98% sequence identity to the sequence of SEQ NO. 93. In several embodiments, the HC CDR3 comprises the sequence of SEQ ID NO. 94. In several embodiments, the HC CDR3 comprises an amino acid sequence with at least about 85%, about 90%, about 95%, or about 98% sequence identity to the sequence of SEQ NO. 94. In several embodiments, the antigen-binding protein does not bind claudins other than CLDN6

Natural Killer Group Domains that Bind Tumor Ligands

In several embodiments, engineered immune cells such as NK cells are leveraged for their ability to recognize and destroy tumor cells. For example, an engineered NK cell may include a CD19-directed chimeric antigen receptor or a nucleic acid encoding said chimeric antigen receptor (or a CAR directed against, for example, one or more of CD123, CD70, Her2, mesothelin, Claudin 6, BCMA, EGFR, etc.). NK cells express both inhibitory and activating receptors on the cell surface. Inhibitory receptors bind self-molecules expressed on the surface of healthy cells (thus preventing immune responses against “self” cells), while the activating receptors bind ligands expressed on abnormal cells, such as tumor cells. When the balance between inhibitory and activating receptor activation is in favor of activating receptors, NK cell activation occurs and target (e.g., tumor) cells are lysed.

Natural killer Group 2 member D (NKG2D) is an NK cell activating receptor that recognizes a variety of ligands expressed on cells. The surface expression of various NKG2D ligands is generally low in healthy cells but is upregulated upon, for example, malignant transformation. Non-limiting examples of ligands recognized by NKG2D include, but are not limited to, MICA, MICB, ULBP1. ULBP2, ULBP3, ULBP4, ULBP5, and ULBP6, as well as other molecules expressed on target cells that control the cytolytic or cytotoxic function of NK cells. In several embodiments, T cells are engineered to express an extracellular domain to binds to one or more tumor ligands and activate the T cell. For example, in several embodiments, T cells are engineered to express an NKG2D receptor as the binder/activation moiety. In several embodiments, engineered cells as disclosed herein are engineered to express another member of the NKG2 family, e.g., NKG2A, NKG2C, and/or NKG2E. Combinations of such receptors are engineered in some embodiments. Moreover, in several embodiments, other receptors are expressed, such as the Killer-cell immunoglobulin-like receptors (KIRs).

In several embodiments, cells are engineered to express a cytotoxic receptor complex comprising a full length NKG2D as an extracellular component to recognize ligands on the surface of tumor cells (e.g., liver cells). In one embodiment, full length NKG2D has the nucleic acid sequence of SEQ ID NO: 27. In several embodiments, the full length NKG2D, or functional fragment thereof is human NKG2D. Additional information about chimeric receptors for use in the presently disclosed methods and compositions can be found in PCT Patent Publication No. WO/2018/183385, which is incorporated in its entirety by reference herein.

In several embodiments, cells are engineered to express a cytotoxic receptor complex comprising a functional fragment of NKG2D as an extracellular component to recognize ligands on the surface of tumor cells or other diseased cells. In one embodiment, the functional fragment of NKG2D has the nucleic acid sequence of SEQ ID NO: 25. In several embodiments, the fragment of NKG2D is at least 70%, at least 75%, at least 80%, at least 85%, at least 90%, or at least 95% homologous with full-length wild-type NKG2D. In several embodiments, the fragment may have one or more additional mutations from SEQ ID NO: 25, but retains, or in some embodiments, has enhanced, ligand-binding function. In several embodiments, the functional fragment of NKG2D comprises the amino acid sequence of SEQ ID NO: 26. In several embodiments, the NKG2D fragment is provided as a dimer, trimer, or other concatameric format, such embodiments providing enhanced ligand-binding activity. In several embodiments, the sequence encoding the NKG2D fragment is optionally fully or partially codon optimized. In one embodiment, a sequence encoding a codon optimized NKG2D fragment comprises the sequence of SEQ ID NO: 28. Advantageously, according to several embodiments, the functional fragment lacks its native transmembrane or intracellular domains but retains its ability to bind ligands of NKG2D as well as transduce activation signals upon ligand binding. A further advantage of such fragments is that expression of DAP10 to localize NKG2D to the cell membrane is not required. Thus, in several embodiments, the cytotoxic receptor complex encoded by the polypeptides disclosed herein does not comprise DAP10. In several embodiments, a NKG2D-targeting CAR comprises the amino acid sequence set forth in SEQ ID NO: 980. In several embodiments, immune cells, such as NK or T cells (e.g., non-alloreactive T cells engineered according to embodiments disclosed herein), are engineered to express one or more chimeric receptors that target, for example CD19, CD123, CD70, Her2, mesothelin, Claudin 6, BCMA, EGFR, and an NKG2D ligand, such as MICA, MICB, ULBP1, ULBP2, ULBP3, ULBP4, ULBP5, and/or ULBP6. Such cells, in several embodiments, also co-express mbIL15.

In several embodiments, the cytotoxic receptor complexes are configured to dimerize. Dimerization may comprise homodimers or heterodimers, depending on the embodiment. In several embodiments, dimerization results in improved ligand recognition by the cytotoxic receptor complexes (and hence the NK cells expressing the receptor), resulting in a reduction in (or lack) of adverse toxic effects. In several embodiments, the cytotoxic receptor complexes employ internal dimers, or repeats of one or more component subunits. For example, in several embodiments, the cytotoxic receptor complexes may optionally comprise a first NKG2D extracellular domain coupled to a second NKG2D extracellular domain, and a transmembrane/signaling region (or a separate transmembrane region along with a separate signaling region).

In several embodiments, the various domains/subdomains are separated by a linker such as, a GS3 linker (SEQ ID NO: 15 and 16, nucleotide and protein, respectively) is used (or a GSn linker). In some embodiments, such as wherein the antigen-binding domain comprises a VH and a VL, the VH and VL are connected by a linker. In some embodiments, such as wherein the antigen-binding domain comprises a VH and a VL, the antigen-binding domain comprises a linker between the VH and VL. In some embodiments, such as wherein the antigen-binding domain comprises multiple (e.g., two) VHHs, the VHHs are connected by a linker. In some embodiments, such as wherein the antigen-binding domain comprises multiple (e.g., two) VHHs, the antigen-binding domain comprises a linker between the VHHs. In several embodiments, the linker comprises SEQ ID NO: 15. In several embodiments, the linker comprises SEQ ID NO: 16. In several embodiments, the linker comprises SEQ ID NO: 1014. In several embodiments, the linker comprises SEQ ID NO: 1015. Other linkers used according to various embodiments disclosed herein include, but are not limited to those encoded by SEQ ID NO: 17, 19, 21 or 23. This provides the potential to separate the various component parts of the receptor complex along the polynucleotide, which can enhance expression, stability, and/or functionality of the receptor complex.

### Cytotoxic Signaling Complex

Some embodiments of the compositions and methods described herein relate to a chimeric receptor, such as a chimeric antigen receptor (e.g., a CAR directed to CD19, CD70, BCMA, Her2, mesothelin, Claudin 6, CD123,or EGFR (among others), or a chimeric receptor directed against an NKG2D ligand, such as MICA, MICB, ULBP1, ULBP2, ULBP3, ULBP4, ULBP5, and/or ULBP6) that includes a cytotoxic signaling complex. As disclosed herein, according to several embodiments, the provided cytotoxic receptor complexes comprise one or more transmembrane and/or intracellular domains that initiate cytotoxic signaling cascades upon the extracellular domain(s) binding to ligands on the surface of target cells.

Thus, in some embodiments, the chimeric receptor comprises an extracellular binding domain (e.g., an antigen-binding domain), a transmembrane domain, and an intracellular signaling domain. In several embodiments, the cytotoxic signaling complex comprises at least one transmembrane domain, at least one co-stimulatory domain, and/or at least one signaling domain. In some embodiments, more than one component part makes up a given domain—e.g., a co-stimulatory domain may comprise two subdomains. In some embodiments, the intracellular signaling domain comprises a primary signaling domain (e.g., CD3zeta). In some embodiments, the intracellular signaling domain comprises a co-stimulatory signaling domain (e.g., OX40, CD28, DAP10, 4-1 BB, or ICOS). In some embodiments, the intracellular signaling domain comprises a primary signaling domain (e.g., CD3zeta) and a co-stimulatory signaling domain (e.g., OX40, CD28, DAP10, 4-1BB, or ICOS). Moreover, in some embodiments, a domain may serve multiple functions, for example, a transmembrane domain may also serve to provide signaling function.

### Transmembrane Domains

Some embodiments of the compositions and methods described herein relate to chimeric receptors (e.g., tumor antigen-directed CARs and/or ligand-directed chimeric receptors) that comprise a transmembrane domain. Some embodiments include a transmembrane domain from NKG2D or another transmembrane protein. In several embodiments in which a transmembrane domain is employed, the portion of the transmembrane protein employed retains at least a portion of its normal transmembrane domain.

The antigen-binding protein generally is linked to one or more intracellular signaling components, such as signaling components that mimic activation through an antigen receptor complex, such as a TCR complex, in the case of a CAR, and/or signal via another cell surface receptor. Thus, in some embodiments, the antigen-binding component (e.g., antibody) is linked to one or more transmembrane and intracellular signaling domains. In some embodiments, the transmembrane domain is fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex. In some embodiments, the transmembrane domain comprises a hinge. In some embodiments, the transmembrane domain comprises a transmembrane region. In some embodiments, the transmembrane domain comprises a hinge and/or a transmembrane region. In some embodiments, the transmembrane domain comprises a hinge and a transmembrane region.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CDS, CD9, CD 16, CD22, CD33, CD37, CD64, CD80, CD86, CD 134, CD137, CD 154 and/or transmembrane regions containing functional variants thereof such as those retaining a substantial portion of the structural, e.g., transmembrane, properties thereof. In some embodiments, the transmembrane domain is a transmembrane domain derived from CD4, CD28, or CD8, e.g., CD8alpha, or functional variant thereof. Alternatively the transmembrane domain in some embodiments is synthetic.

In several embodiments, however, the transmembrane domain comprises at least a portion of CD8, a transmembrane glycoprotein normally expressed on both T cells and NK cells. In several embodiments, the transmembrane domain comprises CD8a. In several embodiments, the transmembrane domain comprises a CD8 (e.g., CD8 a) hinge. In several embodiments, the transmembrane domain comprises a CD8 (e.g., CD8 a) transmembrane region. In several embodiments, the transmembrane domain comprises a CD8 (e.g., CD8 a) hinge and a CD8 (e.g., CD8 a) transmembrane region. In several embodiments, the transmembrane domain comprises a “hinge”, e.g. a CD8a hinge. In several embodiments, the “hinge” of CD8a has the nucleic acid sequence of SEQ ID NO: 1. In several embodiments, the CD8a hinge is truncated or modified and is at least 70%, at least 75%, at least 80%, at least 85%, at least 90%, at least 95% homologous with the CD8a having the sequence of SEQ ID NO: 1. In several embodiments, the “hinge” of CD8a comprises the amino acid sequence of SEQ ID NO: 2. In several embodiments, the CD8a can be truncated or modified, such that it is at least 70%, at least 75%, at least 80%, at least 85%, at least 90%, at least 95% homologous with the sequence of SEQ ID NO: 2.

In several embodiments, the transmembrane domain comprises a CD8a transmembrane region. In several embodiments, the CD8a transmembrane region is encoded by a nucleic acid sequence of SEQ ID NO: 3. In several embodiments, the CD8a transmembrane region is truncated or modified and is encoded by a nucleic acid sequence that has at least 70%, at least 75%, at least 80%, at least 85%, at least 90%, at least 95% sequence identity with the CD8a having the sequence of SEQ ID NO: 3. In several embodiments, the CD8a transmembrane region comprises the amino acid sequence of SEQ ID NO: 4. In several embodiments, the CD8a transmembrane region is truncated or modified and is at least 70%, at least 75%, at least 80%, at least 85%, at least 90%, at least 95% homologous with the CD8a having the sequence of SEQ ID NO: 4.

Taken together in several embodiments, the CD8 transmembrane domain is encoded by the nucleic acid sequence of SEQ ID NO: 13. In several embodiments, the CD8 transmembrane domain is truncated or modified and is at least 70%, at least 75%, at least 80%, at least 85%, at least 90%, at least 95% homologous with the CD8 transmembrane domain having the sequence of SEQ ID NO: 13. In several embodiments, the CD8 transmembrane domain comprises the amino acid sequence of SEQ ID NO: 14. In several embodiments, the CD8 transmembrane domain hinge is truncated or modified and is at least 70%, at least 75%, at least 80%, at least 85%, at least 90%, at least 95% homologous with the CD8 transmembrane domain having the sequence of SEQ ID NO: 14.

In some embodiments, the transmembrane domain comprises a CD28 transmembrane domain or a fragment thereof. In several embodiments, the CD28 transmembrane domain comprises the amino acid sequence of SEQ ID NO: 30. In several embodiments, the CD28 transmembrane domain is truncated or modified and is at least 70%, at least 75%, at least 80%, at least 85%, at least 90%, at least 95% homologous with the CD28 transmembrane domain having the sequence of SEQ ID NO: 30.

### Signaling Domains

Some embodiments of the compositions and methods described herein relate to a chimeric receptor (e.g., tumor antigen-directed CARs and/or tumor ligand-directed chimeric receptors) that includes a signaling domain. Among the intracellular signaling domains are those that mimic or approximate a signal through a natural antigen receptor, a signal through such a receptor in combination with a costimulatory receptor, and/or a signal through a costimulatory receptor alone.

The receptor, e.g., the CAR, generally includes at least one intracellular signaling component or components. In some embodiments, the receptor includes an intracellular component of a TCR complex, such as a TCR CD3 chain that mediates T-cell activation and cytotoxicity, e.g., CD3 zeta chain. Thus, in some aspects, the antigen-binding portion is linked to one or more cell signaling modules. In some embodiments, upon ligation of the CAR or other chimeric receptor, the cytoplasmic domain or intracellular signaling domain of the receptor activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptors to initiate signal transduction following antigen receptor engagement.

In the context of a natural TCR, full activation generally requires not only signaling through the TCR, but also a costimulatory signal. Thus, in some embodiments, to promote full activation, a component for generating secondary or co-stimulatory signal is also included in the receptor.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the receptor includes one or both of such signaling components.

In some aspects, the receptor includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR zeta, FcR gamma, FcR beta, CD3 gamma, CD3 delta, CD3 epsilon, CDS, CD22, CD79a, CD79b, and CD66d. In some embodiments, cytoplasmic signaling molecule(s) in the CAR contain(s) a cytoplasmic signaling domain, portion thereof, or sequence derived from CD3 zeta.

For example, immune cells engineered according to several embodiments disclosed herein may comprise at least one subunit of the CD3 T cell receptor complex (or a fragment thereof). In several embodiments, the signaling domain comprises the CD3 zeta subunit. In several embodiments, the CD3 zeta is encoded by the nucleic acid sequence of SEQ ID NO: 7. In several embodiments, the CD3 zeta can be truncated or modified, such that it is at least 70%, at least 75%, at least 80%, at least 85%, at least 90%, at least 95% homologous with the CD3 zeta having the sequence of SEQ ID NO: 7. In several embodiments, the CD3 zeta domain comprises the amino acid sequence of SEQ ID NO: 8. In several embodiments, the CD3 zeta domain is truncated or modified and is at least 70%, at least 75%, at least 80%, at least 85%, at least 90%, at least 95% homologous with the CD3 zeta domain having the sequence of SEQ ID NO: 8.

In some embodiments, the receptor includes a signaling domain and/or transmembrane portion of a costimulatory receptor, such as CD28, 4-1BB, OX40, DAP10, and ICOS. In some embodiments, the intracellular signaling domain comprises a co-stimulatory domain. In some aspects, the same receptor includes both the activating and costimulatory components. In some embodiments, the intracellular signaling component of the recombinant receptor, such as CAR, comprises a CD3 zeta intracellular domain and a costimulatory signaling region.

In some embodiments, the intracellular signaling region comprises an OX40 co-stimulatory domain. In some embodiments, the intracellular signaling region comprises an OX40 co-stimulatory domain and CD3zeta. In some embodiments, the intracellular signaling domain comprises a chimeric OX40 co-stimulatory domain linked to a CD3 zeta intracellular domain.

In several embodiments, unexpectedly enhanced signaling is achieved through the use of multiple signaling domains whose activities act synergistically. For example, in several embodiments, the signaling domain further comprises an OX40 domain. In several embodiments, the OX40 domain is an intracellular signaling domain. In several embodiments, the OX40 intracellular signaling domain has the nucleic acid sequence of SEQ ID NO: 5. In several embodiments, the OX40 intracellular signaling domain can be truncated or modified, such that it is at least 70%, at least 75%, at least 80%, at least 85%, at least 90%, at least 95% homologous with the OX40 having the sequence of SEQ ID NO: 5. In several embodiments, the OX40 intracellular signaling domain comprises the amino acid sequence of SEQ ID NO: 6. In several embodiments, the OX40 intracellular signaling domain is truncated or modified and is at least 70%, at least 75%, at least 80%, at least 85%, at least 90%, at least 95% homologous with the OX40 intracellular signaling domain having the sequence of SEQ ID NO: 6. In several embodiments, OX40 is used as the sole transmembrane/signaling domain in the construct, however, in several embodiments, OX40 can be used with one or more other domains. For example, combinations of OX40 and CD3zeta are used in some embodiments. By way of further example, combinations of CD28, OX40, 4-1BB, and/or CD3zeta are used in some embodiments.

In some embodiments, the intracellular signaling region comprises a 4-1BB co-stimulatory domain. In some embodiments, the intracellular signaling region comprises a 4-1BB co-stimulatory domain and CD3zeta. In several embodiments, the signaling domain comprises a 4-1BB domain. In several embodiments, the 4-1BB domain is an intracellular signaling domain. In several embodiments, the 4-1BB intracellular signaling domain comprises the amino acid sequence of SEQ ID NO: 29. In several embodiments, the 4-1BB intracellular signaling domain is truncated or modified and is at least 70%, at least 75%, at least 80%, at least 85%, at least 90%, at least 95% homologous with the 4-1BB intracellular signaling domain having the sequence of SEQ ID NO: 29. In several embodiments, 4-1BB is used as the sole transmembrane/signaling domain in the construct, however, in several embodiments, 4-1BB can be used with one or more other domains. For example, combinations of 4-1BB and CD3zeta are used in some embodiments. By way of further example, combinations of CD28, OX40, 4-1BB, and/or CD3zeta are used in some embodiments.

In some embodiments, the intracellular signaling region comprises a CD28 co-stimulatory domain. In some embodiments, the intracellular signaling region comprises a CD28 co-stimulatory domain and CD3zeta. In several embodiments, the signaling domain comprises a CD28 domain. In several embodiments the CD28 domain is an intracellular signaling domain. In several embodiments, the CD28 intracellular signaling domain comprises the amino acid sequence of SEQ ID NO: 31. In several embodiments, the CD28 intracellular signaling domain is truncated or modified and is at least 70%, at least 75%, at least 80%, at least 85%, at least 90%, at least 95% homologous with the CD28 intracellular signaling domain having the sequence of SEQ ID NO: 31. In several embodiments, CD28 is used as the sole transmembrane/signaling domain in the construct, however, in several embodiments, CD28 can be used with one or more other domains. For example, combinations of CD28 and CD3zeta are used in some embodiments. By way of further example, combinations of CD28, OX40, 4-1BB, and/or CD3zeta are used in some embodiments.

In some embodiments, the intracellular signaling region comprises a DAP10 co-stimulatory domain. In some embodiments, the intracellular signaling region comprises a DAP10 co-stimulatory domain and CD3zeta.

In some embodiments, the intracellular signaling region comprises an ICOS co-stimulatory domain. In some embodiments, the intracellular signaling region comprises an ICOS co-stimulatory domain and CD3zeta.

In any of the provided embodiments, the nucleic acid encoding the chimeric receptor, or a portion thereof, is codon-optimized. In some embodiments, the polynucleotides are optimized, or contain certain features designed for optimization, such as for codon usage, to reduce RNA heterogeneity and/or to modify, e.g., increase or render more consistent among cell product lots, expression, such as surface expression, of the encoded receptor. In some embodiments, polynucleotides, encoding chimeric receptors, are modified as compared to a reference polynucleotide, such as to remove cryptic or hidden splice sites, to reduce RNA heterogeneity. In some embodiments, polynucleotides, encoding chimeric receptors, are codon optimized, such as for expression in a mammalian, e.g., human, cell such as in a human T cell. In some aspects, the modified polynucleotides result in in improved, e.g., increased or more uniform or more consistent level of, expression, e.g., surface expression, when expressed in a cell.

### Stimulatory Molecules

In some embodiments, the intracellular signaling domain of a chimeric receptor provided herein comprises a co-stimulatory signaling domain, including any of those as described in the preceding section. Thus, in some embodiments of the compositions and methods described herein relate to chimeric receptors (e.g., tumor antigen-directed CARs and/or tumor ligand-directed chimeric receptors) that comprise an intracellular co-stimulatory molecule.

In addition to the various transmembrane domains and signaling domains (and the combination transmembrane/signaling domains), additional co-activating molecules (“stimulatory molecules”) can be provided, in several embodiments. These can be certain molecules that, for example, further enhance activity of the immune cells. Cytokines may be used in some embodiments. For example, certain interleukins, such as IL-2 and/or IL-15 as non-limiting examples, are used. In some embodiments, the immune cells for therapy are engineered to express such molecules as a secreted form. In additional embodiments, such stimulatory molecules are engineered to be membrane bound, acting as autocrine stimulatory molecules (or even as paracrine stimulators to neighboring cells).

In several embodiments, NK cells are engineered to express interleukin-15 (IL15). In several embodiments, NK cells are engineered to express membrane-bound interleukin 15 (mbIL15). In such embodiments, mbIL15 expression on the NK enhances the cytotoxic effects of the engineered NK cell by enhancing the proliferation and/or longevity of the NK cells. In some embodiments, the IL15 is expressed from a separate cassette on the construct comprising any one of the CARs disclosed herein. In some embodiments, the IL15 is expressed from the same cassette as any one of the CARs disclosed herein. In some embodiments, the chimeric receptor and IL15 are separated by a nucleic acid sequence encoding a cleavage site, for example, a proteolytic cleavage site or a T2A, P2A, E2A. or F2A self-cleaving peptide cleavage site. In some embodiments, the chimeric receptor and the IL15 (e.g., mbIL15) are separated by a 2A self-cleaving peptide. In some embodiments, the chimeric receptor and IL15 are separated by a T2A peptide (e.g., SEQ ID NO:10, encoded by SEQ ID NO:9). In some embodiments, the IL15 is a membrane-bound IL15 (mbIL15). Thus, in some embodiments, the chimeric receptor and the mbIL15 are separated by a T2A peptide.

In several embodiments, T cells, such as the genetically engineered non-alloreactive T cells disclosed herein are engineered to express membrane-bound interleukin 15 (mbIL15). In such embodiments, mbIL15 expression on the T cell enhances the cytotoxic effects of the engineered T cell by enhancing the activity and/or propagation (e.g., longevity) of the engineered T cells.

In some embodiments, the mbIL15 comprises a native IL15 sequence, such as a human native IL15 sequence, and at least one transmembrane domain. In several embodiments, IL15 is encoded by the nucleic acid sequence of SEQ ID NO: 11. In several embodiments, IL15 can be truncated or modified, such that it is encoded by a nucleic acid sequence that has at least 70%, at least 75%, at least 80%, at least 85%, at least 90%, at least 95% sequence identity with the sequence of SEQ ID NO: 11. In several embodiments, the IL15 comprises the amino acid sequence of SEQ ID NO: 12. In several embodiments, the IL15 is truncated or modified and is at least 70%, at least 75%, at least 80%, at least 85%, at least 90%, at least 95% homologous with the IL15 having the sequence of SEQ ID NO: 12.

In some embodiments, the mbIL15 is membrane-bound by virtue of the fusion of IL15 to a transmembrane domain. In some embodiments, the transmembrane domain comprises a hinge. In some embodiments, the transmembrane domain comprises a transmembrane region. In some embodiments, the transmembrane domain comprises a hinge and a transmembrane region. Thus, in some embodiments, mbIL15 comprises a transmembrane domain. In some embodiments, the transmembrane domain comprises a CD8 transmembrane domain (e.g., SEQ ID NO:14). In some embodiments, the CD8 transmembrane domain comprises a CD8 transmembrane region (e.g., SEQ ID NO:4). In some embodiments, the mbIL15 may comprise additional components, such as a leader sequence and/or a hinge sequence. In some embodiments, the leader sequence is a CD8 leader sequence. In some embodiments, the hinge sequence is a CD8 hinge sequence (e.g., SEQ ID NO:2). In some embodiments, the mbIL15 comprises a native IL15 sequence, such as a human native IL15 sequence, and at least one transmembrane domain. In several embodiments, the mbIL15 is truncated or modified and is at least 70%, at least 75%, at least 80%, at least 85%, at least 90%, at least 95% sequency identity with the mbIL15 having the sequence of SEQ ID NO: 391. In several embodiments, the mbIL15 comprises the amino acid sequence of SEQ ID NO: 391. In several embodiments, mbIL15 is encoded by a nucleic acid sequence that is at least 70%, at least 75%, at least 80%, at least 85%, at least 90%, at least 95% sequence identity with the sequence of SEQ ID NO: 390. In several embodiments, mbIL15 is encoded by the nucleic acid sequence of SEQ ID NO: 390. It is contemplated that any of the amino acid sequences provided herein may be provided with or without a signal sequence (e.g., a CD8a signal sequence, such as MALPVTALLLPLALLLHAARP). In several embodiments, the mbIL15 comprises the amino acid sequence set forth in SEQ ID NO:995.

Membrane-bound IL15 sequences are described in PCT publications WO 2018/183385 and WO 2020/056045, each of which is hereby expressly incorporated by reference in its entirety.

In some embodiments, the tumor antigen-directed CARs and/or tumor ligand-directed chimeric receptors are encoded by a polynucleotide that includes one or more cytosolic protease cleavage sites, for example a T2A cleavage site, a P2A cleavage site, an E2A cleavage site, and/or a F2A cleavage site. Such sites are recognized and cleaved by a cytosolic protease, which can result in separation (and separate expression) of the various component parts of the receptor encoded by the polynucleotide. As a result, depending on the embodiment, the various constituent parts of an engineered cytotoxic receptor complex can be delivered to an NK cell or T cell in a single vector or by multiple vectors. Thus, as shown schematically, in the Figures, a construct can be encoded by a single polynucleotide, but also include a cleavage site, such that downstream elements of the constructs are expressed by the cells as a separate protein (as is the case in some embodiments with IL-15). In several embodiments, a T2A cleavage site is used. In several embodiments, a T2A cleavage site is encoded by the nucleic acid sequence of SEQ ID NO: 9. In several embodiments, T2A cleavage site can be truncated or modified, such that it is encoded by a nucleic acid sequence that has at least 70%, at least 75%, at least 80%, at least 85%, at least 90%, at least 95% sequence identity with the sequence of SEQ ID NO: 9. In several embodiments, the T2A cleavage site comprises the amino acid sequence of SEQ ID NO: 10. In several embodiments, the T2A cleavage site is truncated or modified. In several embodiments, the T2A cleavage site is truncated or modified and is at least 70%, at least 75%, at least 80%, at least 85%, at least 90%, at least 95% homologous with the T2A cleavage site having the sequence of SEQ ID NO: 10.

### Cytotoxic Receptor Complex Constructs

Some embodiments of the compositions and methods described herein relate to chimeric antigen receptors, such as a CD19-directed chimeric receptor, as well as chimeric receptors, such as an activating chimeric receptor (ACR) that target a ligand of NKG2D. In some embodiments, the compositions and methods described herein related to CD70-directed chimeric receptors, BCMA-directed chimeric receptors, or cells (e.g., NK cells) expressing the same. In some embodiments, the compositions and methods described herein related to CD70-directed chimeric receptors and cells (e.g., NK cells) expressing the same. In some embodiments, the compositions and methods described herein related to BCMA-directed chimeric receptors and cells (e.g., NK cells) expressing the same. The expression of these cytotoxic receptor complexes in immune cells, such as genetically modified non-alloreactive T cells and/or NK cells, allows the targeting and destruction of particular target cells, such as cancerous cells. Non-limiting examples of such cytotoxic receptor complexes are discussed in more detail below.

### Chimeric Antigen Receptor Cytotoxic Receptor Complex Constructs

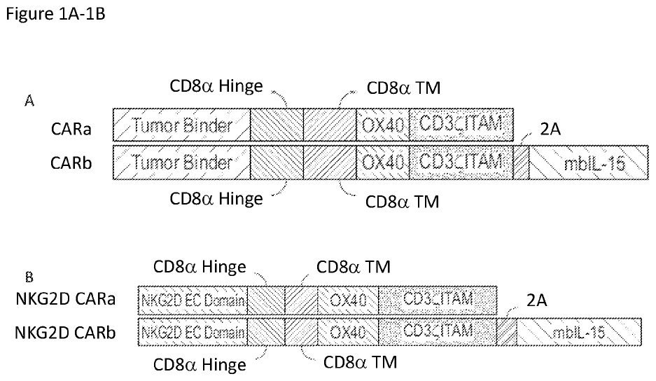

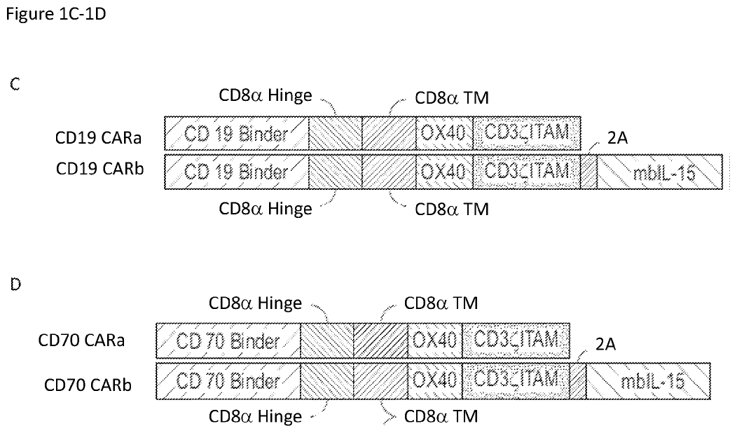

In several embodiments, there are provided for herein a variety of cytotoxic receptor complexes (also referred to as cytotoxic receptors) are provided for herein with the general structure of a chimeric antigen receptor. FIGS. 1A-1D show non-limiting schematics of constructs that include a tumor binding moiety that binds to tumor antigens or tumor-associated antigens expressed on the surface of cancer cells and activates the engineered cell expressing the chimeric antigen receptor.

As shown in FIG. 1A, for example, several embodiments of the chimeric receptor include an anti-tumor binder, a CD8a hinge domain, a CD8a transmembrane domain, an OX40 domain, a CD3(ITAM domain and in some embodiments (see e.g., FIG. 1A, CARb) a 2A cleavage site, and a membrane-bound IL-15 domain (though, as above, in several embodiments soluble IL-15 is used). In the Figures, the CARa is representative of the polypeptide structure as would be expressed by an immune cell such that the tumor binder is extracellular and able to bind the target marker on tumor cells. CARb can be considered as an embodiment of a polynucleotide that bicistronically encodes the CAR polypeptide and mbIL-15, which will be expressed as a separate protein. In several embodiments, the CAR targets NKG2D ligands (see, e.g., FIG. 1B), CD19 (see, e.g., FIG. 1C), or CD70 (see, e.g., FIG. 1D). It shall be appreciated that these schematics are non-limiting and other structural elements of a CAR as disclosed herein can be used, and likewise other tumor markers can be targeted.

In several embodiments, the general structure of the chimeric antigen receptor construct includes a hinge and/or transmembrane domain. These may, in some embodiments, be fulfilled by a single domain, or a plurality of subdomains may be used, in several embodiments. The receptor complex further comprises a signaling domain, which transduces signals after binding of the homing moiety to the target cell, ultimately leading to the cytotoxic effects on the target cell. In several embodiments, the complex further comprises a co-stimulatory domain, which operates, synergistically, in several embodiments, to enhance the function of the signaling domain. Expression of these complexes in immune cells, such as T cells and/or NK cells, allows the targeting and destruction of particular target cells, such as cancerous cells that express a given tumor marker. Some such receptor complexes comprise an extracellular domain comprising an anti-CD19 moiety, or CD19-binding moiety, that binds CD19 on the surface of target cells and activates the engineered cell. The CD3zeta ITAM subdomain may act in concert as a signaling domain. The IL-15 domain, e.g., mbIL-15 domain, may act as a co-stimulatory domain. The IL-15 domain, e.g. mbIL-15 domain, may render immune cells (e.g., NK or T cells) expressing it particularly efficacious against target tumor cells. It shall be appreciated that the IL-15 domain, such as an mbIL-15 domain, can, in accordance with several embodiments, be encoded on a separate construct. Additionally, each of the components may be encoded in one or more separate constructs. In some embodiments, the cytotoxic receptor or CD19-directed receptor comprises a sequence of amino acids that is at least 70%, 75%, 80%, 85%, 90%, 91%, 92%, 93%, 94%, 95%, 96%, 97%, 98%, 99%, or more, or a range defined by any two of the aforementioned percentages, identical to the sequence of SEQ ID NO: 34. In some embodiments, the cytotoxic receptor or CD19-directed receptor comprises the amino acid sequence set forth in SEQ ID NO:34.

Depending on the embodiment, various binders can be used to target CD19. In several embodiments, peptide binders are used, while in some embodiments antibodies, or fragments thereof are used. In several embodiments employing antibodies, antibody sequences are optimized, humanized or otherwise manipulated or mutated from their native form in order to increase one or more of stability, affinity, avidity or other characteristic of the antibody or fragment. In several embodiments, an antibody is provided that is specific for CD19. In several embodiments, an scFv is provided that is specific for CD19. In several embodiments, the antibody or scFv specific for CD19 comprises a heavy chain variable comprising the amino acid sequence of SEQ ID NO: 104 or 106. In some embodiments, the heavy chain variable comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 104 or 106. In some embodiments, the heavy chain variable comprises a sequence of amino acids that is encoded by a polynucleotide that hybridizes under moderately stringent conditions to the complement of a polynucleotide that encodes a heavy chain variable of SEQ ID NO. 104 or 106. In some embodiments, the heavy chain variable domain a sequence of amino acids that is encoded by a polynucleotide that hybridizes under stringent conditions to the complement of a polynucleotide that encodes a heavy chain variable encodes a heavy chain variable of SEQ ID NO. 104 or 106. In several embodiments, the heavy chain variable region comprises the CDR-H1, the CDRH-2, and the CDRH-3 as comprised within the amino acid sequence set forth in SEQ ID NO:104. In several embodiments, the heavy chain variable region comprises the CDR-H1, the CDRH-2, and the CDRH-3 as comprised within the amino acid sequence set forth in SEQ ID NO:106.

In several embodiments, the antibody or scFv specific for CD19 comprises a lightchain variable comprising the amino acid sequence of any of SEQ ID NO. 105 or 107. In several embodiments, the light chain variable comprises a sequence of amino acids that is encoded by a nucleotide sequence that is at least 70%, 75%, 80%, 85%, 90%, 91%, 92%, 93%, 94%, 95%, 96%, 97%, 98%, 99%, or more, identical to the identical to the sequence of SEQ ID NO. 105 or 107. In some embodiments, the light chain variable comprises a sequence of amino acids that is encoded by a polynucleotide that hybridizes under moderately stringent conditions to the complement of a polynucleotide that encodes a light chain variable of SEQ ID NO. 105 or 107. In some embodiments, the light chain variable domain comprises a sequence of amino acids that is encoded by a polynucleotide that hybridizes under stringent conditions to the complement of a polynucleotide that encodes a light chain variable domain of SEQ ID NO. 105 or 107. In several embodiments, the light chain variable region comprises the CDR-L1, the CDRL-2, and the CDRL-3 as comprised within the amino acid sequence set forth in SEQ ID NO:105. In several embodiments, the light chain variable region comprises the CDR-L1, the CDRL-2, and the CDRL-3 as comprised within the amino acid sequence set forth in SEQ ID NO:107.

In several embodiments, the anti-CD19 antibody or scFv comprises one, two, or three heavy chain complementarity determining region (CDR) and one, two, or three light chain CDRs. In several embodiments, a first heavy chain CDR has the amino acid sequence of SEQ ID NO: 111. In some embodiments, the first heavy chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 111. In several embodiments, a second heavy chain CDR has the amino acid sequence of SEQ ID NO: 112, 113, or 114. In some embodiments, the second heavy chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 112, 113, or 114. In several embodiments, a third heavy chain CDR has the amino acid sequence of SEQ ID NO: 115. In some embodiments, the third heavy chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 115.

In several embodiments, a first light chain CDR has the amino acid sequence of SEQ ID NO: 108. In some embodiments, the first light chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 108. In several embodiments, a second light chain CDR has the amino acid sequence of SEQ ID NO: 109. In some embodiments, the second light chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 109. In several embodiments, a third light chain CDR has the amino acid sequence of SEQ ID NO: 110. In some embodiments, the third light chain CDR comprises a sequence of amino acids that is at least 70%, 75%, 80%, 85%, 90%, 91%, 92%, 93%, 94%, 95%, 96%, 97%, 98%, 99% or more, identical to the sequence of SEQ ID NO. 110.

In several embodiments, there is provided an anti-CD19 CAR comprising the amino acid sequence of SEQ ID NO. 116. In some embodiments, there is provided an anti-CD19 CAR comprising a sequence of amino acids that is at least 70%, 75%, 80%, 85%, 90%, 91%, 92%, 93%, 94%, 95%, 96%, 97%, 98%, 99% or more, identical to the sequence of SEQ ID NO. 116.

In several embodiments, there is provided herein an anti-CD19/CD8hinge/CD8TM/4-1BB/CD3zeta chimeric antigen receptor complex. In several embodiments, this receptor complex is encoded by a nucleic acid molecule having the sequence of SEQ ID NO: 85 or a sequence that shares at least about 90%, at least about 94%, at least about 95%, at least about 96%, at least about 97%, at least about 98%, or at least about 99%, sequence identity, homology and/or functional equivalence with SEQ ID NO: 85. In several embodiments, the chimeric receptor comprises the amino acid sequence of SEQ ID NO: 86 or an amino acid sequence that shares at least about 90%, at least about 94%, at least about 95%, at least about 96%, at least about 97%, at least about 98%, or at least about 99%, sequence identity, homology and/or functional equivalence with SEQ ID NO: 86.

In several embodiments, there is provided herein an anti CD19/CD8hinge/CD8TM/OX40/CD3zeta chimeric antigen receptor complex. In several embodiments, there is provided herein an anti CD19/CD8hinge/CD8TM/OX40/CD3zeta/2A/mIL-15 chimeric antigen encoded by a nucleic acid molecule having the sequence of SEQ ID NO: 59 or a nucleic acid sequence encoding a CAR comprises a sequence that shares at least about 90%, at least about 94%, at least about 95%, at least about 96%, at least about 97%, at least about 98%, or at least about 99%, sequence identity, homology and/or functional equivalence with SEQ ID NO: 59. In several embodiments, the chimeric receptor comprises the amino acid sequence of SEQ ID NO: 60 or an amino acid sequence that shares at least about 90%, at least about 94%, at least about 95%, at least about 96%, at least about 97%, at least about 98%, or at least about 99%, sequence identity, homology and/or functional equivalence with SEQ ID NO: 60.

In several embodiments, there is provided herein an anti-CD19/CD8hinge/CD28TM/CD28/CD3zeta chimeric antigen receptor complex. In several embodiments, there is provided herein an anti-CD19moiety/CD8hinge/CD28TM/CD28/CD3zeta/2A/mIL15 chimeric antigen receptor complex. In such embodiments, the polynucleotide comprises or is composed of an anti-CD19 scFv, a CD8a hinge, a CD28 transmembrane domain, CD28 signaling domain, a CD3zeta domain a 2A cleavage site, and an mbIL-15 encoded by a nucleic acid molecule having the sequence of SEQ ID NO: 61 or a sequence that shares at least about 90%, at least about 94%, at least about 95%, at least about 96%, at least about 97%, at least about 98%, or at least about 99%, sequence identity, homology and/or functional equivalence with SEQ ID NO: 61. In several embodiments, the chimeric receptor comprises the amino acid sequence of SEQ ID NO: 62 or an amino acid sequence that shares at least about 90%, at least about 94%, at least about 95%, at least about 96%, at least about 97%, at least about 98%, or at least about 99%, sequence identity, homology and/or functional equivalence with SEQ ID NO: 62.

In several embodiments, provided herein is an anti-CD19moiety/CD8hinge/CD8aTM/ICOS/CD3zeta chimeric antigen receptor complex. In several embodiments, anti-CD19moiety/CD8hinge/CD8aTM/ICOS/CD3zeta/2A/mIL15 chimeric antigen receptor complex encoded by a nucleic acid molecule having the sequence of SEQ ID NO: 63 or a sequence that shares at least about 90%, at least about 94%, at least about 95%, at least about 96%, at least about 97%, at least about 98%, or at least about 99%, sequence identity, homology and/or functional equivalence with SEQ ID NO: 63. In several embodiments, the chimeric receptor comprises the amino acid sequence of SEQ ID NO: 64 or an amino acid sequence that shares at least about 90%, at least about 94%, at least about 95%, at least about 96%, at least about 97%, at least about 98%, or at least about 99%, sequence identity, homology and/or functional equivalence with SEQ ID NO: 64.

In several embodiments, there is provided herein a anti-CD19moiety/CD8hinge/CD8aTM/CD28/4-1BB/CD3zeta chimeric antigen receptor complex. In several embodiments, anti-CD19moiety/CD8hinge/CD8aTM/CD28/4-1BB/CD3zeta/2A/mIL-15 encoded by a nucleic acid molecule having the sequence of SEQ ID NO: 65 or a nucleic acid sequence encoding the chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 65. In several embodiments, the chimeric receptor comprises the amino acid sequence of SEQ ID NO: 66 or an amino acid sequence that shares at least about 90%, at least about 94%, at least about 95%, at least about 96%, at least about 97%, at least about 98%, or at least about 99%, sequence identity, homology and/or functional equivalence with SEQ ID NO: 66

In several embodiments, there is provided herein an anti-CD19moiety/CD8hinge/NKG2DTM/OX40/CD3zeta chimeric antigen receptor complex. In several embodiments, there is provided herein an anti-CD19moiety/CD8hinge/NKG2DTM/OX40/CD3zeta/2A/mIL-15 chimeric antigen receptor complex encoded by a nucleic acid molecule having the sequence of SEQ ID NO: 67 or a sequence that shares at least about 90%, at least about 94%, at least about 95%, at least about 96%, at least about 97%, at least about 98%, or at least about 99%, sequence identity, homology and/or functional equivalence with SEQ ID NO: 67. In several embodiments, the chimeric receptor comprises the amino acid sequence of SEQ ID NO: 68 or an amino acid sequence that shares at least about 90%, at least about 94%, at least about 95%, at least about 96%, at least about 97%, at least about 98%, or at least about 99%, sequence identity, homology and/or functional equivalence with SEQ ID NO: 68.

In several embodiments, there is provided an anti-CD19moiety/CD8hinge/CD8aTM/CD40/CD3zeta chimeric antigen receptor complex. In several embodiments, there is provided an anti-CD19moiety/CD8hinge/CD8aTM/CD40/CD3zeta/2A/mIL-15 chimeric antigen receptor complex encoded by a nucleic acid molecule having the sequence of SEQ ID NO: 69 or a sequence that shares at least about 90%, at least about 94%, at least about 95%, at least about 96%, at least about 97%, at least about 98%, or at least about 99%, sequence identity, homology and/or functional equivalence with SEQ ID NO: 69. In several embodiments, the chimeric receptor comprises the amino acid sequence of SEQ ID NO: 70 or an amino acid sequence that shares at least about 90%, at least about 94%, at least about 95%, at least about 96%, at least about 97%, at least about 98%, or at least about 99%, sequence identity, homology and/or functional equivalence with SEQ ID NO: 70.

In several embodiments, there is provided herein an anti-CD19moiety/CD8hinge/CD8aTM/OX40/CD3zeta/2A/mIL-15/2A/EGFRt chimeric antigen receptor complex encoded by a nucleic acid molecule having the sequence of SEQ ID NO: 71 or a sequence that shares at least about 90%, at least about 94%, at least about 95%, at least about 96%, at least about 97%, at least about 98%, or at least about 99%, sequence identity, homology and/or functional equivalence with SEQ ID NO: 71. In several embodiments, the chimeric receptor comprises the amino acid sequence of SEQ ID NO: 72 or an amino acid sequence that shares at least about 90%, at least about 94%, at least about 95%, at least about 96%, at least about 97%, at least about 98%, or at least about 99%, sequence identity, homology and/or functional equivalence with SEQ ID NO: 72.

In several embodiments, there is provided herein an anti-CD19moiety/CD8hinge/CD8aTM/CD40 chimeric antigen receptor complex. In several embodiments, there is provided herein an anti-CD19moiety/CD8hinge/CD8aTM/CD40/CD3zeta/2A/mIL-15 chimeric antigen receptor complex encoded by a nucleic acid molecule having the sequence of SEQ ID NO: 73 or a sequence that shares at least about 90%, at least about 94%, at least about 95%, at least about 96%, at least about 97%, at least about 98%, or at least about 99%, sequence identity, homology and/or functional equivalence with SEQ ID NO: 73. In several embodiments, the chimeric receptor comprises the amino acid sequence of SEQ ID NO: 74 or an amino acid sequence that shares at least about 90%, at least about 94%, at least about 95%, at least about 96%, at least about 97%, at least about 98%, or at least about 99%, sequence identity, homology and/or functional equivalence with SEQ ID NO: 74.

In several embodiments, there is provided an anti-CD19moiety/CD8hinge/CD8aTM/CD27 chimeric antigen receptor complex. In several embodiments, there is provided an anti-CD19moiety/CD8hinge/CD8aTM/CD27/CD3zeta/2A/mIL-15 chimeric antigen receptor complex encoded by a nucleic acid molecule having the sequence of SEQ ID NO: 75 or a sequence that shares at least about 90%, at least about 94%, at least about 95%, at least about 96%, at least about 97%, at least about 98%, or at least about 99%, sequence identity, homology and/or functional equivalence with SEQ ID NO: 75. In several embodiments, the chimeric receptor comprises the amino acid sequence of SEQ ID NO: 76 or an amino acid sequence that shares at least about 90%, at least about 94%, at least about 95%, at least about 96%, at least about 97%, at least about 98%, or at least about 99%, sequence identity, homology and/or functional equivalence with SEQ ID NO: 76.

In several embodiments, there is provided an anti-CD19moiety/CD8hinge/CD8aTM/CD70/CD3zeta chimeric antigen receptor complex. In several embodiments, there is provided an anti-CD19moiety/CD8hinge/CD8aTM/CD70/CD3zeta/2A/mIL-15 chimeric antigen receptor complex encoded by a nucleic acid molecule having the sequence of SEQ ID NO: 77 or a sequence that shares at least about 90%, at least about 94%, at least about 95%, at least about 96%, at least about 97%, at least about 98%, or at least about 99%, sequence identity, homology and/or functional equivalence with SEQ ID NO: 77. In several embodiments, the chimeric receptor comprises the amino acid sequence of SEQ ID NO: 78 or an amino acid sequence that shares at least about 90%, at least about 94%, at least about 95%, at least about 96%, at least about 97%, at least about 98%, or at least about 99%, sequence identity, homology and/or functional equivalence with SEQ ID NO: 78.

In several embodiments, there is an anti-CD19moiety/CD8hinge/CD8aTM/CD161/CD3zeta chimeric antigen receptor complex. In several embodiments, there is an anti-CD19moiety/CD8hinge/CD8aTM/CD161/CD3zeta/2A/mIL-15 chimeric antigen receptor complex encoded by a nucleic acid molecule having the sequence of SEQ ID NO: 79 or a sequence that shares at least about 90%, at least about 94%, at least about 95%, at least about 96%, at least about 97%, at least about 98%, or at least about 99%, sequence identity, homology and/or functional equivalence with SEQ ID NO: 79. In several embodiments, the chimeric receptor comprises the amino acid sequence of SEQ ID NO: 80 or an amino acid sequence that shares at least about 90%, at least about 94%, at least about 95%, at least about 96%, at least about 97%, at least about 98%, or at least about 99%, sequence identity, homology and/or functional equivalence with SEQ ID NO: 80.

In several embodiments, there is provided an anti-CD19moiety/CD8hinge/CD8aTM/CD40L/CD3zeta chimeric antigen receptor complex. In several embodiments, there is provided an anti-CD19moiety/CD8hinge/CD8aTM/CD40L/CD3zeta/2A/mIL-15 chimeric antigen receptor complex encoded by a nucleic acid molecule having the sequence of SEQ ID NO: 81 or a sequence that shares at least about 90%, at least about 94%, at least about 95%, at least about 96%, at least about 97%, at least about 98%, or at least about 99%, sequence identity, homology and/or functional equivalence with SEQ ID NO: 81. In several embodiments, the chimeric receptor comprises the amino acid sequence of SEQ ID NO: 82 or an amino acid sequence that shares at least about 90%, at least about 94%, at least about 95%, at least about 96%, at least about 97%, at least about 98%, or at least about 99%, sequence identity, homology and/or functional equivalence with SEQ ID NO: 82.

In several embodiments, there is provided an anti-CD19moiety/CD8hinge/CD8aTM/CD44/CD3zeta chimeric antigen receptor complex. In several embodiments, there is provided an anti-CD19moiety/CD8hinge/CD8aTM/CD44/CD3zeta/2A/mIL-15 chimeric antigen receptor complex encoded by a nucleic acid molecule having the sequence of SEQ ID NO: 83 or a sequence that shares at least about 90%, at least about 94%, at least about 95%, at least about 96%, at least about 97%, at least about 98%, or at least about 99%, sequence identity, homology and/or functional equivalence with SEQ ID NO: 83. In several embodiments, the chimeric receptor comprises the amino acid sequence of SEQ ID NO: 84 or an amino acid sequence that shares at least about 90%, at least about 94%, at least about 95%, at least about 96%, at least about 97%, at least about 98%, or at least about 99%, sequence identity, homology and/or functional equivalence with SEQ ID NO: 84.

In several embodiments, there is provided a chimeric receptor comprising an anti-CD123 binding domain, a CD8a hinge, a CD8a transmembrane region, an OX-40 co-stimulatory domain, and CD3zeta. In several embodiments, there is provided a polynucleotide encoding an anti CD123/CD8a hinge/CD8a transmembrane domain/OX40/CD3zeta chimeric antigen receptor complex encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein.

In several embodiments, there is provided a chimeric receptor comprising an anti-CLDN6 binding domain, a CD8a hinge, a CD8a transmembrane region, an OX-40 co-stimulatory domain, and CD3zeta. In several embodiments, there is provided a polynucleotide encoding an anti CLDN6/CD8a hinge/CD8a transmembrane domain/OX40/CD3zeta chimeric antigen receptor complex by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein.

Depending on the embodiment, various binders can be used to target CLDN6. In several embodiments, peptide binders are used, while in some embodiments antibodies, or fragments thereof are used. In several embodiments employing antibodies, antibody sequences are optimized, humanized or otherwise manipulated or mutated from their native form in order to increase one or more of stability, affinity, avidity or other characteristic of the antibody or fragment. In several embodiments, an antibody is provided that is specific for CLDN6. In several embodiments, an scFv is provided that is specific for CLDN6. In several embodiments, the antibody or scFv specific for CLDN6 comprises a heavy chain variable comprising the amino acid sequence of SEQ ID NO. 88. In some embodiments, the heavy chain variable comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 88. In some embodiments, the heavy chain variable comprises a sequence of amino acids that is encoded by a polynucleotide that hybridizes under moderately stringent conditions to the complement of a polynucleotide that encodes a heavy chain variable of SEQ ID NO. 88. In some embodiments, the heavy chain variable domain a sequence of amino acids that is encoded by a polynucleotide that hybridizes under stringent conditions to the complement of a polynucleotide that encodes a heavy chain variable encodes a heavy chain variable of SEQ ID NO. 88.

In several embodiments, the antibody or scFv specific for CLDN6 comprises a light chain variable comprising the amino acid sequence of any of SEQ ID NO. 89, 90, or 91. In several embodiments, the light chain variable comprises a sequence of amino acids that is encoded by a nucleotide sequence that is at least 70%, 75%, 80%, 85%, 90%, 91%, 92%, 93%, 94%, 95%, 96%, 97%, 98%, 99%, or more, identical to the identical to the sequence of SEQ ID NO. 89, 90, or 91. In some embodiments, the light chain variable comprises a sequence of amino acids that is encoded by a polynucleotide that hybridizes under moderately stringent conditions to the complement of a polynucleotide that encodes a light chain variable of SEQ ID NO. 89, 90, or 91. In some embodiments, the light chain variable domain comprises a sequence of amino acids that is encoded by a polynucleotide that hybridizes under stringent conditions to the complement of a polynucleotide that encodes a light chain variable domain of SEQ ID NO. 89, 90, or 91.

In several embodiments, the anti-CLDN6 antibody or scFv comprises one, two, or three heavy chain complementarity determining region (CDR) and one, two, or three light chain CDRs. In several embodiments, a first heavy chain CDR has the amino acid sequence of SEQ ID NO: 92. In some embodiments, the first heavy chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 92. In several embodiments, a second heavy chain CDR has the amino acid sequence of SEQ ID NO: 93. In some embodiments, the second heavy chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 93. In several embodiments, a third heavy chain CDR has the amino acid sequence of SEQ ID NO: 94. In some embodiments, the third heavy chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 94.

In several embodiments, a first light chain CDR has the amino acid sequence of SEQ ID NO: 95, 98, or 101. In some embodiments, the first light chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 95, 98, or 101. In several embodiments, a second light chain CDR has the amino acid sequence of SEQ ID NO: 96, 99, or 102. In some embodiments, the second light chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 96, 99, or 102. In several embodiments, a third light chain CDR has the amino acid sequence of SEQ ID NO: 97, 100, or 103. In some embodiments, the third light chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 97, 100, or 103.

Advantageously, in several embodiments, the CLDN6 CARs are highly specific to CLDN6 and do not substantially bind to any of CLDN3, 4, or 9.

In several embodiments, there is provided a chimeric receptor comprising an anti-BCMA binding domain, a CD8a hinge, a CD8a transmembrane region, an OX-40 co-stimulatory domain, and CD3zeta. In several embodiments, there is provided a polynucleotide encoding an anti BCMA/CD8a hinge/CD8a transmembrane domain/OX40/CD3zeta chimeric antigen receptor complex encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site). Additional information about anti-BCMA CARs for use in the presently disclosed methods and compositions can be found in International Patent Publication No. WO 2021/146147, which is incorporated in its entirety by reference herein. Additional information about anti-BCMA CARs for use in the presently disclosed methods and compositions can also be found in International Patent Publication No. WO2023288185, which is incorporated in its entirety by reference herein.

In several embodiments, there is provided a chimeric receptor comprising an anti-HER2 binding domain, a CD8a hinge, a CD8a transmembrane region, an OX-40 co-stimulatory domain, and CD3zeta. In several embodiments, there is provided a polynucleotide encoding an anti HER2/CD8a hinge/CD8a transmembrane domain/OX40/CD3zeta chimeric antigen receptor complex encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein.

In several embodiments, there is provided a chimeric receptor comprising NKG2D or a fragment thereof, a CD8a hinge, a CD8a transmembrane region, an OX-40 co-stimulatory domain, and CD3zeta. In several embodiments, there is provided a polynucleotide encoding an NKG2D/CD8a hinge/CD8a transmembrane domain/OX40/CD3zeta activating chimeric receptor complex (see FIG. 1B). The polynucleotide comprises or is composed of a fragment of the NKG2D receptor capable of binding a ligand of the NKG2D receptor, a CD8alpha hinge, a CD8a transmembrane domain, an OX40 domain, and a CD3zeta domain as described herein. In several embodiments, this receptor complex is encoded by a nucleic acid molecule comprising the nucleic acid sequence of SEQ ID NO: 145. In yet another embodiment, this chimeric receptor is encoded by the amino acid sequence of SEQ ID NO: 174. In some embodiments, the sequence of the chimeric receptor may vary from SEQ ID NO. 145, but remains, depending on the embodiment, at least 70%, at least 75%, at least 80%, at least 85%, at least 90%, or at least 95% homologous with SEQ ID NO. 145. In several embodiments, while the chimeric receptor may vary from SEQ ID NO. 145, the chimeric receptor retains, or in some embodiments, has enhanced. NK cell activating and/or cytotoxic function. Additionally, in several embodiments, this construct can optionally be designed to co-express mbIL15 (FIG. 1B). Additional information about chimeric receptors for use in the presently disclosed methods and compositions can be found in US Patent Publication No. 2020/0131244, which is incorporated in its entirety by reference herein.

In several embodiments, there is provided a chimeric receptor comprising an anti-CD70 binding domain, a CD8a hinge, a CD8a transmembrane region, an OX-40 co-stimulatory domain, and CD3zeta. In several embodiments, there is provided a polynucleotide encoding an anti CD70/CD8a hinge/CD8a transmembrane domain/OX40/CD3zeta chimeric antigen receptor complex. The polynucleotide comprises or is composed of an anti CD70 binding moiety, a CD8alpha hinge, a CD8a transmembrane domain, an OX40 domain, and a CD3zeta domain as described herein. In several embodiments, this receptor complex is encoded by a nucleic acid molecule comprising a sequence obtained from a combination of sequences disclosed herein or comprises an amino acid sequence obtained from a combination of sequences disclosed herein. In several embodiments, the encoding nucleic acid sequence, or the amino acid sequence, comprises a sequence in accordance with one or more of SEQ ID NOs: 209-374 or 599-681, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS: 209-374 or 599-681 as described herein. It shall be appreciated that certain sequence variability, extensions, and/or truncations of the disclosed sequences may result when combining sequences, as a result of, for example, ease or efficiency in cloning (e.g., for creation of a restriction site). In several embodiments, there is provided a CD70 CAR construct that for which the polynucleotide also comprises mbIL15, as disclosed herein. Additional information about anti-CD70 CAR for use in the presently disclosed methods and compositions can be found in U.S. Patent Publication No. 2022/0002424 (corresponding to International Patent Publication NO. WO2021252804), which is incorporated in its entirety by reference herein.

In several embodiments, there is provided a chimeric receptor comprising an anti-mesothelin binding domain, a CD8a hinge, a CD8a transmembrane region, an OX-40 co-stimulatory domain, and CD3zeta. In several embodiments, there is provided a polynucleotide encoding an anti mesothelin/CD8a hinge/CD8a transmembrane domain/OX40/CD3zeta chimeric antigen receptor complex encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein.

In several embodiments, there is provided a chimeric receptor comprising an anti-PD-L1 binding domain, a CD8a hinge, a CD8a transmembrane region, an OX-40 co-stimulatory domain, and CD3zeta. In several embodiments, there is provided a polynucleotide encoding an anti PD-L1/CD8a hinge/CD8a transmembrane domain/OX40/CD3zeta chimeric antigen receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein.

In several embodiments, there is provided a chimeric receptor comprising an anti-EGFR binding domain, a CD8a hinge, a CD8a transmembrane region, an OX-40 co-stimulatory domain, and CD3zeta. In several embodiments, there is provided a polynucleotide encoding an anti EGFR/CD8a hinge/CD8a transmembrane domain/OX40/CD3zeta chimeric antigen receptor complex encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein.

In several embodiments, an expression vector, such as a MSCV-IRES-GFP plasmid, a non-limiting example of which is provided in SEQ ID NO: 87, is used to express any of the chimeric antigen receptors provided for herein.

### Methods of Treatment

Provided herein are methods of treatment, e.g., comprising administering any of the engineered immune cells described herein or a composition containing engineered immune cells. In some aspects, also provided are methods of administering any of the engineered immune cells described herein or a composition containing engineered immune cells to a subject (e.g., a subject having a disease or condition). In some aspects, there is also provided a use of any of the engineered immune cells described herein or a composition containing the engineered immune cells for treating a disease or condition. In some aspects, there is also provided a use of any of the engineered immune cells described herein or a composition containing the engineered immune cells for the manufacture of a medicament to treat a disease or condition. In some aspects, also provided is any of the engineered immune cells described herein or a composition containing the engineered immune cells for use in treating a disease or condition, or for administration to a subject having a disease or condition.

Also provided herein are methods of using any of the chimeric receptors described herein in the preparation of a medicament for treating a disease or condition. Also provided herein is use of any of the chimeric receptors described herein for treating a disease or condition.

Diseases and disorders include tumors, including solid tumors, hematologic malignancies, and melanoma, and include local and metastatic tumors; infectious diseases, such as infection by a virus or other pathogen, e.g., HIV, HCV, HBV, CMV, HPV and parasitic diseases; and autoimmune and inflammatory diseases. In some embodiments, the disease or condition is a tumor, cancer, malignancy, neoplasm, or other proliferative disease or condition. In some embodiments, the disease or condition is an infectious disease or condition, such as, but not limited to, viral, retroviral, bacterial and protozoal infections, immunodeficiency, Cytomegalovirus (CMV), epstein-barr virus (EBV), adenovirus, BK polyoma virus. In some embodiments, the disease or condition is an autoimmune or inflammatory disease or condition, such as arthritis (e.g., Rheumatoid Arthritis (RA)), type I diabetes, Systemic Lupus Erythematosus (SLE), inflammatory bowel disease, psoriasis, scleroderma, autoimmune thyroid disease, Graces' disease, Crohn's disease, multiple sclerosis (MS), asthma, and/or a disease or condition associated with transplantation. In some embodiments, the disease or condition is an autoimmune disease. In some embodiments, the disease or condition is RA. In some embodiments, the disease or disease is SLE. In some embodiments, the disease is MS.

In some embodiments, the disease or condition is cancer. Some embodiments relate to a method of treating, ameliorating, inhibiting, or preventing cancer with a cell or immune cell comprising a chimeric antigen receptor and/or an activating chimeric receptor, as disclosed herein. In some embodiments, the method includes treating or preventing cancer. In some embodiments, the method includes administering a therapeutically effective amount of immune cells expressing a tumor-directed chimeric antigen receptor and/or tumor-directed chimeric receptor as described herein. Examples of types of cancer that may be treated as such are described herein.

Disclosed herein are methods of treating a disease or condition (e.g., cancer) in a subject. In some embodiments, the methods comprise administering to the subject any one of the immune cells described herein or a composition comprising the same, and a therapeutic agent. In some embodiments, the therapeutic agent is selected from the group consisting of a chemotherapeutic agent, a monoclonal antibody, a NK cell engager, or a combination thereof. In some embodiments, the therapeutic agent is a chemotherapeutic agent. In some embodiments, the chemotherapeutic agent comprises an antimetabolite, an alkylating agent, a topoisomerase inhibitor, a mitotic inhibitor, an antibiotic, a protein kinase inhibitor, a proteasome inhibitor, an inhibitor of poly (ADP-ribose) polymerase (PARP), or any combination thereof. In some embodiments, the therapeutic agent is an antibody (e.g., a monoclonal antibody). In some embodiments, the monoclonal antibody comprises an anti-CD20 antibody, an anti-CTLA4 antibody, an anti-EGFR antibody, an anti-HER2/neu antibody, an anti-PD1 antibody, an anti-PD-L1 antibody, an anti-VEGF antibody, or any combination thereof. In some embodiments, the therapeutic agent is a NK cell engager. In some embodiments, the NK cell engager binds to an activating receptor on an NK cell and an antigen expressed by cells of the cancer. In some embodiments, the activating receptor is selected from the group consisting of CD16, NKp30, NKp46, NKG2D, and any combination thereof.

In some embodiments, the therapeutic agent is administered prior to, concurrent with, and/or after administration of genetically engineered cells. In some embodiments, the therapeutic agent is administered prior to administration of genetically engineered cells. In some embodiments, the therapeutic agent is administered prior to treatment with a lymphodepleting therapy. In some embodiments, the therapeutic agent is administered after treatment with a lymphodepleting therapy and prior to administration of the genetically engineered cells. In some embodiments, the therapeutic agent is administered concurrently with administration of genetically engineered cells. In some embodiments, the therapeutic agent is administered after administration of genetically engineered cells.

In certain embodiments, treatment of a subject with a genetically engineered cell(s) described herein achieves one, two, three, four, or more of the following effects, including, for example: (i) reduction or amelioration the severity of disease or symptom associated therewith; (ii) reduction in the duration of a symptom associated with a disease; (iii) protection against the progression of a disease or symptom associated therewith; (iv) regression of a disease or symptom associated therewith; (v) protection against the development or onset of a symptom associated with a disease; (vi) protection against the recurrence of a symptom associated with a disease; (vii) reduction in the hospitalization of a subject; (viii) reduction in the hospitalization length; (ix) an increase in the survival of a subject with a disease; (x) a reduction in the number of symptoms associated with a disease; and (xi) an enhancement, improvement, supplementation, complementation, or augmentation of the prophylactic or therapeutic effect(s) of another therapy. Advantageously, the non-alloreactive engineered T cells disclosed herein further enhance one or more of the above. Administration can be by a variety of routes, including, without limitation, intravenous, intra-arterial, subcutaneous, intramuscular, intrahepatic, intraperitoneal and/or local delivery to an affected tissue.

### Cancer Types

Some embodiments of the compositions and methods described herein relate to administering immune cells comprising a tumor-directed chimeric antigen receptor and/or tumor-directed chimeric receptor to a subject with cancer. Various embodiments provided for herein include treatment or prevention of the following non-limiting examples of cancers. Examples of cancer include, but are not limited to, acute lymphoblastic leukemia (ALL), acute myeloid leukemia (AML), adrenocortical carcinoma, Kaposi sarcoma, lymphoma, gastrointestinal cancer, appendix cancer, central nervous system cancer, basal cell carcinoma, bile duct cancer, bladder cancer, bone cancer, brain tumors (including but not limited to astrocytomas, spinal cord tumors, brain stem glioma, craniopharyngioma, ependymoblastoma, ependymoma, medulloblastoma, medulloepithelioma), breast cancer, bronchial tumors, Burkitt lymphoma, cervical cancer, colon cancer, chronic lymphocytic leukemia (CLL), chronic myelogenous leukemia (CML), chronic myeloproliferative disorders, ductal carcinoma, endometrial cancer, esophageal cancer, gastric cancer, Hodgkin lymphoma, non-Hodgkin lymphoma, hairy cell leukemia, renal cell cancer, leukemia, oral cancer, nasopharyngeal cancer, liver cancer, lung cancer (including but not limited to, non-small cell lung cancer, (NSCLC) and small cell lung cancer), pancreatic cancer, bowel cancer, lymphoma, melanoma, ocular cancer, ovarian cancer, pancreatic cancer, prostate cancer, pituitary cancer, uterine cancer, and vaginal cancer.

In some embodiments, the cancer comprises a tumor. In some embodiments, the cancer comprises a solid tumor. In some embodiments, the cancer is a hematologic malignancy.

In some embodiments, the cancer is multiple myeloma. In some embodiments, the cancer is a leukemia or a lymphoma. In some embodiments, the cancer is a leukemia. In some embodiments, the leukemia is ALL, AML, CLL, or CML. In some embodiments, the cancer is a lymphoma. In some embodiments, the lymphoma is Hodgkin lymphoma or non-Hodgkin lymphoma (NHL). In some embodiments, the cancer is NHL. In some embodiments, the NHL is large B-cell lymphoma (LBCL). In some embodiments, the NHL is aggressive NHL. In some embodiments the NHL is a diffuse large B-cell lymphoma (DLBCL), Burkitt lymphoma, follicular lymphoma (FL, including grade 1, 2, 3a, and 3b), small lymphocytic lymphoma (SLL), mantle cell lymphoma, or marginal zone lymphoma. In some embodiments, the NHL is DLBCL. In some embodiments, the NHL is FL. In some embodiments, the NHL is mantle cell lymphoma. In some embodiments, the NHL is marginal zone lymphoma.

In some embodiments, the cancer is relapsed/refractory. In some embodiments, the cancer is relapsed refractory to a prior line of therapy. In some embodiments, the prior line of therapy comprises one prior line of therapy. In some embodiments, the prior line of therapy comprises two prior lines of therapy. In some embodiments, the prior line of therapy comprises three prior lines of therapy. n some embodiments, the prior line of therapy comprises four prior lines of therapy.

In some embodiments, the prior line of therapy comprises CAR T cells. In some embodiments, the subject is CAR T cell exposed. In some embodiments, the prior line of therapy does not comprise CAR T cells. In some embodiments, the subject is CAR T cell naïve. In some embodiments, the CAR T cells are anti-CD19 CAR T cells. In some embodiments, the CAR T cells are anti-CD70 CAR T cells. In some embodiments, the CAR T cells are anti-BCMA CAR T cells.

### Cancer Targets

The disease or condition to be treated can be any disease or condition in which expression of an antigen is associated with and/or involved in the etiology of the disease or condition, e.g., causing, exacerbating or otherwise participating in such disease or condition. Exemplary diseases and disorders can include diseases or disorders associated with malignancies or cellular transformation (e.g., cancer), autoimmune or inflammatory diseases or infectious diseases caused by, for example, bacteria, viruses, or other pathogens. Exemplary antigens are described herein, including antigens associated with various diseases and disorder that can be treated. In particular embodiments, the chimeric receptor specifically binds to an antigen associated with the disease or condition.

Some embodiments of the compositions and methods described herein relate to immune cells comprising a chimeric receptor that targets a cancer antigen. Non-limiting examples of target antigens include: CD19; CD70, TNF receptor family member B cell maturation (BCMA); CD38; DLL3; G protein coupled receptor class C group 5, member D (GPRC5D); epidermal growth factor receptor (EGFR) CD138; prostate-specific membrane antigen (PSMA); Fms Like Tyrosine Kinase 3 (FLT3); KREMEN2 (Kringle Containing Transmembrane Protein 2) CD123; CD22; CD30; CD171; CS1 (also referred to as CD2 subset 1, CRACC, SLAMF7, CD319, and 19A24); CD5, C-type lectin-like molecule-1 (CLL-1 or CLECL1); CD33; epidermal growth factor receptor variant III (EGFRviii); ganglioside G2 (GD2); ganglioside GD3 (aNeu5Ac(2-8)aNeu5Ac(2-3)bDGalp(1-4)bDGlcp(1-1)Cer); Tn antigen ((Tn Ag) or (GalNAca-Ser/Thr)); Receptor tyrosine kinase-like orphan receptor 1 (ROR1); Tumor-associated glycoprotein 72 (TAG72); CD44v6; a glycosylated CD43 epitope expressed on acute leukemia or lymphoma but not on hematopoietic progenitors, a glycosylated CD43 epitope expressed on non-hematopoietic cancers, Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha (FRa or FRI); Folate receptor beta (FRb); Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-1 receptor), carbonic anhydrase IX (CAIX); Proteasome (Prosome, Macropain) Subunit, Beta Type, 9 (LMP2); glycoprotein 100 (gplOO); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); sialyl Lewis adhesion molecule (sLe); ganglioside GM3 (aNeu5Ac(2-3)bDClalp(1-4)bDGlcp(1-1)Cer); transglutaminase 5 (TGS5); high molecular weight-melanoma associated antigen (HMWMAA); o-acetyl-GD2 ganglioside (OAcGD2); tumor endothelial marker 1 (TEM1/CD248); tumor endothelial marker 7-related (TEM7R); claudin 6 (CLDN6); thyroid stimulating hormone receptor (TSHR); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-la); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCT A-1 or Galectin 8), melanoma antigen recognized by T cells 1 (MelanA or MARTI); Rat sarcoma (Ras) mutant; human Telomerase; reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 IB 1 (CYPIB 1); CCCTC-Binding Factor (Zinc Finger Protein)-Like (BORIS or Brother of the Regulator of imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Gly cation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1), MPL, Biotin, c-MYC epitope Tag, CD34, LAMP1 TROP2, GFRalpha4, CDH17, CDH6, NYBR1, CDH19, CD200R, Slea (CA19.9; Sialyl Lewis Antigen); Fucosyl-GMl, PTK7, gpNMB, CDH1-CD324, CD276/1B7H3, ILl lRa, IL13Ra2, CD179b-IGLll, TCRgamma-delta, NKG2D, CD32 (FCGR2A), Tn ag, Timl-/HVCR1, CSF2RA (GM-CSFR-alpha), TGFbetaR2, Lews Ag, TCR-betal chain, TCR-beta2 chain, TCR-gamma chain, TCR-delta chain, FITC, Leutenizing hormone receptor (LHR), Follicle stimulating hormone receptor (FSHR), Gonadotropin Hormone receptor (CGHR or GR), CCR4, GD3, SLAMF6, SLAMF4, HIV1 envelope glycoprotein, HTLVI-Tax, CMV pp65, EBV-EBNA3c, KSHV K8.1, KSHV-gH, influenza A hemagglutinin (HA), GAD, PDL1, Guanylyl cyclase C (GCC), auto antibody to desmoglein 3 (Dsg3), auto antibody to desmoglein 1 (Dsgl), HLA, HLA-A, HLA-A2, HLA-B, HLA-C, HLA-DP, HLA-DM, HLA-DOA, HLA-DOB, HLA-DQ, HLA-DR, HLA-G, IgE, CD99, Ras G12V, Tissue Factor 1 (TF1), AFP, Claudinl 8.2 (CLD18A2 or CLDN18A.2), P-glycoprotein, STEAP1, Livl, Nectin-4, Cripto, gpA33, BST1/CD157, low conductance chloride channel, and the antigen recognized by TNT antibody. In some embodiments, the antigen is CD19. In some embodiments, the antigen is a ligand of NKG2D. In some embodiments, the antigen is CD70. In some embodiments, the antigen is BCMA. In several embodiments, BCMA and CD138 are targeted in tandem, for example using two populations of immune cells expressing an anti-BCMA CAR or an anti-CD138 CAR or through use of a bi-specific CAR.

### Administration and Dosing

Further provided herein are methods of treating a subject having cancer, comprising administering to the subject a composition comprising immune cells (such as NK and/or T cells) engineered to express a cytotoxic receptor complex as disclosed herein. For example, some embodiments of the compositions and methods described herein relate to use of a tumor-directed chimeric antigen receptor and/or tumor-directed chimeric receptor, or use of cells expressing a tumor-directed chimeric antigen receptor and/or tumor-directed chimeric receptor, for treating a cancer patient. Uses of such engineered immune cells for treating cancer are also provided.

In certain embodiments, treatment of a subject with a genetically engineered cell(s) described herein achieves one, two, three, four, or more of the following effects, including, for example: (i) reduction or amelioration the severity of disease or symptom associated therewith; (ii) reduction in the duration of a symptom associated with a disease; (iii) protection against the progression of a disease or symptom associated therewith; (iv) regression of a disease or symptom associated therewith; (v) protection against the development or onset of a symptom associated with a disease; (vi) protection against the recurrence of a symptom associated with a disease; (vii) reduction in the hospitalization of a subject; (viii) reduction in the hospitalization length; (ix) an increase in the survival of a subject with a disease; (x) a reduction in the number of symptoms associated with a disease; and (xi) an enhancement, improvement, supplementation, complementation. or augmentation of the prophylactic or therapeutic effect(s) of another therapy. Each of these comparisons are versus, for example, a different therapy for a disease, which includes a cell-based immunotherapy for a disease using cells that do not express the constructs disclosed herein.

In some embodiments, the immune cells are obtained from a subject (e.g., a first subject) other than the subject that will receive or ultimately receives the cell therapy. In some embodiments, the immune cells are obtained from a healthy subject (e.g., a subject who does not have cancer). In such embodiments, the cells are then administered to a different subject of the same species, e.g., a second subject. In some embodiments, the immune cells are allogeneic to the subject to be treated.

In some embodiments, the first and second subjects are genetically similar. In some embodiments, the second subject expresses the same HLA class or supertype as the first subject.

Advantageously, the non-alloreactive engineered T cells disclosed herein further enhance one or more of the above.

In some embodiments, the immune cells are obtained from a subject to be treated. Thus, in some embodiments, the immune cells are autologous to the subject to be treated.

Administration can be by a variety of routes, including, without limitation, intravenous, intra-arterial, subcutaneous, intramuscular, intrahepatic, intraperitoneal and/or local delivery to an affected tissue. In some embodiments, administration of the immune cells is intravenous. In some embodiments, a given dose is administered by a single infusion of cells. In some embodiments, a given dose is administered by multiple infusions of cells, or by continuous infusion of cells. In some embodiments, administration of the cell dose or any other therapy (e.g., lymphodepletion therapy and/or combination therapy) is by outpatient delivery. In some embodiments, at least one dose of the immune cells is administrated by outpatient delivery. In some embodiments, each dose of the immune cells is administrated by outpatient delivery.

Doses of immune cells such as NK and/or T cells can be readily determined for a given subject based on their body mass, disease type and state, and desired aggressiveness of treatment, but range, depending on the embodiments, from about 10⁵ cells per kg to about 10¹² cells per kg (e.g., 10⁵-10⁷, 10⁷-10¹⁰, 10¹⁰-10¹² and overlapping ranges therein). In one embodiment, a dose escalation regimen is used. In several embodiments, a range of immune cells such as NK and/or T cells is administered, for example between about 1×10⁶ cells/kg to about 1×10⁸ cells/kg. In several embodiments, a range of NK cells is administered, for example between about 1×10⁶ cells/kg to about 1×10⁸ cells/kg. In several embodiments, a range of T cells is administered, for example between about 1×10⁶ cells/kg to about 1×10⁸ cells/kg.

In some embodiments, a dose of engineered cells comprises between about 100×10⁶ and 3×10⁹ NK cells. In some embodiments, a dose of engineered cells comprises between about 300×10⁶ and 1.5×10⁹ NK cells. In some embodiments, a dose of engineered cells comprises about 100×10⁶ NK cells. In some embodiments, a dose of engineered cells comprises about 300×10⁶ NK cells. In some embodiments, a dose of engineered cells comprises about 500×10⁶ NK cells. In some embodiments, a dose of engineered cells comprises about 1×10⁹ NK cells. In some embodiments, a dose of engineered cells comprises about 1.25×10⁹ NK cells. In some embodiments, a dose of engineered cells comprises about 1.5×10⁹ NK cells. In some embodiments, a dose of engineered cells comprises about 2×10⁹ NK cells. In some embodiments, a dose of engineered cells comprises about 2.5×10⁹ NK cells. In some embodiments, a dose of engineered cells comprises about 3×10⁹ NK cells.

In some embodiments, a dose of engineered cells comprises between about 1×10⁸ and 1×10¹⁰ chimeric receptor-expressing NK cells. In some embodiments, a dose of engineered cells comprises between about 300×10⁶ and 1.5×10⁹ chimeric receptor-expressing NK cells. In some embodiments, a dose of engineered cells comprises about 100×10⁶ chimeric receptor-expressing NK cells. In some embodiments, a dose of engineered cells comprises about 300×10⁶ chimeric receptor-expressing NK cells. In some embodiments, a dose of engineered cells comprises about 500×10⁶ chimeric receptor-expressing NK cells. In some embodiments, a dose of engineered cells comprises about 1×10⁹ chimeric receptor-expressing NK cells. In some embodiments, a dose of engineered cells comprises about 1.25×10⁹ chimeric receptor-expressing NK cells. In some embodiments, a dose of engineered cells comprises about 1.5×10⁹ chimeric receptor-expressing NK cells. In some embodiments, a dose of engineered cells comprises about 1.75×10⁹ chimeric receptor-expressing NK cells. In some embodiments, a dose of engineered cells comprises about 2×10⁹ chimeric receptor-expressing NK cells. In some embodiments, a dose of engineered cells comprises about 2.25×10⁹ chimeric receptor-expressing NK cells. In some embodiments, a dose of engineered cells comprises about 2.5×10⁹ chimeric receptor-expressing NK cells. In some embodiments, a dose of engineered cells comprises about 2.75×10⁹ chimeric receptor-expressing NK cells. In some embodiments, a dose of engineered cells comprises about 3×10⁹ chimeric receptor-expressing NK cells. In some embodiments, a dose of engineered cells comprises about 4×10⁹ chimeric receptor-expressing NK cells. In some embodiments, a dose of engineered cells comprises about 5×10⁹ chimeric receptor-expressing NK cells. In some embodiments, a dose of engineered cells comprises about 6×10⁹ chimeric receptor-expressing NK cells. In some embodiments, a dose of engineered cells comprises about 7×10⁹ chimeric receptor-expressing NK cells. In some embodiments, a dose of engineered cells comprises about 8×10⁹ chimeric receptor-expressing NK cells. In some embodiments, a dose of engineered cells comprises about 39×10⁹ chimeric receptor-expressing NK cells. In some embodiments, a dose of engineered cells comprises about 1×10¹⁰ chimeric receptor-expressing NK cells.

In some embodiments, a dose of engineered cells comprises between about 100×10⁶ and 3×10⁹ T cells. In some embodiments, a dose of engineered cells comprises between about 300×10⁶ and 1.5×10⁹ T cells. In some embodiments, a dose of engineered cells comprises about 100×10⁶ T cells. In some embodiments, a dose of engineered cells comprises about 300×10⁶ T cells. In some embodiments, a dose of engineered cells comprises about 500×10⁶ T cells. In some embodiments, a dose of engineered cells comprises about 1×10⁹T cells. In some embodiments, a dose of engineered cells comprises about 1.25×10⁹ T cells. In some embodiments, a dose of engineered cells comprises about 1.5×10⁹ T cells. In some embodiments, a dose of engineered cells comprises about 2×10⁹ T cells. In some embodiments, a dose of engineered cells comprises about 2.5×10⁹ T cells. In some embodiments, a dose of engineered cells comprises about 3×10⁹ T cells.

In some embodiments, a dose of engineered cells comprises both NK and T cells. In some embodiments, a dose of engineered cells comprises between about 100×10⁶and 3×10⁹ NK cells and between about 100×10⁶ and 3×10⁹ T cells. In some embodiments, a dose of engineered cells comprises between about 300×10⁶ and 1.5×10⁹ NK cells and between about 300×10⁶ and 1.5×10⁹ T cells. In some embodiments, a dose comprises about an equal number of NK cells and T cells. In some embodiments, a dose comprises more NK cells than T cell. In some embodiments, a dose comprises more T cells than NK cells.

In some embodiments, the immune cells are administered in a dosing cycle comprising a dose. In several embodiments, multiple doses are used, for example, two, three, four, or more doses within a dosing cycle. In some embodiments, the immune cells are administered in a dosing cycle comprising between two doses and five doses. In some embodiments, the immune cells are administered in a dosing cycle comprising two doses. In some embodiments, the immune cells are administered in a dosing cycle comprising three doses. In some embodiments, the immune cells are administered in a dosing cycle comprising four doses. In some embodiments, the immune cells are administered in a dosing cycle comprising five doses. Such multi-dose cycles can be repeated one or more times, as needed to treat a tumor and/or prevent disease progression. For example, in some embodiments, if a subject exhibits a clinical response (e.g., complete response), an additional dosing cycle is administered as a consolidation treatment. In some embodiments, if a subject exhibits a clinical response (e.g., complete response) but subsequently exhibits disease progression, an additional dosing cycle is administered as retreatment. In some embodiments, between one and five dosing cycles are administered to the subject. In some embodiments, one dosing cycle is administered to the subject. In some embodiments, two dosing cycles are administered to the subject. In some embodiments, three dosing cycles are administered to the subject. In some embodiments, four dosing cycles are administered to the subject. In some embodiments, five dosing cycles are administered to the subject. In some embodiments, no more than five dosing cycles are administered to the subject.

In some embodiments, a dosing cycle is between about 7 days and about 56 days, between about 14 days and about 42 days, or between about 21 days and about 35 days. In some embodiments, a dosing cycle is about 14 days. In some embodiments, a dosing cycle is about 21 days. In some embodiments, a dosing cycle is about 28 days. In some embodiments, a dosing cycle is about 35 days. In some embodiments, a dosing cycle is about 42 days. In some embodiments, a dosing cycle is about 49 days. In some embodiments, a dosing cycle is about 56 days.

In several embodiments, the doses of NK cells are administered over about 7 days, about 8 days, about 9 days, about 10 days, about 11 days, about 12 days, about 13 days, or about 14 days. In some embodiments, the doses of NK cells are administered over about 7 days. In some embodiments, the doses of NK cells are administered over about 8 days. In some embodiments, the doses of NK cells are administered over about 9 days. In some embodiments, the doses of NK cells are administered over about 10 days. In some embodiments, the doses of NK cells are administered over about 11 days. In some embodiments, the doses of NK cells are administered over about 12 days. In some embodiments, the doses of NK cells are administered over about 13 days. In some embodiments, the doses of NK cells are administered over about 14 days.

In some embodiments, each dose of NK cells is administered between about 5-10 days apart. In some embodiments, a second dose of NK cells is administered about 5-10 days after administration of the first dose. In some embodiments, a third dose of NK cells is administered about 5-10 days after administration of the second dose. In some embodiments, a second dose of NK cells is administered about 5-10 days after administration of the first dose, and a third dose of NK cells is administered about 5-10 days after administration of the second dose. In some embodiments, a second dose of NK cells is administered about 7 days after administration of the first dose. In some embodiments, a third dose of NK cells is administered about 7 days after administration of the second dose. In some embodiments, a second dose of NK cells is administered about 7 days after administration of the first dose, and a third dose of NK cells is administered about 7 days after administration of the second dose. In several embodiments, a dosing cycle comprises administration of three doses of NK cells, wherein the second dose is administered about 5-10 (e.g., 7) days after administration of the first dose, the third dose is administered about 5-10 (e.g., 7) days after administration of the second dose.

In several embodiments, dosing is, for example, 3 doses of about 1.0×10⁸ NK cells or about 3×10⁹ NK cells administered over about 14 days. In several embodiments, dosing is, for example, 3 doses of about 1.0×10⁸ NK cells or about 3×10⁹ NK cells administered over about 21 to 28 days. In several embodiments, dosing is, for example, 3 doses of about 1.0×10⁹ NK cells or about 1.5×10⁹ NK cells administered over about 14 days. In several embodiments, dosing is, for example, 3 doses of about 1.0×10⁹ NK cells or about 1.5×10⁹ NK cells administered over about 21 to 28 days. In several embodiments, a dosing cycle comprises 3 doses of about 3×10⁸ NK cells administered over about 14 days. In several embodiments, a dosing cycle comprises 3 doses of about 3×10⁸ NK cells administered over about 21 days. In several embodiments, a dosing cycle comprises 3 doses of about 3×10⁸ NK cells administered over about 28 days. In several embodiments, a dosing cycle comprises 3 doses of about 1.0×10⁹ NK cells administered over about 14 days. In several embodiments, a dosing cycle comprises 3 doses of about 1.0×10⁹ NK cells administered over about 21 days. In several embodiments, a dosing cycle comprises 3 doses of about 1.0×10⁹ NK cells administered over about 28 days. In several embodiments, a dosing cycle comprises 3 doses of about 1.5×10⁹ NK cells administered over about 14 days. In several embodiments, a dosing cycle comprises 3 doses of about 1.5×10⁹ NK cells administered over about 21 days. In several embodiments, a dosing cycle comprises 3 doses of about 1.5×10⁹ NK cells administered over about 28 days. In several embodiments, a dosing cycle comprises 3 doses of about 2×10⁹ NK cells administered over about 14 days. In several embodiments, a dosing cycle comprises 3 doses of about 2×10⁹ NK cells administered over about 21 days. In several embodiments, a dosing cycle comprises 3 doses of about 2×10⁹ NK cells administered over about 28 days. In several embodiments, a dosing cycle comprises 3 doses of about 2.5×10⁹ NK cells administered over about 14 days. In several embodiments, a dosing cycle comprises 3 doses of about 2.5×10⁹ NK cells administered over about 21 days. In several embodiments, a dosing cycle comprises 3 doses of about 2.5×10⁹ NK cells administered over about 28 days. In several embodiments, a dosing cycle comprises 3 doses of about 3×10⁹ NK cells administered over about 14 days. In several embodiments, a dosing cycle comprises 3 doses of about 3×10⁹ NK cells administered over about 21 days. In several embodiments, a dosing cycle comprises 3 doses of about 3×10⁹ NK cells administered over about 28 days. In some embodiments, the NK cells are chimeric receptor-expressing NK cells.

In some embodiments, each dose of NK cells is administered between about 2-4 days apart. In some embodiments, a second dose of NK cells is administered about 2-4 days after administration of the first dose. In some embodiments, a third dose of NK cells is administered about 2-4 days after administration of the second dose. In some embodiments, a second dose of NK cells is administered about 2-4 days after administration of the first dose, and a third dose of NK cells is administered about 2-4 days after administration of the second dose. In some embodiments, a second dose of NK cells is administered about 3 days after administration of the first dose. In some embodiments, a third dose of NK cells is administered about 4 days after administration of the second dose. In some embodiments, a second dose of NK cells is administered about 3 days after administration of the first dose, and a third dose of NK cells is administered about 4 days after administration of the second dose. In several embodiments, a dosing cycle comprises administration of three doses of NK cells, wherein the second dose is administered about 2-4 (e.g., 3) days after administration of the first dose, the third dose is administered about 2-4 (e.g., 4) days after administration of the second dose.

In several embodiments, the administration of engineered NK cells is preceded by one or more preparatory treatments. In several embodiments, the administration of engineered NK cells is preceded by lymphodepletion. In several embodiments, each dosing cycle is preceded by lymphodepletion. In several embodiments, a lymphodepletion process is performed prior to the first dose. In several embodiments, a combination of chemotherapeutic agents is used for lymphodepletion. In several embodiments, a single chemotherapeutic agent is used for lymphodepletion. In several embodiments, wherein a combination of chemotherapeutic agents is used, agents with different mechanisms of actions are optionally used. In several embodiments, different classes of agents are optionally used. In several embodiments, an antimetabolic agent is used. In several embodiments, the antimetabolic agent inhibits and/or prevents cell replication.

In several embodiments, cyclophosphamide, an alkylating agent that reduces tumor growth, is used in lymphodepletion. In several embodiments, the lymphodepletion comprises cyclophosphamide. In several embodiments, a dose of between about 200 and 1000 mg/m² cyclophosphamide is administered, including doses of about 200 mg/m², about 225 mg/m², about 250 mg/m², about 275 mg/m², about 300 mg/m², about 325 mg/m², about 350 mg/m², about 400 mg/m², about 450 mg/m², about 475 mg/m², about 500 mg/m², about 525 mg/m², about 550 mg/m², about 600 mg/m², about 700 mg/m², about 800 mg/m², about 900 mg/m², about 1000 mg/m², or any dose between those listed. In several embodiments, a dose of about 300 mg/m² cyclophosphamide is administered. In several embodiments, a dose of about 500 mg/m² cyclophosphamide is administered. In several embodiments, the dose of cyclophosphamide is given daily for days (e.g., prior to chimeric receptor-expressing NK administration). In several embodiments, the dose of cyclophosphamide is given daily for at least about 2 days, about 3 days, about 4 days, about 5 days, about 6 days, or about 7 days (e.g., prior to chimeric receptor-expressing NK administration). In several embodiments, the cyclophosphamide is given daily for 3 days. In several embodiments, if necessary, the dose can be split and given, for example, twice daily. In several embodiments, the cyclophosphamide is given daily for 3 days, starting 5 days prior to the first administration of a chimeric receptor-expressing immune cell. In several embodiments, the cyclophosphamide is given at a dose of about 300 mg/m² daily for 3 days, starting 5 days prior to the first administration of a chimeric receptor-expressing immune cell. In several embodiments, the cyclophosphamide is given at a dose of about 500 mg/m² daily for 3 days, starting 5 days prior to the first administration of chimeric receptor-expressing immune cells. In several embodiments, a dose (e.g., a single dose) of about 1000 mg/m² cyclophosphamide is administered. In several embodiments, a single dose of cyclophosphamide is administered about 5 days, about 4 days, about 3 days, or about 2 days prior to the first administration of chimeric receptor-expressing immune cells. In several embodiments, a single dose of cyclophosphamide is administered about 3 days prior to the first administration of chimeric receptor-expressing immune cells. In several embodiments, cyclophosphamide is administered in combination with another lymphodepleting agent.

In several embodiments, the additional lymphodepleting agent is also an antimetabolite. In several embodiments, the additional agent inhibits one or more of DNA polymerase alpha, ribonucleotide reductase and/or DNA primase, thus inhibiting DNA synthesis. In several embodiments, the additional agent is fludarabine. In several embodiments, a dose of between about 5.0 mg/m²-about 200 mg/m² fludarabine is administered, including doses of about 5.0 mg/m², about 10.0 mg/m², about 15.0 mg/m², about 20.0 mg/m², about 25.0 mg/m², about 30.0 mg/m², about 35.0 mg/m², about 40.0 mg/m², about 45.0 mg/m², about 50.0 mg/m², about 60.0 mg/m², about 70.0 mg/m², about 80.0 mg/m², about 90.0 mg/m², about 100.0 mg/m², about 125.0 mg/m², about 150.0 mg/m², about 175.0 mg/m², about 200.0 mg/m², or any dose between those listed. In several embodiments, a dose of about 30 mg/m² fludarabine is administered. In several embodiments, the dose of fludarabine is given daily for at least about 2 days, about 3 days, about 4 days, about 5 days, about 6 days, or about 7 days. In several embodiments, the dose of fludarabine is given daily for about 3 days. In several embodiments, about 30 mg/m² fludarabine is given daily for about 3 days. In several embodiments, about 30 mg/m² fludarabine is given daily for about 5 days. In several embodiments, if necessary, the dose can be split and given, for example, twice daily.

In several embodiments, about 300 mg/m² cyclophosphamide and about 30 mg/m² fludarabine is each given daily for about 3 days. In several embodiments, prior to each dosing cycle, about 300 mg/m² cyclophosphamide and about 30 mg/m² fludarabine is each given daily for about 3 days. In several embodiments, about 500 mg/m² cyclophosphamide and about 30 mg/m² fludarabine is each given daily for about 3 days. In several embodiments, prior to each dosing cycle, about 500 mg/m² cyclophosphamide and about 30 mg/m² fludarabine is each given daily for about 3 days. In several embodiments, prior to each dosing cycle, about 1000 mg/m² cyclophosphamide is given once.

In several embodiments, an additional therapeutic agent is administered at least once during the lymphodepletion and/or the dosing cycle. In several embodiments, an additional therapeutic agent is administered at least once during the lymphodepletion. In several embodiments, an additional therapeutic agent is administered at least once during the dosing cycle. For example, in several embodiments, cyclophosphamide, cytosine arabinoside (Ara-C), an anti-CD20 antibody (a non-limiting embodiment of which is rituximab), and/or an anti-EGFR antibody (a non-limiting embodiment of which is cetuximab) is administered in connection with treatment. In several embodiments, cyclosphosphamide is administered in connection with the treatment. In several embodiments, cytosine arabinoside is administered in connection with the treatment. In several embodiments, an anti-CD20 antibody (e.g., rituximab) is administered in connection with the treatment. In several embodiments, an anti-EGFR antibody (e.g., cetuximab) is administered in connection with the treatment.

Depending on the embodiment, various types of cancer can be treated. In several embodiments, hepatocellular carcinoma is treated. Additional embodiments provided for herein include treatment or prevention of the following non-limiting examples of cancers including, but not limited to, acute lymphoblastic leukemia (ALL), acute myeloid leukemia (AML), adrenocortical carcinoma, Kaposi sarcoma, lymphoma, gastrointestinal cancer, appendix cancer, central nervous system cancer, basal cell carcinoma, bile duct cancer, bladder cancer, bone cancer, brain tumors (including but not limited to astrocytomas, spinal cord tumors, brain stem glioma, glioblastoma, craniopharyngioma, ependymoblastoma, ependymoma, medulloblastoma, medulloepithelioma), breast cancer, bronchial tumors, Burkitt lymphoma, cervical cancer, colon cancer, chronic lymphocytic leukemia (CLL), chronic myelogenous leukemia (CML), chronic myeloproliferative disorders, ductal carcinoma, endometrial cancer, esophageal cancer, gastric cancer, Hodgkin lymphoma, non-Hodgkin lymphoma, hairy cell leukemia, renal cell cancer, leukemia, oral cancer, nasopharyngeal cancer, liver cancer, lung cancer (including but not limited to, non-small cell lung cancer, (NSCLC) and small cell lung cancer), pancreatic cancer, bowel cancer, lymphoma, melanoma, ocular cancer, ovarian cancer, pancreatic cancer, prostate cancer, pituitary cancer, uterine cancer, and vaginal cancer.

In some embodiments, also provided herein are nucleic acid and amino acid sequences that have sequence identity and/or homology of at least 80%, 85%, 90%, 95%, 96%, 97%, 98%, 99% (and ranges therein) as compared with the respective nucleic acid or amino acid sequences of SEQ ID NOS. 1-976 (or combinations of two or more of SEQ ID NOS: 1-976) and that also exhibit one or more of the functions as compared with the respective SEQ ID NOS. 1-976 (or combinations of two or more of SEQ ID NOS: 1-976) including but not limited to, (i) enhanced proliferation, (ii) enhanced activation, (iii) enhanced cytotoxic activity against cells presenting ligands to which NK cells harboring receptors encoded by the nucleic acid and amino acid sequences bind, (iv) enhanced homing to tumor or infected sites, (v) reduced off target cytotoxic effects, (vi) enhanced secretion of immunostimulatory cytokines and chemokines (including, but not limited to IFNg, TNFa, IL-22, CCL3, CCL4, and CCL5), (vii) enhanced ability to stimulate further innate and adaptive immune responses, and (viii) combinations thereof. In some embodiments, also provided herein are nucleic acid and amino acid sequences that have sequence identity and/or homology of at least 80%, 85%, 90%, 95%, 96%, 97%, 98%, 99% (and ranges therein) as compared with the respective nucleic acid or amino acid sequences of SEQ ID NOS. 1-1011 (or combinations of two or more of SEQ ID NOS: 1-1011) and that also exhibit one or more of the functions as compared with the respective SEQ ID NOS. 1-1011 (or combinations of two or more of SEQ ID NOS: 1-1011) including but not limited to, (i) enhanced proliferation, (ii) enhanced activation, (iii) enhanced cytotoxic activity against cells presenting ligands to which NK cells harboring receptors encoded by the nucleic acid and amino acid sequences bind, (iv) enhanced homing to tumor or infected sites, (v) reduced off target cytotoxic effects, (vi) enhanced secretion of immunostimulatory cytokines and chemokines (including, but not limited to IFNg, TNFa, IL-22, CCL3, CCL4, and CCL5), (vii) enhanced ability to stimulate further innate and adaptive immune responses, and (viii) combinations thereof. In some embodiments, also provided herein are nucleic acid and amino acid sequences that have sequence identity and/or homology of at least 80%, 85%, 90%, 95%, 96%, 97%, 98%, 99% (and ranges therein) as compared with the respective nucleic acid or amino acid sequences of SEQ ID NOS. 1-1089 (or combinations of two or more of SEQ ID NOS: 1-1089) and that also exhibit one or more of the functions as compared with the respective SEQ ID NOS. 1-1089 (or combinations of two or more of SEQ ID NOS: 1-1089) including but not limited to, (i) enhanced proliferation, (ii) enhanced activation, (iii) enhanced cytotoxic activity against cells presenting ligands to which NK cells harboring receptors encoded by the nucleic acid and amino acid sequences bind, (iv) enhanced homing to tumor or infected sites, (v) reduced off target cytotoxic effects, (vi) enhanced secretion of immunostimulatory cytokines and chemokines (including, but not limited to IFNg, TNFa, IL-22, CCL3, CCL4, and CCL5), and (vii) enhanced ability to stimulate further innate and adaptive immune responses, and (viii) combinations thereof.

In Advantageously, in several embodiments, the therapies and dosing regimens provided for herein provide effective anti-cancer treatment without certain CAR-T cell toxicities, such as cytokine release syndrome, immune effector cell-associated neurotoxicity syndrome (ICANS) or neurotoxicity, or graft-versus host disease (GVHD). In several embodiments, complete remission is achieved. In several embodiments, complete response (CR) is achieved. In several embodiments, partial response (PR) is achieved. In several embodiments, stable disease (SD) or limited progression of disease is accomplished.

Additionally, in several embodiments, there are provided amino acid sequences that correspond to any of the nucleic acids disclosed herein, while accounting for degeneracy of the nucleic acid code. Furthermore, those sequences (whether nucleic acid or amino acid) that vary from those expressly disclosed herein, but have functional similarity or equivalency are also contemplated within the scope of the present disclosure. The foregoing includes mutants, truncations, substitutions, or other types of modifications.

In several embodiments, polynucleotides encoding the disclosed cytotoxic receptor complexes are mRNA. In some embodiments, the polynucleotide is DNA. In some embodiments, the polynucleotide is operably linked to at least one regulatory element for the expression of the cytotoxic receptor complex.

Additionally provided, according to several embodiments, is a vector comprising the polynucleotide encoding any of the polynucleotides provided for herein, wherein the polynucleotides are optionally operatively linked to at least one regulatory element for expression of a cytotoxic receptor complex. In several embodiments, the vector is a viral vector. In several embodiments, the vector is a retrovirus.

Further provided herein are engineered immune cells (such as NK and/or T cells) comprising the polynucleotide, vector, or cytotoxic receptor complexes as disclosed herein. Further provided herein are compositions comprising a mixture of engineered immune cells (such as NK cells and/or engineered T cells), each population comprising the polynucleotide, vector, or cytotoxic receptor complexes as disclosed herein. Additionally, there are provided herein compositions comprising a mixture of engineered immune cells (such as NK cells and/or engineered T cells), each population comprising the polynucleotide, vector, or cytotoxic receptor complexes as disclosed herein and the T cell population having been genetically modified to reduce/eliminate GvHD and/or HvGD. In some embodiments, the NK cells and the T cells are from the same donor. In some embodiments, the NK cells and the T cells are from different donors.

Doses of immune cells such as NK cells or T cells can be readily determined for a given subject based on their body mass, disease type and state, and desired aggressiveness of treatment, but range, depending on the embodiments, from about 10⁵ cells per kg to about 10¹² cells per kg (e.g., 105-10⁷, 10⁷ 10¹⁰, 10¹⁰-10¹² and overlapping ranges therein). In one embodiment, a dose escalation regimen is used. In some embodiments, a dose of immune cells comprises NK cells. In some embodiments, a dose of immune cells comprises T cells. In some embodiments, a dose of immune cells comprises NK cells and T cells. In several embodiments, a range of NK cells is administered, for example between about 1×10⁶ cells/kg to about 1×10⁸ cells/kg. In several embodiments, dosing is, for example, 3 doses of about 1.0×10⁹ NK cells or about 1.5×10⁹ NK cells administered over about 21 to 28 days. In several embodiments, a range of T cells is administered, for example between about 1×10⁶ cells/kg to about 1×10⁸ cells/kg.

For the prevention or treatment of cancer, the appropriate dosage may depend on the type of cancer to be treated, the type of cells or recombinant receptors, the severity and course of the cancer, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

In some embodiments, the engineered immune cells are administered as part of a combination treatment, such as simultaneously with or sequentially with, in any order, another or additional therapeutic intervention, such as an antibody or engineered cell or receptor or agent, such as a cytotoxic or therapeutic agent. The engineered immune cells in some embodiments are co-administered with one or more additional therapeutic agents or in connection with another therapeutic intervention, either simultaneously or sequentially in any order. In some embodiments, the additional therapeutic agent is any interventions or agents described herein, such as any interventions or agents descried that can ameliorate symptoms of toxicity. In some contexts, the engineered immune cells are co-administered with another therapy sufficiently close in time such that the engineered immune cell populations enhance the effect of one or more additional therapeutic agents, or vice versa. In some embodiments, the engineered immune cells are administered prior to the one or more additional therapeutic agents. In some embodiments, the engineered immune cells are administered after the one or more additional therapeutic agents. In some embodiments, the one or more additional agents include a cytokine, such as IL-2, for example, to enhance persistence. In some embodiments, the methods comprise administration of a chemotherapeutic agent. In some embodiments, the methods comprise administration of a chemotherapeutic agent, e.g., a conditioning chemotherapeutic agent, for example, to reduce tumor burden prior to the administration, including as described in the subsequent section.

The dose of the additional agent can be any therapeutically effective amount, e.g., any dose amount described herein, and the appropriate dosage of the additional agent may depend on the type of disease to be treated, the type, dose and/or frequency of the binding molecule, recombinant receptor, cell and/or composition administered, the severity and course of the disease, previous therapy, the patient's clinical history and response to cell therapy, and the discretion of the attending physician.

In several embodiments, one or more of cyclophosphamide, cytosine arabinoside, an anti-CD20 antibody (a non-limiting embodiment of which is rituximab), and/or an anti-EGFR antibody (a non-limiting embodiment of which is cetuximab) is administered on connection with treatment. Depending on the embodiment, various types of cancer or infection disease can be treated.

### Compositions and Formulations

Provided herein are compositions and formulations comprising a population of immune cells (e.g., NK cells) as described herein. In some embodiments, a cell dose comprising a population of immune cells (e.g., NK cells expressing a chimeric receptor such as a CAR) is provided as a composition or formulation, such as a pharmaceutical composition or formulation. Such compositions can be used in accordance with and/or with provided articles or compositions, such as for the prevention or treatment of diseases, conditions, and disorders, or in detection, diagnosis, and prognosis methods.

The term “pharmaceutical formulation” refers to a formulation in a form that allows the biological activity of the active ingredient contained therein to be effective and that is free of additional components having unacceptable toxicity to the subject to which the formulation will be applied.

By “pharmaceutically acceptable carrier” is meant an ingredient of a pharmaceutical formulation that is non-toxic to a subject, except for the active ingredient. Pharmaceutically acceptable carriers include, but are not limited to, buffers, excipients, stabilizers, or preservatives.

In some aspects, the choice of carrier depends in part on the particular cell or agent and/or the method of administration. Thus, there are a variety of suitable formulations. For example, the pharmaceutical composition may contain a preservative. Suitable preservatives may include, for example, methyl paraben, propyl paraben, sodium benzoate and benzalkonium chloride. In some aspects, a mixture of two or more preservatives is used. Preservatives or mixtures thereof are typically present in amounts of about 0.0001% to about 2% by weight of the total composition Pharmaceutically acceptable carriers are generally non-toxic to recipients at the dosages and concentrations used, and include, but are not limited to: buffers such as phosphate, citrate and other organic acids; antioxidants, including ascorbic acid and methionine; preservatives (such as octadecyl dimethyl benzyl ammonium chloride; hexamethonium chloride; benzalkonium chloride; benzethonium chloride; phenol, butanol or benzyl alcohol; alkyl parabens, such as methyl or propyl paraben, catechol; resorcinol; cyclohexanol; 3-pentanol; and m-cresol); low molecular weight (less than about 10 residues) polypeptides; proteins, such as serum albumin, gelatin or immunoglobulins; hydrophilic polymers such as polyvinylpyrrolidone; amino acids such as glycine, glutamine, asparagine, histidine, arginine or lysine; monosaccharides, disaccharides, and other carbohydrates including glucose, mannose, or dextrins; chelating agents, such as EDTA; sugars such as sucrose, mannitol, trehalose or sorbitol; salt-forming counterions, such as sodium; metal complexes (e.g., zinc-protein complexes); and/or a non-ionic surfactant, such as polyethylene glycol (PEG).

In some aspects, a buffer is included in the composition. Suitable buffering agents include, for example, citric acid, sodium citrate, phosphoric acid, potassium phosphate, and various other acids and salts. In some aspects, a mixture of two or more buffers is used. The buffering agent or mixtures thereof are typically present in an amount of from about 0.001% to about 4% by weight of the total composition. Methods for preparing administrable pharmaceutical compositions are known.

The formulation or composition may also contain more than one active ingredient that may be used for a particular indication, disease or condition that is prevented or treated with the cell or agent, where the respective activities do not adversely affect each other. Such active ingredients are present in combination in an amount effective for the intended purpose in a suitable manner. Thus, in some embodiments, the pharmaceutical composition further comprises other pharmaceutically active agents or drugs, such as chemotherapeutic agents, e.g., asparaginase, busulfan, carboplatin, cetuximab, cisplatin, daunomycin, doxorubicin, fluorouracil, gemcitabine, hydroxyurea, methotrexate, paclitaxel, rituximab, vinblastine, vincristine, and the like. In some embodiments, the agent or cell is administered in the form of a salt (e.g., a pharmaceutically acceptable salt). Suitable pharmaceutically acceptable acid addition salts include those derived from inorganic acids (such as hydrochloric, hydrobromic, phosphoric, metaphosphoric, nitric and sulfuric) and organic acids (such as tartaric, acetic, citric, malic, lactic, fumaric, benzoic, glycolic, gluconic, succinic and arylsulfonic, e.g., p-toluenesulfonic acid).

In some embodiments, the pharmaceutical composition contains the agent or cell in an amount effective to treat or prevent the disease or condition (e.g., a therapeutically effective amount or a prophylactically effective amount). In some embodiments, treatment or prevention efficacy is monitored by periodic assessment of the treated subject. For repeated administrations over several days or longer, depending on the condition, the treatment is repeated until the desired suppression of disease symptoms occurs. However, other dosage regimens may be useful and may be determined. The desired dose may be delivered by administering the composition as a single bolus, by administering the composition as multiple boluses, or by administering the composition as a continuous infusion.

Formulations include those for oral, intravenous, intraperitoneal, subcutaneous, pulmonary, transdermal, intramuscular, intranasal, buccal, sublingual, or suppository administration. In some embodiments, the agent or cell population is administered to the subject by intravenous, intraperitoneal, or subcutaneous injection using peripheral systemic delivery.

In some embodiments, the compositions are provided as sterile liquid formulations (e.g., isotonic aqueous solutions, suspensions, emulsions, dispersions, or viscous compositions), which in some aspects may be buffered to a selected pH. Liquid formulations are generally easier to prepare than gels, other viscous compositions, and solid compositions. In addition, liquid compositions are somewhat more convenient to administer, particularly by injection. The liquid composition can comprise a carrier, which can be a solvent or dispersion medium containing, for example, water, saline, phosphate buffered saline, polyols (e.g., glycerol, propylene glycol, liquid polyethylene glycol), and suitable mixtures thereof.

Sterile injectable solutions can be prepared by incorporating the agent or cell into a solvent, such as an admixture with a suitable carrier, diluent, or excipient (e.g., sterile water, saline, glucose, dextrose, and the like).

Formulations for in vivo administration are typically sterile. Sterility can be readily achieved, for example, by filtration through sterile filtration membranes. In some embodiments, the dose of engineered cells administered is in a cryopreserved composition. In some aspects, the composition is administered after thawing the cryopreserved composition

### Articles and Kits

Also provided are articles of manufacture and kits comprising (i) any of the compositions described herein; and (ii) instructions for administering the composition to a subject.

In some embodiments, an article of manufacture or kit comprises one or more containers (typically a plurality of containers), packaging material, and a label or package insert located on or associated with the one or more containers and/or packages, the label or package insert typically comprising instructions for performing any of the methods provided herein, e.g., for administering a population of immune cells to a subject. In some embodiments, the instructions provide guidance or assignment methods for assessing whether a subject is likely or suspected to be likely to respond prior to receiving the engineered immune and/or the extent or level of response after administration of the population of immune cells. In some aspects, the article of manufacture may contain a dose or composition of immune cells.

The articles provided herein contain packaging materials. Packaging materials for packaging provided materials are well known to those skilled in the art. Examples of packaging materials include, but are not limited to, blister packs, bottles, tubes, inhalers, pumps, bags, vials, containers, syringes, disposable laboratory items (e.g., pipette tips and/or plastic sheets), or bottles. The article or kit may include means to facilitate dispensing of materials or to facilitate use in a high throughput or large-scale manner, for example to facilitate use in a robotic device. Typically, the package does not react with the composition contained therein.

In some embodiments, the reagents and/or cell compositions are packaged separately. In some embodiments, each vessel may have a single compartment. In some embodiments, the other components of the article of manufacture or kit are packaged separately, or together in a single compartment.

### Definitions

The terms described below, or elsewhere herein, shall be understood to have their ordinary meaning and shall also be understood to have the meanings specifically described herein, unless otherwise specifically indicated.

The terms “polypeptide” and “protein” are used interchangeably to refer to a polymer of amino acid residues, and are not limited to a minimum length. Polypeptides, including the provided receptors and other polypeptides, e.g., linkers or peptides, may include amino acid residues including natural and/or non-natural amino acid residues. The terms also include post-expression modifications of the polypeptide, for example, glycosylation, sialylation, acetylation, and phosphorylation. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

As used herein, a “subject” is a mammal, such as a human or other animal, and typically is human. In some embodiments, the subject, e.g., patient, to whom the agent or agents, cells, cell populations, or compositions are administered, is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

“Preventing” (and grammatical variations thereof such as “prevent” or “prevention”) as used herein, includes providing prophylaxis with respect to the occurrence or recurrence of a disease in a subject that may be predisposed to the disease but has not yet been diagnosed with the disease. In some embodiments, the provided cells and compositions are used to delay development of a disease or to slow the progression of a disease.

A “therapeutically effective amount” of an agent, e.g., a pharmaceutical formulation or cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the cells and/or compositions at effective amounts, e.g., therapeutically effective amounts.

As used herein, the singular forms “a,” “an,” and “the” include plural referents unless the context clearly dictates otherwise. For example, “a” or “an” means “at least one” or “one or more.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. Language such as “up to,” “at least,” “greater than,” “less than,” “between,” and the like includes the number recited. Numbers preceded by a term such as “about” or “approximately” include the recited numbers. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the corresponding value that is readily known. Reference herein to “about” a value or parameter includes (and describes) embodiments that relate to that value or parameter per se. For example, a description referring to “about X” includes a description of “X”. In certain embodiments, “about X” refers to a value of ±25%, ±10%, ±5%, ±2%, ±1%, ±0.1% or ±0.01% of X.

In addition, when a sequence is disclosed as “comprising” a nucleotide or amino acid sequence, such a reference shall also include, unless otherwise indicated, that the sequence “comprises”, “consists of” or “consists essentially of” the recited sequence.

As used herein, a statement that a cell or population of cells is “positive” for a particular marker refers to the detectable presence of the particular marker (typically a surface marker) on or in the cell. When referring to a surface marker, the term refers to the presence of surface expression as detected by flow cytometry, for example by staining with an antibody that specifically binds to the marker and detecting the antibody, wherein the staining is detectable by flow cytometry at a level that is substantially higher than the staining detected by the same procedure with an isotype matched control under otherwise identical conditions, and/or that is substantially similar to the level of cells known to be positive for the marker, and/or that is substantially higher than the level of cells known to be negative for the marker.

As used herein, a statement that a cell or cell population is “negative” for a particular marker means that the particular marker (typically a surface marker) is not present on or in the cell in a substantially detectable presence. When referring to a surface marker, the term refers to the absence of surface expression as detected by flow cytometry, for example by staining with an antibody that specifically binds to the marker and detecting the antibody, wherein the staining is not detected by flow cytometry at a level that is substantially higher than the staining detected by the same procedure with an isotype matched control under otherwise identical conditions, and/or at a level that is substantially lower than the level of cells known to be positive for the marker, and/or at a level that is substantially similar compared to the level of cells known to be negative for the marker.

As used herein, “percent” (%) amino acid sequence identity” and “percent identity,” when used in reference to an amino acid sequence (a reference polypeptide sequence), is defined as the percentage of amino acid residues in a candidate sequence (e.g., a subject antibody or fragment) that are identical to the amino acid residues in the reference polypeptide sequence, after aligning the sequences and introducing gaps, if necessary, to achieve the maximum percent sequence identity, and without considering any conservative substitutions as part of the sequence identity. Alignment for the purpose of determining percent amino acid sequence identity can be accomplished in a variety of known ways, for example, using publicly available computer software. Appropriate parameters for aligning the sequences can be determined, including any algorithms necessary to achieve maximum alignment over the full length of the sequences being compared.

Amino acid substitutions can include the substitution of one amino acid for another in a polypeptide. Substitutions may be conservative amino acid substitutions or non-conservative amino acid substitutions. Amino acid substitutions may be introduced into the binding molecule of interest (e.g., an antibody) and the product screened for the desired activity (e.g., retained/improved antigen binding, reduced immunogenicity, or improved ADCC or CDC).

Amino acids can be generally grouped according to the following common side chain properties.

In some embodiments, conservative substitutions may include exchanging a member of one of these classes for another member of the same class. In some embodiments, a non-conservative amino acid substitution may involve exchanging a member of one of these classes for another class.
(1) hydrophobicity: Norleucine, Met, Ala, Val, Leu, Ile;
(2) neutral hydrophilicity: Cys, Ser, Thr, Asn, Gln;
(3) acidity: Asp and Glu;
(4) alkalinity: His, Lys, Arg;
(5) residues that influence chain orientation: Gly, Pro;
(6) aromatic: Trp, Tyr, Phe.

As used herein, a composition refers to any mixture of two or more products, substances, or compounds, including cells. It may be a solution, a suspension, liquid, powder, a paste, aqueous, non-aqueous or any combination thereof.

### Non-Limiting Embodiments

Among the embodiments provided herein are:
1. A population of genetically engineered and gene edited immune cells, comprising genetically engineered immune cells that express a cytotoxic receptor comprising an extracellular ligand binding domain, a transmembrane domain, and a cytotoxic signaling complex, wherein:
the extracellular ligand binding domain targets an antigen expressed by cells of a target tumor or cancer;
the immune cells are genetically edited within a target sequence in a MED12 gene comprising any one of SEQ ID NOS: 938-944 or 996-998;
the edit yields reduced expression and/or function of the MED12 protein encoded by the MED12 gene, as compared to an immune cell not edited within the target sequence in the MED12 gene; and
the edit to the MED12 gene is made using an RNA-guided endonuclease.
2. A population of genetically engineered and gene edited immune cells, comprising genetically engineered immune cells that express a cytotoxic receptor comprising an extracellular ligand binding domain, a transmembrane domain, and a cytotoxic signaling complex, wherein:
the immune cells are natural killer (NK) cells;
the extracellular ligand binding domain targets an antigen expressed by cells of a target tumor or cancer;
the immune cells are genetically edited within a target sequence in a MED12 gene comprising any one of SEQ ID NOS: 938-944 or 996-998;
the edit yields reduced expression and/or function of the MED12 protein encoded by the MED12 gene, as compared to an immune cell not edited within the target sequence in the MED12 gene; and
the edit to the MED12 gene is made using an RNA-guided endonuclease.
3. A population of genetically engineered and gene edited immune cells, comprising genetically engineered immune cells that express a cytotoxic receptor comprising an extracellular ligand binding domain, a transmembrane domain, and a cytotoxic signaling complex, wherein:
the extracellular ligand binding domain targets an antigen expressed by cells of a target tumor or cancer;
the immune cells are genetically edited within a target sequence in a MED12 gene and within a target sequence in a CISH gene;
the edits yield reduced expression and/or function of each of the MED12 protein encoded by the MED12 gene and the CIS protein encoded by the CISH gene, as compared to an immune cell not edited within the target sequence in the MED12 gene and the target sequence within the CISH gene.
4. The population of genetically engineered and gene edited immune cells of Embodiment 3, wherein the edits to the MED12 and CISH genes are made using an RNA-guided endonuclease.
5. The population of genetically engineered and gene edited immune cells of any one of Embodiments 1, 3, and Embodiment 4, wherein the immune cells are natural killer (NK) cells.
6. A population of genetically engineered and gene edited immune cells, comprising:
genetically engineered immune cells that express a cytotoxic receptor comprising an extracellular ligand binding domain, a transmembrane domain, and a cytotoxic signaling complex,
wherein the extracellular ligand binding domain targets a tumor marker expressed by a target tumor cell,
wherein the immune cells are genetically edited at one or more locations in an ADAM17 target gene that encodes a corresponding protein,
wherein the edits yield reduced expression and/or function of a corresponding ADAM17 protein as compared to an immune cell not edited at the location or locations in the ADAM17 gene;
wherein the immune cells are optionally edited at one or more additional target sites in the genome of the immune cell to yield reduced levels of expression of the protein which is encoded by a gene which comprises an edited target site as compared to a non-edited immune cell,
wherein the edits to the target gene or target genes are made using an RNA-guided endonuclease, and
wherein the genetically engineered and edited immune cells exhibit one or more of enhanced expansion capability, enhanced cytotoxicity against target tumor cells, and enhanced persistence, as compared to immune cells that do not comprise said genetically edited target site or sites.
7. A population of genetically engineered and gene edited immune cells, comprising:
genetically engineered immune cells that express a cytotoxic receptor comprising an extracellular ligand binding domain, a transmembrane domain, and a cytotoxic signaling complex,
wherein the extracellular ligand binding domain targets a tumor marker expressed by a target tumor cell,
wherein the immune cells are genetically edited at one or more locations in an HIF1-a target gene that encodes a corresponding HIF1-a protein,
wherein the edits yield reduced expression and/or function of the corresponding HIF1-a protein as compared to an immune cell not edited at the location or locations in the HIF1-a gene;
wherein the immune cells are optionally edited at one or more additional target sites in the genome of the immune cell to yield reduced levels of expression of the protein which is encoded by a gene which comprises an edited target site as compared to a non-edited immune cell,
wherein the edits to the target gene or target genes are made using an RNA-guided endonuclease, and
wherein the genetically engineered and edited immune cells exhibit one or more of enhanced expansion capability, enhanced cytotoxicity against target tumor cells, and enhanced persistence, as compared to immune cells that do not comprise said genetically edited target site or sites.
8. The population of genetically engineered and gene edited immune cells of any one of Embodiments 1-7, wherein the immune cells are edited within an additional target sequence in a target gene to yield reduced levels of expression of a protein encoded by the target gene, as compared to an immune cell not edited within the additional target sequence.
9. A population of genetically engineered and gene edited immune cells, comprising:
genetically engineered immune cells that express a cytotoxic receptor comprising an extracellular ligand binding domain, a transmembrane domain, and a cytotoxic signaling complex,
wherein the extracellular ligand binding domain targets a tumor marker expressed by a target tumor cell,
wherein the immune cells are genetically edited at one or more locations in a target gene selected from ADAM17, HIF-1a, DGKz, GSK-3B, LAG3, TIM3, TRIM29, IL-1R8, CD38, FBP-1, INSIG1, MED12, MED 13, CCNC, CDK8, TD3, SOX4, or any combination thereof,
wherein each of said genes encodes a corresponding protein
wherein the edits yield reduced expression and/or function of the corresponding protein as compared to an immune cell not edited at the location or locations in the respective gene;
wherein the immune cells are edited at one or more additional target sites in the genome of the immune cell to yield reduced levels of expression of the protein which is encoded by a gene which comprises an edited target site as compared to a non-edited immune cell,
wherein the edits to the target gene or target genes are made using an RNA-guided endonuclease, and
wherein the genetically engineered and edited immune cells exhibit one or more of enhanced expansion capability, enhanced cytotoxicity against target tumor cells, and enhanced persistence, as compared to immune cells that do not comprise said genetically edited target site or sites.
10. The population of genetically engineered and gene edited immune cells of any one of Embodiments 1-9, wherein the edit(s) are made using a Crispr/Cas9 system.
11. The population of genetically engineered and gene edited immune cells of any one of Embodiments 1 to 10, wherein the extracellular ligand binding domains targets an antigen selected from BCMA, a NKG2D ligand, CD19, and CD70.
12. The population of genetically engineered and gene edited immune cells of any one of Embodiments 1 to 11, wherein the extracellular ligand binding domains target BCMA.
13. A population of genetically engineered and gene edited immune cells, comprising:
genetically engineered immune cells that express a cytotoxic receptor comprising an extracellular ligand binding domain, a transmembrane domain, and a cytotoxic signaling complex,
wherein the extracellular ligand binding domain targets a tumor marker expressed by a target tumor cell,
wherein the immune cells are genetically edited at one or more locations in a target gene selected from ADAM17, HIF-1a, DGKz, GSK-3B, LAG3, TIM3, TRIM29, IL-1R8, CD38, FBP-1, INSIG1, MED12, MED13, CCNC, CDK8, ID3, SOX4, or any combination thereof,
wherein each of said genes encodes a corresponding protein
wherein the edits yield reduced expression and/or function of the corresponding protein as compared to an immune cell not edited at the location or locations in the respective gene;
wherein the immune cells are edited at one or more additional target sites in the genome of the immune cell to yield reduced levels of expression of the protein which is encoded by a gene which comprises an edited target site as compared to a non-edited immune cell,
wherein the edits to the target gene or target genes are made using a Crispr/Cas9 system, and
wherein the genetically engineered and edited immune cells exhibit one or more of enhanced expansion capability, enhanced cytotoxicity target tumor cells, and enhanced persistence, as compared to immune cells that do not comprise said genetically edited target site or sites.
14. A population of genetically engineered and gene edited immune cells, comprising:
genetically engineered immune cells that express a cytotoxic receptor comprising an extracellular ligand binding domain, a transmembrane domain, and a cytotoxic signaling complex,
wherein the extracellular ligand binding domain targets a tumor marker expressed by a target tumor cell,
wherein the tumor marker expressed by the target tumor cell is selected from a ligand of the NKG2D receptor, CD19, or CD70;
wherein the immune cells are genetically edited at one or more locations in a target gene selected from ADAM17, HIF-1a, DGKz, GSK-3B, LAG3, TIM3, TRIM29, IL-1R8, CD38, FBP-1, INSIG1, MED12, MED13, CCNC, CDK8, ID3, SOX4, or any combination thereof,
wherein each of said genes encodes a corresponding protein
wherein the edits yield reduced expression and/or function of the corresponding protein as compared to an immune cell not edited at the location or locations in the respective gene;
wherein the immune cells are edited at one or more additional target sites in the genome of the immune cell to yield reduced levels of expression of the protein which is encoded by a gene which comprises an edited target site as compared to a non-edited immune cell,
wherein the edits to the target gene or target genes are made using a Crispr/Cas system, and
wherein the genetically engineered and edited immune cells exhibit one or more of enhanced expansion capability, enhanced cytotoxicity against target tumor cells, and enhanced persistence, as compared to immune cells that do not comprise said genetically edited target site or sites.
15. A population of genetically engineered and gene edited immune cells, comprising genetically engineered immune cells that express a cytotoxic receptor comprising an extracellular ligand binding domain, a transmembrane domain, and a cytotoxic signaling complex, wherein:
the extracellular ligand binding domain targets an antigen expressed by cells of a target tumor or cancer selected from BCMA, a NKG2D ligand, CD19, and CD70;
the immune cells are genetically edited within a target sequence in a target gene selected from MED12, ADAM17, HIF-1a, DGKz, GSK-3B, LAG3, TIM3, TRIM29, IL-1R8, CD38, FBP-1, INSIG1, MED13, CCNC, CDK8, ID3, SOX4, and any combination thereof;
the edit yields reduced expression and/or function of the protein encoded by the target gene, as compared to an immune cell not edited within the target sequence in the target gene;
the immune cells are edited within an additional target sequence in a target gene to yield reduced levels of expression of the protein encoded by the target gene, as compared to an immune cell not edited within the additional target sequence; and
the edit(s) are made using a Crispr/Cas system.
16. The population of genetically engineered and gene edited immune cells of any one of Embodiments 1 to 5, 8, and 10-12, wherein the genetically engineered and gene edited immune cells exhibit enhanced expansion capability, enhanced cytotoxicity against target tumor cells, enhanced persistence, or any combination thereof, as compared to immune cells that do not comprise the edit(s).
17. The population of genetically engineered and gene edited immune cells of any one of Embodiments 1 to 18, wherein ADAM17 is edited and wherein a guide sequence of any of SEQ ID NO: 682-687 is used to target the ADAM17 gene.
18. The population of genetically engineered and gene edited immune cells of any one of Embodiments 1 to 17, wherein HIF-1a is edited and wherein a guide sequence of any of SEQ ID NO: 750-760 is used to target the HIF-1a gene.
19. The population of genetically engineered and gene edited immune cells of any one of Embodiments 1 to 18, wherein DGKz is edited and wherein a guide sequence of any of SEQ ID NO: 688-723 is used to target the DGKz gene.
20. The population of genetically engineered and gene edited immune cells of any one of Embodiments 1 to 19, wherein GSK-3B is edited and wherein a guide sequence of any of SEQ ID NO: 724-749 is used to target the GSK-3B gene.
21. The population of genetically engineered and gene edited immune cells of any one of Embodiments 1 to 209, wherein LAG3 is edited and wherein a guide sequence of any of SEQ ID NO: 761-789 is used to target the LAG3 gene.
22. The population of genetically engineered and gene edited immune cells of any one of Embodiments 1 to 21, wherein TIM3 is edited and wherein a guide sequence of any of SEQ ID NO: 790-825 is used to target the TIM3 gene.
23. The population of genetically engineered and gene edited immune cells of any one of Embodiments 1 to 22, wherein TRIM29 is edited and wherein a guide sequence of any of SEQ ID NO: 826-835 or 1009-1011 is used to target the TRIM29 gene.
24. The population of genetically engineered and gene edited immune cells of any one of Embodiments 1 to 23, wherein IL-1R8 is edited and wherein a guide sequence of any of SEQ ID NO: 836-865 is used to target the IL-1R8 gene.
25. The population of genetically engineered and gene edited immune cells of any one of Embodiments 1 to 24, wherein CD38 is edited and wherein a guide sequence of any of SEQ ID NO: 866-874 is used to target the CD38 gene.
26. The population of genetically engineered and gene edited immune cells of any one of Embodiments 1 to 25, wherein FBP-1 is edited and wherein a guide sequence of any of SEQ ID NO: 875-889 is used to target the FBP-1 gene.
27. The population of genetically engineered and gene edited immune cells of any one of Embodiments 1 to 26, wherein INSIG1 is edited and wherein a guide sequence of any of SEQ ID NO: 890-934 is used to target the INSIG1 gene.
28. The population of genetically engineered and gene edited immune cells of any one of Embodiments 1 to 27, wherein MED12 is edited and wherein a guide sequence of any of SEQ ID NO: 938-944 or 996-998 is used to target the MED12 gene.
29. The population of genetically engineered and gene edited immune cells of any one of Embodiments 1 to 28, wherein CDK8 is edited and wherein a guide sequence of any of SEQ ID NO: 949-955 is used to target the CDK8 gene.
30. The population of genetically engineered and gene edited immune cells of any one of Embodiments 1 to 29, wherein CCNC is edited and wherein a guide sequence of any of SEQ ID NO: 956-961 is used to target the CCNC gene.
31. The population of genetically engineered and gene edited immune cells of any one of Embodiments 1 to 30, wherein ID3 is edited and wherein a guide sequence of any of SEQ ID NO: 963-969 is used to target the ID3 gene.
32. The population of genetically engineered and gene edited immune cells of any one of Embodiments 1 to 31, wherein SOX4 is edited and wherein a guide sequence of any of SEQ ID NO: 970-976 is used to target the SOX4 gene.
33. The population of genetically engineered and gene edited immune cells of any of Embodiments 1 to 32, wherein the immune cells are further edited at a CISH gene that encodes a CIS protein.
34. The population of genetically engineered and gene edited immune cells of Embodiment 33, wherein a guide sequence of any of SEQ ID NO: 153-157, 463-466, or 1012-1013 is used to target the CISH gene.
35. The population of genetically engineered and gene edited immune cells of any one of Embodiments 1 to 34, wherein the cells are edited at an additional target site in a CBLB gene.
36. The population of genetically engineered and gene edited immune cells of Embodiment 35, wherein a guide sequence of any of SEQ ID NO: 164 to 166, 453-456, or 1005-1008 is used to target the CBLB gene.
37. The population of genetically engineered and gene edited immune cells of any one of Embodiments 1 to 36, wherein the cells are optionally edited at a gene encoding CD70.
38. The population of genetically engineered and gene edited immune cells of any one of Embodiments 1 to 37, wherein the cells are optionally edited at a TGFBR2 gene, a TIGIT gene, an adenosine A2 receptor gene, a SMAD3 gene, a MAPKAPK3 gene, a CEACAM1 gene, a DDIT4 gene, an NKG2A gene, a SOCS2 gene, a B2M gene, a PD-1gene, and/or a TCR alpha gene.
39. The population of genetically engineered and gene edited immune cells of any one of Embodiments 1 to 38, wherein the transmembrane domain comprises CD8, CD28, or a portion thereof, optionally wherein the transmembrane domain comprises CD8a or a portion thereof.
40. The population of genetically engineered and gene edited immune cells of any one of Embodiments 1 to 39, wherein the cytotoxic signaling complex comprises a CD3zeta domain and an intracellular signaling domain of an OX40, 4-1 BB, CD28, or a signaling portion thereof, optionally an intracellular signaling domain of OX40 or a signaling portion thereof.
41. The population of genetically engineered and gene edited immune cells of any one of Embodiments 1 to 40, wherein at least a portion of the genetically engineered immune cells are engineered to express membrane bound IL-15.
42. The population of genetically engineered and gene edited immune cells of Embodiment 41, wherein the cytotoxic receptor and the mbIL15 are encoded by the same nucleic acid molecule, optionally wherein the nucleic acid sequences encoding the cytotoxic receptor and the mbIL15 are separated by a nucleic acid sequence encoding a 2A peptide.
43. The population of genetically engineered and gene edited immune cells of any one of Embodiments 1 to 42, wherein the immune cells comprise Natural Killer (NK) cells, T cells, induced pluripotent stem cells (iPSCs), iPSC-derived NK cells, NK-92 cells, or combinations thereof.
44. The population of genetically engineered and gene edited immune cells of any one of Embodiments 1 to 43, wherein the immune cells comprise NK cells.
45. The population of genetically engineered and gene edited immune cells of any one of Embodiments 1 to 44, wherein the genetically engineered and edited immune cells are suitable for use in allogeneic cancer cell therapy and wherein the cells maintain enhanced cytotoxicity and/or persistence in a hypoxic tumor microenvironment.
46. A composition comprising the population of genetically engineered and gene edited immune cells of any one of Embodiments 1 to 45.
47. The composition of Embodiment 46, further comprising a pharmaceutically acceptable excipient.
48. A method for the treatment of cancer in a subject comprising administering to the subject at least a portion of population of immune cells according to any of Embodiments 1 to 45.
49. Use of genetically engineered and edited immune cells according to any of Embodiments 1 to 45 for the treatment of cancer.
50. Use of genetically engineered and edited immune cells according to any of Embodiments 1 to 45 for the preparation of a medicament for the treatment of cancer.
51. A method for the treatment of a subject having a disease or condition comprising administering to the subject a population of genetically engineered and gene edited natural killer (NK) cells that express a cytotoxic receptor comprising an extracellular ligand binding domain, a transmembrane domain, and a cytotoxic signaling complex, wherein:
the extracellular ligand binding domain targets an antigen expressed by cells of the disease or condition;
the immune cells are genetically edited within a target sequence in a MED12 gene comprising any one of SEQ ID NOS: 938-944 or 996-998;
the edit yields reduced expression and/or function of the MED12 protein encoded by the MED12 gene, as compared to an immune cell not edited within the target sequence in the MED12 gene; and
the edit to the MED12 gene is made using an RNA-guided endonuclease.
52. A method for the treatment of a subject having a disease or condition comprising administering to the subject a population of genetically engineered and gene edited natural killer (NK) cells that express a cytotoxic receptor comprising an extracellular ligand binding domain, a transmembrane domain, and a cytotoxic signaling complex, wherein:
the extracellular ligand binding domain targets an antigen expressed by cells of the disease or condition;
the immune cells are genetically edited within a target sequence in a MED12 gene and within a target sequence in a CISH gene;
the edits yield reduced expression and/or function of each of the MED12 protein encoded by the MED12 gene and the CIS protein encoded by the CISH gene, as compared to a NK cell not edited within the target sequence in the MED12 gene and the target sequence within the CISH gene.
53. The method of any one of Embodiments 48, 51, and 52, or the use of Embodiment 49 or 50, wherein the disease or condition is a cancer.
54. The method of any one of Embodiments 48 and 51-53 or the use of any one of Embodiments 49, 50, and 53. wherein the immune cells are allogeneic to the subject.
55. A method of manufacturing a population of genetically edited immune cells for cancer immunotherapy, comprising:
contacting the population of immune cells with a targeted endonuclease,
wherein the targeted endonuclease cuts nucleic acid at two or more target sites in a target gene selected from ADAM17, HIF-1a, DGKz, GSK-3B, LAG3, TIM3, TRIM29, IL-1R8, CD38, FBP-1, INSIG1, MED12, MED13, CCNC, CDK8, ID3, SOX4, CISH, CBLB, or any combination thereof, and
wherein the genetically edited immune cells exhibit one or more of enhanced expansion capability, enhanced cytotoxicity against target tumor cells, and enhanced persistence, as compared to immune cells that do not comprise said genetically edited target site or sites.
56. A method of manufacturing a population of genetically edited immune cells for cancer immunotherapy, comprising:
contacting the population of immune cells with a RNA guided endonuclease,
wherein the RNA guided endonuclease edits at one or more target sites in a target gene selected from ADAM17, HIF-1a, DGKz, GSK-3B, LAG3, TIM3, TRIM29, IL-1R8, CD38, FBP-1, INSIG1, MED12, MED13, CCNC, CDK8, ID3, SOX4 or any combination thereof, and
wherein the genetically edited immune cells exhibit one or more of enhanced expansion capability, enhanced cytotoxicity against target tumor cells, and enhanced persistence, as compared to immune cells that do not comprise said genetically edited target site or sites.
57. A method of manufacturing a population of genetically edited immune cells for cancer immunotherapy, comprising:
contacting the population of immune cells with a Cas-gRNA ribonucleoprotein complex (RNP),
wherein the RNP edits at one or more target sites in a target gene selected from ADAM17, HIF-1a, DGKz, GSK-3B, LAG3, TIM3, TRIM29, IL-1R8, CD38, FBP-1, INSIG1, MED12, MED13, CCNC, CDK8, ID3, SOX4 or any combination thereof,
wherein the Cas of the RNP comprises Cas9, CasX, CasY, or combinations thereof, and
wherein the genetically edited immune cells exhibit one or more of enhanced expansion capability, enhanced cytotoxicity against target tumor cells, and enhanced persistence, as compared to immune cells that do not comprise said genetically edited target site or sites.
58. A method of manufacturing a population of genetically edited immune cells for cancer immunotherapy, comprising:
contacting the population of immune cells with a first RNA guided endonuclease,
wherein the endonuclease edits at one or more target sites in a target gene selected from ADAM17, HIF-1a, DGKz, GSK-3B, LAG3. TIM3, TRIM29, IL-1R8, CD38, FBP-1, INSIG1, MED12, MED13, CCNC, CDK8, ID3, SOX4, or any combination thereof; and
contacting the population of immune cells with a second RNA guided endonuclease,
wherein the second endonuclease edits at one or more target sites in a in a CISH gene of the immune cell to yield reduced levels of expression of CIS protein encoded by the CISH gene as compared to an immune cell not edited at the CISH gene, and
wherein the genetically edited immune cells exhibit one or more of enhanced expansion capability, enhanced cytotoxicity against target tumor cells, and enhanced persistence, as compared to immune cells that do not comprise said genetically edited target site or sites.
59. A method of manufacturing a population of genetically edited immune cells for cancer immunotherapy, comprising:
contacting the population of immune cells with a first Cas-gRNA ribonucleoprotein complex (RNP),
wherein the RNP edits at one or more target sites wherein the RNP edits at one or more target sites in a target gene selected from ADAM17, HIF-1a, DGKz, GSK-3B, LAG3, TIM3, TRIM29, IL-1R8, CD38, FBP-1, INSIG1, MED12, MED13, CCNC, CDK8, ID3, SOX4, or any combination thereof
wherein the Cas of the RNP comprises Cas9, CasX, CasY, or combinations thereof, and
contacting the population of immune cells with a second RNP complex,
wherein the second RNP edits at one or more target sites in a in a CISH gene of the immune cell to yield reduced levels of expression of CIS protein encoded by the CISH gene as compared to an immune cell not edited at the CISH gene,
wherein the Cas of the RNP comprises Cas9, CasX, CasY, or combinations thereof, and
wherein the genetically edited immune cells exhibit one or more of enhanced expansion capability, enhanced cytotoxicity against target tumor cells, and enhanced persistence, as compared to immune cells that do not comprise said genetically edited target site or sites.
60. A method of manufacturing a population of genetically edited immune cells for cancer immunotherapy, comprising:
contacting the population of immune cells with a first Cas-gRNA ribonucleoprotein complex (RNP),
wherein the RNP edits at one or more target sites wherein the RNP edits at one or more target sites in a target gene selected from ADAM17, HIF-1a, DGKz, GSK-3B, LAG3, TIM3, TRIM29, IL-1R8, CD38, FBP-1, INSIG1, MED12, MED13, CCNC, CDK8, ID3, SOX4, or any combination thereof
wherein the Cas of the RNP comprises Cas9, CasX, CasY, or combinations thereof, and
contacting the population of immune cells with a second RNP complex,
wherein the second RNP edits at one or more target sites in a in a CBLB gene of the immune cell to yield reduced levels of expression of CBLB protein encoded by the CBLB gene as compared to an immune cell not edited at the CBLB gene,
wherein the Cas of the RNP comprises Cas9, CasX, CasY, or combinations thereof, and
wherein the genetically edited immune cells exhibit one or more of enhanced expansion capability, enhanced cytotoxicity against target tumor cells, and enhanced persistence, as compared to immune cells that do not comprise said genetically edited target site or sites.
61. A method of manufacturing a population of genetically edited immune cells for cancer immunotherapy, comprising:
contacting the population of immune cells with a first RNA-guided endonuclease,
wherein the first endonuclease edits at one or more target sites wherein the RNP edits at one or more target sites in a target gene selected from ADAM17, HIF-1a, DGKz, GSK-3B, LAG3, TIM3, TRIM29, IL-1R8, CD38, FBP-1, INSIG1, MED12, MED13, CCNC, CDK8, ID3, SOX4, or any combination thereof, and
contacting the population of immune cells with a second and a third RNA-guided endonuclease,
wherein the second RNA-guided endonuclease edits at one or more target sites in a in a CISH gene of the immune cell to yield reduced levels of expression of CIS protein encoded by the CISH gene as compared to an immune cell not edited at the CISH gene,
wherein the third RNA-guided endonuclease edits at one or more target sites in CBLB gene of the immune cell to yield reduced levels of expression of CBLB protein encoded by the CBLB gene as compared to an immune cell not edited at the CBLB gene, and
wherein the genetically edited immune cells exhibit one or more of enhanced expansion capability, enhanced cytotoxicity against target tumor cells, and enhanced persistence, as compared to immune cells that do not comprise said genetically edited target site or sites.
62. A method of manufacturing a population of genetically edited immune cells for cancer immunotherapy, comprising:
contacting the population of immune cells with a first Cas-gRNA ribonucleoprotein complex (RNP),
wherein the RNP edits at one or more target sites wherein the RNP edits at one or more target sites in a target gene selected from ADAM17, HIF-1a, DGKz, GSK-3B, LAG3, TIM3, TRIM29, IL-1R8, CD38, FBP-1, INSIG1, MED12, MED13, CCNC, CDK8, ID3, SOX4, or any combination thereof, and
contacting the population of immune cells with a second and a third RNP complex,
wherein the second RNP edits at one or more target sites in a in a CISH gene of the immune cell to yield reduced levels of expression of CIS protein encoded by the CISH gene as compared to an immune cell not edited at the CISH gene,
wherein the third RNP edits at one or more target sites in CBLB gene of the immune cell to yield reduced levels of expression of CBLB protein encoded by the CBLB gene as compared to an immune cell not edited at the CBLB gene,
wherein the Cas of each of the RNP comprises Cas9, CasX, CasY, or combinations thereof and
wherein the genetically edited immune cells exhibit one or more of enhanced expansion capability, enhanced cytotoxicity against target tumor cells, and enhanced persistence, as compared to immune cells that do not comprise said genetically edited target site or sites.
63. A method of manufacturing a population of genetically edited immune cells for cancer immunotherapy, comprising:
contacting the population of immune cells with a plurality of Cas-gRNA ribonucleoprotein complex (RNP),
wherein the plurality of RNP induces edits at one or more target sites in a CISH gene of the immune cell to yield reduced levels of expression of CIS protein encoded by the CISH gene as compared to an immune cell not edited at the CISH gene,
wherein the plurality of RNP induces edits at one or more target sites in CBLB gene of the immune cell to yield reduced levels of expression of CBLB protein encoded by the CBLB gene as compared to an immune cell not edited at the CBLB gene,
wherein the plurality of RNP induces edits at one or more target sites in a target gene selected from ADAM17, HIF-1a, DGKz, GSK-3B, LAG3, TIM3, TRIM29, IL-1R8, CD38, FBP-1, INSIG1, MED12, MED13, CCNC, CDK8, ID3, SOX4, or any combination thereof,
wherein the Cas of each of the plurality of RNP comprises Cas9, CasX, CasY, or combinations thereof, and
wherein the genetically edited immune cells exhibit one or more of enhanced expansion capability, enhanced cytotoxicity against target tumor cells, and enhanced persistence, as compared to immune cells that do not comprise said genetically edited target site or sites.
64. The method of any one of Embodiments 55 to 63, further comprising contacting the population of immune cells with a vector comprising a polynucleotide encoding a cytotoxic receptor comprising an extracellular ligand binding domain, a transmembrane domain, and a cytotoxic signaling complex.
65. The method of any one of Embodiments 55 to 64, wherein ADAM17 is edited and wherein a guide sequence of any of SEQ ID NO: 682-687 is used to target the ADAM17 gene.
66. The method of any one of Embodiments 55 to 65, wherein HIF-1a is edited and wherein a guide sequence of any of SEQ ID NO: 750-760 is used to target the HIF-1a gene.
67. The method of any one of Embodiments 55 to 66, wherein DGKz is edited and wherein a guide sequence of any of SEQ ID NO: 688-723 is used to target the DGKz gene.
68. The method of any one of Embodiments 55 to 67, wherein GSK-3B is edited and wherein a guide sequence of any of SEQ ID NO: 724-749 is used to target the GSK-3B gene.
69. The method of any one of Embodiments 55 to 68, wherein LAG3 is edited and wherein a guide sequence of any of SEQ ID NO: 761-789 is used to target the LAG3 gene.
70. The method of any one of Embodiments 55 to 69, wherein TIM3 is edited and wherein a guide sequence of any of SEQ ID NO: 790-825 is used to target the TIM3 gene.
71. The method of any one of Embodiments 55 to 70, wherein TRIM29 is edited and wherein a guide sequence of any of SEQ ID NO: 826-835 or 1009-1011 is used to target the TRIM29 gene.
72. The method of any one of Embodiments 55 to 71, wherein IL-1R8 is edited and wherein a guide sequence of any of SEQ ID NO: 836-865 is used to target the IL-1R8 gene.
73. The method of any one of Embodiments 55 to 72, wherein CD38 is edited and wherein a guide sequence of any of SEQ ID NO: 866-874 is used to target the CD38 gene.
74. The method of any one of Embodiments 55 to 73, wherein FBP-1 is edited and wherein a guide sequence of any of SEQ ID NO: 875-889 is used to target the FBP-1 gene.
75. The method of any one of Embodiments 55 to 74, wherein INSIG1 is edited and wherein a guide sequence of any of SEQ ID NO: 890-934 is used to target the INSIG1 gene.
76. The method of any one of Embodiments 55 to 75, wherein MED12 is edited and wherein a guide sequence of any of SEQ ID NO: 938-944 or 996-998 is used to target the MED12 gene, and optionally wherein MED13 is edited and wherein a guide sequence of any of SEQ ID NO: 945-948 is used to target the MED13 gene.
77. The method of any one of Embodiments 55 to 76, wherein CDK8 is edited and wherein a guide sequence of any of SEQ ID NO: 949-955 is used to target the CDK8 gene.
78. The method of any one of Embodiments 55 to 77, wherein CCNC is edited and wherein a guide sequence of any of SEQ ID NO: 956-962 is used to target the CCNC gene.
79. The method of any one of Embodiments 55 to 78, wherein ID3 is edited and wherein a guide sequence of any of SEQ ID NO: 963-969 is used to target the ID3 gene.
80. The method of any one of Embodiments 55 to 79, wherein SOX4 is edited and wherein a guide sequence of any of SEQ ID NO: 970-976 is used to target the SOX4 gene.
81. The method of any one of Embodiments 55 to 80, wherein CISH is edited and wherein a guide sequence of any of SEQ ID NO: 153-157, 463-466 or 1012-1013 is used to target the CISH gene.
82. The method of any one of Embodiments 55 to 81, wherein CBLB is edited and wherein a guide sequence of any of SEQ ID NO: 164 to 166, 453-456, or 105-108 is used to target the CBLB gene.
83. The method of any one of Embodiments 55 to 82, wherein the cells are optionally edited at a gene encoding CD70.
84. The method of any one of Embodiments 55 to 83, wherein the cells are optionally edited at a TGFBR2 gene, a TIGIT gene, an adenosine A2 receptor gene, a SMAD3 gene, a MAPKAPK3 gene, a CEACAM1 gene, a DDIT4 gene, an NKG2A gene, a SOCS2 gene, a B2M gene, a PD-1gene, and/or a TCR alpha gene.
85. The method of any one of Embodiments 55 to 84, wherein at least a portion of the genetically engineered immune cells are engineered to express membrane bound IL-15.
86. The method of any one of Embodiments 55 to 85, wherein the cells are edited within target sequences in the CISH and MED12 genes.
87. The method of any one of Embodiments 55 to 86, wherein the cells are edited within target sequences in the CISH, CBLB, and MED12 genes.
88. The method of any one of Embodiments 55 to 85, wherein the cells are edited at CISH, CBLB, and ADAM17.
89. The method of any one of Embodiments 55 to 85, wherein the cells are edited at CISH, CBLB, and HIF1a.
90. The method of any one of Embodiments 55 to 85, wherein the cells are edited at CISH, CBLB, and FBP-1.
91. The method of any one of Embodiments 64 to 90, wherein the extracellular ligand binding domain targets BCMA, CD19, CD70, or a NKG2D ligand.
92. The method of any one of Embodiments 64 to 91, wherein the extracellular ligand binding domain targets BCMA.
93. The method of any one of Embodiments 55 to 92, wherein the cells are also edited at CD70 and wherein the method further comprises contacting the population of immune cells with a vector comprising a polynucleotide encoding a cytotoxic receptor comprising an extracellular ligand binding domain that targets CD70, a transmembrane domain, and a cytotoxic signaling complex.
94. The method of any one of Embodiments 55 to 92, wherein the method further comprises contacting the population of immune cells with a vector comprising a polynucleotide encoding a cytotoxic receptor comprising an extracellular ligand binding domain that targets a tumor marker, a transmembrane domain, and a cytotoxic signaling complex.
95. The method of Embodiment 94, wherein the method does not comprise editing a gene encoding CD70.
96. The method of Embodiment 94 or 95, wherein the cytotoxic receptor does not target CD19.
97. The method of Embodiment 94, 95, or 96, wherein the cytotoxic receptor does not target NKG2D ligands.
98. The method of any one of Embodiments 55 to 97, wherein the immune cells comprise Natural Killer (NK) cells, T cells, induced pluripotent stem cells (iPSCs), iPSC-derived NK cells, NK-92 cells, or combinations thereof.
99. The method of any one of Embodiments 55 to 97, wherein the immune cells comprise Natural Killer (NK) cells.
100. A method for treating cancer in a subject comprising,
   administering to the subject a population of genetically engineered immune cells,
   wherein the immune cells express a cytotoxic receptor comprising an extracellular ligand binding domain, a transmembrane domain, and a cytotoxic signaling complex,
   wherein the immune cells are genetically edited at one or more target locations in a CISH gene that encodes a CIS protein, wherein the edits yield reduced expression and/or function of CIS as compared to an immune cell not edited at the location or locations in the CISH gene;
wherein the immune cells are edited at one or more target locations in one more target genes selected from ADAM17, HIF-1a, DGKz, GSK-3B, LAG3, TIM3, TRIM29, IL-1R8, CD38, FBP-1, INSIG1, MED12, MED13, CCNC, CDK8, ID3, SOX4, or any combination thereof,
wherein the edits are made using an RNA-guided endonuclease, and
wherein the genetically engineered and edited immune cells exhibit one or more of enhanced expansion capability, enhanced cytotoxicity against target tumor cells, and enhanced persistence, particularly in a hypoxic tumor microenvironment, as compared to immune cells that do not comprise said genetically edited target site or sites.
101. The method of embodiment 100, wherein the immune cells are genetically edited within a target sequence in the CBLB gene, wherein the edit yields reduced expression and/or function of the CBLB protein as compared to an immune cell that has not been edited within the target sequence in the CBLB gene.
102. A method for treating cancer in a subject comprising,
administering to the subject a population of genetically engineered immune cells,
wherein the immune cells express a cytotoxic receptor comprising an extracellular ligand binding domain, a transmembrane domain, and a cytotoxic signaling complex,
wherein the immune cells are genetically edited at one or more target locations in a CISH gene that encodes a CIS protein, wherein the edits yield reduced expression and/or function of CIS as compared to an immune cell not edited at the location or locations in the CISH gene;
wherein the immune cells are genetically edited at one or more target locations in a CBLB gene that encodes a CBLB protein, wherein the edits yield reduced expression and/or function of CIS as compared to an immune cell not edited at the location or locations in the CBLB gene,
wherein the immune cells are edited at one or more target locations in one more target genes selected from ADAM17, HIF-1a, DGKz, GSK-3B, LAG3, TIM3, TRIM29, IL-1R8, CD38, FBP-1, INSIG1, MED12, MED13, CCNC, CDK8, ID3, SOX4, or any combination thereof,
wherein the edits are made using an RNA-guided endonuclease, and
wherein the genetically engineered and edited immune cells exhibit one or more of enhanced expansion capability, enhanced cytotoxicity against target tumor cells, and enhanced persistence, particularly in a hypoxic tumor microenvironment, as compared to immune cells that do not comprise said genetically edited target site or sites.
103. The method of any one of Embodiments 100-102, wherein the RNA-guided endonuclease is a Crispr/CasX, Crispr/CasY, or Crispr/Cas9 system.
104. The method of any one of Embodiments 100-103, wherein the cells are edited at an additional target gene encoding ADAR2A, SMAD3, MAPKAPK3, CEACAM1, DDIT4, NKG2A, SOCS2, B2M, PD-1, TIM-3, CD38, or TCR alpha.
105. The method of any one of Embodiments 100-104, further comprising editing the immune cells at a gene encoding CD70, if the cytotoxic receptor targets CD70.
106. The method of any one of Embodiments 100-105, wherein the cytotoxic receptor expressed by the immune cells binds to one or more epitopes of CD19, CD70, ligands of the NKG2D receptor, BCMA, CD38, GPRC5D, CD138 DLL3, EGFR, PSMA, FLT3, KREMEN2, or combinations thereof.
107. The method of any one of Embodiments 100 to 106, wherein the wherein the immune cells comprise Natural Killer (NK) cells, T cells, induced pluripotent stem cells (iPSCs), iPSC-derived NK cells, NK-92 cells, or combinations thereof.
108. The method of any one of Embodiments 100 to 107, wherein the immune cells comprise Natural Killer (NK) cells.
109. The method of Embodiment 107, wherein the immune cells comprise a mixture of NK cells and T cells or a mixture of iPSC-derived NK cells and T cells.
110. The method of any one of Embodiments 100 to 109, wherein the administered immune cells are allogeneic with respect the subject.
111. The method of any one of Embodiments 100 to 110, further comprising administering IL2.
112. A population of genetically engineered and gene edited immune cells, comprising:
genetically engineered immune cells that express a cytotoxic receptor comprising an extracellular ligand binding domain, a transmembrane domain, and a cytotoxic signaling complex, wherein:
the extracellular ligand binding domain targets an antigen expressed by cells of a target tumor or cancer;
the immune cells are genetically edited within a target sequence in a MED12 gene and within a target sequence in a CISH gene; and
the edits yield reduced expression and/or function of each of the MED12 protein encoded by the MED12 gene and the CIS protein encoded by the CISH gene, as compared to an immune cell not edited within the target sequence in the MED12 gene and the target sequence within the CISH gene.
113. The population of genetically engineered and gene edited immune cells of Embodiment 112, wherein the target sequence within the MED12 gene comprises any one of SEQ ID NOS: 997, 938-944, 996, or 998.
114. The population of genetically engineered and gene edited immune cells of Embodiment 112, wherein the target sequence in the CISH gene comprises any one of SEQ ID NOS: 1013, 153-157 or 463-466 or 1012.
115. The population of genetically engineered and gene edited immune cells of Embodiment 112, wherein the extracellular ligand binding domains targets an antigen selected from BCMA, a NKG2D ligand, CD19, and CD70.
116. The population of genetically engineered and gene edited immune cells of Embodiment 112, wherein the extracellular ligand binding domains target BCMA.
117. The population of genetically engineered and gene edited immune cells of Embodiment 112, wherein the transmembrane domain comprises CD8, CD28, or a portion thereof, optionally wherein the transmembrane domain comprises CD8a or a portion thereof.
118. The population of genetically engineered and gene edited immune cells of Embodiment 112, wherein the cytotoxic signaling complex comprises a CD3zeta domain; and/or the cytotoxic signaling complex comprises an intracellular signaling domain of an OX40, 4-1BB, CD28, or a signaling portion thereof, optionally an intracellular signaling domain of OX40 or a signaling portion thereof.
119. The population of genetically engineered and gene edited immune cells of Embodiment 112, wherein at least a portion of the genetically engineered immune cells are engineered to express membrane bound IL-15 (mbIL15).
120. The population of genetically engineered and gene edited immune cells of Embodiment 119, wherein the cytotoxic receptor and the mbIL15 are encoded by the same nucleic acid molecule, optionally wherein the nucleic acid sequences encoding the cytotoxic receptor and the mbIL15 are separated by a nucleic acid sequence encoding a 2A peptide.
121. The population of genetically engineered and gene edited immune cells of Embodiment 112, wherein the cells are further genetically edited within a target sequence in the CBLB gene, wherein the target sequence in the CBLB gene comprises any one of SEQ ID NOS: 164, 165-166 or 453-456 or 1005-1008.
122. The population of genetically engineered and gene edited immune cells of Embodiment 112, wherein the cells are further genetically edited within a target sequence in a disintegrin and metalloproteinase domain-containing protein 17 (ADAM17) gene comprising any one of SEQ ID NOS: 682-687.
123. The population of genetically engineered and gene edited immune cells of Embodiment 112, wherein the cells are further genetically edited within a target sequence in a hypoxia-inducible factor 1-alpha (HIFI-a) gene comprising any one of SEQ ID NOS: 750-760.
124. The population of genetically engineered and gene edited immune cells of Embodiment 112, wherein the cells are further genetically edited within a target sequence in a DGKz gene, and the target sequence comprises any one of SEQ ID NOS: 688-723.
125. The population of genetically engineered and gene edited immune cells of Embodiment 112, wherein the cells are further genetically edited within a target sequence in a GSK-3B gene, and the target sequence comprises any one of SEQ ID NOS: 724-749.
126. The population of genetically engineered and gene edited immune cells of Embodiment 112, wherein the cells are further genetically edited within a target sequence in a LAG3 gene, and the target sequence comprises any one of SEQ ID NOS: 761-789.
127. The population of genetically engineered and gene edited immune cells of Embodiment 112, wherein the cells are further genetically edited within a target sequence in a TIM3 gene, and the target sequence comprises any one of SEQ ID NOS: 790-825.
128. The population of genetically engineered and gene edited immune cells of Embodiment 112, wherein the cells are further genetically edited within a target sequence in a TRIM29 gene, and the target sequence comprises any one of SEQ ID NOS: 826-835 or 1009-1011.
129. The population of genetically engineered and gene edited immune cells of Embodiment 112, wherein the cells are further genetically edited within a target sequence in a IL-1R8 gene, and the target sequence comprises any one of SEQ ID NOS: 836-865.
130. The population of genetically engineered and gene edited immune cells of Embodiment 112, wherein the cells are further genetically edited within a target sequence in a CD38 gene, and the target sequence comprises any one of SEQ ID NOS: 866-874.
131. The population of genetically engineered and gene edited immune cells of Embodiment 112, wherein the cells are further genetically edited within a target sequence in a FBP-1 gene, and the target sequence comprises any one of SEQ ID NOS: 875-889.
132. The population of genetically engineered and gene edited immune cells of Embodiment 112, wherein the cells are further genetically edited within a target sequence in a INSIG1 gene, and the target sequence comprises any one of SEQ ID NOS: 890-934.
133. The population of genetically engineered and gene edited immune cells of Embodiment 112, wherein the cells are further genetically edited within a target sequence in a CDK8 gene, and the target sequence comprises any one of SEQ ID NOS: 949-955.
134. The population of genetically engineered and gene edited immune cells of Embodiment 112, wherein the cells are further genetically edited within a target sequence in a CCNC gene, and the target sequence comprises any one of SEQ ID NOS: 956-961 or 999-1001.
135. The population of genetically engineered and gene edited immune cells of Embodiment 112, wherein the cells are further genetically edited within a target sequence in a ID3 gene, and the target sequence comprises any one of SEQ ID NOS: 963-969.
136. The population of genetically engineered and gene edited immune cells of Embodiment 112, wherein the cells are further genetically edited within a target sequence in a SOX4 gene, and the target sequence comprises any one of SEQ ID NOS: 970-976.
137. A population of genetically engineered and gene edited immune cells according to any one of Embodiments 112 to 136, wherein the edit to the target sequence or target sequences is made using an RNA-guided endonuclease.
138. The population of genetically engineered and gene edited immune cells of Embodiment 137 wherein the edit to the target sequence or target sequences is made using a Crispr/Cas9 system.
139. A population of genetically engineered and gene edited immune cells according to any one of Embodiments 112 to 138, wherein the immune cells comprise Natural Killer (NK) cells, T cells, induced pluripotent stem cells (iPSCs), iPSC-derived NK cells, iPSC-derived T cells, NK-92 cells, or any combination thereof.
140. The population of genetically engineered and gene edited immune cells of Embodiment 139, wherein the immune cells are natural killer (NK) cells.
141. A composition comprising the population of genetically engineered and gene edited immune cells of any one of Embodiments 112 to 140.
142. A method for the treatment of a subject having a disease or condition comprising administering to the subject the population of genetically engineered and gene edited immune cells of any of Embodiments 112 to 140 or the composition of Embodiment 141.
143. Use of the population of genetically engineered and edited immune cells of any of Embodiments 112 to 141 or the composition of Embodiment 142 for the treatment of a subject having a disease or condition.
144. The method of Embodiment 142 or the use of Embodiment 143, wherein the disease or condition is an infectious disease, an autoimmune disease, a cancer, or a tumor.
145. A population of gene edited immune cells, wherein the immune cells are genetically edited within a target sequence in a MED12 gene and within a target sequence in a CISH gene; and the edits yield reduced expression and/or function of each of the MED12 protein encoded by the MED12 gene and the CIS protein encoded by the CISH gene, as compared to an immune cell not edited within the target sequence in the MED12 gene and the target sequence within the CISH gene.
146. The population of gene edited immune cells of Embodiment 145, wherein the gene edited immune cells are genetically engineered immune cells that express a cytotoxic receptor comprising an extracellular ligand binding domain, a transmembrane domain, and a cytotoxic signaling complex; and the extracellular ligand binding domain targets an antigen expressed by cells of a target tumor or cancer.
147. A population of genetically engineered and gene edited immune cells that express a cytotoxic receptor comprising an extracellular ligand binding domain, a transmembrane domain, and a cytotoxic signaling complex, wherein the extracellular ligand binding domain targets an antigen expressed by cells of a target tumor or cancer; the immune cells are genetically edited within a target sequence in a MED12 gene, wherein the target sequence within the MED12 gene comprises any one of SEQ ID NOS: 997, 938-944, 996, or 998; and the edits yield reduced expression and/or function of the MED12 protein encoded by the MED12 gene, as compared to an immune cell not edited within the target sequence in the MED12 gene.
148. The population of genetically engineered and/or gene edited immune cells of Embodiment 147, wherein the immune cells are NK cells.

### EXAMPLES

The following are non-limiting descriptions of experimental methods and materials that were used in examples disclosed below.

As provided for herein, gene editing (e.g., using a Crispr/Cas system or other guided endonuclease) is used to enhance the functionality (e.g., persistence, cytotoxicity, and/or other characteristics) of immune cells, such as NK cells and/or T cells. Edits may be made at a single gene to reduce, substantially reduce, or eliminate expression and/or function of the protein encoded by the gene. Additionally, edits may be made at multiple gene targets (e.g., two, three, four, or more targets) to reduce, substantially reduce, or eliminate expression and/or function of the protein encoded by each of the genes edited. In several embodiments, the lack of expression or function of the protein (or multiple proteins) imparts to the edited cells an unexpectedly effective phenotype, either in terms of persistence, degree of cytotoxic signaling (either amount and/or duration), resistance to tumor microenvironment suppressive effects, and/or other beneficial characteristics. In several embodiments, immune cells, such as NK cells are edited at one or more of the following target genes: CISH, CBLB, DGKz, GSK-3B, HIF-1a, ADAM17, LAG3, TIM3, TRIM29, IL-1R8, CD38, FBP-1, INSIG1, TIGIT, TGFBR2, MED12, CCNC, CDK8, ID3, and SOX4, and/or any other targets of gene editing disclosed herein. In several embodiments, the gene edited cells are also engineered to express a CAR as disclosed herein, for example those schematically depicted in FIG. 1.

### Example 1—Screening of Singular Editing Targets in CD19 CAR Expressing Cells

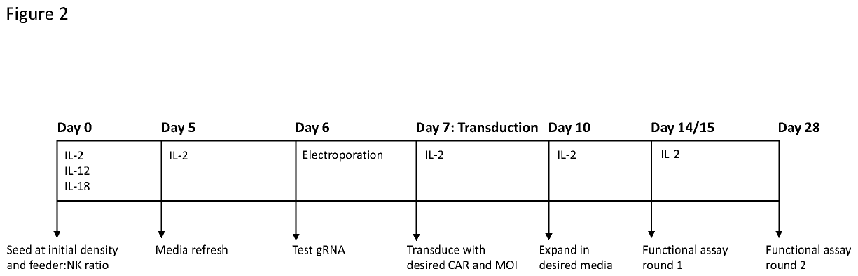

As discussed herein, gene editing targets can be targeted by, for example, a CRISPR/Cas system to reduce or eliminate the protein encoded by the target gene and enhance the functionality (e.g., cytotoxicity) or lifespan (e.g., persistence). Other editing modalities may also be used, depending on the embodiment, including other Cas enzymes, TALENs, zinc fingers, transposases, etc. This example relates to the screening of several single gene knock out (KO) candidates. FIG. 2 shows a schematic workflow for assessing the edits in, by way of example, NK cells. At day 0, NK cells were seeded at a density ranging from, for example about 0.1-1×10⁶ cells per cm². They were cultured in a media comprising IL2, IL12, and IL18 for 5 days (additional information regarding expansion of NK cells can be found in U.S. Pat. Nos. 7,435,596, or 8,026,087, US Patent Publication No. 2020/0016208, and U.S. patent application Ser. No. 17/628,105, each of which is incorporated by reference in its entirety herein). After 5 days, a partial media change was made, and an increased concentration of IL2 was added to the media in advance of electroporation to deliver the gene editing machinery (e.g., gRNA(s) and Crispr/Cas (e.g., Cas9)). Two days later, using elevated IL2 in the media, the cells were transduced with a viral vector encoding a CAR as provided for herein. This example, by way of a non-limiting embodiment, employed a CD19 targeting CAR. As in accordance with several embodiments disclosed herein, these cells were also engineered to express membrane-bound interleukin 15 (mbIL15). Three days after transduction, the edited and engineered cells were moved to a lower concentration IL2 media for 5 additional days, where a portion of the cell culture was collected for a first round of functional assays. Additional cells were cultured for another 14 days and then harvested for a second round of assays (hypoxia, cytotoxicity, expansion count, expression of edited target genes and/or CARs).

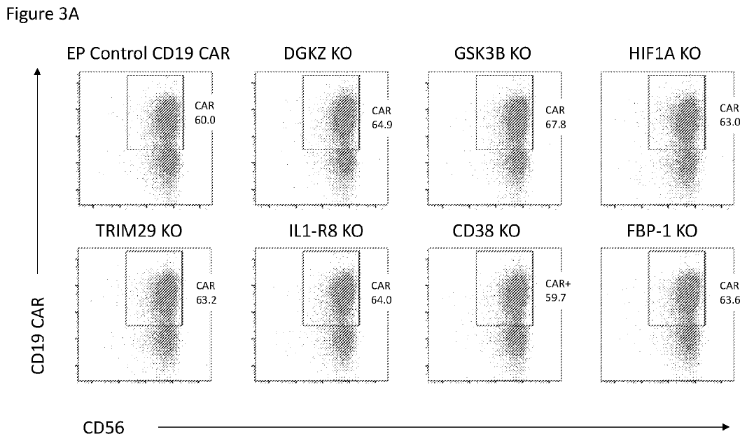

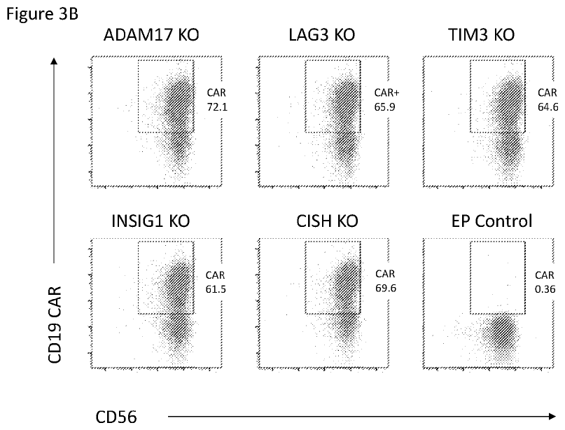

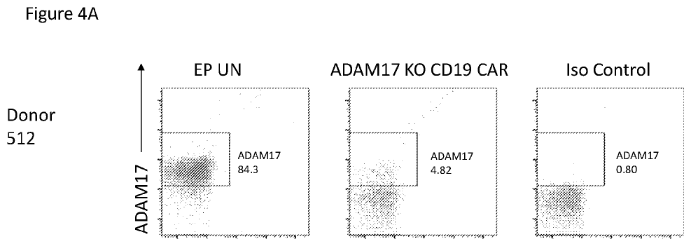

FIG. 3A shows data related to expression of a non-limiting embodiment of a CD19 targeting CAR by NK cells (CD56 positive) edited at the indicated target gene. As shown in FIG. 3A editing of any of DKGz, GSK-3B, HIF-1a, TRIM29, IL-1R8, CD38. or FBP-1 did not negatively impact the expression of the CD19 CAR. Likewise, as shown in FIG. 3B, neither did editing of ADAM17, LAG3, TIM3, INSIG1, or CISH. As controls, NK cells were subjected to mock electroporation and mock transduction (EP Control) or mock electroporation followed by transduction with the CD19 targeting CAR (EP Control CD19 CAR). Each of the edited cell populations exhibited similar CD19 CAR expression as the EP Control CD19 CAR shown in FIG. 3A, indicating that these candidate gene editing targets are not incompatible with CAR expression. Similar results were seen with another donor (data not shown).

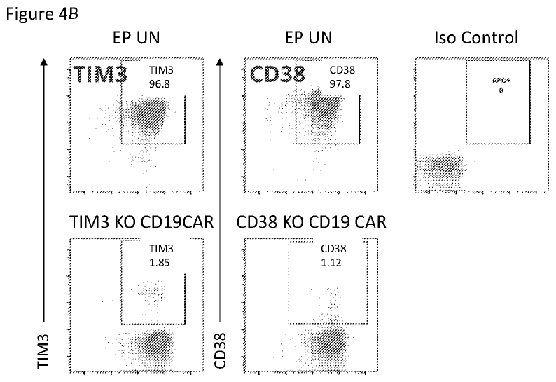

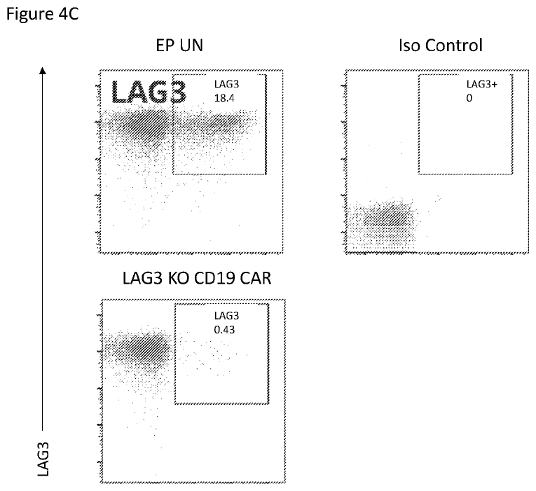

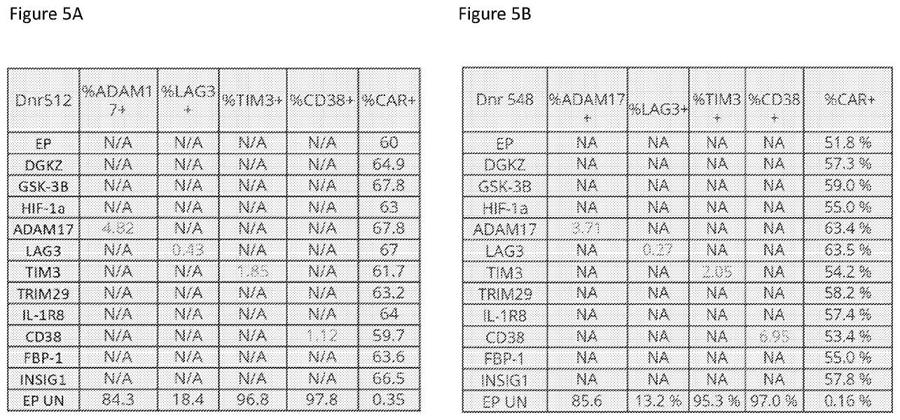

An additional set of experiments set out to evaluate the efficiency of gene editing to knock out, for example, ADAM17, LAG3, TIM3, or CD38. These data are shown in FIG. 4A-4C and FIG. 5 summarizes the expression data for selected gene edits on NK cells from two donors, namely ADAM17, LAG3, TIM3, and CD38. As controls, NK cells were subjected to mock electroporation and mock transduction (EP UN) or mock electroporation followed by transduction with the CD19 targeting CAR (EP). As can be seen in this data, the editing of any of these targets did not appear to adversely impact CAR expression (as measured at day 11), and in fact, appeared in some instances to improve CAR expression (see e.g., ADAM17 in D11% CAR+ column in left and middle panels of FIG. 5). Again, these data indicate that the editing of indicated targets is not incompatible with robust CAR expression and in some instances can enhance CAR expression (and/or function).

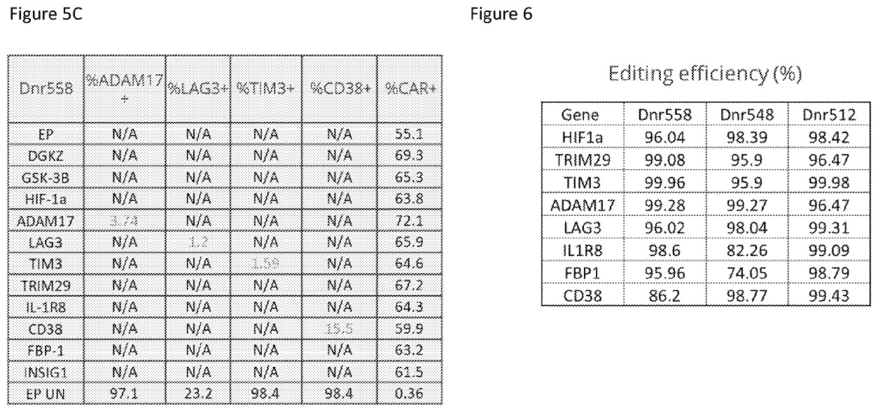

Genome editing outcomes were analyzed with the CRISPR software pipeline to determine gene editing efficiency. FIG. 6 shows that editing efficiency % was similar across cells from three donors for all genes edited, with only slightly lower percentages seen for IL1R8 (82.26%) and FBP1 (74.05%) in cells from donor 548. These data indicate that highly efficient gene editing of selected targets can be accomplished while maintaining significant levels of expression of a tumor-directed CAR.

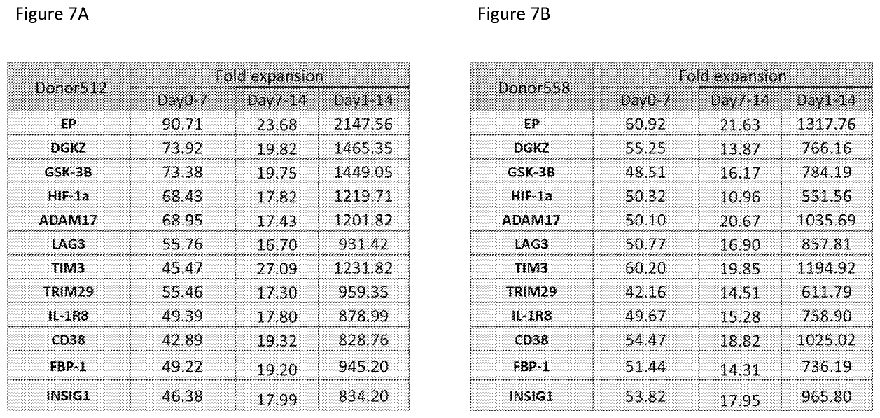

In several embodiments, the additional edits impart other beneficial characteristics to the edited cells (and in some embodiments, multiple edits further enhance cytotoxicity, as discussed below). An evaluation of the expansion capacity of edited NK cells was also undertaken. As shown in FIG. 7, the editing of each of the targets in this example still allowed for robust expansion from day 1 to 14, with EP control cells expanding over 2100 fold over 14 days and cells edited at a single target gene expanding from about 830-fold (CD38 edited) to nearly 1500-fold (DGKz edited). A similar expansion pattern was seen with a second donor. Thus, expression of a CAR as well as editing at a variety of target sites does not disrupt the robust expansion of the cells culture, rather, this expansion capacity is maintained across the panel of examples of editing targets.

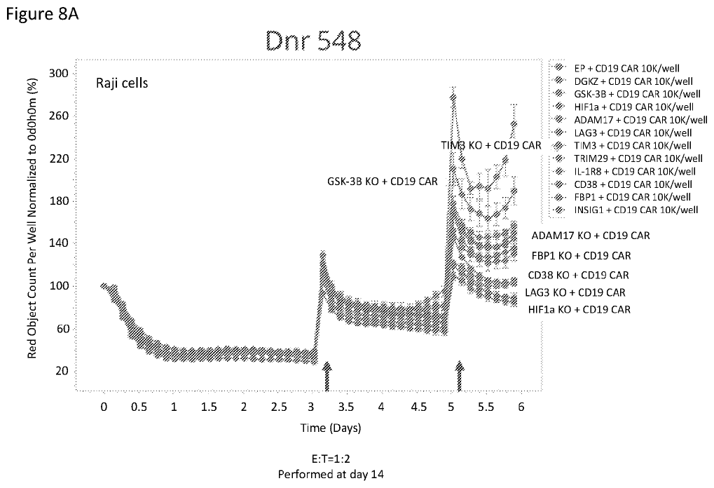

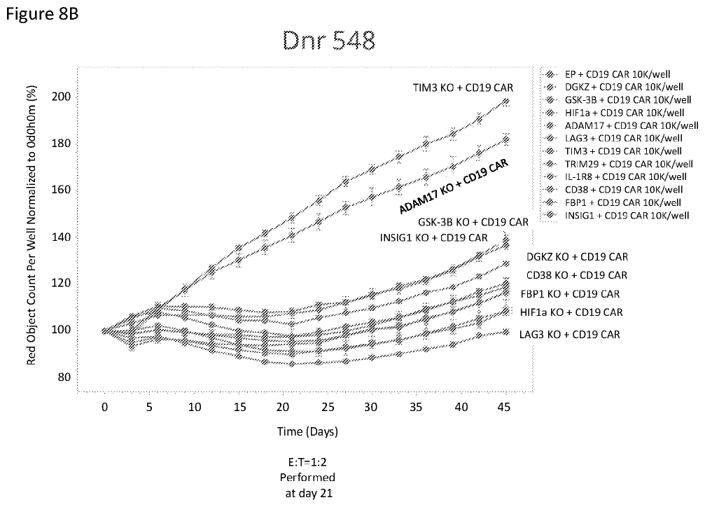

Moving to an assessment of the cytotoxicity of cells edited at various targets, Raji tumor cells were cocultured with NK cells expressing a CD19 CAR and edited as indicated at day 14. Data are shown in FIG. 8A, where an E:T of 1:2 was used. Two re-challenges of the edited CD19 CAR-expressing NK cells with fresh Raji cells (arrows) were performed in order to attempt to separate the anti-tumor effects of the NK cells (which were quite similar up to the second re-challenge). While TRIM29, TIM3, IL1-R8 and INSIG1 editing did not appear to positively influence cytotoxicity (though in some embodiments, they may enhance other aspects of the edited cells, such as persistence), edits to ADAM17, CD38, FBP-1, HIF-1a and LAG3 appeared to enhance cytotoxicity over unedited cells expressing a CD19 CAR (EP+CD19 CAR) at day 14 (FIG. 8A) and day 21 (FIG. 8B). Taken together, these data indicated that the candidate gene editing targets disclosed herein are efficiently disrupted, when edited, do not disrupt CAR expression, allow for robust expansion capacity of host cells (e.g., NK cells), and can enhance cytotoxicity against target tumor cells.

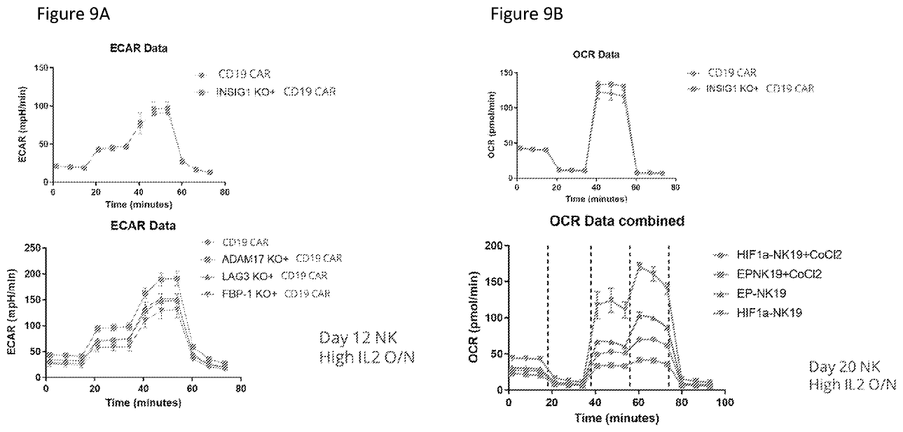

As discussed herein, the TME is an environment which is generated as a result of tumor cell function and is a pro-tumor/anti-immune cell environment. One such aspect relates to the below normal oxygen levels in the TME, in some instances by hypoxia. Hypoxia can trigger various signaling pathways in immune cells that result in the dysfunction of immune cells (thereby aiding the tumor cells in avoiding detection/destruction). Assays were performed to measure glycolytic capacity in cells over time, as determined by extracellular acidification rate (ECAR). After taking basal measurements, test cells are exposed to glucose to fuel glycolytic metabolism. Glucose treatment was followed by oligomycin, an inhibitor of the mitochondrial ATP synthase, which shifts energy production to glycolysis and reveals maximum glycolytic capacity. Thereafter 2-DG was added, which shuts down glycolysis and allows determination of glycolytic reserve by comparing maximal glycolytic capacity induced by oligomycin, with glycolysis induced by glucose. During the glycolysis stress test, ADAM17 KO edited cells (squares) showed higher levels of glycolysis than other edits and unedited CD19-CAR NK cells (circles) (FIG. 9A, lower panel).

FIG. 9B includes data from an assay designed to measure the oxygen consumption rate (OCR) of cells over time (and other parameters of mitochondrial respiration, separated from non-mitochondrial oxygen consumption). After taking basal measurements, test cells were exposed to oligomycin, an inhibitor of the mitochondrial ATP synthase, which leaves only non-mitochondrial oxygen consumption leakage across the inner mitochondrial membrane (by proton gradient) as the only sources of oxygen consumption. Next p-trifluoro-methoxyphenyl hydrazone (FCCP) was added, which depolarizes the mitochondria and results in maximal oxygen consumption. Finally, electron transport chain inhibitors (rotenone/antimycin A) prevent the flow of electrons through the electron transport chain (meaning ADP cannot be converted to ATP) and stop mitochondrial oxygen consumption. Importantly, the spare capacity (the difference between the basal respiration rate and the maximal respiration rate) is indicative of the relative ability of the cell to endure the stresses of a hypoxic environment. Cobalt chloride (CoCl2) was used as a hypoxia mimetic and induced a hypoxic environment for testing of various gene editing groups. FIG. 9B (lower panel) shows oxygen consumption data from four experimental groups: HIF-1a KO/CD19CAR—CoCl2 hypoxia, EP-CD19CAR—CoCl2 hypoxia, EP-CD19 CAR—normoxia, and HIF-1a-CD19 CAR—normoxia. As can be seen in FIG. 9B, in normoxic conditions, the knockout of HIF-1a (inverted triangles) results in a substantially larger spare oxygen consumption capacity as compared to those EP controls (triangles). While the OCR values are lower, due to the hypoxic conditions, the knockout of HIF-1a (circles) imparts to those cells a greater spare capacity as compared to EP controls (squares). This added spare capacity due to knockout of HIF-l a is indicative of an NK cell more adaptable and better able to survive and function in the hypoxic tumor microenvironment.

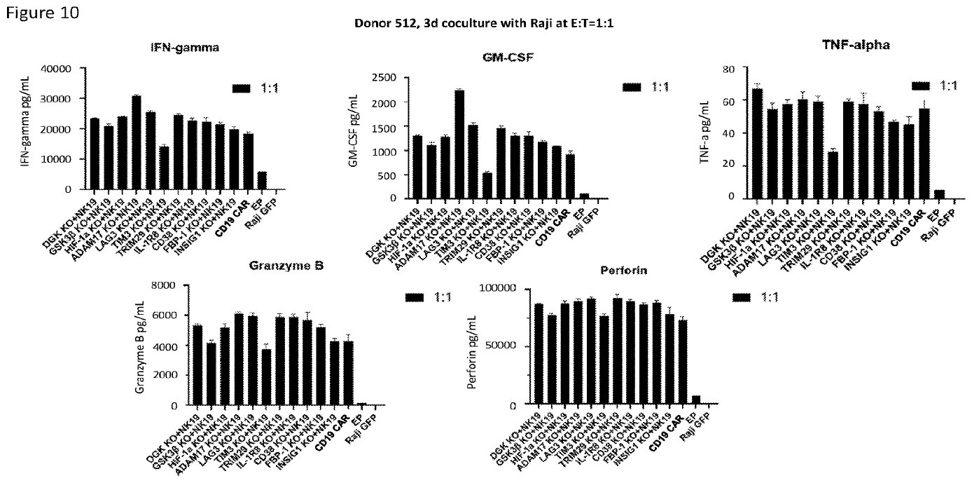

Next, effector cytokine production of various gene edited groups expressing the non-limiting embodiments of a CD19-CAR was assessed. Gene edited cells were cocultured for three days with Raji cells at an effector to target cell ratio (E:T) of 1:1 before cytokine levels were measured. ADAM17 edited cells demonstrated higher levels of GM-CSF and interferon (IFN) gamma compared to other edited groups (FIG. 10). Other gene edited groups showed cytokine levels similar to or slightly higher than those of cells expressing CD19-CAR without editing (CD19 CAR) and substantially elevated levels as compared to EP control cells (EP) (FIG. 10).

### Example 2—Screening of Singular Additional Editing Targets in CD70 CAR Expressing Cells

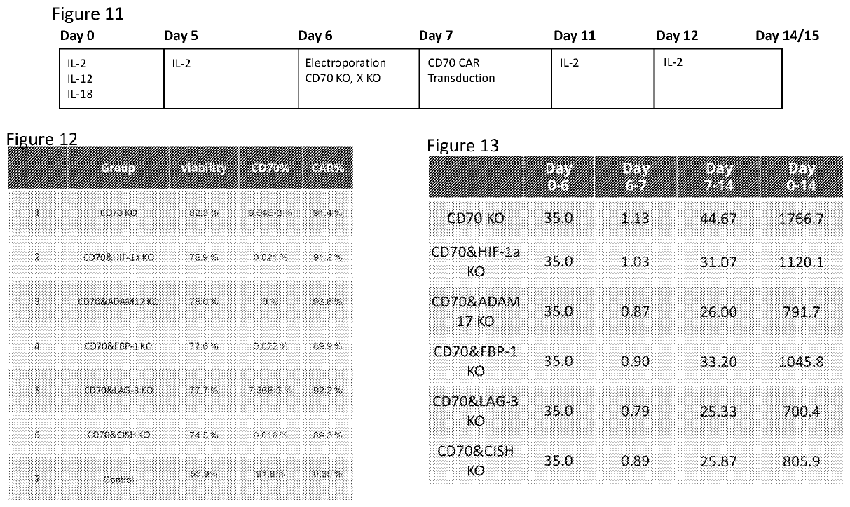

As an additional non-limiting embodiment, screening of various non-limiting examples of targets for gene editing were undertaken in cells engineered to express a CD70-targeting CAR. These cells, in accordance with several embodiments disclosed herein, were also engineered to express mbIL15. In this example, to avoid cellular fratricide, NK cells were edited to knockout CD70 expression, as well as the additional singular target genes. FIG. 11 shows a schematic workflow for assessing the edits in, by way of example, NK cells. Cells were cultured in a media comprising IL2, IL12, and IL18 for 5 days. After 5 days, a partial media change was made, and an increased concentration of IL2 was added to the media in advance of electroporation to deliver the gene editing machinery. At day 6, CD70 with or without an additional gene editing target, was knocked out. On day 7, using elevated IL2 in the media, the cells were transduced with a viral vector encoding, as a non-limiting example, a CD70 targeting CAR as provided for herein. Four days after transduction, the edited and engineered cells were moved to a lower concentration IL2 media for 1 additional day. On day 12, IL2 concentration was increased, and on day 14/15 a portion of cells was collected for a first round of functional assay (Incucyte®, ECAR, OCR, tracking cell numbers).

Next, cell viability, efficiency of CD70 knock out, and CD70 CAR expression were evaluated in the groups of edited cells. These data are summarized in FIG. 12. As can be seen in the data, the editing of any of these targets did not appear to adversely impact cell viability (as measured at day 11). Additionally, CD70 knockdown (from control levels to <1%) and CD70 CAR expression (averaging about 91%) were successful. Taken together, these data indicated that the CD70 target was efficiently disrupted, the gene edits performed did not disrupt CAR expression and did not negatively impact cell survival.

As shown in FIG. 13, the editing of each of the targets in this example still allowed for robust expansion from day 0 to 14, with CD70 KO cells expanding over 1700 fold over 14 days and cells edited at an additional target gene expanding from about 700-fold (LAG3 edited) to about 1120 fold (HIF-l a edited). Thus, expression of a CAR as well as editing at a variety of target sites, in addition to CD70 KO, does not disrupt the ability of the cells to expand robustly in culture, rather, this ability is maintained across the panel of examples of editing targets.

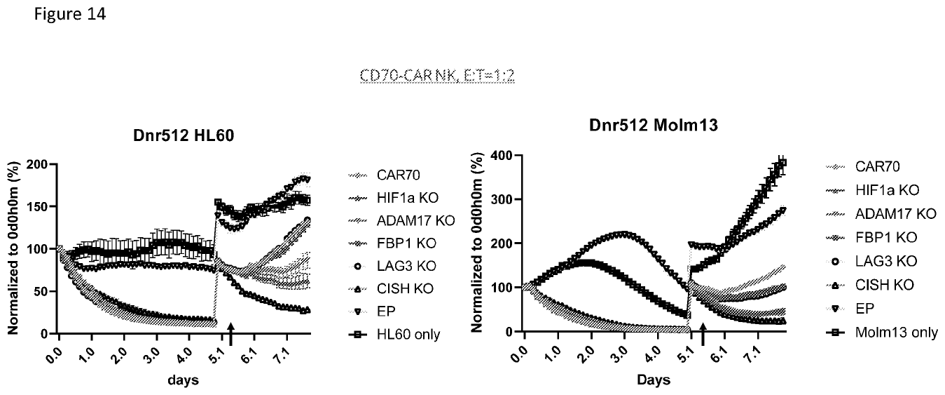

Moving to an assessment of the cytotoxicity of cells edited at various targets, HL60 (left panel) and Molm13 cells (right panel) were cocultured with NK cells expressing a CD70 CAR and edited as indicated at day 14. Data are shown in FIG. 14, where an E:T of 1:2 was used. Edits to CISH, HIF-1a and ADAM17 showed increased cytotoxicity than other groups and appeared to enhance cytotoxicity over cells edited at CD70 and expressing a CD70 CAR (CD70 KO) at day 14.

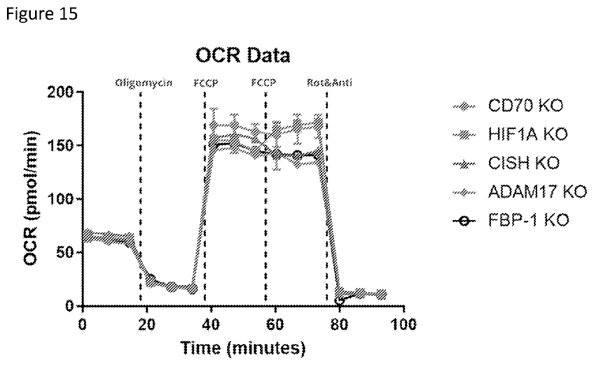

NK cells expressing a non-limiting embodiment of a CD70 targeting CAR and edited as indicated were subjected to a cell mitochondrial stress test following three days of treatment with lactate. OCR was assessed by seahorse analysis and no significant differences were found in the test results among the differently edited groups (FIG. 15). Thus, none of the gene edits performed here negatively impacted mitochondrial function. Taken together, these data show that CD70-edited and CD70 CAR-expressing NK cells can further be edited to decrease expression of an additional target gene and thereby impart further advantageous functionality to the NK cells.

### Example 3—Multiplex Editing Targets in CD70 CAR Expressing Cells

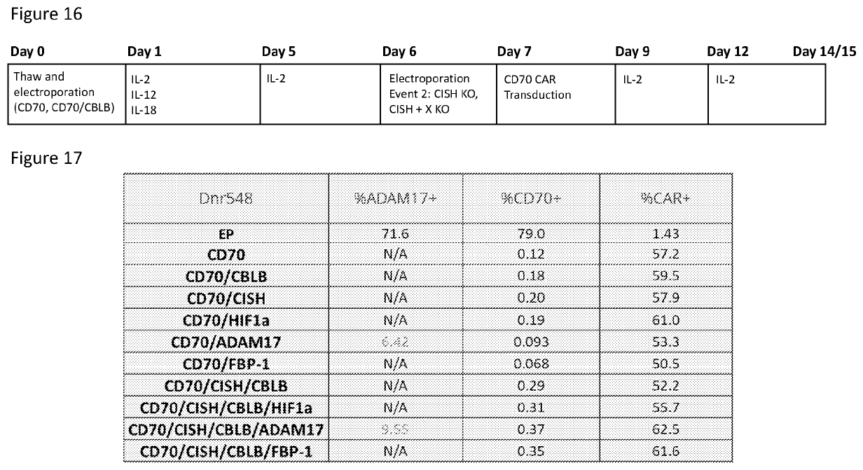

FIG. 16 shows a schematic workflow for assessing edits to multiple targets (“multiplex gene edits”) in, by way of example, NK cells. Cells were cultured in a media comprising IL2, IL12, and IL18 for 5 days. After 5 days, a partial media change was made, and an increased concentration of IL2 was added to the media in advance of electroporation to deliver the gene editing machinery. At day 6, gene edits were made to knock out targets including CD70, CISH. CBLB, HIF-1a, ADAM17, and/or FBP-1, with double, triple and quadruple knockouts being generated. These cells, in accordance with several embodiments disclosed herein, were also engineered to express mbIL15. On day 7, using elevated IL2 in the media, the cells were transduced with a viral vector encoding a CD70 targeting CAR as provided for herein. Four days after transduction, the edited and engineered cells were moved to a lower concentration IL2 media for 1 additional day. On day 12, IL-2 concentration was increased, and on day 14/15 a portion of cells was collected for a first round of functional assay (injection, Incucyte®, hypoxia, ECAR, OCR, tracking cell numbers).

Next, efficiency of ADAM17 and CD70 knock down, and CD70-targeting CAR expression were evaluated in the groups of edited cells. These data are summarized in FIG. 17. As can be seen in the data, edits to ADAM17 reduced the percentage of cells expressing ADAM17 from 71.6% (mock electroporation control; EP) to 6.42% (CD70/ADAM17 KO) or 9.55% (CD70/CISH/CBLB/ADAM17 KO). Additionally, CD70 knockdown and CD70 CAR expression were successful. The percentage of cells expressing CD70 was reduced from 79.0% (EP) to less than 1.0% in all edited groups. CD70-targeting CAR expression averaged around 60%. Taken together, these data indicated that the CD70 and ADAM17 targets were efficiently disrupted, and the edits performed did not disrupt CAR expression.

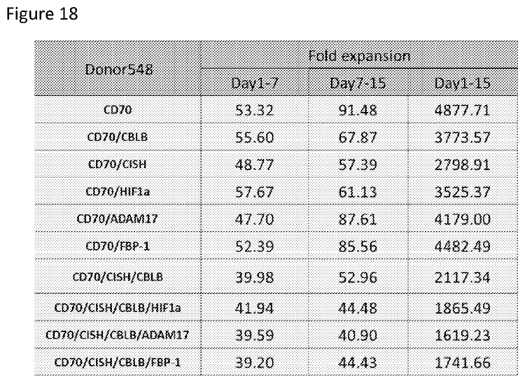

Effects of the multiplex gene edits on cell expansion were also evaluated. As shown in FIG. 18, the editing of each of the targets in this example still allowed for robust expansion from day 1 to 15, with CD70 KO cells expanding over 4800-fold over 14 days and cells edited at an additional target gene expanding from about 1600-fold (CD70/CISH/CBLB/ADAM17 edited) to about 4400-fold (CD80/FBP-1 edited). Thus, expression of a CD70-directed CAR as well as editing at a variety of target sites, in addition to CD70 KO, does not disrupt the ability of the cells to expand robustly in culture, rather, this ability is maintained across the panel of editing targets.

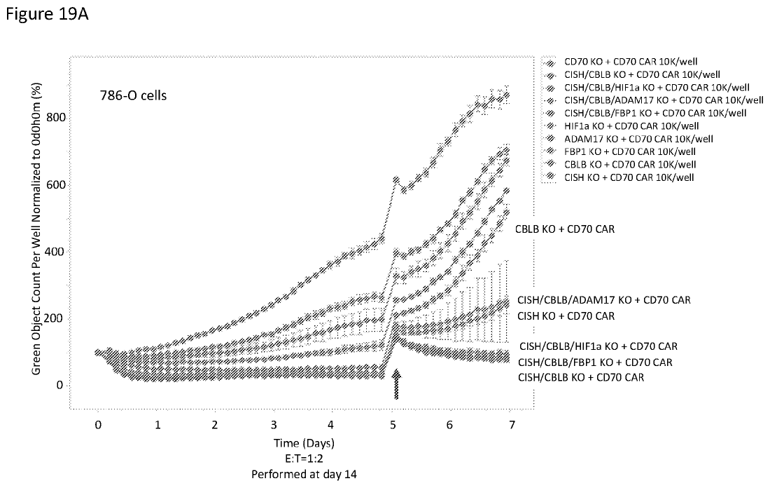

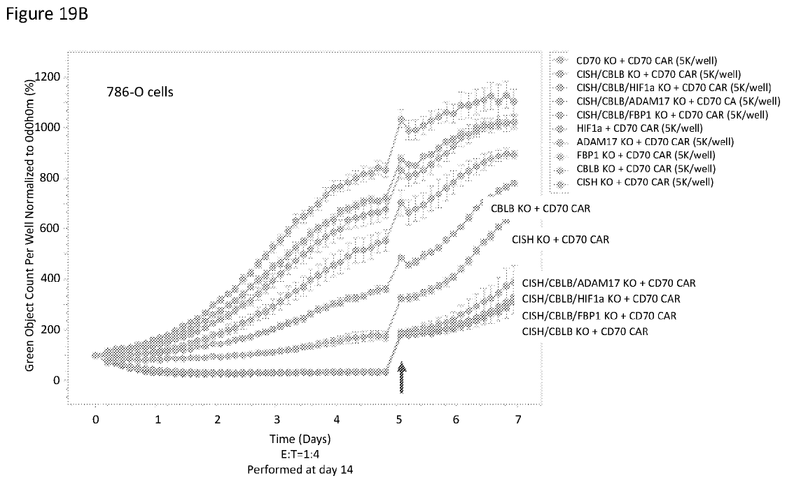

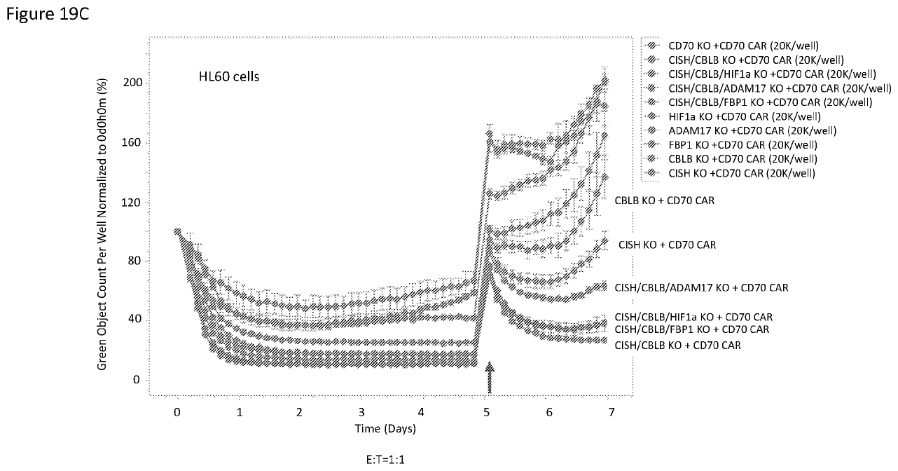

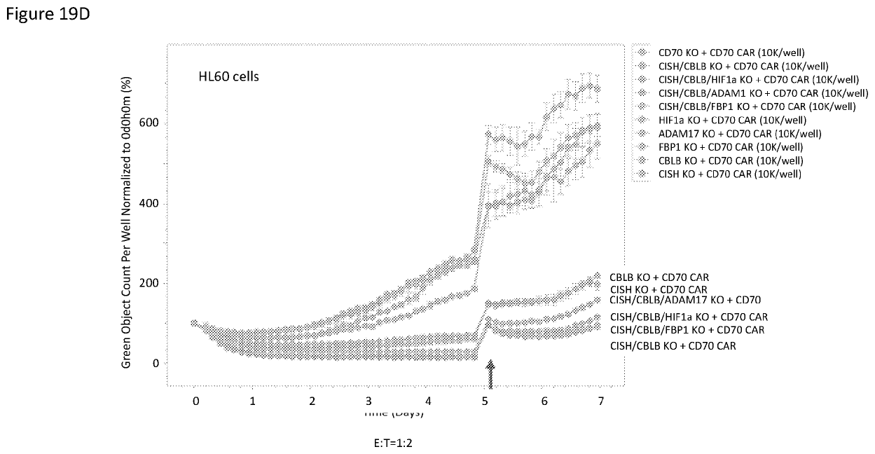

Cytotoxicity of the cells edited at various targets indicated in FIGS. 17 and 18 was assessed next. 786-0 cells were cocultured with NK cells expressing a non-limiting embodiment of a CD70 targeting CAR and edited as indicated in FIG. 19A-19B at day 14. Data are shown in FIG. 19A, where an E:T of 1:2 was used, and FIG. 19B where an E:T of 1:4 was used. Edits to HIF-1a, FBP-1 and ADAM17 in addition to CISH and CISH/CBLB showed better cytotoxicity than other groups, consistent with the single edit data for HIF-1a and ADAM17 shown previously. Additionally, a cytotoxicity assay was performed where the edited cells were incubated with HL60 cells at a 1:1 (FIG. 19C) or 1:2 ratio (FIG. 19D). Cytotoxicity results with HL60 cells were consistent with 786-0 cells, where cells edited at HIF-1a, FBP-1 and ADAM17 in addition to CISH/CBLB showed improved cytotoxicity over other groups.

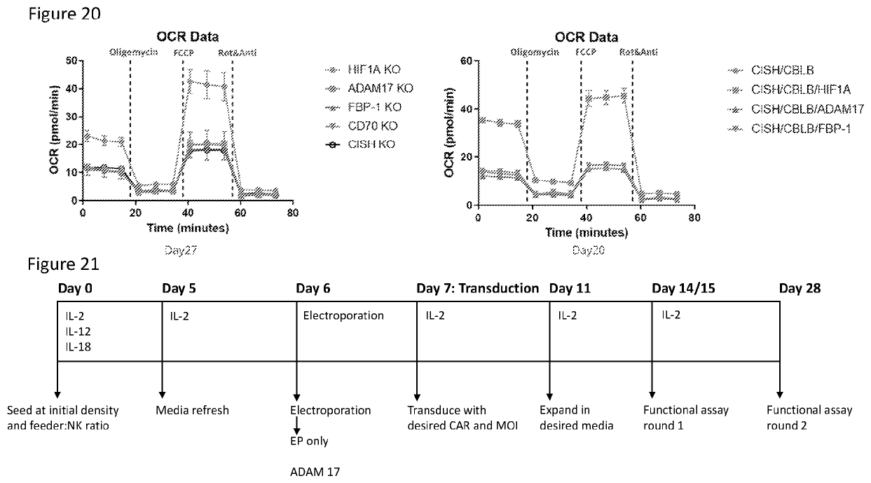

NK cells expressing a non-limiting embodiment of a CD70-directed CAR and edited as indicated in FIG. 20 were evaluated for response to mitochondrial stress under hypoxic conditions induced by CoCl2. NK cells expressing a non-limiting embodiment of a CD70-directed CAR and edited at HIF1a showed much higher respiratory capacity during hypoxic conditions (FIG. 20, left panel). A similar trend was identified when cells were edited at multiple targets (FIG. 20, right panel). As indicated previously in Example 1, the higher spare respiratory capacity in HIF-1a edited cells suggests that these NK cells would be better adapted to the hypoxic tumor microenvironment.

Taken together, these data demonstrate that multiplex editing, even to the point of editing four (or more) genes is feasible, still allows for robust expression of CARs targeting a desired tumor, and imparts to immune cells, such as NK cells, robust anti-tumor effects and/or enhanced persistence (e.g., survival and/or activity in the hypoxic TME).

### Example 4—Investigation of the Phenotypes, Proliferation and Cytotoxicity of ADAM17KO, CD19 or CD70 CAR NK Cells

FIG. 21 shows a schematic workflow for assessing the edits in, by way of example, NK cells. Cells were cultured in a media comprising IL2, IL12, and IL18 for 5 days. After 5 days, a partial media change was made, and an increased concentration of IL2 was added to the media in advance of electroporation to deliver the gene editing machinery. At day 6, ADAM17 was knocked out. On day 7, using elevated IL2 in the media, the cells were transduced with a viral vector encoding, in a non-limiting embodiment, a CD19-targeting CAR as provided for herein. Four days after transduction, the edited and engineered cells were moved to a lower concentration IL2 media for 1 additional day. On day 11, IL-2 concentration was increased, and on day 14/15 a portion of cells was collected for a first round of functional assay (Incucyte®, tracking cell numbers, NK cell phenotype).

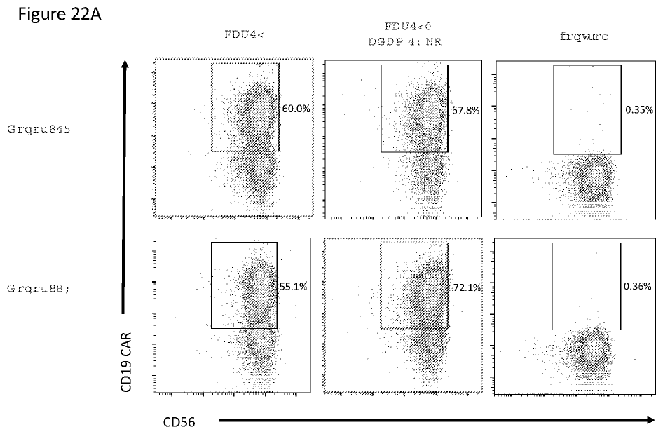

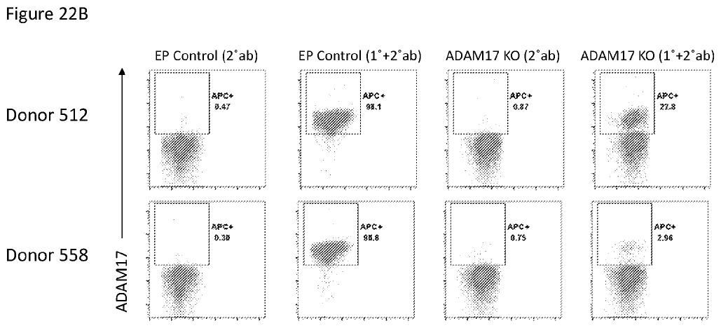

CAR expression was evaluated on day 4 post transduction. As a control, NK cells were subjected to mock electroporation and transduction (EP UN). CD19-directed CAR expression (labeled as CD19 CAR) was found to be between 55-72% when assessing cells from two different donors (FIG. 22A). Therefore, editing of ADAM17 still allows for robust expression of the CD19-directed CAR. Additionally, efficiency of ADAM17 knock out was evaluated and ADAM17 was found to decrease from about 98% in mock electroporated controls (EP Control) to about 3-23% (FIG. 22B). Taken together, the data indicates that it was possible to generate cells that both featured a CD19-directed CAR and successful ADAM17 editing.

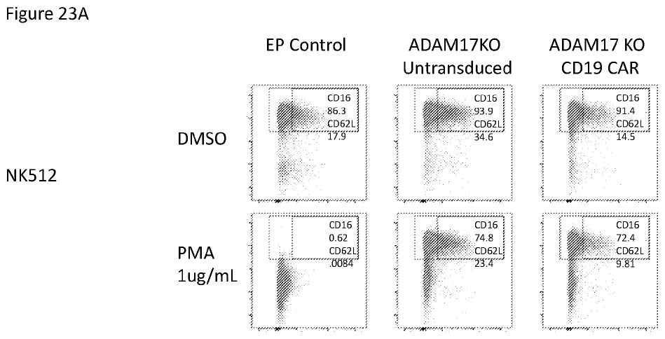

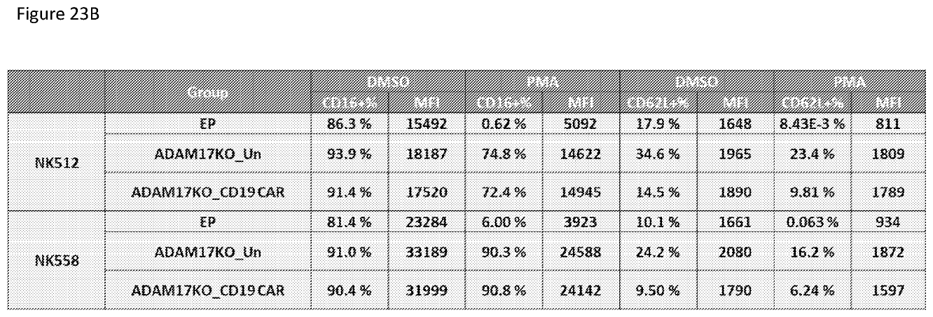

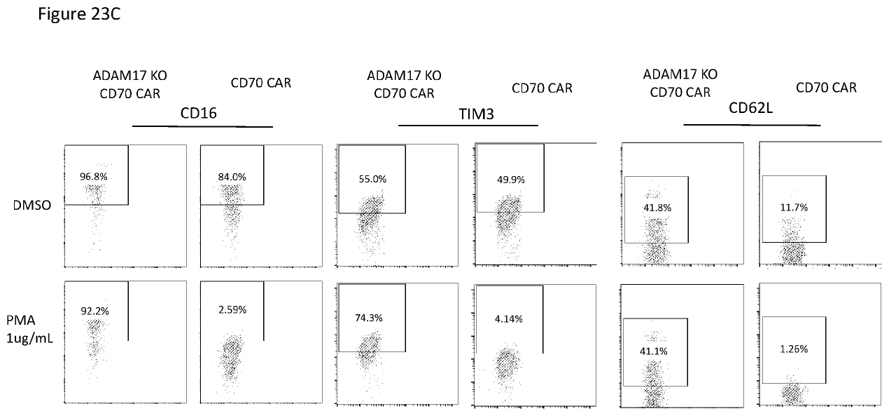

ADAM17 is responsible for the rapid cleavage of the activating FcγRIIIa (CD16a) from the surface of NK cells after activation which can result in temporary inhibition of antibody dependent cytotoxicity (ADCC) and the loss of the ensuing NK cytotoxicity. While chemical inhibitors of ADAM17 exist, directed reduction in ADAM17 could enhance ADCC (by removing a disruptor of that process) and avoid potential toxicities associated with chemical inhibitors. Therefore, NK cells were edited to knock out ADAM17, with or without expression of a CD19-directed CAR, and EP controls were stimulated with phorhol myristate acetate (PMA) at lug/mL or DMSO for 1 hour before CD16 and CD62L (ADAM17 substrates) expression were evaluated. PMA is a nonspecific activator of NK cells that induces shedding of ADAM17 substrates. As shown in FIG. 23A, and summarized in FIG. 23B, CD16 and CD62L expression decreased on short term PMA stimulated EP Control NK cells but not ADAM17KO NK cells, whether mock transduced (Untransduced) or engineered to express the exemplary CD19-directed CAR (ADAM17 KO CD19 CAR). EP controls stimulated with PMA exhibited CD16 positive percentages between 0.62-6.0%, whereas cells with edited ADAM17 showed CD16 positive % from 72.4-92.0%. In a related experiment, NK cells expressing an exemplary CD70-directed CAR were edited at ADAM17 (ADAM17 KO) and stimulated with 1 ug/mL PMA or DMSO for 1 hour before expression of various ADAM17 substrates was evaluated. As shown in FIG. 23C, CD16, TIM3, and CD62L expression decreased with PMA stimulation in unedited CD70 CAR NK cells, but not in ADAM17KO CD70 CAR NK cells. These data indicate that ADAM17 editing prevented loss of CD16, TIM3 and CD62L expression following PMA stimulation in edited NK cells expressing non-limiting embodiments of CD19 or CD70 CARs.

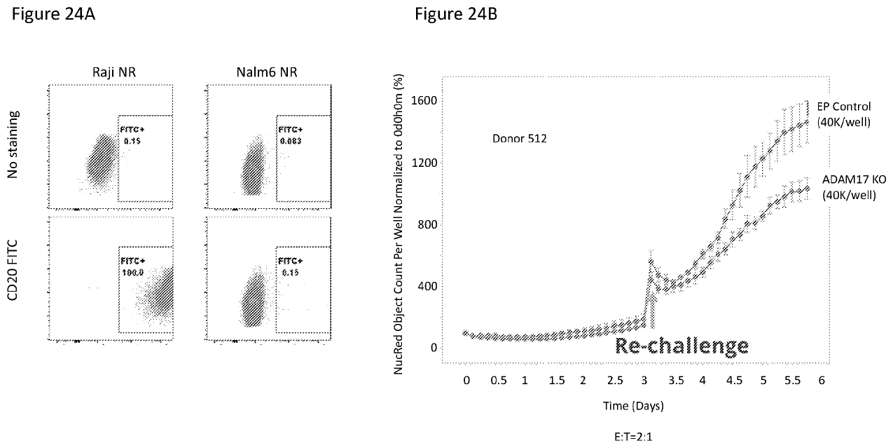

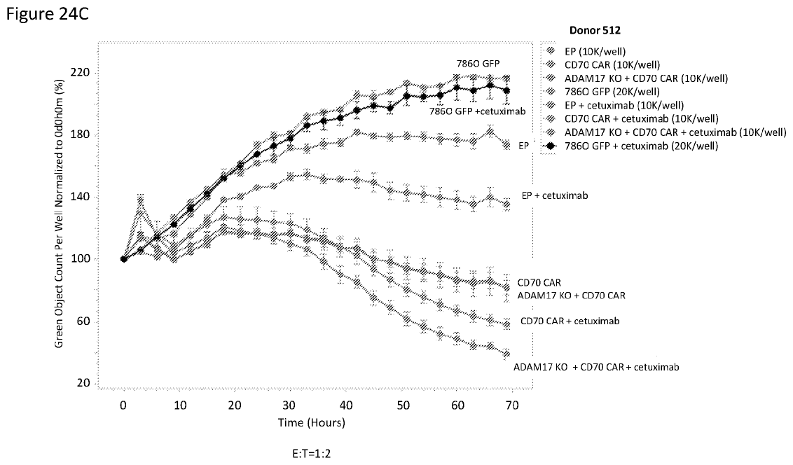

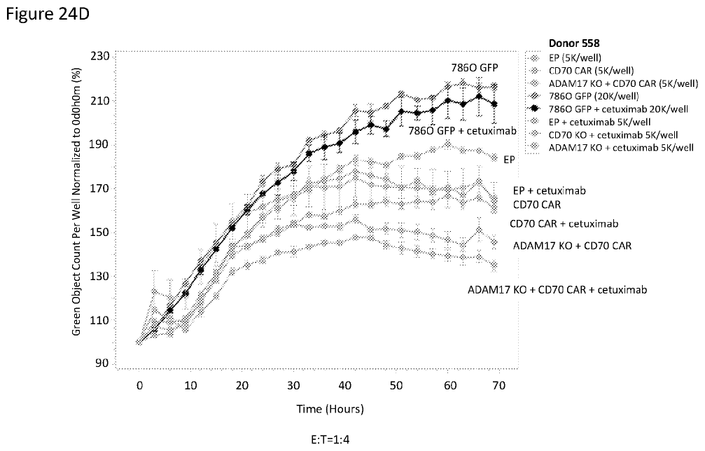

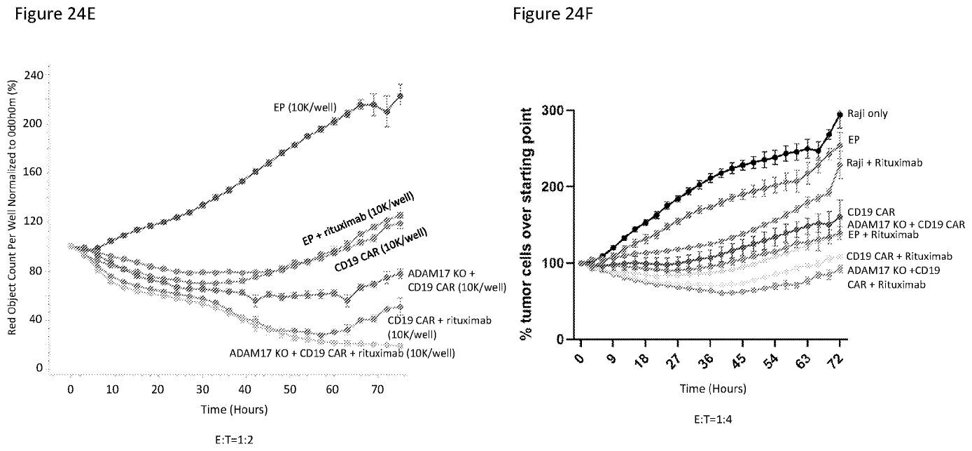

Having determined that ADAM17 editing disrupted reduction in surface CD16 expression following PMA treatment, a new experiment was designed to address whether the Fc binding properties of CD16 could be harnessed to impart increased ADCC upon ADAM17 edited NK cells. First, an assay was performed where Raji and Nalm6 cells were evaluated for CD20 expression by flow cytometry. As shown in FIG. 24A, CD20 expressing Raji cells were 100% positive, whereas Nalm6 cells expressed virtually undetectable levels of CD20. Next, cytotoxicity of ADAM17 edited NK cells against Raji cells was confirmed to be higher than EP control (FIG. 24B). FIG. 24C-24G show experiments where Raji cells were precoated with Rituximab (anti-CD20) or Cetuximab (anti-EGFR) for 30 minutes before cytotoxicity was assessed to determine if ADAM17 editing enhanced ADCC. Without being bound by theory, the putative mechanism of action resulting from ADAM17 editing is to maintain CD16 cell surface expression (because the normal ADAM17-induced cleavage is reduced), which allows for CD16 to bind to the Fc region of the non-limiting examples of anti-cancer antibodies (here, rituximab or cetuximab) and thereby induce synergistic cytotoxic effects in combination with the CD70-directed CAR NK cells. ADAM17 editing was found to enhance ADCC mediated by Cetuximab at E:T of 1:2 (FIG. 24C) and 1:4 (FIG. 24D). Similar results were achieved with Rituximab both with (FIG. 24E-F) and without (FIG. 24G) expression of a CD19-directed CAR. Thus, editing of ADAM17 reduced CD16 cleavage, improving ADCC over cytotoxicity levels achieved with cells expressing a CD19-directed CAR without ADAM17 editing.

### Example 5—Investigation of the Phenotypes, Proliferation and Cytotoxicity of CD70 CAR NK Cells

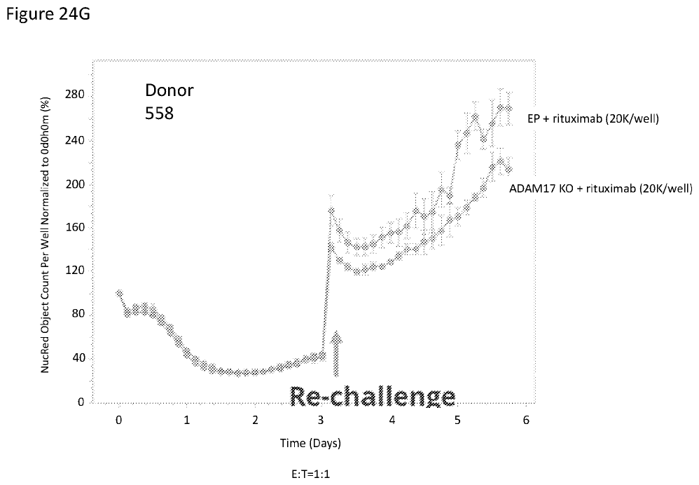

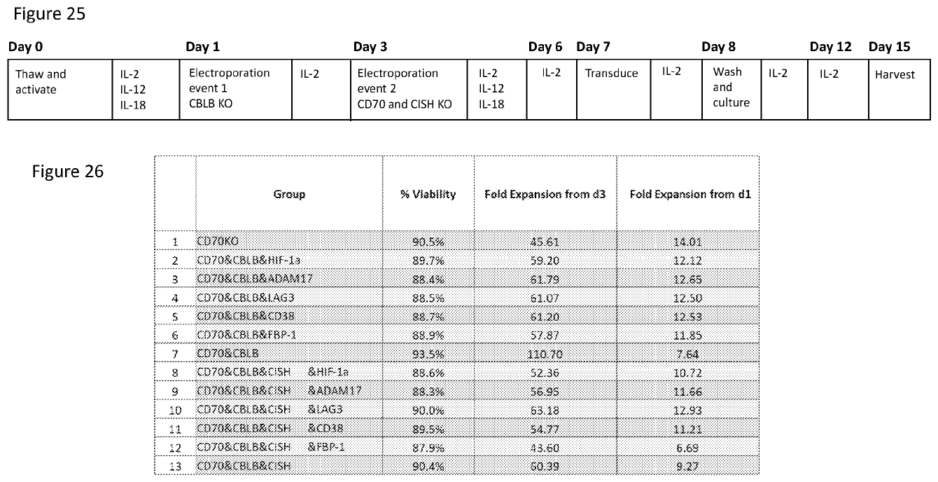

FIG. 25 shows a schematic workflow for assessing edits to multiple targets in, by way of example, NK cells. Cells were cultured in a media comprising IL2, IL12, and IL18 for 1 day. On day 1, CBLB editing was performed. On day 2, an increased concentration of IL2 was added to the media in advance of electroporation to deliver the gene editing machinery. On day 3, CD70 and CISH editing was performed. On days 4, cells were cultured in a media comprising IL2, IL12, and IL18 until day 6 when an increased concentration of IL2 was added to the media in advance of electroporation to deliver the gene editing machinery. Transduction of the CD70-directed CAR occurred on day 7. Cells were maintained with an elevated level of IL-2 until washing and culture at day 8. Cells were kept at low IL2 concentration until day 12 where IL2 concentration was elevated in advance of harvest at day 15.

NK cells edited for CD70/CBLB with ADAM17, HIF-1a, LAG3, CD38, or FBP-1 KO, with, or without edited CISH were evaluated for viability and fold expansion from day 1 and day 3 at day 7 as summarized in FIG. 26. Viability ranged from about 88 to about 94% across the edited groups. Fold expansion from day 1, before EP, ranged from about 7 to about 14-fold. Fold expansion from day 3. after EP, ranged from about 45 to over 120-fold. Thus, the multiplex editing performed in these groups still allows significant NK cell survival and expansion.

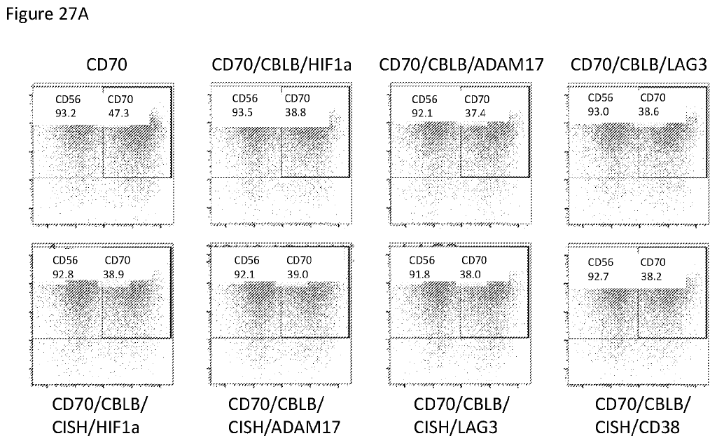

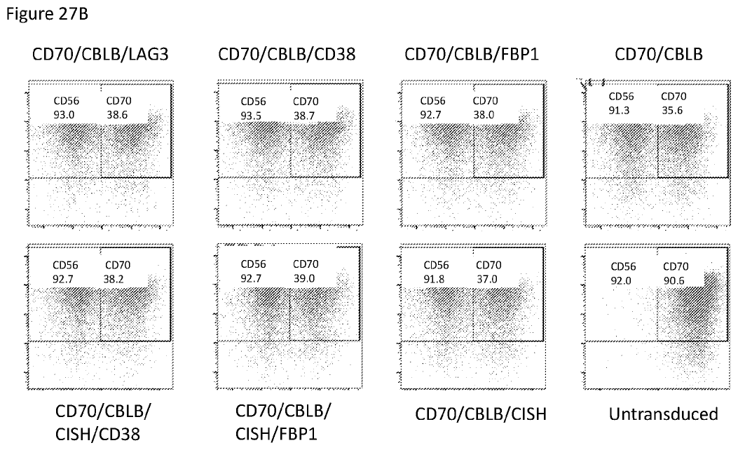

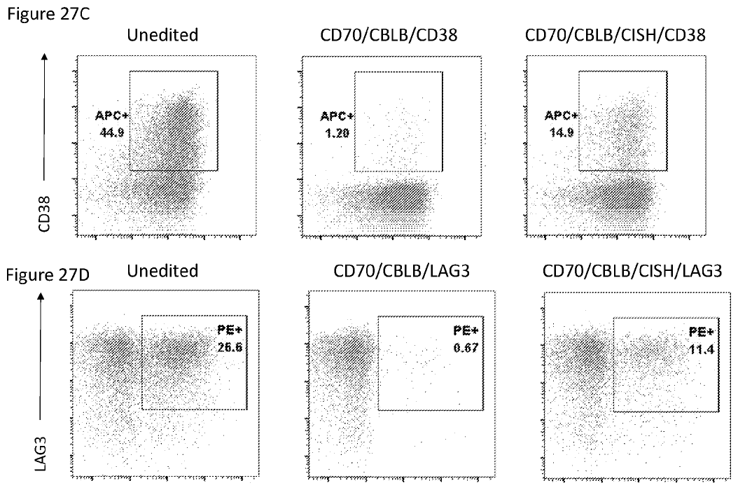

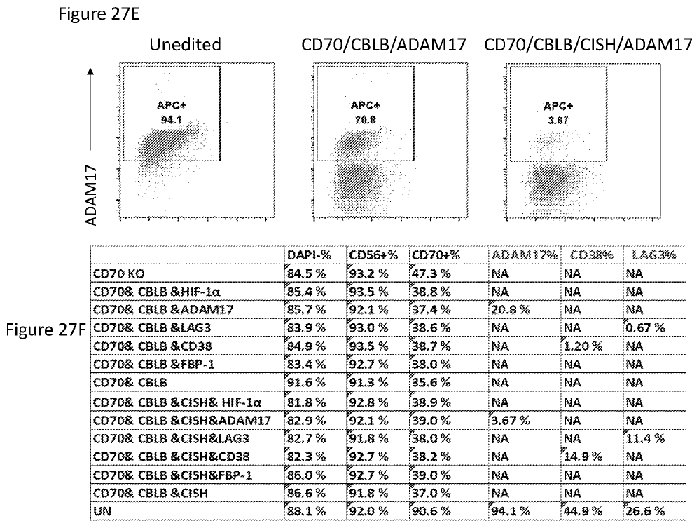

Next, editing efficiency was evaluated. As shown in FIG. 27A-27B CD70 editing to prevent fratricide resulted in a reduction in CD70 expression from 90.6% (FIG. 27B) to approximately 39%. Additionally, CD38 (FIG. 27C), LAG3 (FIG. 27D), and ADAM17 (FIG. 27E) editing successfully reduced expression of the respective target in both triple and quadruple knock outs compared to unedited controls. Flow cytometry results are summarized in FIG. 27F, indicating that the multiplex gene editing was successful.

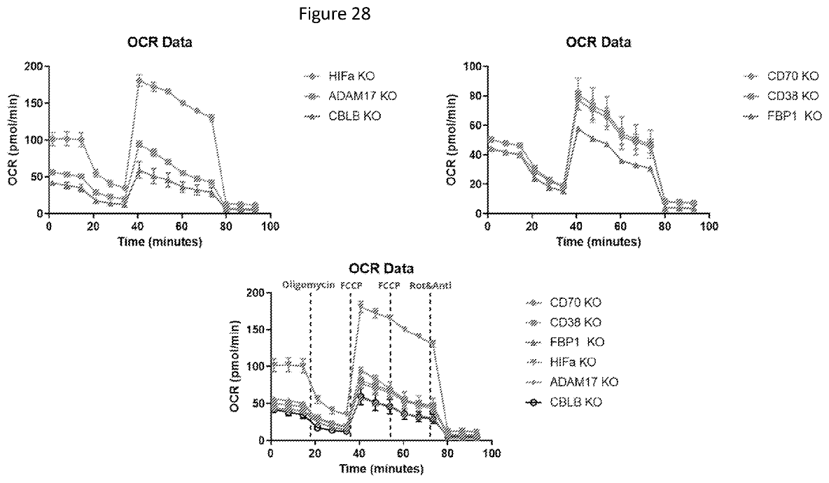

Edited CD70-CAR expressing NK cells were evaluated for response to mitochondrial stress under hypoxic conditions induced by CoCl2. Similar to data in FIG. 20, above, CAR-NK cells with edited HIF-1a showed much higher respiratory capacity during hypoxic conditions compared to other edits (FIG. 28).

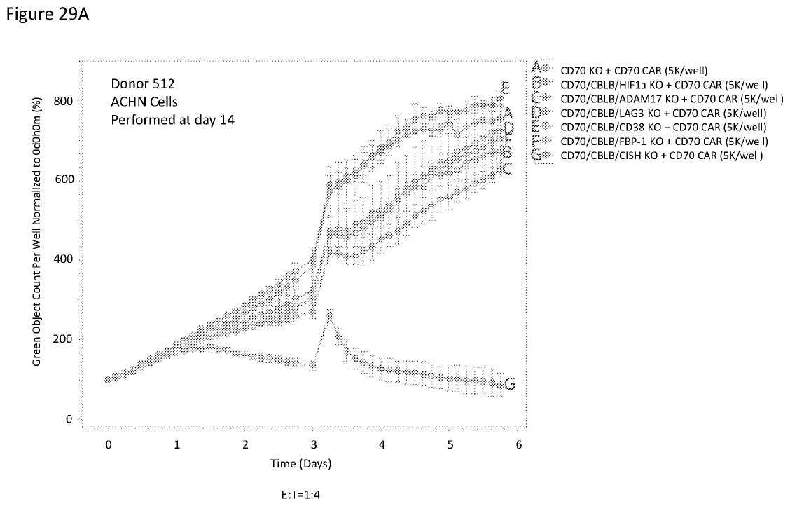

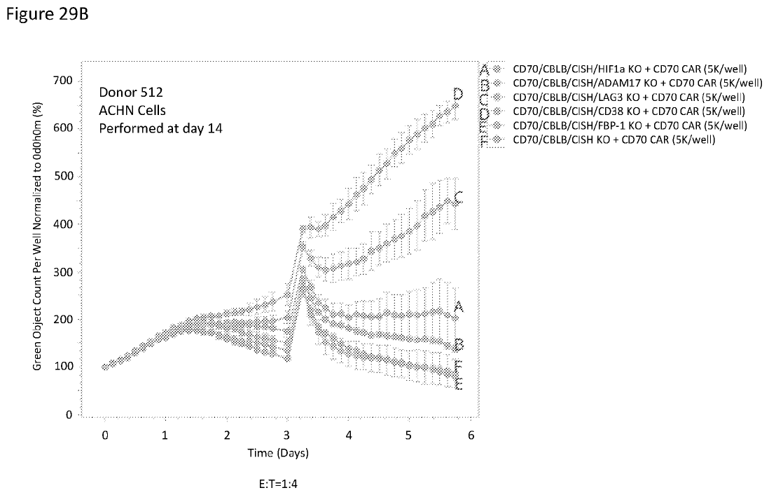

Cytotoxicity of the triple and quadruple knockout edited cells was assessed against the ACHN renal adenocarcinoma cell line (CD70 rich). NK cells expressing a CD70-directed CAR and edited as indicated were incubated with ACHN cells at a 1:4 E:T ratio at day 14. As shown in FIG. 29A, the cells expressing a CD70 CAR and edited at CD70/CBLB/CISH demonstrated the highest cytotoxicity amongst the triple knockouts. Amongst the quadruple knockouts in FIG. 29B, the CD70-directed CAR expressing ADAM17, HIF-1a, and FBP-1 edited cells (which were all further edited at CD70/CBLB/CISH) demonstrated enhanced levels of cytotoxicity.

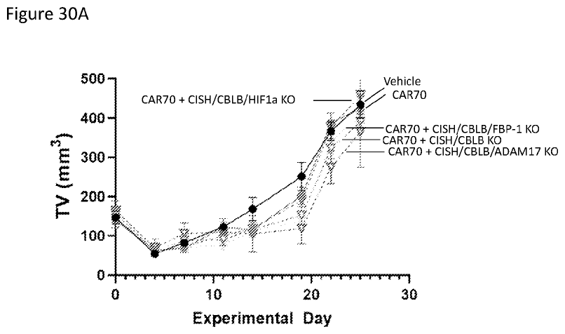

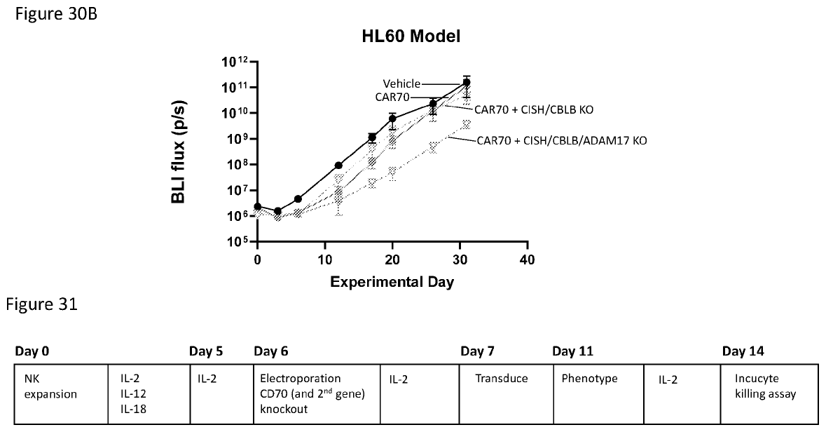

Multiplex edited NK cells from a single donor were next evaluated in an in vivo anti-tumor activity assay where mice were injected with 786-0 cells at day −7. Multiplex edited NK cells expressing a CD70-directed CAR and edited as indicated were injected at day 0 and tumor volume (TV) was assessed over a 25 day period. As shown in FIG. 30A, cells edited to express a CD70-directed CAR in addition to CISH/CBLB/ADAM17KO were the most effective at controlling tumor volume. The multiplex edited NK cells from the same donor were evaluated in an additional in vivo anti-tumor activity assay where mice were injected with HL60 cells at day −2. Multiplex edited NK cells expressing a CD70-directed CAR were injected at day 0 and tumor volume (TV) was assessed over a 30-day period. Similar to the 786-0 model, cells expressing a CD70-directed CAR and knocked out for CISH/CBLB/ADAM17 were the most effective at controlling tumor volume (FIG. 30B). The expansion of the multiplex edited cells used in the 786-0 and HL60 tumor model experiments is shown below in Table E1.

TABLE E1
Expansion of Multiplex Edited NK Cells

| | Fold Expansion | | |
|---|---|---|---|
| Edit | Day 1-7 | Day 7-15 | Day 1-15 |
| CD70 | 53.32 | 91.48 | 4877.71 |
| CD70/BLB | 55.60 | 67.87 | 3773.57 |
| CD70/CISH | 48.77 | 57.39 | 2798.91 |
| CD70/HIF1a | 57.57 | 61.13 | 3525.37 |
| CD70/ADAM17 | 47.70 | 87.61 | 4179.00 |
| CD70/FBP-1 | 52.39 | 85.56 | 4482.49 |
| CD70/CISH/CBLB | 39.98 | 52.96 | 2117.34 |
| CD70/CISH/CBLB/HIF1a | 41.94 | 44.48 | 1865.49 |
| CD70/CISH/CBLB/ADAM17 | 39.59 | 40.90 | 1619.23 |
| CD70/CISH/CBLB/FBP-1 | 39.20 | 44.43 | 1741.66 |

Taken together, these data indicated that the multiplex candidate gene editing strategy described herein disrupted target genes efficiently, allowed for survival and robust expansion capacity of host cells, and enhanced cytotoxicity against target cancer cells in both in vitro and in vivo assays.

### Example 6—Screening of Additional Singular Editing Targets in CD70 CAR Expressing Cells

FIG. 31 shows a schematic workflow for assessing the edits in, by way of example, NK cells. Cells were cultured in a media comprising IL2, IL12, and IL18 for 5 days. After 5 days, a partial media change was made, and an increased concentration of IL2 was added to the media in advance of electroporation to deliver the gene editing machinery. At day 6, CD70 with or without an additional gene editing target was knocked out. Additional gene editing targets included MED12, CCNC, CDK8, ID3, and SOX4. Table E2 below shows non-limiting examples of gRNAs for these genes. Other gRNAs may be used in additional embodiments (see e.g., SEQ ID NOs: 941-948, 952-955, 959-962, 966-969, and/or 973-976).

TABLE E2
Additional Example Guide RNAs

| SEQ ID | | | |
|---|---|---|---|
| NO | Name | Sequence | Target |
| 938 | MED12 gRNA1 | TGCAATAATGCTGCTGAAGT | Exon 3 |
| 939 | MED12 gRNA2 | AGTTATCCTTCTGGTTCACT | Exon 3 |
| 940 | MED12 gRNA3 | GTCAGTGAACCAAGTGTTAA | Exon 3 |
| 996 | MED12 gRNA4 | AGGATTGAAGCTGACGTTCT | Exon 2 |
| 997 | MED12 gRNA5 | TAACTGCTCCCATAAGTACT | Exon 5 |
| 998 | MED12 gRNA6 | GTGGGATTACACCGAGAAGC | Exon 5 |
| 949 | CDK8 gRNA1 | AAGTGAAGCTGAGCAGCGAG | Exon 1 |
| 950 | CDK8 gRNA2 | AGACGTGACCATAAGTGCCT | Exon 1 |
| 951 | CDK8 gRNA3 | GCGCCGCCCAGCGCGGACAC | Intron 1 |
| 956 | CCNC gRNA1 | ATTGGTTCAAATTGTATAGT | Intron 2 |
| 957 | CCNC gRNA2 | AGAGAAACTTTAAATCCTTT | Exon 2 |
| 958 | CCNC gRNA3 | TTCTAGTTTGCAATGGATTT | Exon 2 |
| 999 | CCNC gRNA4 | TAGGCAAAGATCCGTTCTGT | Exon 9 |
| 961 | CCNC gRNA5 | TCTGTTGAAGGAGCGCCAAA | Exon 2 |
| 1000 | CCNC gRNA6 | ACCTTTGCTCCAGTATGTGC | Exon 8 |
| 1001 | CCNC gRNA7 | ATACCTAAAGCTATCATGAA | Exon 9 |
| 963 | ID3 gRNA1 | CTCCGGGTACCAGTTCCCGC | Exon 1 |
| 964 | ID3 gRNA2 | CTCAGCGGCTCCTCAGCTGC | Exon 1 |
| 965 | ID3 gRNA3 | CAGCATGAAGGCGCTGAGCC | Exon 1 |
| 969 | ID3 gRNA7 | TGGCCAGACTGCGTTCCGAC | Exon 1 |
| 970 | SOX4 gRNA1 | TTCCGTGTTCTCGGCATTGT | Exon 1 |
| 971 | SOX4 gRNA2 | GGCGATTCCCAGCTCGAGGC | Exon 1 |
| 972 | SOX4 gRNA4 | GCTGGTGCAAGACCCCGAGT | Exon 1 |
| 1012 | CISH gRNA9 | GCAGGCACCAGGATGCCTGG | Exon 3 |
| 1013 | CISH gRNA10 | GCATAGAGCTGGTCTCACTG | Exon 3 |

On day 7, using elevated IL2 in the media, the cells were transduced with a viral vector encoding a CD70 targeting CAR as provided for herein. At day 11, phenotype was evaluated, and cells were maintained at a low concentration of IL2 until functional assays were performed at day 14.

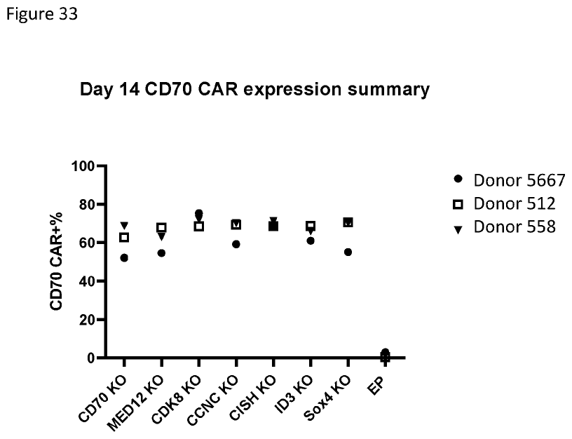

FIG. 33 shows data related to expression of a non-limiting embodiment of a CD70 targeting CAR by NK cells from three different donors edited at the indicated target genes by CRISPR/Cas9 and target-directed gRNA. Edits to MED12, CDK8, CCNC, ID3 and SOX4 were made using MED 12 gRNA pool (SEQ ID NOs: 938-940), CDK8 gRNA pool (SEQ ID NOs: 949-951), CCNC gRNA pool (SEQ ID NOs: 956-958), ID3 gRNA pool (SEQ ID NOs: 963-965) and SOX4 gRNA pool (SEQ ID NOs: 970-972) respectively. Edits to CISH were made using commercially available CISH-targeting gRNA sequences. As shown in FIG. 33, editing of any one of MED12, CDK8, CCNC, CISH, ID3 or SOX4 did not negatively impact the expression of CD70 CAR. Additionally, genome editing outcomes of particular targets were analyzed to determine gene editing efficiency. Table E3 shows that editing efficiency (% cells edited) was similar across cells from three donors for edited MED12 and ID3 genes. These data indicate that gene editing of selected targets can be accomplished while maintaining significant levels of expression of a tumor-directed CAR.

TABLE E3
Editing Efficiency

| | gRNAs | | | |
|---|---|---|---|---|
| Target | (SEQ ID NOS) | Donor 5667 | Donor 512 | Donor 558 |
| MED12 | 938-940 | 74.33% | 57.98% | 72.28% |
| ID3 | 963-965 | 98.94% | 98.73% | 98.73% |

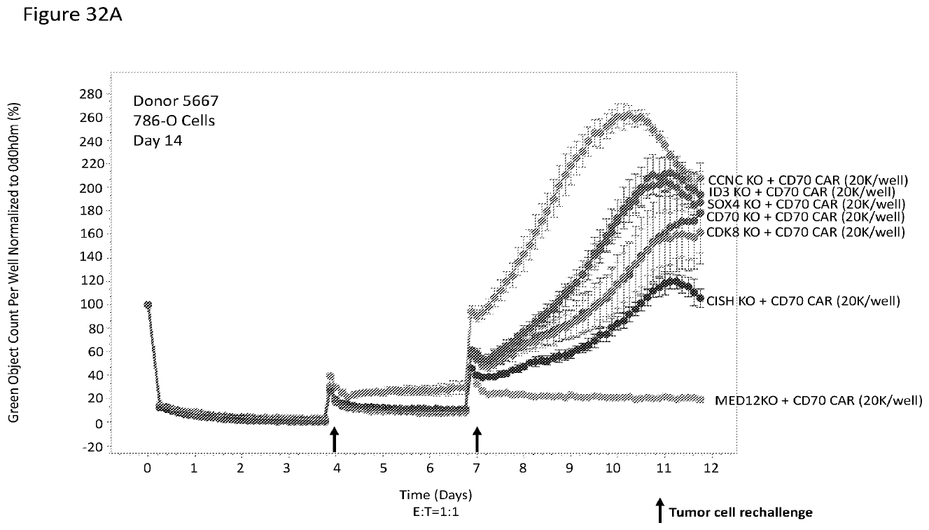

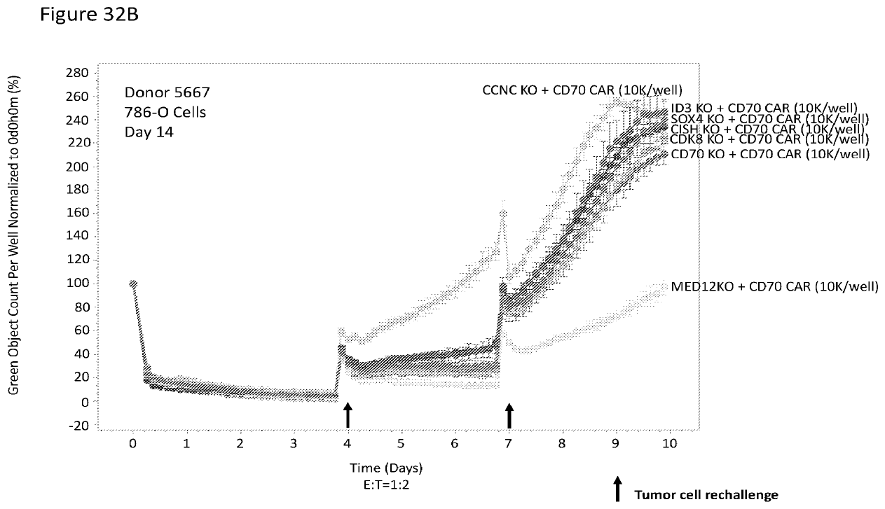

Next, cytotoxicity of the edited NK cells was assessed against the 786-0 adenocarcinoma cell line. NK cells expressing a CD70-directed CAR and edited on day 6 as indicated were incubated with 786-0 cells at a 1:1 (FIG. 32A), or 1:2 (FIG. 32B) E:T ratio beginning at day 14. Tumor cells were re-provided as a rechallenge 4 and 7 days later. As shown in FIG. 32A-32B, the cells expressing a CD70 CAR and edited at MED12 demonstrated the highest cytotoxicity at both E:T ratios. Thus, MED12, CCNC, CDK8, ID3, and SOX4 represented additional editing targets that can enhance cytotoxicity of CAR expressing NK cells. Similar results were seen with two other donors (data not shown).

Effects of these singular edits on cell expansion were also evaluated. Cells from a healthy donor were expanded as a single lot until editing at various targets with the example gRNA sequences provided in Table E2 on Day 6. As shown in Table E4, the editing of each of the targets in this example still allowed for robust expansion from day 1 to 14, with CD70 KO cells expanding over 1900-fold over 14 days and cells edited at an additional target gene expanding from about 1500-fold (CD70/CISH edited) to about 2500-fold (CD70/CDK8 edited). A similar expansion pattern was seen with a second donor (data not shown). Thus, expression of a CD70-directed CAR as well as editing at a variety of target sites, in addition to CD70 KO, does not reduce the ability of the cells to expand robustly in culture. Rather, this ability is maintained across the panel of editing targets.

TABLE E4
Expansion of Additional Target Edited NK Cells from Donor 512

Fold expansion

| Target | Day 0-6 | Day 7-14 | Day 0-14 |
|---|---|---|---|
| CD70 KO | 84.3 | 23.56 | 1985.83 |
| CD70/MED12 KO | 84.3 | 25.09 | 2115.38 |
| CD70/CDK8 KO | 84.3 | 29.22 | 2463.50 |
| CD70/CCNC KO | 84.3 | 27.95 | 2356.16 |
| CD70/CISH KO | 84.3 | 18.02 | 1519.09 |
| CD70/ID3 KO | 84.3 | 26.27 | 2214.59 |
| CD70/SOX4 KO | 84.3 | 26.79 | 2258.16 |

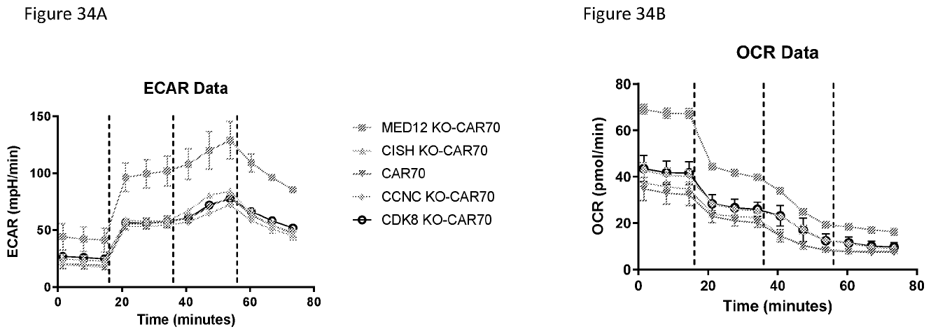

Next, glycolytic capacity in NK cells expressing a non-limiting embodiment of a CD70-targeting CAR and edited as indicated was determined by assessing extracellular acidification rate (ECAR) and oxygen consumption rate (OCR) as described in Example 1. During the glycolysis stress test. MED12 KO edited CD70 CAR NK cells (squares) showed higher levels of glycolysis FIG. 34A and glycolytic capacity FIG. 34B than other edited and unedited CD70 CAR NK cells.

Taken together, these data indicate that editing of the exemplary gene targets does not disrupt CD70-CAR expression, allows for robust expansion capacity of host cells (e.g., NK cells), and can enhance cytotoxicity against target tumor cells.

### Example 7—Screening of Additional Singular and Multiplex Editing Targets in CD19 CAR Expressing Cells

As an additional non-limiting embodiment, screening of various non-limiting examples of singular and multiplex editing targets were undertaken in NK cells from a healthy donor that were engineered to express a CD19-targeting CAR. These cells, in accordance with several embodiments disclosed herein, were also engineered to express mbIL15. Cells were engineered, edited, and assessed substantially as described in Example 2 and illustrated in FIG. 11, except using a non-limiting example of a CD19 CAR. MED12, CISH, and CBLB were knocked out separately or in combination, with single, double and triple knockouts being generated. As controls, NK cells from the same donor were subjected to mock electroporation and transduction (EP) or mock electroporation and transduction with the CD19 CAR (EP+CD19 CAR).

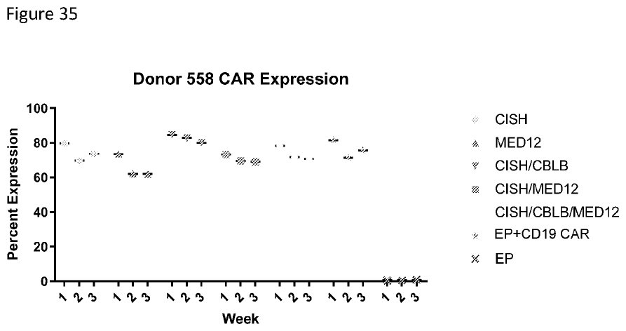

FIG. 35 shows data related to expression of a non-limiting embodiment of a CD19 targeting CAR by NK cells edited at the indicated target genes. As shown in FIG. 35, single, double or triple knockouts of any of MED12, CISH, and/or CBLB resulted in comparable expression of the CD19 CAR as compared to NK cells subjected to mock electroporation (EP+CD19 CAR). The data indicate that these candidate gene editing targets are compatible with CAR engineering and expression. Similar results were seen with another donor (data not shown).

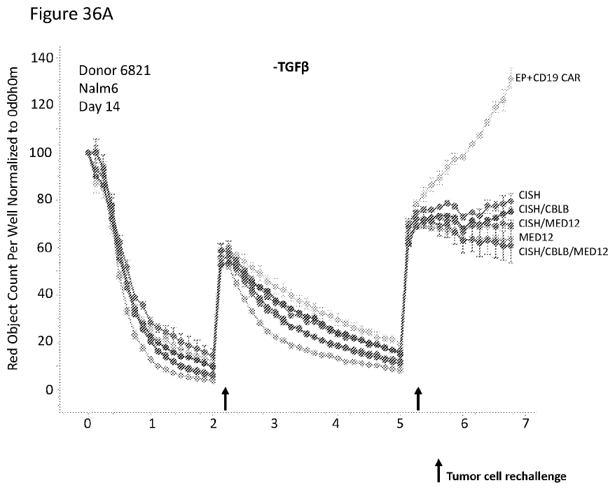

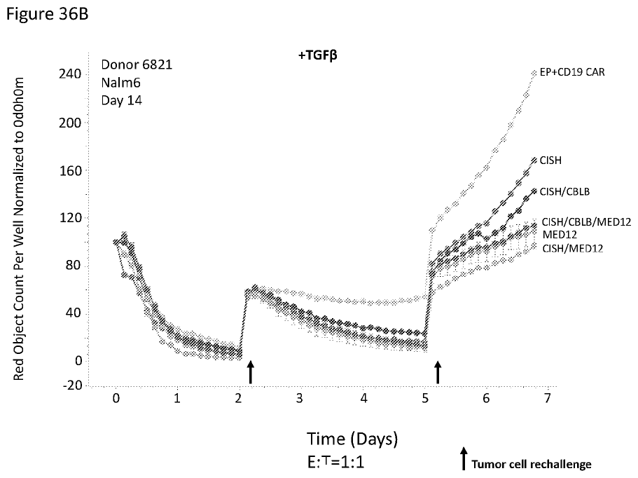

Next, cytotoxicity of the edited NK cells was assessed against the Nalm6 cancer cell line in the absence (FIG. 36A) or presence (FIG. 36B) of TGF-P. NK cells expressing a CD19-directed CAR were edited at indicated at day 6 and subsequently incubated with target Nalm6 cells at a 1:1 E:T ratio beginning at day 14. The NK cells were re-challenged with fresh Nalm6 cells approximately 2 and 5 days later. As shown in FIGS. 36A and 36B, the cells expressing a CD19 CAR and edited at MED12 alone (MED12), CISH and MED12 (CISH/MED12), or CISH, CBLB, and MED12 (CISH/CBLB/MED12) demonstrated the highest cytotoxicity. Thus, MED12, CBLB and CISH represent editing targets that can enhance cytotoxicity of CAR expressing NK cells, including in the presence of cytokines such as TGF-B. Similar results were seen with another donor (data not shown).

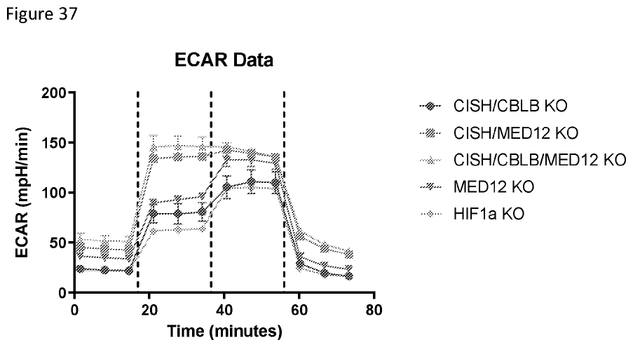

To test glycolysis of the NK cells expressing a non-limiting embodiment of a CD19-targeting CAR and edited as indicated, the NK cells were assessed for ECAR as previously described. NK cells expressing the exemplary CD19-directed CAR that were dual edited at CISH/MED12 (squares) or triple edited at CISH/CBLB/MED12 (triangles) showed higher levels of glycolysis compared to NK cells expressing the CD19-directed CAR that were dually edited at CISH/CBLB (circles) or singly edited at MED12 (inverted triangles) or HIF1alpha (diamonds) (FIG. 37).

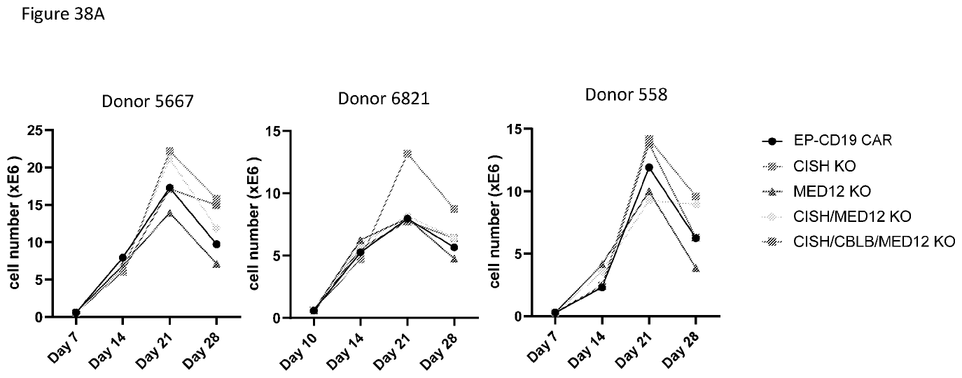

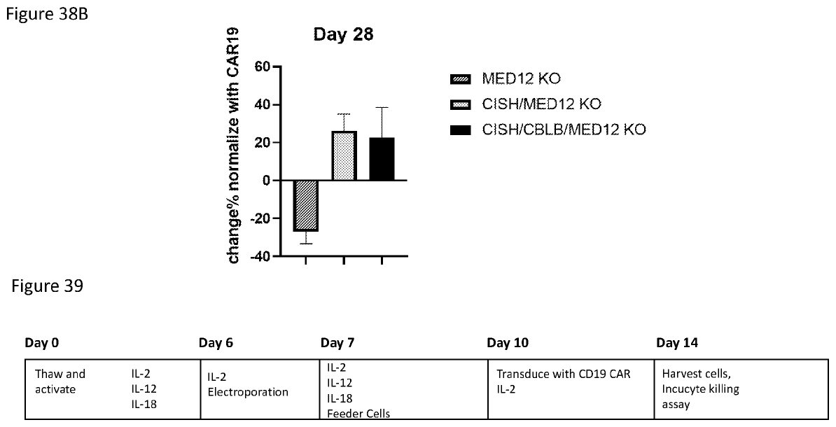

To test whether the edits as indicated affect the proliferative ability of the NK cells, proliferation of edited CD19-CAR NK cells from three different healthy donors was monitored over a period of 4 weeks. Edits to CISH, MED12, and CBLB were made using the CISH gRNA10 (SEQ ID NO:1013; GCATAGAGCTGGTCTCACTG), MED12 gRNA5 (SEQ ID NO:997; TAACTGCTCCCATAAGTACT), and CBLB gRNA7 (SEQ ID NO:1008; TAATCTGGTGGACCTCATGA), respectively. As illustrated in FIG. 38A and quantified in FIG. 38B, proliferation of MED12 edited CD19-CAR NK cells (triangles) was lowest in all three donors, but this effect appeared to be at least partially rescued by CISH KO (circles).

In a further experiment, NK cells isolated from three different healthy donors were either unmodified, engineered to express a non-limiting embodiment of a CD19 CAR, or engineered to express a non-limiting embodiment of a CD19 CAR and edited at MED12 (e.g., using MED12 gRNA5). Cells were subjected to genotyping and whole genome karyotyping by KaryoStat® assay. No chromosomal aberrations were observed in any of the donor cells, regardless of CAR engineering and MED12 editing status (data not shown).

### Example 8—Investigation of the Phenotypes, Proliferation and Cytotoxicity of Multiplex Edited CD19 CAR NK Cells

FIG. 39 shows a schematic workflow for assessing edits to multiple targets in, by way of example, CD19-CAR NK cells in vivo. Cells were cultured in a media comprising IL2, IL12, and IL18 for 6 days. On day 6, an increased concentration of IL2 was added to the media in advance of electroporation to deliver the gene editing machinery to edit MED12, CISH, and CBLB using non-limiting embodiments of MED12 gRNA5, CISH gRNA10 and CBLB gRNA7 corresponding to SEQ ID NOS: 997, 1013 and 1008, respectively, with single, double and triple knockouts being generated. Table E2 shows other non-limiting examples of gRNAs that can be used to edit these genes. On day 7, cells were cultured in a media comprising IL-2, IL12, and ILl 8 in the presence of feeder cells until day 10 when an increased concentration of IL2 was added to the media. Transduction of the CD19 directed CAR occurred on day 10. Cells were maintained with an elevated level of IL-2 until day 13, and then moved to a lower IL2 concentration for 1 or more additional day(s), before the cells were collected for functional assays. As controls, NK cells were subjected to mock electroporation and transduced with the exemplary CD19-directed CAR (EP).

Genome editing outcomes were analyzed to determine gene editing efficiency. Table E5 shows that in vivo editing efficiency (provided as % edited cells) of each target was similar across single, double or triple edits of MED12, CISH and CBLB genes that were knocked down using MED12 gRNA5, CISH gRNA10 and CBLB gRNA7. These data indicate that efficient gene editing of selected targets can be accomplished regardless of whether single or multiple targets are being edited.

TABLE E5
Editing Efficiency of Multiplex targets in vivo

| | MED12 | CISH | CBLB |
|---|---|---|---|
| Target | KO % | KO % | KO % |
| CISH | — | 93.3% | — |
| MED12 | 79.0% | — | — |
| CBLB | — | — | 90.3% |
| CISH-B3/CBLB | — | 90.8% | 90.5% |
| CISH-B3/MED12 | 89.5% | 92.9% | — |
| CBLB/MED12 | 90.1% | — | 96.2% |
| CISH/CBLB/MED12 | 95.9% | 95.9% | 97.3% |

Next, an evaluation of the expansion capacity of NK cells isolated from a healthy donor sample and edited on Day 6 was undertaken. As shown in Table E6, single, double or triple editing of the targets using the gRNAs sequences provided above (MED12 gRNA5, CISH gRNA10 and CBLB gRNA7) allowed for robust expansion from day 1 to 14, with cells expanding from about 2500-fold (MED12 edited) to nearly 6000-fold (CISH/MED12 edited) or to nearly 4800-fold (CISH/CBLB/MED12 edited). In particular, NK cells edited at CISH/MED12 exhibited expansion comparable to that of NK cells subjected to mock electroporation (EP).

TABLE E6
Expansion of Multiplex Edited NK Cells in vivo

Fold Expansion

| Target | D 0-6 | D 6-10 | D 10-14 | Total |
|---|---|---|---|---|
| CISH | 68 | 5.13 | 9.10 | 3176.5 |
| MED12 | 68 | 4.27 | 8.68 | 2521.6 |
| CBLB | 68 | 5.22 | 8.93 | 3165.5 |
| CISH/CBLB | 68 | 7.56 | 8.13 | 4176.9 |
| CISH/MED12 | 68 | 10.64 | 8.40 | 6075.8 |
| CBLB/MED12 | 68 | 11.34 | 7.80 | 6016.4 |
| CISH/CBLB/MED12 | 68 | 10.61 | 6.68 | 4815.9 |
| EP | 68 | 12.86 | 6.88 | 6013.6 |

These data indicate that gene editing of selected targets can be accomplished while maintaining the expansion capacity across the panel of examples of editing targets.

Table E7 shows data related to expression of a non-limiting embodiment of a CD19 targeting CAR by NK cells edited at the indicated target genes. As shown in Table E7, single, double or triple knockouts of any of MED12, CISH, or CBLB (using MED12 gRNA5, CISH gRNA10 and CBLB gRNA7), did not negatively impact the percentage of cells expressing the CD19 CAR. Each of the edited cell populations exhibited an increased percentage of cells expressing the CD19 CAR as compared to the untransduced NK cells, indicating that multiple edits are compatible with CAR expression.

TABLE E7
Expression of Multiplex Edited NK Cells in vivo

| | % Cells Expressing |
|---|---|
| Target | CD19 CAR |
| CISH KO | 70 |
| CBLB KO | 68.7 |
| MED12 KO | 63.3 |
| CISH/CBLB KO | 66.3 |
| CBLB/MED12 KO | 61.2 |
| CISH/MED12 KO | 61 |
| CISH/CBLB/MED12 KO | 56.7 |
| EP + CD19 CAR | 66.4 |
| Untransduced | 3.42 (background) |

As shown in Table E8, cells expressing a CD19 CAR and edited at MED12 alone or in combination with other edits showed an increase in the CD56dim cell population.

TABLE E8
CD56^{dim} Cell Population in Multiplex Edited NK Cells in vivo

| Target | % CD56^{dim} cell population |
|---|---|
| CISH KO | 16 |
| CBLB KO | 12.8 |
| MED12 KO | 44.6 |
| CISH/CBLB KO | 13.1 |
| CBLB/MED12 KO | 49 |
| CISH/MED12 KO | 48.8 |
| CISH/CBLB/MED12 KO | 48 |
| EP + CD19 CAR | 8.96 |
| Untransduced | 19.3 |

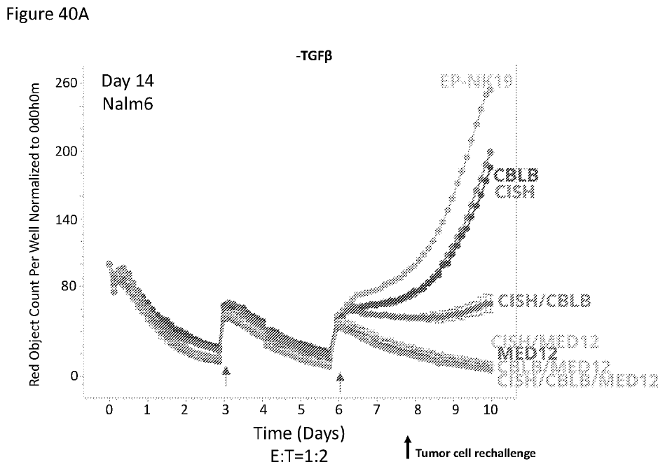

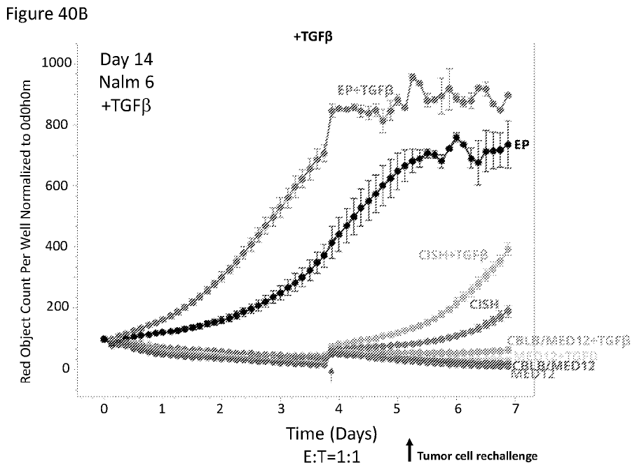

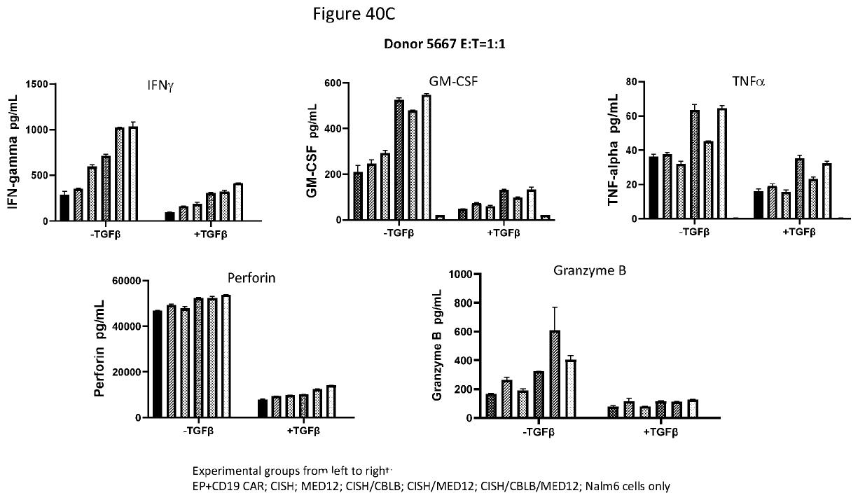

Next, cytotoxicity of the edited NK cells was assessed in vitro. NK cells expressing a CD19-directed CAR were edited at day 6 using the gRNA sequences provided above and incubated beginning on day 14 in the absence (FIG. 40A) or presence (FIG. 40B) of TGF-β with Nalm6 target cells at a 1:2 or 1:1 E:T ratio, respectively. As shown in FIG. 40A, the cells expressing a CD19 CAR and edited at MED12 alone, or in combination with CBLB, CISH. or CISH and CBLB demonstrated the highest cytotoxicity. Similar results were observed when co-culture of NK and Nalm6 target cells was extended to Day 54. As shown in FIG. 40B, even in the presence of TGF-P, the cells expressing a CD19 CAR and edited at MED12 alone or in combination with CBLB demonstrated the highest cytotoxicity.

NK cells from a healthy donor were engineered to express a non-limiting embodiment of a CD19-directed CAR and edited on day 6 at CISH, MED12, CISH/CBLB, CISH/MED12, or CISH/CBLB/MED12 (using MED12 gRNA5, CISH gRNA10 and CBLB gRNA7). As a control, NK cells from the same donor were engineered to express the CD19-directed CAR and mock electroporated on day 6 (EP+CD19 CAR). Beginning on day 21, the NK cells were co-cultured with Nalm6 target cells at an E:T ratio of 1:1 in the presence or absence of TGF-B. After three days, cytokine production was assessed in co-culture supernatant by Luminex® multiplex assay. Knockout of MED12 alone or in combination with CBLB and/or CISH was generally observed to increase secretion of cytokines and cytotoxic proteins by NK cells (FIG. 40C). Similar results were observed with NK cells from a second donor (data not shown).

CD19 CAR-expressing NK cells that were edited at MED12 or mock electroporated were analyzed for cell surface expression of various activation markers. CD19 CAR NK cells edited at MED12 demonstrated increased levels of activation markers such as CD25, NKp44, NKp46, and CD69 compared to the EP NK cells (data not shown).

### Example 9—In Vivo Analysis of Multiplex Edited CAR NK Cells

NK cells were isolated from healthy human donors and were edited by CRISPR/Cas9 to disrupt MED12, CBLB, CISH, and combinations thereof using the MED12 gRNA5, CBLB gRNA7, and CISH gRNA10 sequences described in the preceding Examples. Edited cells were also engineered to express a non-limiting embodiment of a CD19-directed CAR and optionally a membrane-bound interleukin 15 (mbIL15).

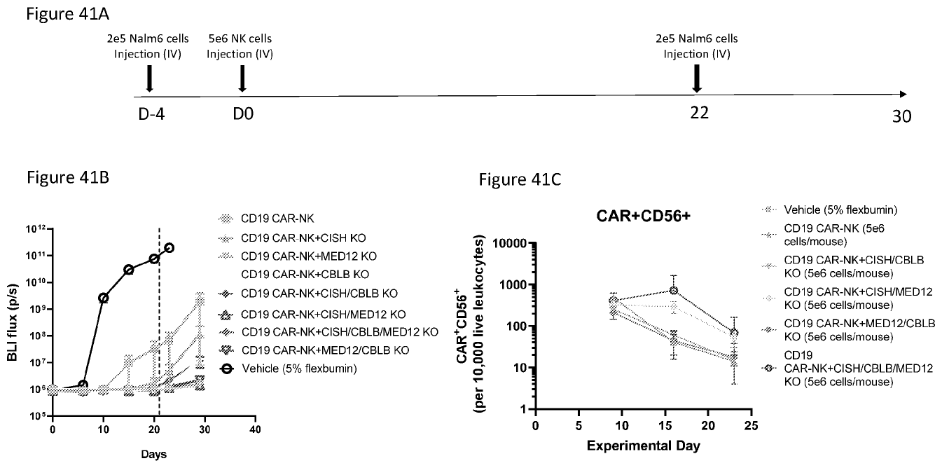

2×10⁵ Nalm6 cells were injected into NOD scid gamma (NSG) mice at day −4 and day 22. Vehicle or 5×10⁶ NK cells expressing the CD19-directed CAR and edited as indicated were injected into mice at day 0 and tumor burden was assessed over a 30-day period by bioluminescence imaging (BLI) as illustrated in FIG. 41A. As shown in FIG. 41B, cells edited to express a CD19-directed CAR and edited at MED12 alone, or in combination with CBLB, CISH, or CISH and CBLB were the most effective at controlling tumor burden. Persistence of the injected NK cells was also assessed in the mice. As shown in FIG. 41C, cells engineered to express a CD19-directed CAR and edited at CISH/MED12 (closed circle) or at CISH/CBLB/MED12 (open circle) displayed an increased persistence compared to the other groups. Together, these data are consistent with a finding that CAR NK cells edited at MED12 alone or in combination with other targets such as CISH and CBLB displayed in vivo persistence and tumor control.

### Example 10—Gene Editing of BCMA CAR NK Cells

As an additional non-limiting embodiment, screening of various non-limiting examples of singular and multiplex editing targets were undertaken in NK cells from two different healthy donors that were engineered to express a CAR targeting B cell maturation antigen (BCMA) and optionally a membrane-bound interleukin 15 (mbIL15). Cells were engineered, edited, and assessed substantially as described in Example 6 and illustrated in FIG. 31, except using non-limiting example of BCMA CARs and editing the cells at genetic loci encoding MED12 and CISH.

The NK cells were knocked out for MED12 alone (MED12 KO) or in combination with CISH (MED12/CISH KO) on Day 6 of culture via electroporation to deliver target-directed gRNAs and CRISPR/Cas9. On Day 7, the edited NK cells were transduced with a vector encoding a BCMA-targeting CAR incorporating an extracellular antigen-binding domain comprised of two anti-BCMA V_{H}Hs targeting different epitopes (e.g., SEQ ID NOS: 1087 and 1088) and connected by a Gly-Ser linker (e.g., SEQ ID NO:1015), a CD8-derived hinge and transmembrane domain (e.g., SEQ ID NO:14), either a 4-1BB (“BCMA1”; e.g., SEQ ID NO:1089) or OX-40 (“BCMA2”; e.g., SEQ ID NO:6) co-stimulatory domain, and a CD3zeta primary signaling domain (e.g., SEQ ID NO:8). As controls, NK cells from the same donor were subjected to mock electroporation and transduction with the BCMA CAR (BCMA CAR only).

The phenotype of the engineered and edited NK cells was assessed beginning on Day 10 of culture. Knockout of MED12 and CISH was confirmed in NK cells from both donors and was not observed to significantly affect the expansion or viability of the NK cells in culture. Between approximately 42-62% of the edited NK cells were observed to express the BCMA1 CAR, and between approximately 65-82% of edited NK cells were observed the express the BCMA2 CAR.

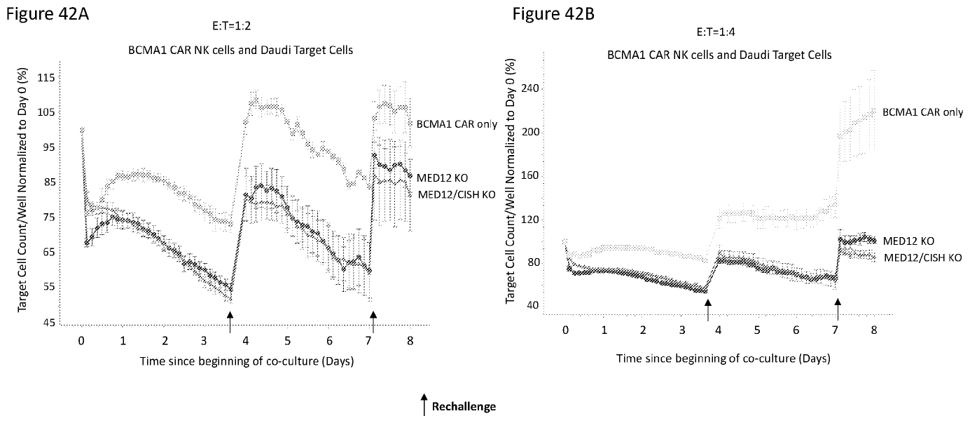

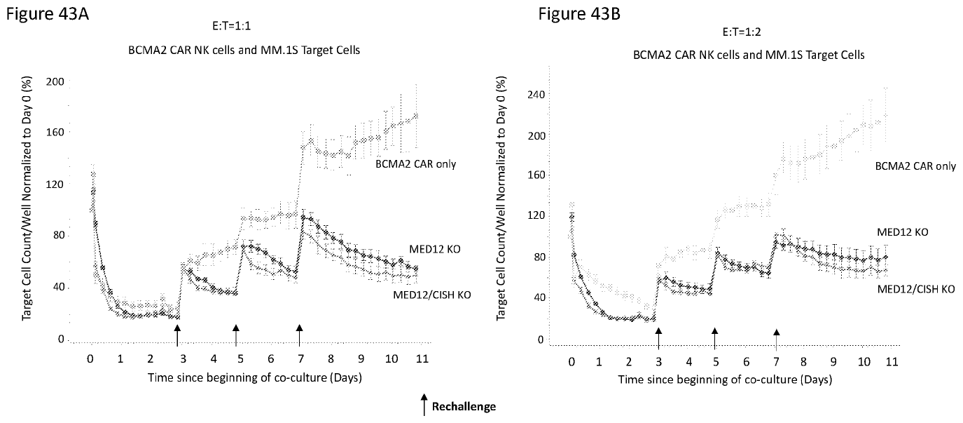

Beginning on Day 14, the engineered and edited NK cells were co-cultured with BCMA-expressing target cells and Incucyte® assays were performed to monitor the cytotoxicity of the NK cells against the target cells. Specifically, engineered and edited NK cells from two different donors were cultured with relatively low BCMA-expressing Daudi target cells or relatively high BCMA-expressing MM.1S target cells at various effector-to-target cell (E:T) ratios. Daudi target cells were re-provided as a rechallenge approximately four and seven days later, while MM.1S target cells were re-provided as a rechallenge approximately three, five, and seven days later. NK cells expressing the BCMA1 CAR and knocked out for MED12 alone or in combination with CISH demonstrated increased cytotoxicity against Daudi target cells at E:T ratios of 1:2 and 1:4 as compared to unedited BCMA1 CAR-expressing NK cells (representative data for both donors shown in FIGS. 42A-B). NK cells expressing the BCMA2 CAR and knocked out for MED12 alone or in combination with CISH demonstrated increased cytotoxicity against MM.1S target cells at E:T ratios of 1:1 and 1:2 as compared to unedited BCMA2 CAR-expressing NK cells (representative data for both donors shown in FIGS. 43A-B). Similar results were observed when BCMA2 CAR-expressing NK cells and MM.1S target cells were co-cultured at a 1:1 E:T ratio beginning on Day 21 (data not shown).

Without wishing to be bound by theory, the data indicate that knockout of MED 12 alone or in combination with CISH consistently increases target-specific cytotoxicity by NK cells, including when different CARs and CAR targets are assessed.

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. In addition, where features or aspects of the disclosure are described in terms of Markush groups, those skilled in the art will recognize that the disclosure is also thereby described in terms of any individual member or subgroup of members of the Markush group.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

### Sequences

In several embodiments, there are provided amino acid sequences that correspond to any of the nucleic acids disclosed herein (and/or included in the accompanying sequence listing), while accounting for degeneracy of the nucleic acid code. Furthermore, those sequences (whether nucleic acid or amino acid) that vary from those expressly disclosed herein (and/or included in the accompanying sequence listing), but have functional similarity or equivalency are also contemplated within the scope of the present disclosure. The foregoing includes mutants, truncations, substitutions, codon optimization, or other types of modifications.

In accordance with some embodiments described herein, any of the sequences may be used, or a truncated or mutated form of any of the sequences disclosed herein (and/or included in the accompanying sequence listing) may be used and in any combination. Sequences provided for herein that include an identifier, such as a tag or other detectable sequence (e.g., a Flag tag) are also provided for herein with the absence of such a tag or other detectable sequence (e.g., excluding the Flag tag from the listed sequence). A Sequence Listing in electronic format is submitted herewith. Some of the sequences provided in the Sequence Listing may be designated as Artificial Sequences by virtue of being non-naturally occurring fragments or portions of other sequences, including naturally occurring sequences. Some of the sequences provided in the Sequence Listing may be designated as Artificial Sequences by virtue of being combinations of sequences from different origins, such as humanized antibody sequences.

1-82. (canceled)

83. A population of gene edited natural killer (NK) cells, wherein:
the NK cells are genetically edited within a target sequence in a MED12 gene and within a target sequence in a CISH gene; and
the edits yield reduced expression of each of the MED12 protein encoded by the MED12 gene and the CIS protein encoded by the CISH gene, as compared to a NK cell not edited within the target sequence in the MED12 gene and the target sequence within the CISH gene.

84. The population of gene edited NK cells of claim 83, wherein the target sequence within the MED12 gene comprises any one of SEQ ID NOS: 938-944 and 996-998.

85. The population of gene edited NK cells of claim 83, wherein the target sequence in the CISH gene comprises any one of SEQ ID NOS: 153-157, 463-466, 1012, and 1013.

86. The population of gene edited NK cells of claim 83, wherein the NK cells express a cytotoxic receptor comprising an extracellular ligand binding domain, a transmembrane domain, and a cytotoxic signaling complex.

87. The population of gene edited NK cells of claim 86, wherein the cytotoxic signaling complex comprises a CD3zeta domain and an intracellular signaling domain of OX40 or a signaling portion thereof.

88. The population of gene edited NK cells of claim 83, wherein the NK cells express a membrane-bound interleukin-15 (mbIL15).

89. A composition comprising the population of gene edited NK cells of claim 83.

90. A method for the treatment of a subject having a disease or condition comprising administering to the subject the composition of claim 89.

91. A composition comprising the population of gene edited NK cells of claim 88.

92. A method for the treatment of a subject having a disease or condition comprising administering to the subject the composition of claim 91.

93. A population of gene edited immune cells, wherein:
the immune cells are genetically edited within a target sequence in a MED12 gene comprising any one of SEQ ID NOS: 938-944 and 996-998; and
the edit yields reduced expression of a MED12 protein encoded by the MED12 gene, as compared to an immune cell not edited within the target sequence in the MED12 gene.

94. A composition comprising the population of gene edited immune cells of claim 93.

95. A method for the treatment of a subject having a disease or condition comprising administering to the subject the composition of claim 94.

96. A population of gene edited immune cells, wherein:
the immune cells are genetically edited within a target sequence in a disintegrin and metalloproteinase domain-containing protein 17 (ADAM17) gene comprising any one of SEQ ID NOS: 682-687; and
the edit yields reduced expression of an ADAM17 protein encoded by the ADAM17 gene, as compared to an immune cell not edited within the target sequence in the ADAM17 gene.

97. A composition comprising the population of gene edited immune cells of claim 96.

98. A population of gene edited immune cells, wherein:
the immune cells are genetically edited within a target sequence in a hypoxia-inducible factor 1-alpha (HIF1-a) gene comprising any one of SEQ ID NOS: 750-760; and
the edit yields reduced expression of the HIF1-a protein encoded by the HIF1-a gene, as compared to an immune cell not edited within the target sequence in the HIF1-a gene.

99. A composition comprising the population of gene edited immune cells of claim 98.

100. A method of manufacturing a population of genetically edited natural killer (NK) cells comprising:
(a) contacting a population of NK cells with a first RNA-guided endonuclease,
wherein the RNA-guided endonuclease edits within a target sequence in the MED12 gene to yield reduced levels of expression of the MED12 protein encoded by the MED12 gene, as compared to an immune cell not edited within the target sequence in the MED12 gene; and
(b) contacting the population of NK cells with a second RNA-guided endonuclease,
wherein the second RNA guided endonuclease edits within a target sequence in the CISH gene to yield reduced levels of expression of the CIS protein encoded by the CISH gene, as compared to an immune cell not edited within the target sequence in the CISH gene.

101. The method of claim 100, wherein the target sequence within the MED12 gene comprises any one of SEQ ID NOS: 938-944 and 996-998.

102. The method of claim 100, wherein the target sequence in the CISH gene comprises any one of SEQ ID NOS: 153-157, 463-466, 1012, and 1013.
